SEQUENCE LISTING (262 sequences)

SEQ ID NO: 1 - protein, 5 aa, synthetic construct
SYGMH

SEQ ID NO: 2 - protein, 5 aa, synthetic construct
GYYWS

SEQ ID NO: 3 - protein, 5 aa, synthetic construct
GYYWS

SEQ ID NO: 4 - protein, 5 aa, synthetic construct
GYYWS

SEQ ID NO: 5 - protein, 5 aa, synthetic construct
SYYWS

SEQ ID NO: 6 - protein, 5 aa, synthetic construct
NYWVG

SEQ ID NO: 7 - protein, 5 aa, synthetic construct
SYAMS

SEQ ID NO: 8 - protein, 5 aa, synthetic construct
DYAMS

SEQ ID NO: 9 - protein, 5 aa, synthetic construct
NYWMH

SEQ ID NO: 10 - protein, 5 aa, synthetic construct
SYYWS

SEQ ID NO: 11 - protein, 7 aa, synthetic construct
TTGYHWN

SEQ ID NO: 12 - protein, 7 aa, synthetic construct
TTGYHWN

SEQ ID NO: 13 - protein, 5 aa, synthetic construct
DYVIN

SEQ ID NO: 14 - protein, 17 aa, synthetic construct
FIRYDGSNKYYADSVKG

SEQ ID NO: 15 - protein, 16 aa, synthetic construct
EIDHSGSTNYNPSLKS

SEQ ID NO: 16 - protein, 16 aa, synthetic construct
EIDHSGSTNYNPSLKS

SEQ ID NO: 17 - protein, 16 aa, synthetic construct
EIDHSGSTNYNPSLKS

SEQ ID NO: 18 - protein, 16 aa, synthetic construct
HISYSGSANYNPSLKS

SEQ ID NO: 19 - protein, 17 aa, synthetic construct
IIYPGDSDTRYSPSFQG

SEQ ID NO: 20 - protein, 17 aa, synthetic construct
AISGSGGSTYYADSVKG

SEQ ID NO: 21 - protein, 17 aa, synthetic construct
YNSFSVGSTDYADSVKG

SEQ ID NO: 22 - protein, 17 aa, synthetic construct
EIDPSDIYTNYAQKFQG

SEQ ID NO: 23 - protein, 16 aa, synthetic construct
HISYSGSANYNPSLKS

SEQ ID NO: 24 - protein, 16 aa, synthetic construct
YIYSSGSTSYNPSLKS

SEQ ID NO: 25 - protein, 16 aa, synthetic construct
YIYSSGSTSYNPSLKS

SEQ ID NO: 26 - protein, 17 aa, synthetic construct
EIYPGSGTNYYNEKFKA

SEQ ID NO: 27 - protein, 12 aa, synthetic construct
DRGLGDGTYFDY

SEQ ID NO: 28 - protein, 9 aa, synthetic construct
ARGPWSFDP

SEQ ID NO: 29 - protein, 9 aa, synthetic construct
ARGPWSFDP

SEQ ID NO: 30 - protein, 9 aa, synthetic construct
ARGPWSFDP

SEQ ID NO: 31 - protein, 7 aa, synthetic construct
WDDAFNI

SEQ ID NO: 32 - protein, 14 aa, synthetic construct
LTMFRGIIIGYFDY

SEQ ID NO: 33 - protein, 9 aa, synthetic construct
ELYREYMDY

SEQ ID NO: 34 - protein, 12 aa, synthetic construct
HSGNYYTGPFHY

SEQ ID NO: 35 - protein, 13 aa, synthetic construct
GIYDGYYVYGMDY

SEQ ID NO: 36 - protein, 7 aa, synthetic construct
WEEAFNI

SEQ ID NO: 37 - protein, 8 aa, synthetic construct
GDWHYFDY

SEQ ID NO: 38 - protein, 8 aa, synthetic construct
GDWHYFDY

SEQ ID NO: 39 - protein, 11 aa, synthetic construct
RGRYGLYAMDY

SEQ ID NO: 40 - protein, 13 aa, synthetic construct
SGSSSNIGNNAVN

SEQ ID NO: 41 - protein, 11 aa, synthetic construct
RASQSISSWLA

SEQ ID NO: 42 - protein, 11 aa, synthetic construct
RASQSISSWLA

SEQ ID NO: 43 - protein, 11 aa, synthetic construct
RASQSISSWLA

SEQ ID NO: 44 - protein, 12 aa, synthetic construct
RASQSVSSSYLA

SEQ ID NO: 45 - protein, 11 aa, synthetic construct
RASQSVSSYLA

SEQ ID NO: 46 - protein, 11 aa, synthetic construct
QGDSLRSYYAS

SEQ ID NO: 47 - protein, 11 aa, synthetic construct
RASQGISSYLA

SEQ ID NO: 48 - protein, 11 aa, synthetic construct
RSSQDISNYLN

SEQ ID NO: 49 - protein, 11 aa, synthetic construct
RASQDISGALN

SEQ ID NO: 50 - protein, 11 aa, synthetic construct
SGEKLSDKYVH

SEQ ID NO: 51 - protein, 11 aa, synthetic construct
SGEKLSDKYVH

SEQ ID NO: 52 - protein, 11 aa, synthetic construct
RASQDISNYLN

SEQ ID NO: 53 - protein, 7 aa, synthetic construct
YDDLLPS

SEQ ID NO: 54 - protein, 7 aa, synthetic construct
KASSLES

SEQ ID NO: 55 - protein, 7 aa, synthetic construct
KASSLES

SEQ ID NO: 56 - protein, 7 aa, synthetic construct
KASSLES

SEQ ID NO: 57 - protein, 7 aa, synthetic construct
GASSRAT

SEQ ID NO: 58 - protein, 7 aa, synthetic construct
DASNRAT

SEQ ID NO: 59 - protein, 7 aa, synthetic construct
GKNNRPS

SEQ ID NO: 60 - protein, 7 aa, synthetic construct
AASSLES

SEQ ID NO: 61 - protein, 7 aa, synthetic construct
YTSRLHS

SEQ ID NO: 62 - protein, 7 aa, synthetic construct
AASSLQS

SEQ ID NO: 63 - protein, 7 aa, synthetic construct
ENDRRPS

SEQ ID NO: 64 - protein, 7 aa, synthetic construct
ENDRRPS

SEQ ID NO: 65 - protein, 7 aa, synthetic construct
YTSRLHS

SEQ ID NO: 66 - protein, 11 aa, synthetic construct
AAWDDSLNGPV

SEQ ID NO: 67 - protein, 9 aa, synthetic construct
QQYNSYPIT

SEQ ID NO: 68 - protein, 8 aa, synthetic construct
QQYNSYPT

SEQ ID NO: 69 - protein, 9 aa, synthetic construct
QQYGSFPIT

SEQ ID NO: 70 - protein, 9 aa, synthetic construct
QQYGSSPWT

SEQ ID NO: 71 - protein, 9 aa, synthetic construct
QQRSNWPWT

SEQ ID NO: 72 - protein, 12 aa, synthetic construct
NSRDSFSIHQNV

SEQ ID NO: 73 - protein, 9 aa, synthetic construct
QQSYSTPIT

SEQ ID NO: 74 - protein, 9 aa, synthetic construct
QQGKTLPRT

SEQ ID NO: 75 - protein, 9 aa, synthetic construct
QQANSFPLT

SEQ ID NO: 76 - protein, 10 aa, synthetic construct
QFWDSTNSAV

SEQ ID NO: 77 - protein, 10 aa, synthetic construct
QFWDSTNSAV

SEQ ID NO: 78 - protein, 9 aa, synthetic construct
QQGNTRPWT

SEQ ID NO: 79 - protein, 121 aa, synthetic construct
QVQLVESGGGLVKPGGSLRLSCAASGFTFSSYGMHWVRQAPGKGLEWVAFIRYDGSNKYYADSVKGRFTISRDNSKNTKYLQMNSLRAEDTAVYYCAKDRGLGDGTYFDYWGQGTTVTVSS

SEQ ID NO: 80 - protein, 117 aa, synthetic construct
QVQLQQWGAGLLKPSETLSLTCAVYGGSFSGYYWSWIRQPPGKGLEWIGEIDHSGSTNYNPSLKSRVTISVDTSKNQFSLKLSSVTAADTAVYYCARARGPWSFDPWGQGTLVTVSS

SEQ ID NO: 81 - protein, 117 aa, synthetic construct; identical to SEQ ID NO: 80

SEQ ID NO: 82 - protein, 117 aa, synthetic construct; identical to SEQ ID NO: 80

SEQ ID NO: 83 - protein, 115 aa, synthetic construct
QVHLQESGPGLVKPSETLSLTCTVSDDSISSYYWSWIRQPPGKGLEWIGHISYSGSANYNPSLKSRVTISVDTSKNQFSLKLSSVTAADTAVYYCANWDDAFNIWGQGTMVTVSS

SEQ ID NO: 84 - protein, 123 aa, synthetic construct
EVQLVQSGAEVKEPGESLKISCKNSGYSFTNYWVGWVRQMPGKGLEWMGIIYPGDSDTRYSPSFQGQVTISADKSINTAYLQWSSLKASDTAMYYCGRLTMFRGIIIGYFDYWGQGTLVTVSS

SEQ ID NO: 85 - protein, 118 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKELYREYMDYWGQGTLVTVSS

SEQ ID NO: 86 - protein, 121 aa, synthetic construct
EVQLLESGGGLVQPGGSLRLSCATSGFTFSDYAMSWVRQAPGKGLEWVSYNSFSVGSTDYADSVKGRFTISRDNSKNTLYLQMNSLRAEDTAVYYCSKHSGNYYTGPFHYWGQGTLVTVSS

SEQ ID NO: 87 - protein, 122 aa, synthetic construct
QVQLVQSGAEVKKPGASVKVSCKASGYTFTNYWMHWVRQAPGQGLEWMGEIDPSDIYTNYAQKFQGRVTMTRDTSTSTVYMELSSLRSEDTAVYYCARGIYDGYYVYGMDYWGQGTTVTVSS

SEQ ID NO: 88 - protein, 115 aa, synthetic construct
QVHLQESGPGLVKPSETLSLTCTVSEESISSYYWSWIRQPPGKGLEWIGHISYSGSANYNPSLKSRVTISVDTSKNQFSLKLSSVTAADTAVYYCANWEEAFNIWGQGTMVTVSS

SEQ ID NO: 89 - protein, 118 aa, synthetic construct
EIQLLESGGGLVQPGGSLRLSCAVSGFSITTTGYHWNWVRQAPGKGLEWVGYIYSSGSTSYNPSLKSRFTISRDTSKNTFYLQMNSLRAEDTAVYYCARGDWHYFDYWGQGTMVTVSS

SEQ ID NO: 90 - protein, 118 aa, synthetic construct; identical to SEQ ID NO: 89

SEQ ID NO: 91 - protein, 120 aa, synthetic construct
QVQLQQSGPELVKPGASVKMSCKASGYTFTDYVINWGKQRSGQGLEWIGEIYPGSGTNYYNEKFKAKATLTADKSSNIAYMQLSSLTSEDSAVYFCARRGRYGLYAMDYWGQGTSVTVSS

SEQ ID NO: 92 - protein, 110 aa, synthetic construct
QSALTQPASVSGSPGQSITISCSGSSSNIGNNAVNWYQQLPGKAPKLLIYYDDLLPSGVSDRFSGSKSGTSAFLAISGLQSEDEADYYCAAWDDSLNGPVFGGGTKLTVL

SEQ ID NO: 93 - protein, 107 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYNSYPITFGGGTKVEIK

SEQ ID NO: 94 - protein, 106 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYNSYPTFGGGTKVEIK

SEQ ID NO: 95 - protein, 107 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYGSFPITFGGGTKVEIK

SEQ ID NO: 96 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 97 - protein, 107 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPWTFGQGTKVEIK

SEQ ID NO: 98 - protein, 109 aa, synthetic construct
SSELTQDPAVSVALGQTVRITCQGDSLRSYYASWYQQKPGQAPVLVIYGKNNRPSGIPDRFSGSSSGNTASLTITGAQAEDEADYYCNSRDSFSIHQNVFGGGTKLTVL

SEQ ID NO: 99 - protein, 107 aa, synthetic construct
DIQLTQSPSFLSASVGDRVTITCRASQGISSYLAWYQQKPGKAPKLLIYAASSLESGVPSRFSGSGSGTDFTLTISSLQPEDFATYYCQQSYSTPITFGQGTRLEIK

SEQ ID NO: 100 - protein, 107 aa, synthetic construct
YIQMTQSPSSLSASVGDRVTITCRSSQDISNYLNWYQQKPGKAPKLLIYYTSRLHSGVPSRFSGSGSGTDFTFTISSLQPEDIATYYCQQGKTLPRTFGGGTKVEIK

SEQ ID NO: 101 - protein, 107 aa, synthetic construct
DIQMTQSPSSLSASVGDRVTITCRASQDISGALNWYQQKPGKAPNLLIYAASSLQSGVPSRFSGSGSGTDYTLTISSLQPEDFATYYCQQANSFPLTFGGGTKVEIK

SEQ ID NO: 102 - protein, 107 aa, synthetic construct
SSETTQPPSVSVSPGQTASITCSGEKLSDKYVHWYQQKPGQSPVMVIYENDRRPSGIPERFSGSNSGNTATLTISGTQAMDEADYFCQFWDSTNSAVFGGGTQLTVL

SEQ ID NO: 103 - protein, 108 aa, synthetic construct
DSVTTQSPLSLPVTLGQPASISCSGEKLSDKYVHWYQQRPGQSPRMLIYENDRRPSGVPDRFSGSNSGNDATLKISRVEAEDVGVYFCQFWDSTNSAVFGGGTKVEIK

SEQ ID NO: 104 - protein, 107 aa, synthetic construct
DIQMTQTTSSLSASLGDRVTISCRASQDISNYLNWYQQKPDGTVKLLIYYTSRLHSGVPSRFSGSGSGTDYSLTINNLEQEDIATYFCQQGNTRPWTFGGGTKLEIK

SEQ ID NO: 105 - protein, 328 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 106 - protein, 328 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 107 - protein, 107 aa, Homo sapiens [sequence omitted]

SEQ ID NO: 108 - protein, 105 aa, Homo sapiens
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTEC

SEQ ID NO: 109 - protein, 449 aa, synthetic construct
QVQLVESGGGLVKPGGSLRLSCAASGFTFSSYGMHWVRQAPGKGLEWVAFIRYDGSNKYYADSVKGRFTISRDNSKNTKYLQMNSLRAEDTAVYYCAKDRGLGDGTYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 110 - protein, 445 aa, synthetic construct
QVQLQQWGAGLLKPSETLSLTCAVYGGSFSGYYWSWIRQPPGKGLEWIGEIDHSGSTNYNPSLKSRVTISVDTSKNQFSLKLSSVTAADTAVYYCARARGPWSFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 111 - protein, 445 aa, synthetic construct; identical to SEQ ID NO: 110

SEQ ID NO: 112 - protein, 445 aa, synthetic construct; identical to SEQ ID NO: 110

SEQ ID NO: 113 - protein, 443 aa, synthetic construct
QVHLQESGPGLVKPSETLSLTCTVSDDSISSYYWSWIRQPPGKGLEWIGHISYSGSANYNPSLKSRVTISVDTSKNQFSLKLSSVTAADTAVYYCANWDDAFNIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 114 - protein, 451 aa, synthetic construct
EVQLVQSGAEVKEPGESLKISCKNSGYSFTNYWVGWVRQMPGKGLEWMGIIYPGDSDTRYSPSFQGQVTISADKSINTAYLQWSSLKASDTAMYYCGRLTMFRGIIIGYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 115 - protein, 446 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKELYREYMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 116 - protein, 449 aa, synthetic construct
EVQLLESGGGLVQPGGSLRLSCATSGFTFSDYAMSWVRQAPGKGLEWVSYNSFSVGSTDYADSVKGRFTISRDNSKNTLYLQMNSLRAEDTAVYYCSKHSGNYYTGPFHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 117 - protein, 450 aa, synthetic construct
QVQLVQSGAEVKKPGASVKVSCKASGYTFTNYWMHWVRQAPGQGLEWMGEIDPSDIYTNYAQKFQGRVTMTRDTSTSTVYMELSSLRSEDTAVYYCARGIYDGYYVYGMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 118 - protein, 443 aa, synthetic construct
QVHLQESGPGLVKPSETLSLTCTVSEESISSYYWSWIRQPPGKGLEWIGHISYSGSANYNPSLKSRVTISVDTSKNQFSLKLSSVTAADTAVYYCANWEEAFNIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 119 - protein, 446 aa, synthetic construct
EIQLLESGGGLVQPGGSLRLSCAVSGFSITTTGYHWNWVRQAPGKGLEWVGYIYSSGSTSYNPSLKSRFTISRDTSKNTFYLQMNSLRAEDTAVYYCARGDWHYFDYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 120 - protein, 446 aa, synthetic construct; identical to SEQ ID NO: 119

SEQ ID NO: 121 - protein, 448 aa, synthetic construct
QVQLQQSGPELVKPGASVKMSCKASGYTFTDYVINWGKQRSGQGLEWIGEIYPGSGTNYYNEKFKAKATLTADKSSNIAYMQLSSLTSEDSAVYFCARRGRYGLYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 122 - protein, 449 aa, synthetic construct
QVQLVESGGGLVKPGGSLRLSCAASGFTFSSYGMHWVRQAPGKGLEWVAFIRYDGSNKYYADSVKGRFTISRDNSKNTKYLQMNSLRAEDTAVYYCAKDRGLGDGTYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 123 - protein, 445 aa, synthetic construct
QVQLQQWGAGLLKPSETLSLTCAVYGGSFSGYYWSWIRQPPGKGLEWIGEIDHSGSTNYNPSLKSRVTISVDTSKNQFSLKLSSVTAADTAVYYCARARGPWSFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 124 - protein, 445 aa, synthetic construct; identical to SEQ ID NO: 123

SEQ ID NO: 125 - protein, 445 aa, synthetic construct; identical to SEQ ID NO: 123

SEQ ID NO: 126 - protein, 443 aa, synthetic construct
QVHLQESGPGLVKPSETLSLTCTVSDDSISSYYWSWIRQPPGKGLEWIGHISYSGSANYNPSLKSRVTISVDTSKNQFSLKLSSVTAADTAVYYCANWDDAFNIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 127 - protein, 451 aa, synthetic construct
EVQLVQSGAEVKEPGESLKISCKNSGYSFTNYWVGWVRQMPGKGLEWMGIIYPGDSDTRYSPSFQGQVTISADKSINTAYLQWSSLKASDTAMYYCGRLTMFRGIIIGYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 128 - protein, 446 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKELYREYMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 129 - protein, 449 aa, synthetic construct
EVQLLESGGGLVQPGGSLRLSCATSGFTFSDYAMSWVRQAPGKGLEWVSYNSFSVGSTDYADSVKGRFTISRDNSKNTLYLQMNSLRAEDTAVYYCSKHSGNYYTGPFHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 130 - protein, 450 aa, synthetic construct
QVQLVQSGAEVKKPGASVKVSCKASGYTFTNYWMHWVRQAPGQGLEWMGEIDPSDIYTNYAQKFQGRVTMTRDTSTSTVYMELSSLRSEDTAVYYCARGIYDGYYVYGMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 131 - protein, 443 aa, synthetic construct
QVHLQESGPGLVKPSETLSLTCTVSEESISSYYWSWIRQPPGKGLEWIGHISYSGSANYNPSLKSRVTISVDTSKNQFSLKLSSVTAADTAVYYCANWEEAFNIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 132 - protein, 446 aa, synthetic construct
EIQLLESGGGLVQPGGSLRLSCAVSGFSITTTGYHWNWVRQAPGKGLEWVGYIYSSGSTSYNPSLKSRFTISRDTSKNTFYLQMNSLRAEDTAVYYCARGDWHYFDYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 133 - protein, 446 aa, synthetic construct; identical to SEQ ID NO: 132

SEQ ID NO: 134 - protein, 448 aa, synthetic construct
QVQLQQSGPELVKPGASVKMSCKASGYTFTDYVINWGKQRSGQGLEWIGEIYPGSGTNYYNEKFKAKATLTADKSSNIAYMQLSSLTSEDSAVYFCARRGRYGLYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 135 - protein, 215 aa, synthetic construct
QSALTQPASVSGSPGQSITISCSGSSSNIGNNAVNWYQQLPGKAPKLLIYYDDLLPSGVSDRFSGSKSGTSAFLAISGLQSEDEADYYCAAWDDSLNGPVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTEC

SEQ ID NO: 136 - protein, 214 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYNSYPITFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 137 - protein, 213 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYNSYPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 138 - protein, 214 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYGSFPITFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 139 - protein, 215 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 140 - protein, 214 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 141 - protein, 214 aa, synthetic construct
SSELTQDPAVSVALGQTVRITCQGDSLRSYYASWYQQKPGQAPVLVIYGKNNRPSGIPDRFSGSSSGNTASLTITGAQAEDEADYYCNSRDSFSIHQNVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTEC

SEQ ID NO: 142 - protein, 214 aa, synthetic construct
DIQLTQSPSFLSASVGDRVTITCRASQGISSYLAWYQQKPGKAPKLLIYAASSLESGVPSRFSGSGSGTDFTLTISSLQPEDFATYYCQQSYSTPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 143 - protein, 214 aa, synthetic construct
YIQMTQSPSSLSASVGDRVTITCRSSQDISNYLNWYQQKPGKAPKLLIYYTSRLHSGVPSRFSGSGSGTDFTFTISSLQPEDIATYYCQQGKTLPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 144 - protein, 214 aa, synthetic construct
DIQMTQSPSSLSASVGDRVTITCRASQDISGALNWYQQKPGKAPNLLIYAASSLQSGVPSRFSGSGSGTDYTLTISSLQPEDFATYYCQQANSFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 145 - protein, 212 aa, synthetic construct
SSETTQPPSVSVSPGQTASITCSGEKLSDKYVHWYQQKPGQSPVMVIYENDRRPSGIPERFSGSNSGNTATLTISGTQAMDEADYFCQFWDSTNSAVFGGGTQLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTEC

SEQ ID NO: 146 - protein, 215 aa, synthetic construct
DSVTTQSPLSLPVTLGQPASISCSGEKLSDKYVHWYQQRPGQSPRMLIYENDRRPSGVPDRFSGSNSGNDATLKISRVEAEDVGVYFCQFWDSTNSAVFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 147 - protein, 214 aa, synthetic construct
DIQMTQTTSSLSASLGDRVTISCRASQDISNYLNWYQQKPDGTVKLLIYYTSRLHSGVPSRFSGSGSGTDYSLTINNLEQEDIATYFCQQGNTRPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 148 - protein, 237 aa, Homo sapiens
ITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 149 - protein, 65 aa, synthetic construct
ITCPPPMSVEHADIWVKSYSLYSRERYICNSGFKRKAGTSSLTECVLNKATNVAHWTTPSLKCIR

SEQ ID NO: 150 - protein, 78 aa, synthetic construct
ITCPPPMSVEHADIWVKSYSLYSRERYICNSGFKRKAGTSSLTECVLNKATNVAHWTTPSLKCIRDPALVHQRPAPPS

SEQ ID NO: 151 - protein, 114 aa, synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 152 - protein, 29 aa, synthetic construct
MRISKPHLRSISIQCYLCLLLNSHFLTEA

SEQ ID NO: 153 - protein, 19 aa, synthetic construct
GIHVFILGCFSAGLPKTEA

SEQ ID NO: 154 - protein, 193 aa, synthetic construct
ITCPPPMSVEHADIWVKSYSLYSRERYICNSGFKRKAGTSSLTECVLNKATNVAHWTTPSLKCIRGGGGSGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 155 - protein, 206 aa, synthetic construct
ITCPPPMSVEHADIWVKSYSLYSRERYICNSGFKRKAGTSSLTECVLNKATNVAHWTTPSLKCIRDPALVHQRPAPPSGGGGSGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 156 - protein, 647 aa, synthetic construct
QVQLVESGGGLVKPGGSLRLSCAASGFTFSSYGMHWVRQAPGKGLEWVAFIRYDGSNKYYADSVKGRFTISRDNSKNTKYLQMNSLRAEDTAVYYCAKDRGLGDGTYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSITCPPPMSVEHADIWVKSYSLYSRERYICNSGFKRKAGTSSLTECVLNKATNVAHWTTPSLKCIRGGGGSGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 157 - protein, 643 aa, synthetic construct
QVQLQQWGAGLLKPSETLSLTCAVYGGSFSGYYWSWIRQPPGKGLEWIGEIDHSGSTNYNPSLKSRVTISVDTSKNQFSLKLSSVTAADTAVYYCARARGPWSFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSITCPPPMSVEHADIWVKSYSLYSRERYICNSGFKRKAGTSSLTECVLNKATNVAHWTTPSLKCIRGGGGSGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 158 - protein, 643 aa, synthetic construct; identical to SEQ ID NO: 157

SEQ ID NO: 159 - protein, 643 aa, synthetic construct; identical to SEQ ID NO: 157

SEQ ID NO: 160 - protein, 641 aa, synthetic construct
QVHLQESGPGLVKPSETLSLTCTVSDDSISSYYWSWIRQPPGKGLEWIGHISYSGSANYNPSLKSRVTISVDTSKNQFSLKLSSVTAADTAVYYCANWDDAFNIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSITCPPPMSVEHADIWVKSYSLYSRERYICNSGFKRKAGTSSLTECVLNKATNVAHWTTPSLKCIRGGGGSGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 161 - protein, 649 aa, synthetic construct
EVQLVQSGAEVKEPGESLKISCKNSGYSFTNYWVGWVRQMPGKGLEWMGIIYPGDSDTRYSPSFQGQVTISADKSINTAYLQWSSLKASDTAMYYCGRLTMFRGIIIGYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSITCPPPMSVEHADIWVKSYSLYSRERYICNSGFKRKAGTSSLTECVLNKATNVAHWTTPSLKCIRGGGGSGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 162 - protein, 644 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKELYREYMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSITCPPPMSVEHADIWVKSYSLYSRERYICNSGFKRKAGTSSLTECVLNKATNVAHWTTPSLKCIRGGGGSGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 163 - protein, 647 aa, synthetic construct
EVQLLESGGGLVQPGGSLRLSCATSGFTFSDYAMSWVRQAPGKGLEWVSYNSFSVGSTDYADSVKGRFTISRDNSKNTLYLQMNSLRAEDTAVYYCSKHSGNYYTGPFHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSITCPPPMSVEHADIWVKSYSLYSRERYICNSGFKRKAGTSSLTECVLNKATNVAHWTTPSLKCIRGGGGSGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 164 - protein, 648 aa, synthetic construct
QVQLVQSGAEVKKPGASVKVSCKASGYTFTNYWMHWVRQAPGQGLEWMGEIDPSDIYTNYAQKFQGRVTMTRDTSTSTVYMELSSLRSEDTAVYYCARGIYDGYYVYGMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSITCPPPMSVEHADIWVKSYSLYSRERYICNSGFKRKAGTSSLTECVLNKATNVAHWTTPSLKCIRGGGGSGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 165 - protein, 641 aa, synthetic construct
QVHLQESGPGLVKPSETLSLTCTVSEESISSYYWSWIRQPPGKGLEWIGHISYSGSANYNPSLKSRVTISVDTSKNQFSLKLSSVTAADTAVYYCANWEEAFNIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSITCPPPMSVEHADIWVKSYSLYSRERYICNSGFKRKAGTSSLTECVLNKATNVAHWTTPSLKCIRGGGGSGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 166 - protein, 644 aa, synthetic construct
EIQLLESGGGLVQPGGSLRLSCAVSGFSITTTGYHWNWVRQAPGKGLEWVGYIYSSGSTSYNPSLKSRFTISRDTSKNTFYLQMNSLRAEDTAVYYCARGDWHYFDYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSITCPPPMSVEHADIWVKSYSLYSRERYICNSGFKRKAGTSSLTECVLNKATNVAHWTTPSLKCIRGGGGSGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 167 - protein, 644 aa, synthetic construct; identical to SEQ ID NO: 166

SEQ ID NO: 168 - protein, 646 aa, synthetic construct
QVQLQQSGPELVKPGASVKMSCKASGYTFTDYVINWGKQRSGQGLEWIGEIYPGSGTNYYNEKFKAKATLTADKSSNIAYMQLSSLTSEDSAVYFCARRGRYGLYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSITCPPPMSVEHADIWVKSYSLYSRERYICNSGFKRKAGTSSLTECVLNKATNVAHWTTPSLKCIRGGGGSGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 169 - protein, 413 aa, synthetic construct
QSALTQPASVSGSPGQSITISCSGSSSNIGNNAVNWYQQLPGKAPKLLIYYDDLLPSGVSDRFSGSKSGTSAFLAISGLQSEDEADYYCAAWDDSLNGPVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECGGGGSITCPPPMSVEHADIWVKSYSLYSRERYICNSGFKRKAGTSSLTECVLNKATNVAHWTTPSLKCIRGGGGSGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 170 - protein, 412 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYNSYPITFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSITCPPPMSVEHADIWVKSYSLYSRERYICNSGFKRKAGTSSLTECVLNKATNVAHWTTPSLKCIRGGGGSGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 171 - protein, 411 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYNSYPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSITCPPPMSVEHADIWVKSYSLYSRERYICNSGFKRKAGTSSLTECVLNKATNVAHWTTPSLKCIRGGGGSGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 172 - protein, 412 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYGSFPITFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSITCPPPMSVEHADIWVKSYSLYSRERYICNSGFKRKAGTSSLTECVLNKATNVAHWTTPSLKCIRGGGGSGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 173 - protein, 413 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSITCPPPMSVEHADIWVKSYSLYSRERYICNSGFKRKAGTSSLTECVLNKATNVAHWTTPSLKCIRGGGGSGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 174 - protein, 412 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSITCPPPMSVEHADIWVKSYSLYSRERYICNSGFKRKAGTSSLTECVLNKATNVAHWTTPSLKCIRGGGGSGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 175 - protein, 412 aa, synthetic construct
SSELTQDPAVSVALGQTVRITCQGDSLRSYYASWYQQKPGQAPVLVIYGKNNRPSGIPDRFSGSSSGNTASLTITGAQAEDEADYYCNSRDSFSIHQNVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECGGGGSITCPPPMSVEHADIWVKSYSLYSRERYICNSGFKRKAGTSSLTECVLNKATNVAHWTTPSLKCIRGGGGSGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 176 - protein, 412 aa, synthetic construct
DIQLTQSPSFLSASVGDRVTITCRASQGISSYLAWYQQKPGKAPKLLIYAASSLESGVPSRFSGSGSGTDFTLTISSLQPEDFATYYCQQSYSTPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSITCPPPMSVEHADIWVKSYSLYSRERYICNSGFKRKAGTSSLTECVLNKATNVAHWTTPSLKCIRGGGGSGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 177 - protein, 412 aa, synthetic construct
YIQMTQSPSSLSASVGDRVTITCRSSQDISNYLNWYQQKPGKAPKLLIYYTSRLHSGVPSRFSGSGSGTDFTFTISSLQPEDIATYYCQQGKTLPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSITCPPPMSVEHADIWVKSYSLYSRERYICNSGFKRKAGTSSLTECVLNKATNVAHWTTPSLKCIRGGGGSGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 178 - protein, 412 aa, synthetic construct
DIQMTQSPSSLSASVGDRVTITCRASQDISGALNWYQQKPGKAPNLLIYAASSLQSGVPSRFSGSGSGTDYTLTISSLQPEDFATYYCQQANSFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSITCPPPMSVEHADIWVKSYSLYSRERYICNSGFKRKAGTSSLTECVLNKATNVAHWTTPSLKCIRGGGGSGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 179 - protein, 410 aa, synthetic construct
SSETTQPPSVSVSPGQTASITCSGEKLSDKYVHWYQQKPGQSPVMVIYENDRRPSGIPERFSGSNSGNTATLTISGTQAMDEADYFCQFWDSTNSAVFGGGTQLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECGGGGSITCPPPMSVEHADIWVKSYSLYSRERYICNSGFKRKAGTSSLTECVLNKATNVAHWTTPSLKCIRGGGGSGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 180 - protein, 413 aa, synthetic construct
DSVTTQSPLSLPVTLGQPASISCSGEKLSDKYVHWYQQRPGQSPRMLIYENDRRPSGVPDRFSGSNSGNDATLKISRVEAEDVGVYFCQFWDSTNSAVFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSITCPPPMSVEHADIWVKSYSLYSRERYICNSGFKRKAGTSSLTECVLNKATNVAHWTTPSLKCIRGGGGSGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 181 - protein, 412 aa, synthetic construct
DIQMTQTTSSLSASLGDRVTISCRASQDISNYLNWYQQKPDGTVKLLIYYTSRLHSGVPSRFSGSGSGTDYSLTINNLEQEDIATYFCQQGNTRPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSITCPPPMSVEHADIWVKSYSLYSRERYICNSGFKRKAGTSSLTECVLNKATNVAHWTTPSLKCIRGGGGSGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 182 - protein, 5 aa, synthetic construct
GGGGS

SEQ ID NO: 183 - protein, 14 aa, synthetic construct
GGGGSGGGSGGGGS

SEQ ID NO: 184 - protein, 6 aa, synthetic construct
TVAAPS

SEQ ID NO: 185 - protein, 7 aa, synthetic construct
ASTKGPS

SEQ ID NO: 186 - protein, 6 aa, synthetic construct
  VARIANT 6: note = Residue may be deleted.
  REPEAT 1..5: note = There may be between 1 and 5 repeats of these residues.
GGGGSG

SEQ ID NO: 187 - protein, 5 aa, synthetic construct
  VARIANT 5: note = Residue may be deleted.
  REPEAT 1..4: note = There may be between 1 and 5 repeats of these residues.
GGGSG

SEQ ID NO: 188 - protein, 647 aa, synthetic construct
QVQLVESGGGLVKPGGSLRLSCAASGFTFSSYGMHWVRQAPGKGLEWVAFIRYDGSNKYYADSVKGRFTISRDNSKNTLYLQMNSLRAEDTAVYYCAKDRGLGDGTYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSITCPPPMSVEHADIWVKSYSLYSRERYICNSGFKRKAGTSSLTECVLNKATNVAHWTTPSLKCIRGGGGSGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 189 - protein, 658 aa, synthetic construct
EVQLVQSGAEVKEPGESLKISCKNSGYSFTNYWVGWVRQMPGKGLEWMGIIYPGDSDTRYSPSFQGQVTISADKSINTAYLQWSSLKASDTAMYYCGRLTMFRGIIIGYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGSGGGGSITCPPPMSVEHADIWVKSYSLYSRERYICNSGFKRKAGTSSLTECVLNKATNVAHWTTPSLKCIRGGGGSGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 190 - protein, 650 aa, synthetic construct
ITCPPPMSVEHADIWVKSYSLYSRERYICNSGFKRKAGTSSLTECVLNKATNVAHWTTPSLKCIRGGGGSGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGSEVQLVQSGAEVKEPGESLKISCKNSGYSFTNYWVGWVRQMPGKGLEWMGIIYPGDSDTRYSPSFQGQVTISADKSINTAYLQWSSLKASDTAMYYCGRLTMFRGIIIGYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 191 - protein, 649 aa, synthetic construct
EVQLVQSGAEVKEPGESLKISCKNSGYSFTNYWVGWVRQMPGKGLEWMGIIYPGDSDTRYSPSFQGQVTISADKSINTAYLQWSSLKASDTAMYYCGRLTMFRGIIIGYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSITCPPPMSVEHADIWVKSYSLYSRERYICNSGFKRKAGTSSLTECVLNKATNVAHWTTPSLKCIRGGGGSGGGSGGGGSNWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTS

SEQ ID NO: 192 - protein, 650 aa, synthetic construct
EVQLVQSGAEVKEPGESLKISCKNSGYSFTNYWVGWVRQMPGKGLEWMGIIYPGDSDTRYSPSFQGQVTISADKSINTAYLQWSSLKASDTAMYYCGRLTMFRGIIIGYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPTVAAPSITCPPPMSVEHADIWVKSYSLYSRERYICNSGFKRKAGTSSLTECVLNKATNVAHWTTPSLKCIRGGGGSGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 193 - protein, 579 aa, synthetic construct
EVQLVQSGAEVKEPGESLKISCKNSGYSFTNYWVGWVRQMPGKGLEWMGIIYPGDSDTRYSPSFQGQVTISADKSINTAYLQWSSLKASDTAMYYCGRLTMFRGIIIGYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 194 - protein, 393 aa, synthetic construct
QVQLVESGGGLVKPGGSLRLSCAASGFTFSSYGMHWVRQAPGKGLEWVAFIRYDGSNKYYADSVKGRFTISRDNSKNTLYLQMNSLRAEDTAVYYCAKDRGLGDGTYFDYWGQGTTVTVSSGGGGSGGGGSGGGGSGGGGSQSALTQPASVSGSPGQSITISCSGSSSNIGNNAVNWYQQLPGKAPKLLIYYDDLLPSGVSDRFSGSKSGTSAFLAISGLQSEDEADYYCAAWDDSLNGPVFGGGTKLTVLGGSSGSSGSSHHHHHHHHGGSSGSSGSSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 195 - protein, 641 aa, synthetic construct
EVQLVQSGAEVKEPGESLKISCKNSGYSFTNYWVGWVRQMPGKGLEWMGIIYPGDSDTRYSPSFQGQVTISADKSINTAYLQWSSLKASDTAMYYCGRLTMFRGIIIGYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSITCPPPMSVEHADIWVKSYSLYSRERYICNSGFKRKAGTSSLTECVLNKATNVAHWTTPSLKCIRTVAAPSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 196 - protein, 435 aa, synthetic construct
ITCPPPMSVEHADIWVKSYSLYSRERYICNSGFKRKAGTSSLTECVLNKATNVAHWTTPSLKCIRGGGGSGGGGSGGGGSGGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSEPKSSDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 197 - protein, 444 aa, synthetic construct
QVHLQESGPGLVKPSETLSLTCTVSDDSISSYYWSWIRQPPGKGLEWIGHISYSGSANYNPSLKSRVTISVDTSKNQFSLKLSSVTAADTAVYYCANWDDAFNIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREQMTKNQVKLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 198 - protein, 435 aa, synthetic construct
ITCPPPMSVEHADIWVKSYSLYSRERYICNSGFKRKAGTSSLTECVLNKATNVAHWTTPSLKCIRGGGGSGGGGSGGGGSGGGGSGGGGSNWVDVISDLKKIEDLIQSMHIDATLYTESDVHPSCKVTAMKCFLLELQVISLESGDASIHDTVEDLIILANNSLSSNGNVTESGCKECEELEEKNIKEFLQSFVHIVQMFINTSEPKSSDKTHTCPPCPAPPVAGPSVFLFPPKPKDTLMISRTPEVTCVVVDVKHEDPEVKFNWYVDGVEVHNAKTKPREEEYNSTYRVVSVLTVLHQDWLNGKEYKCKVSNKALPAPIEKTISKAKGQPREPQVYTLPPSREEMTKNQVSLTCDVSGFYPSDIAVEWESDGQPENNYKTTPPVLDSDGSFFLYSKLTVDKSRWEQGDVFSCSVMHEALHNHYTQKSLSLSPGK

SEQ ID NO: 199 - protein, 413 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTVAAPSITCPPPMSVEHADIWVKSYSLYSRERYICNSGFKRKAGTSSLTECVLNKATNVAHWTTPSLKCIRGGGGSGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 200 - protein, 667 aa, synthetic construct
EVQLVQSGAEVKEPGESLKISCKNSGYSFTNYWVGWVRQMPGKGLEWMGIIYPGDSDTRYSPSFQGQVTISADKSINTAYLQWSSLKASDTAMYYCGRLTMFRGIIIGYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 201 - protein, 13 aa, synthetic construct
DPALVHQRPAPPS

SEQ ID NO: 202 - protein, 121 aa, synthetic construct
QVQLVESGGGLVKPGGSLRLSCAASGFTFSSYGMHWVRQAPGKGLEWVAFIRYDGSNKYYADSVKGRFTISRDNSKNTLYLQMNSLRAEDTAVYYCAKDRGLGDGTYFDYWGQGTTVTVSS

SEQ ID NO: 203 - protein, 328 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 204 - protein, 6 aa, synthetic construct
  REPEAT >5: note = The sequence of amino acids 1-5 can be repeated from 1 to 6 times
  VARIANT 6: note = X is G or is absent
GGGGSX

SEQ ID NO: 205 - protein, 5 aa, synthetic construct
  REPEAT >4: note = The sequence of amino acids 1-4 can be repeated from 1 to 6 times
  VARIANT 5: note = X is G or is absent
GGGSX

SEQ ID NO: 206 - protein, 185 aa, synthetic construct
ITCPPPMSVEHADIWVKSYSLYSRERYICNSGFKRKAGTSSLTECVLNKATNVAHWTTPSLKCIRTVAAPSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 207 - protein, 211 aa, synthetic construct
ITCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 208 - protein, 226 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 209 - protein, 15 aa, synthetic construct
GGGGSGGGGSGGGGS

SEQ ID NO: 210 - protein, 439 aa, synthetic construct
ITCPPPMSVEHADIWVKSYSLYSRERYICNSGFKRKAGTSSLTECVLNKATNVAHWTTPSLKCIRGGGGSGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGS

SEQ ID NO: 211 - protein, 246 aa, synthetic construct
QVQLVESGGGLVKPGGSLRLSCAASGFTFSSYGMHWVRQAPGKGLEWVAFIRYDGSNKYYADSVKGRFTISRDNSKNTKYLQMNSLRAEDTAVYYCAKDRGLGDGTYFDYWGQGTTVTVSSGGGGSGGGGSGGGGSQSALTQPASVSGSPGQSITISCSGSSSNIGNNAVNWYQQLPGKAPKLLIYYDDLLPSGVSDRFSGSKSGTSAFLAISGLQSEDEADYYCAAWDDSLNGPVFGGGTKLTVL

SEQ ID NO: 212 - protein, 239 aa, synthetic construct
QVQLQQWGAGLLKPSETLSLTCAVYGGSFSGYYWSWIRQPPGKGLEWIGEIDHSGSTNYNPSLKSRVTISVDTSKNQFSLKLSSVTAADTAVYYCARARGPWSFDPWGQGTLVTVSSGGGGSGGGGSGGGGSDIQMTQSPSTLSASVGDRVTITCRASQSISSWLAWYQQKPGKAPKLLIYKASSLESGVPSRFSGSGSGTEFTLTISSLQPDDFATYYCQQYNSYPITFGGGTKVEIK

SEQ ID NO: 213 - protein, 238 aa, synthetic construct
QVQLQQWGAGLLKPSETLSLTCAVYGGSFSGYYWSWIRQPPGKGLEWIGEIDHSGSTNYNPSLKSRVTISVDTSKNQFSLKLSSVTAADTAVYYCARARGPWSFDPWGQGTLVTVSSGGGGSGGGGSGGGGSDIQMTQSPSTLSASVGDRVTITCRASQSISSWLAWYQQKPGKAPKLLIYKASSLESGVPSRFSGSGSGTEFTLTISSLQPDDFATYYCQQYNSYPTFGGGTKVEIK

SEQ ID NO: 214 - protein, 239 aa, synthetic construct
QVQLQQWGAGLLKPSETLSLTCAVYGGSFSGYYWSWIRQPPGKGLEWIGEIDHSGSTNYNPSLKSRVTISVDTSKNQFSLKLSSVTAADTAVYYCARARGPWSFDPWGQGTLVTVSSGGGGSGGGGSGGGGSDIQMTQSPSTLSASVGDRVTITCRASQSISSWLAWYQQKPGKAPKLLIYKASSLESGVPSRFSGSGSGTEFTLTISSLQPDDFATYYCQQYGSFPITFGGGTKVEIK

SEQ ID NO: 215 - protein, 238 aa, synthetic construct
QVHLQESGPGLVKPSETLSLTCTVSDDSISSYYWSWIRQPPGKGLEWIGHISYSGSANYNPSLKSRVTISVDTSKNQFSLKLSSVTAADTAVYYCANWDDAFNIWGQGTMVTVSSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 216 - protein, 245 aa, synthetic construct
EVQLVQSGAEVKEPGESLKISCKNSGYSFTNYWVGWVRQMPGKGLEWMGIIYPGDSDTRYSPSFQGQVTISADKSINTAYLQWSSLKASDTAMYYCGRLTMFRGIIIGYFDYWGQGTLVTVSSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPWTFGQGTKVEIK

SEQ ID NO: 217 - protein, 242 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKELYREYMDYWGQGTLVTVSSGGGGSGGGGSGGGGSSSELTQDPAVSVALGQTVRITCQGDSLRSYYASWYQQKPGQAPVLVIYGKNNRPSGIPDRFSGSSSGNTASLTITGAQAEDEADYYCNSRDSFSIHQNVFGGGTKLTVL

SEQ ID NO: 218 - protein, 243 aa, synthetic construct
EVQLLESGGGLVQPGGSLRLSCATSGFTFSDYAMSWVRQAPGKGLEWVSYNSFSVGSTDYADSVKGRFTISRDNSKNTLYLQMNSLRAEDTAVYYCSKHSGNYYTGPFHYWGQGTLVTVSSGGGGSGGGGSGGGGSDIQLTQSPSFLSASVGDRVTITCRASQGISSYLAWYQQKPGKAPKLLIYAASSLESGVPSRFSGSGSGTDFTLTISSLQPEDFATYYCQQSYSTPITFGQGTRLEIK

SEQ ID NO: 219 - protein, 244 aa, synthetic construct
QVQLVQSGAEVKKPGASVKVSCKASGYTFTNYWMHWVRQAPGQGLEWMGEIDPSDIYTNYAQKFQGRVTMTRDTSTSTVYMELSSLRSEDTAVYYCARGIYDGYYVYGMDYWGQGTTVTVSSGGGGSGGGGSGGGGSYIQMTQSPSSLSASVGDRVTITCRSSQDISNYLNWYQQKPGKAPKLLIYYTSRLHSGVPSRFSGSGSGTDFTFTISSLQPEDIATYYCQQGKTLPRTFGGGTKVEIK

SEQ ID NO: 220 - protein, 237 aa, synthetic construct
QVHLQESGPGLVKPSETLSLTCTVSEESISSYYWSWIRQPPGKGLEWIGHISYSGSANYNPSLKSRVTISVDTSKNQFSLKLSSVTAADTAVYYCANWEEAFNIWGQGTMVTVSSGGGGSGGGGSGGGGSDIQMTQSPSSLSASVGDRVTITCRASQDISGALNWYQQKPGKAPNLLIYAASSLQSGVPSRFSGSGSGTDYTLTISSLQPEDFATYYCQQANSFPLTFGGGTKVEIK

SEQ ID NO: 221 - protein, 240 aa, synthetic construct
EIQLLESGGGLVQPGGSLRLSCAVSGFSITTTGYHWNWVRQAPGKGLEWVGYIYSSGSTSYNPSLKSRFTISRDTSKNTFYLQMNSLRAEDTAVYYCARGDWHYFDYWGQGTMVTVSSGGGGSGGGGSGGGGSSSETTQPPSVSVSPGQTASITCSGEKLSDKYVHWYQQKPGQSPVMVIYENDRRPSGIPERFSGSNSGNTATLTISGTQAMDEADYFCQFWDSTNSAVFGGGTQLTVL

SEQ ID NO: 222 - protein, 241 aa, synthetic construct
EIQLLESGGGLVQPGGSLRLSCAVSGFSITTTGYHWNWVRQAPGKGLEWVGYIYSSGSTSYNPSLKSRFTISRDTSKNTFYLQMNSLRAEDTAVYYCARGDWHYFDYWGQGTMVTVSSGGGGSGGGGSGGGGSDSVTTQSPLSLPVTLGQPASISCSGEKLSDKYVHWYQQRPGQSPRMLIYENDRRPSGVPDRFSGSNSGNDATLKISRVEAEDVGVYFCQFWDSTNSAVFGGGTKVEIK

SEQ ID NO: 223 - protein, 242 aa, synthetic construct
QVQLQQSGPELVKPGASVKMSCKASGYTFTDYVINWGKQRSGQGLEWIGEIYPGSGTNYYNEKFKAKATLTADKSSNIAYMQLSSLTSEDSAVYFCARRGRYGLYAMDYWGQGTSVTVSSGGGGSGGGGSGGGGSDIQMTQTTSSLSASLGDRVTISCRASQDISNYLNWYQQKPDGTVKLLIYYTSRLHSGVPSRFSGSGSGTDYSLTINNLEQEDIATYFCQQGNTRPWTFGGGTKLEIK

SEQ ID NO: 224 - protein, 246 aa, synthetic construct
QSALTQPASVSGSPGQSITISCSGSSSNIGNNAVNWYQQLPGKAPKLLIYYDDLLPSGVSDRFSGSKSGTSAFLAISGLQSEDEADYYCAAWDDSLNGPVFGGGTKLTVLGGGGSGGGGSGGGGSQVQLVESGGGLVKPGGSLRLSCAASGFTFSSYGMHWVRQAPGKGLEWVAFIRYDGSNKYYADSVKGRFTISRDNSKNTKYLQMNSLRAEDTAVYYCAKDRGLGDGTYFDYWGQGTTVTVSS

SEQ ID NO: 225 - protein, 239 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYNSYPITFGGGTKVEIKGGGGSGGGGSGGGGSQVQLQQWGAGLLKPSETLSLTCAVYGGSFSGYYWSWIRQPPGKGLEWIGEIDHSGSTNYNPSLKSRVTISVDTSKNQFSLKLSSVTAADTAVYYCARARGPWSFDPWGQGTLVTVSS

SEQ ID NO: 226 - protein, 238 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYNSYPTFGGGTKVEIKGGGGSGGGGSGGGGSQVQLQQWGAGLLKPSETLSLTCAVYGGSFSGYYWSWIRQPPGKGLEWIGEIDHSGSTNYNPSLKSRVTISVDTSKNQFSLKLSSVTAADTAVYYCARARGPWSFDPWGQGTLVTVSS

SEQ ID NO: 227 - protein, 239 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYGSFPITFGGGTKVEIKGGGGSGGGGSGGGGSQVQLQQWGAGLLKPSETLSLTCAVYGGSFSGYYWSWIRQPPGKGLEWIGEIDHSGSTNYNPSLKSRVTISVDTSKNQFSLKLSSVTAADTAVYYCARARGPWSFDPWGQGTLVTVSS

SEQ ID NO: 228 - protein, 238 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIKGGGGSGGGGSGGGGSQVHLQESGPGLVKPSETLSLTCTVSDDSISSYYWSWIRQPPGKGLEWIGHISYSGSANYNPSLKSRVTISVDTSKNQFSLKLSSVTAADTAVYYCANWDDAFNIWGQGTMVTVSS

SEQ ID NO: 229 - protein, 245 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPWTFGQGTKVEIKGGGGSGGGGSGGGGSEVQLVQSGAEVKEPGESLKISCKNSGYSFTNYWVGWVRQMPGKGLEWMGIIYPGDSDTRYSPSFQGQVTISADKSINTAYLQWSSLKASDTAMYYCGRLTMFRGIIIGYFDYWGQGTLVTVSS

SEQ ID NO: 230 - protein, 242 aa, synthetic construct
SSELTQDPAVSVALGQTVRITCQGDSLRSYYASWYQQKPGQAPVLVIYGKNNRPSGIPDRFSGSSSGNTASLTITGAQAEDEADYYCNSRDSFSIHQNVFGGGTKLTVLGGGGSGGGGSGGGGSEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKELYREYMDYWGQGTLVTVSS

SEQ ID NO: 231 - protein, 243 aa, synthetic construct
DIQLTQSPSFLSASVGDRVTITCRASQGISSYLAWYQQKPGKAPKLLIYAASSLESGVPSRFSGSGSGTDFTLTISSLQPEDFATYYCQQSYSTPITFGQGTRLEIKGGGGSGGGGSGGGGSEVQLLESGGGLVQPGGSLRLSCATSGFTFSDYAMSWVRQAPGKGLEWVSYNSFSVGSTDYADSVKGRFTISRDNSKNTLYLQMNSLRAEDTAVYYCSKHSGNYYTGPFHYWGQGTLVTVSS

SEQ ID NO: 232 - protein, 244 aa, synthetic construct
YIQMTQSPSSLSASVGDRVTITCRSSQDISNYLNWYQQKPGKAPKLLIYYTSRLHSGVPSRFSGSGSGTDFTFTISSLQPEDIATYYCQQGKTLPRTFGGGTKVEIKGGGGSGGGGSGGGGSQVQLVQSGAEVKKPGASVKVSCKASGYTFTNYWMHWVRQAPGQGLEWMGEIDPSDIYTNYAQKFQGRVTMTRDTSTSTVYMELSSLRSEDTAVYYCARGIYDGYYVYGMDYWGQGTTVTVSS

SEQ ID NO: 233 - protein, 465 aa, synthetic construct
DIQMTQSPSSLSASVGDRVTITCRASQDISGALNWYQQKPGKAPNLLIYAASSLQSGVPSRFSGSGSGTDYTLTISSLQPEDFATYYCQQANSFPLTFGGGTKVEIKEVQLLESGGGLVQPGGSLRLSCATSGFTFSDYAMSWVRQAPGKGLEWVSYNSFSVGSTDYADSVKGRFTISRDNSKNTLYLQMNSLRAEDTAVYYCSKHSGNYYTGPFHYWGQGTLVTVSSGGGGSGGGGSGGGGSDIQLTQSPSFLSASVGDRVTITCRASQGISSYLAWYQQKPGKAPKLLIYAASSLESGVPSRFSGSGSGTDFTLTISSLQPEDFATYYCQQSYSTPITFGQGTRLEIKQVHLQESGPGLVKPSETLSLTCTVSEESISSYYWSWIRQPPGKGLEWIGHISYSGSANYNPSLKSRVTISVDTSKNQFSLKLSSVTAADTAVYYCANWEEAFNIWGQGTMVTVSS

SEQ ID NO: 234 - protein, 469 aa, synthetic construct
SSETTQPPSVSVSPGQTASITCSGEKLSDKYVHWYQQKPGQSPVMVIYENDRRPSGIPERFSGSNSGNTATLTISGTQAMDEADYFCQFWDSTNSAVFGGGTQLTVLQVQLVQSGAEVKKPGASVKVSCKASGYTFTNYWMHWVRQAPGQGLEWMGEIDPSDIYTNYAQKFQGRVTMTRDTSTSTVYMELSSLRSEDTAVYYCARGIYDGYYVYGMDYWGQGTTVTVSSGGGGSGGGGSGGGGSYIQMTQSPSSLSASVGDRVTITCRSSQDISNYLNWYQQKPGKAPKLLIYYTSRLHSGVPSRFSGSGSGTDFTFTISSLQPEDIATYYCQQGKTLPRTFGGGTKVEIKEIQLLESGGGLVQPGGSLRLSCAVSGFSITTTGYHWNWVRQAPGKGLEWVGYIYSSGSTSYNPSLKSRFTISRDTSKNTFYLQMNSLRAEDTAVYYCARGDWHYFDYWGQGTMVTVSS

SEQ ID NO: 235 - protein, 241 aa, synthetic construct
DSVTTQSPLSLPVTLGQPASISCSGEKLSDKYVHWYQQRPGQSPRMLIYENDRRPSGVPDRFSGSNSGNDATLKISRVEAEDVGVYFCQFWDSTNSAVFGGGTKVEIKGGGGSGGGGSGGGGSEIQLLESGGGLVQPGGSLRLSCAVSGFSITTTGYHWNWVRQAPGKGLEWVGYIYSSGSTSYNPSLKSRFTISRDTSKNTFYLQMNSLRAEDTAVYYCARGDWHYFDYWGQGTMVTVSS

SEQ ID NO: 236 - protein, 242 aa, synthetic construct
DIQMTQTTSSLSASLGDRVTISCRASQDISNYLNWYQQKPDGTVKLLIYYTSRLHSGVPSRFSGSGSGTDYSLTINNLEQEDIATYFCQQGNTRPWTFGGGTKLEIKGGGGSGGGGSGGGGSQVQLQQSGPELVKPGASVKMSCKASGYTFTDYVINWGKQRSGQGLEWIGEIYPGSGTNYYNEKFKAKATLTADKSSNIAYMQLSSLTSEDSAVYFCARRGRYGLYAMDYWGQGTSVTVSS

SEQ ID NO: 237 - protein, 685 aa, synthetic construct
ITCPPPMSVEHADIWVKSYSLYSRERYICNSGFKRKAGTSSLTECVLNKATNVAHWTTPSLKCIRGGGGSGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSQVQLVESGGGLVKPGGSLRLSCAASGFTFSSYGMHWVRQAPGKGLEWVAFIRYDGSNKYYADSVKGRFTISRDNSKNTKYLQMNSLRAEDTAVYYCAKDRGLGDGTYFDYWGQGTTVTVSSGGGGSGGGGSGGGGSQSALTQPASVSGSPGQSITISCSGSSSNIGNNAVNWYQQLPGKAPKLLIYYDDLLPSGVSDRFSGSKSGTSAFLAISGLQSEDEADYYCAAWDDSLNGPVFGGGTKLTVL

SEQ ID NO: 238 - protein, 678 aa, synthetic construct
ITCPPPMSVEHADIWVKSYSLYSRERYICNSGFKRKAGTSSLTECVLNKATNVAHWTTPSLKCIRGGGGSGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSQVQLQQWGAGLLKPSETLSLTCAVYGGSFSGYYWSWIRQPPGKGLEWIGEIDHSGSTNYNPSLKSRVTISVDTSKNQFSLKLSSVTAADTAVYYCARARGPWSFDPWGQGTLVTVSSGGGGSGGGGSGGGGSDIQMTQSPSTLSASVGDRVTITCRASQSISSWLAWYQQKPGKAPKLLIYKASSLESGVPSRFSGSGSGTEFTLTISSLQPDDFATYYCQQYNSYPITFGGGTKVEIK

SEQ ID NO: 239 - protein, 677 aa, synthetic construct
ITCPPPMSVEHADIWVKSYSLYSRERYICNSGFKRKAGTSSLTECVLNKATNVAHWTTPSLKCIRGGGGSGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSQVQLQQWGAGLLKPSETLSLTCAVYGGSFSGYYWSWIRQPPGKGLEWIGEIDHSGSTNYNPSLKSRVTISVDTSKNQFSLKLSSVTAADTAVYYCARARGPWSFDPWGQGTLVTVSSGGGGSGGGGSGGGGSDIQMTQSPSTLSASVGDRVTITCRASQSISSWLAWYQQKPGKAPKLLIYKASSLESGVPSRFSGSGSGTEFTLTISSLQPDDFATYYCQQYNSYPTFGGGTKVEIK

SEQ ID NO: 240 - protein, 678 aa, synthetic construct
ITCPPPMSVEHADIWVKSYSLYSRERYICNSGFKRKAGTSSLTECVLNKATNVAHWTTPSLKCIRGGGGSGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSQVQLQQWGAGLLKPSETLSLTCAVYGGSFSGYYWSWIRQPPGKGLEWIGEIDHSGSTNYNPSLKSRVTISVDTSKNQFSLKLSSVTAADTAVYYCARARGPWSFDPWGQGTLVTVSSGGGGSGGGGSGGGGSDIQMTQSPSTLSASVGDRVTITCRASQSISSWLAWYQQKPGKAPKLLIYKASSLESGVPSRFSGSGSGTEFTLTISSLQPDDFATYYCQQYGSFPITFGGGTKVEIK

SEQ ID NO: 241 - protein, 677 aa, synthetic construct
ITCPPPMSVEHADIWVKSYSLYSRERYICNSGFKRKAGTSSLTECVLNKATNVAHWTTPSLKCIRGGGGSGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSQVHLQESGPGLVKPSETLSLTCTVSDDSISSYYWSWIRQPPGKGLEWIGHISYSGSANYNPSLKSRVTISVDTSKNQFSLKLSSVTAADTAVYYCANWDDAFNIWGQGTMVTVSSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 242 - protein, 684 aa, synthetic construct
ITCPPPMSVEHADIWVKSYSLYSRERYICNSGFKRKAGTSSLTECVLNKATNVAHWTTPSLKCIRGGGGSGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSEVQLVQSGAEVKEPGESLKISCKNSGYSFTNYWVGWVRQMPGKGLEWMGIIYPGDSDTRYSPSFQGQVTISADKSINTAYLQWSSLKASDTAMYYCGRLTMFRGIIIGYFDYWGQGTLVTVSSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPWTFGQGTKVEIK

SEQ ID NO: 243 - protein, 681 aa, synthetic construct
ITCPPPMSVEHADIWVKSYSLYSRERYICNSGFKRKAGTSSLTECVLNKATNVAHWTTPSLKCIRGGGGSGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKELYREYMDYWGQGTLVTVSSGGGGSGGGGSGGGGSSSELTQDPAVSVALGQTVRITCQGDSLRSYYASWYQQKPGQAPVLVIYGKNNRPSGIPDRFSGSSSGNTASLTITGAQAEDEADYYCNSRDSFSIHQNVFGGGTKLTVL

SEQ ID NO: 244 - protein, 682 aa, synthetic construct
ITCPPPMSVEHADIWVKSYSLYSRERYICNSGFKRKAGTSSLTECVLNKATNVAHWTTPSLKCIRGGGGSGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSEVQLLESGGGLVQPGGSLRLSCATSGFTFSDYAMSWVRQAPGKGLEWVSYNSFSVGSTDYADSVKGRFTISRDNSKNTLYLQMNSLRAEDTAVYYCSKHSGNYYTGPFHYWGQGTLVTVSSGGGGSGGGGSGGGGSDIQLTQSPSFLSASVGDRVTITCRASQGISSYLAWYQQKPGKAPKLLIYAASSLESGVPSRFSGSGSGTDFTLTISSLQPEDFATYYCQQSYSTPITFGQGTRLEIK

SEQ ID NO: 245 - protein, 683 aa, synthetic construct
ITCPPPMSVEHADIWVKSYSLYSRERYICNSGFKRKAGTSSLTECVLNKATNVAHWTTPSLKCIRGGGGSGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSQVQLVQSGAEVKKPGASVKVSCKASGYTFTNYWMHWVRQAPGQGLEWMGEIDPSDIYTNYAQKFQGRVTMTRDTSTSTVYMELSSLRSEDTAVYYCARGIYDGYYVYGMDYWGQGTTVTVSSGGGGSGGGGSGGGGSYIQMTQSPSSLSASVGDRVTITCRSSQDISNYLNWYQQKPGKAPKLLIYYTSRLHSGVPSRFSGSGSGTDFTFTISSLQPEDIATYYCQQGKTLPRTFGGGTKVEIK

SEQ ID NO: 246 - protein, 676 aa, synthetic construct
ITCPPPMSVEHADIWVKSYSLYSRERYICNSGFKRKAGTSSLTECVLNKATNVAHWTTPSLKCIRGGGGSGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSQVHLQESGPGLVKPSETLSLTCTVSEESISSYYWSWIRQPPGKGLEWIGHISYSGSANYNPSLKSRVTISVDTSKNQFSLKLSSVTAADTAVYYCANWEEAFNIWGQGTMVTVSSGGGGSGGGGSGGGGSDIQMTQSPSSLSASVGDRVTITCRASQDISGALNWYQQKPGKAPNLLIYAASSLQSGVPSRFSGSGSGTDYTLTISSLQPEDFATYYCQQANSFPLTFGGGTKVEIK

SEQ ID NO: 247 - protein, 679 aa, synthetic construct
ITCPPPMSVEHADIWVKSYSLYSRERYICNSGFKRKAGTSSLTECVLNKATNVAHWTTPSLKCIRGGGGSGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSEIQLLESGGGLVQPGGSLRLSCAVSGFSITTTGYHWNWVRQAPGKGLEWVGYIYSSGSTSYNPSLKSRFTISRDTSKNTFYLQMNSLRAEDTAVYYCARGDWHYFDYWGQGTMVTVSSGGGGSGGGGSGGGGSSSETTQPPSVSVSPGQTASITCSGEKLSDKYVHWYQQKPGQSPVMVIYENDRRPSGIPERFSGSNSGNTATLTISGTQAMDEADYFCQFWDSTNSAVFGGGTQLTVL

SEQ ID NO: 248 - protein, 680 aa, synthetic construct
ITCPPPMSVEHADIWVKSYSLYSRERYICNSGFKRKAGTSSLTECVLNKATNVAHWTTPSLKCIRGGGGSGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSEIQLLESGGGLVQPGGSLRLSCAVSGFSITTTGYHWNWVRQAPGKGLEWVGYIYSSGSTSYNPSLKSRFTISRDTSKNTFYLQMNSLRAEDTAVYYCARGDWHYFDYWGQGTMVTVSSGGGGSGGGGSGGGGSDSVTTQSPLSLPVTLGQPASISCSGEKLSDKYVHWYQQRPGQSPRMLIYENDRRPSGVPDRFSGSNSGNDATLKISRVEAEDVGVYFCQFWDSTNSAVFGGGTKVEIK

SEQ ID NO: 249 - protein, 681 aa, synthetic construct
ITCPPPMSVEHADIWVKSYSLYSRERYICNSGFKRKAGTSSLTECVLNKATNVAHWTTPSLKCIRGGGGSGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSQVQLQQSGPELVKPGASVKMSCKASGYTFTDYVINWGKQRSGQGLEWIGEIYPGSGTNYYNEKFKAKATLTADKSSNIAYMQLSSLTSEDSAVYFCARRGRYGLYAMDYWGQGTSVTVSSGGGGSGGGGSGGGGSDIQMTQTTSSLSASLGDRVTISCRASQDISNYLNWYQQKPDGTVKLLIYYTSRLHSGVPSRFSGSGSGTDYSLTINNLEQEDIATYFCQQGNTRPWTFGGGTKLEIK

SEQ ID NO: 250 - protein, 685 aa, synthetic construct
ITCPPPMSVEHADIWVKSYSLYSRERYICNSGFKRKAGTSSLTECVLNKATNVAHWTTPSLKCIRGGGGSGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSQSALTQPASVSGSPGQSITISCSGSSSNIGNNAVNWYQQLPGKAPKLLIYYDDLLPSGVSDRFSGSKSGTSAFLAISGLQSEDEADYYCAAWDDSLNGPVFGGGTKLTVLGGGGSGGGGSGGGGSQVQLVESGGGLVKPGGSLRLSCAASGFTFSSYGMHWVRQAPGKGLEWVAFIRYDGSNKYYADSVKGRFTISRDNSKNTKYLQMNSLRAEDTAVYYCAKDRGLGDGTYFDYWGQGTTVTVSS

SEQ ID NO: 251 - protein, 678 aa, synthetic construct
ITCPPPMSVEHADIWVKSYSLYSRERYICNSGFKRKAGTSSLTECVLNKATNVAHWTTPSLKCIRGGGGSGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSDIQMTQSPSTLSASVGDRVTITCRASQSISSWLAWYQQKPGKAPKLLIYKASSLESGVPSRFSGSGSGTEFTLTISSLQPDDFATYYCQQYNSYPITFGGGTKVEIKGGGGSGGGGSGGGGSQVQLQQWGAGLLKPSETLSLTCAVYGGSFSGYYWSWIRQPPGKGLEWIGEIDHSGSTNYNPSLKSRVTISVDTSKNQFSLKLSSVTAADTAVYYCARARGPWSFDPWGQGTLVTVSS

SEQ ID NO: 252 - protein, 677 aa, synthetic construct
ITCPPPMSVEHADIWVKSYSLYSRERYICNSGFKRKAGTSSLTECVLNKATNVAHWTTPSLKCIRGGGGSGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSDIQMTQSPSTLSASVGDRVTITCRASQSISSWLAWYQQKPGKAPKLLIYKASSLESGVPSRFSGSGSGTEFTLTISSLQPDDFATYYCQQYNSYPTFGGGTKVEIKGGGGSGGGGSGGGGSQVQLQQWGAGLLKPSETLSLTCAVYGGSFSGYYWSWIRQPPGKGLEWIGEIDHSGSTNYNPSLKSRVTISVDTSKNQFSLKLSSVTAADTAVYYCARARGPWSFDPWGQGTLVTVSS

SEQ ID NO: 253 - protein, 678 aa, synthetic construct
ITCPPPMSVEHADIWVKSYSLYSRERYICNSGFKRKAGTSSLTECVLNKATNVAHWTTPSLKCIRGGGGSGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSDIQMTQSPSTLSASVGDRVTITCRASQSISSWLAWYQQKPGKAPKLLIYKASSLESGVPSRFSGSGSGTEFTLTISSLQPDDFATYYCQQYGSFPITFGGGTKVEIKGGGGSGGGGSGGGGSQVQLQQWGAGLLKPSETLSLTCAVYGGSFSGYYWSWIRQPPGKGLEWIGEIDHSGSTNYNPSLKSRVTISVDTSKNQFSLKLSSVTAADTAVYYCARARGPWSFDPWGQGTLVTVSS

SEQ ID NO: 254 - protein, 677 aa, synthetic construct
ITCPPPMSVEHADIWVKSYSLYSRERYICNSGFKRKAGTSSLTECVLNKATNVAHWTTPSLKCIRGGGGSGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIKGGGGSGGGGSGGGGSQVHLQESGPGLVKPSETLSLTCTVSDDSISSYYWSWIRQPPGKGLEWIGHISYSGSANYNPSLKSRVTISVDTSKNQFSLKLSSVTAADTAVYYCANWDDAFNIWGQGTMVTVSS

SEQ ID NO: 255 - protein, 684 aa, synthetic construct
ITCPPPMSVEHADIWVKSYSLYSRERYICNSGFKRKAGTSSLTECVLNKATNVAHWTTPSLKCIRGGGGSGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPWTFGQGTKVEIKGGGGSGGGGSGGGGSEVQLVQSGAEVKEPGESLKISCKNSGYSFTNYWVGWVRQMPGKGLEWMGIIYPGDSDTRYSPSFQGQVTISADKSINTAYLQWSSLKASDTAMYYCGRLTMFRGIIIGYFDYWGQGTLVTVSS

SEQ ID NO: 256 - protein, 681 aa, synthetic construct
ITCPPPMSVEHADIWVKSYSLYSRERYICNSGFKRKAGTSSLTECVLNKATNVAHWTTPSLKCIRGGGGSGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSSSELTQDPAVSVALGQTVRITCQGDSLRSYYASWYQQKPGQAPVLVIYGKNNRPSGIPDRFSGSSSGNTASLTITGAQAEDEADYYCNSRDSFSIHQNVFGGGTKLTVLGGGGSGGGGSGGGGSEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKELYREYMDYWGQGTLVTVSS

SEQ ID NO: 257 - protein, 682 aa, synthetic construct
ITCPPPMSVEHADIWVKSYSLYSRERYICNSGFKRKAGTSSLTECVLNKATNVAHWTTPSLKCIRGGGGSGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSDIQLTQSPSFLSASVGDRVTITCRASQGISSYLAWYQQKPGKAPKLLIYAASSLESGVPSRFSGSGSGTDFTLTISSLQPEDFATYYCQQSYSTPITFGQGTRLEIKGGGGSGGGGSGGGGSEVQLLESGGGLVQPGGSLRLSCATSGFTFSDYAMSWVRQAPGKGLEWVSYNSFSVGSTDYADSVKGRFTISRDNSKNTLYLQMNSLRAEDTAVYYCSKHSGNYYTGPFHYWGQGTLVTVSS

SEQ ID NO: 258 - protein, 683 aa, synthetic construct
ITCPPPMSVEHADIWVKSYSLYSRERYICNSGFKRKAGTSSLTECVLNKATNVAHWTTPSLKCIRGGGGSGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSYIQMTQSPSSLSASVGDRVTITCRSSQDISNYLNWYQQKPGKAPKLLIYYTSRLHSGVPSRFSGSGSGTDFTFTISSLQPEDIATYYCQQGKTLPRTFGGGTKVEIKGGGGSGGGGSGGGGSQVQLVQSGAEVKKPGASVKVSCKASGYTFTNYWMHWVRQAPGQGLEWMGEIDPSDIYTNYAQKFQGRVTMTRDTSTSTVYMELSSLRSEDTAVYYCARGIYDGYYVYGMDYWGQGTTVTVSS

SEQ ID NO: 259 - protein, 904 aa, synthetic construct
ITCPPPMSVEHADIWVKSYSLYSRERYICNSGFKRKAGTSSLTECVLNKATNVAHWTTPSLKCIRGGGGSGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSDIQMTQSPSSLSASVGDRVTITCRASQDISGALNWYQQKPGKAPNLLIYAASSLQSGVPSRFSGSGSGTDYTLTISSLQPEDFATYYCQQANSFPLTFGGGTKVEIKEVQLLESGGGLVQPGGSLRLSCATSGFTFSDYAMSWVRQAPGKGLEWVSYNSFSVGSTDYADSVKGRFTISRDNSKNTLYLQMNSLRAEDTAVYYCSKHSGNYYTGPFHYWGQGTLVTVSSGGGGSGGGGSGGGGSDIQLTQSPSFLSASVGDRVTITCRASQGISSYLAWYQQKPGKAPKLLIYAASSLESGVPSRFSGSGSGTDFTLTISSLQPEDFATYYCQQSYSTPITFGQGTRLEIKQVHLQESGPGLVKPSETLSLTCTVSEESISSYYWSWIRQPPGKGLEWIGHISYSGSANYNPSLKSRVTISVDTSKNQFSLKLSSVTAADTAVYYCANWEEAFNIWGQGTMVTVSS

SEQ ID NO: 260 - protein, 908 aa, synthetic construct
ITCPPPMSVEHADIWVKSYSLYSRERYICNSGFKRKAGTSSLTECVLNKATNVAHWTTPSLKCIRGGGGSGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSSSETTQPPSVSVSPGQTASITCSGEKLSDKYVHWYQQKPGQSPVMVIYENDRRPSGIPERFSGSNSGNTATLTISGTQAMDEADYFCQFWDSTNSAVFGGGTQLTVLQVQLVQSGAEVKKPGASVKVSCKASGYTFTNYWMHWVRQAPGQGLEWMGEIDPSDIYTNYAQKFQGRVTMTRDTSTSTVYMELSSLRSEDTAVYYCARGIYDGYYVYGMDYWGQGTTVTVSSGGGGSGGGGSGGGGSYIQMTQSPSSLSASVGDRVTITCRSSQDISNYLNWYQQKPGKAPKLLIYYTSRLHSGVPSRFSGSGSGTDFTFTISSLQPEDIATYYCQQGKTLPRTFGGGTKVEIKEIQLLESGGGLVQPGGSLRLSCAVSGFSITTTGYHWNWVRQAPGKGLEWVGYIYSSGSTSYNPSLKSRFTISRDTSKNTFYLQMNSLRAEDTAVYYCARGDWHYFDYWGQGTMVTVSS

SEQ ID NO: 261 - protein, 680 aa, synthetic construct
ITCPPPMSVEHADIWVKSYSLYSRERYICNSGFKRKAGTSSLTECVLNKATNVAHWTTPSLKCIRGGGGSGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSDSVTTQSPLSLPVTLGQPASISCSGEKLSDKYVHWYQQRPGQSPRMLIYENDRRPSGVPDRFSGSNSGNDATLKISRVEAEDVGVYFCQFWDSTNSAVFGGGTKVEIKGGGGSGGGGSGGGGSEIQLLESGGGLVQPGGSLRLSCAVSGFSITTTGYHWNWVRQAPGKGLEWVGYIYSSGSTSYNPSLKSRFTISRDTSKNTFYLQMNSLRAEDTAVYYCARGDWHYFDYWGQGTMVTVSS

SEQ ID NO: 262 - protein, 681 aa, synthetic construct
ITCPPPMSVEHADIWVKSYSLYSRERYICNSGFKRKAGTSSLTECVLNKATNVAHWTTPSLKCIRGGGGSGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSDIQMTQTTSSLSASLGDRVTISCRASQDISNYLNWYQQKPDGTVKLLIYYTSRLHSGVPSRFSGSGSGTDYSLTINNLEQEDIATYFCQQGNTRPWTFGGGTKLEIKGGGGSGGGGSGGGGSQVQLQQSGPELVKPGASVKMSCKASGYTFTDYVINWGKQRSGQGLEWIGEIYPGSGTNYYNEKFKAKATLTADKSSNIAYMQLSSLTSEDSAVYFCARRGRYGLYAMDYWGQGTSVTVSS

(19) United States
(12) Patent Application Publication
(10) Pub. No.: US 2026/0232829 A1
(43) Pub. Date: Aug. 13, 2026

(54) NOVEL IMMUNOCONJUGATES

(71) Applicant: CYTOSPIRE THERAPEUTICS LIMITED, Cheshire (GB)

(72) Inventors: Natalie Marianne Mount, Cheshire (GB); Mark Uden, Cheshire (GB)

(21) Appl. No.: 19/155,231

(22) PCT Filed: Feb. 9, 2024
(86) PCT No.: PCT/EP2024/053368

§ 371 Date: Aug. 8, 2025

(30) Foreign Application Priority Data
Feb. 10, 2023 (GB) 2301945.8

(51) Int. Cl. A61K 47/68; A61P 35/00; C07K 16/28

(52) U.S. Cl. CPC A61K 47/6813; A61K 47/6879; A61K 47/6889; A61P 35/00; C07K 16/2803; C07K 16/2851

(57) ABSTRACT

The invention provides immunoconjugates comprising at least two IL-15 polypeptides, each comprising an IL-15 cytokine and IL-15 receptor alpha (CD215) or a functional fragment thereof; and a multivalent antibody or antigen-binding fragment thereof that specifically binds to an antigen selected from the group consisting of NKG2D, NKp30, NKp46, NKp44 and DNAM-1. Also provided are uses of said immunoconjugates and methods of using said immunoconjugates.

### FIELD OF THE INVENTION

In general, this invention relates to immunoconjugates, particularly immunoconjugates comprising interleukin-15 (IL-15) polypeptides, and a multivalent antibody that specifically binds to an antigen selected from the group consisting of NKG2D (natural killer group 2, member D receptor), NKp30 (natural killer protein 30), NKp46 (natural killer protein 46), NKp44 (natural killer protein 44) and DNAM-1 (DNAX accessory molecule-1). Additionally, this invention relates to polynucleotide molecules encoding said immunoconjugates. It also relates to vectors containing such polynucleotides and host cells comprising such polynucleotides. This invention additionally relates to methods for producing and purifying said immunoconjugates. Further, this invention relates to pharmaceutical compositions comprising said immunoconjugates, and uses thereof. The invention additionally relates to the use of such immunoconjugates in medicine, for example in the treatment of cancer.

### BACKGROUND

### IL-15

Human Interleukin-15 is a four-helix bundle cytokine first described as a T-cell proliferation factor. It is constitutively expressed by many types of cells such as macrophages, monocytes, dendritic cells (DCs), T-cells, as well as epithelial cells, fibroblasts, keratinocytes, and nerve cells. IL-15 is involved in lymphocyte and NK cell functioning. It signals through a heterotrimeric receptor which consist of three subunits, the IL-15 receptor α (IL-5Rα or CD215) specific subunit, IL2/IL-15 receptor β subunit (IL2/IL-15Rβ; CD122), and γ (CD132) subunit which is a common chain for other cytokines including IL-2, IL-4, IL-7, IL-9, and IL-21.

IL-15 is not typically released as a free cytokine but is instead bound to IL-15Rα. As such IL-15 functions mainly in a cell-to-cell or more specifically membrane-to-membrane, contact-dependent manner. Typically, membrane-bound IL-15Rα-IL-15 complexes are presented to responding cells that have the intermediate-affinity IL-2/IL-15β-γc (CD122-CD132) receptor complex at their surface. This process of activation involving two cells that come into contact is called “trans-presentation.” Signalling by “cis-presentation” (membrane-to-membrane interactions on same cell) or through soluble complexes of IL-15Rα-IL-15 can also contribute to IL-15 induced responses.

IL-15 signalling requires Jak-1 and Jak-3 to phosphorylate and activate STAT3 and STAT5. Additionally, IL-15 stimulates the PI3K/AKT and RAS/MAPK pathways. IL-15 acts on many cells of the immune system. It can increase proliferation of B cells and secretion of immunoglobulins. It is also critical for the ontogeny of NK and CD8+ T cells, inducing cell activation, proliferation, cytolytic activity, and the production of cytokines such as interferon-γ (IFN-γ). In summary, IL-15 confers broad-ranging pleiotropic effects via multiple signalling pathways upon multiple cell types.

Administration of IL-15 to mouse tumour models has shown to confer favourable effects. However co-administration of IL-15 associated with soluble IL-15Rα (sIL-15Rα) can sometimes induce a greater antitumour responses in such mouse models (e.g. Epardaud M, et al (2008) Cancer Res. 2008; 68:2972-83). This is likely because IL-15/sIL-15Rα mimics the aforementioned trans-presentation and increases the biostability/activity of IL-15.

Because soluble IL-15 cytokine-based products have been characterized by modest biological activity, suboptimal tumour targeting properties, and ultra-rapid clearance (Corbellari et al (2021), Mol Cancer Ther. 20: 859-871) a variety of IL-15 based “superagonists” have been developed (for example see Rubinstein et al 2006, PNAS June 13, vol. 103 no. 24 9166-9171). Typically, such superagonists comprise the IL-15 cytokine which is then further modified to enhance biological activity. As mentioned above, one approach to superagonism involves associating or fusing the IL-15 cytokine or functional portion thereof to the IL-15Rα receptor or functional portion thereof such as the IL-15Rα sushi domain. The sushi domain is under one hundred amino acids in length and often considered the shortest region of the IL-15 receptor alpha (IL-15Rα) capable of binding to IL-15 such that the resulting complex or fusion ‘presents’ the IL-15 cytokine more effectively the IL-15 receptor complex. These so-called IL-15 superagonists thus enhance the therapeutic capacity of this cytokine. Said IL-15 superagonists exhibit appreciable stability and activity in vivo, and have been shown to promote considerable activation of immune cells. Superagonists may also comprise additional modification to the IL-15 cytokine such to create more active “muteins”. One well-known, non-limiting example of a mutation to enhance activity is the IL-15 N72D mutation. The IL-15N72D mutein exhibits superagonist activity through improved binding ability at least to the human IL-15Rβ chain of the receptor complex (Zhu et al 2009; J Immunol. 15; 183(6): 3598. doi:10.4049/jimmunol.0901244).

By contrast, other groups have focused on disabling, disrupting or impairing function of IL-15 in a variety of ways. Such approaches generate a different class of IL-15 muteins that partially block, disable, deactivate or disrupt full wild-type functionality of the cytokine and/or binding to one or more of the CD215, CD122 and CD132 receptor component parts. Non-limiting examples include those reported by Bernard et al 2004 (doi.org/10.1074/jbc.M312458200) and Quemener et al 2020 (doi.org/10.1074/jbc.M312458200) such as D8S, L45D, E46K, L47D, S58K, N65K, L66D, L66E. And one example disabling mutein studied in depth contains the Q108E mutation e.g. see Yu et al (2019, WO2019173832A2). For example, therein, the focus is on reducing the activity of the IL-15 with aid of masking peptides alongside mutations such as Q108E which can reduce biological activity by at least 5 times through to at least 100 times. Of further relevance, and prior to this, Kim et al 1998; J Immunol. 160(12): 5742-5748 also mutated IL-15 at position Q108D alongside Q101D to create antagonistic IL-15 mutants that inhibits stimulatory and proliferative effects conferred by IL-15. Said muteins were so designed to block delayed-type hypersensitive responses. Alternative approaches to impacting IL-15 functionality include mutations to cysteines—typically cysteines form structurally important disulfide bridges.

Further modifications include examples by Kermer et al who fused either the IL-15 wild-type cytokine or a superagonists comprising IL-15/IL-15Rα to the binding domains of an antibody targeting the tumour stromal fibroblast activation protein (FAP). The resulting molecules exhibited antibody-mediated specific binding and cytokine activity (Kermer et al 2010, 2012; (2010) CIMT, 8th annual meeting: abstract No. 113, 163; (2012) Mol Cancer Ther Mol 6:1279-88). Other groups have fused IL-15/IL-15Rα polypeptide to Fc domains for enhanced effects (e.g. see XmAb®24306, Bernett et MJ al 2020, Cancer Res. 2018; 78:5565, ALT-803, Knudson K M et al, Expert Opin Biol Ther 20: 705-709, 2020).

Nevertheless, and as recently highlighted by Isvoranu et al 2021 (Exp. And Therapeutic Medicine 22: 675), despite all such attempts, barely a fraction of cancer patients respond to these new therapeutic approaches. Isvoranu et al explain further that this is likely because the immunosuppressive tumour micro-environment restricts both innate and adaptive antitumour responses. They also emphasize that whilst there have been more than 6,000 papers and more than 170 clinical trials involving IL-15 cytokine based polypeptides, and whilst the National Cancer Institute (NCI) ranked IL-15 among the top 20 immunotherapeutic agents in cancer therapy over a decade ago, there is still need for improvement. Indeed these authors highlight what they term the ‘dark side’ of IL-15 given its involvement in the pathogenesis of several autoimmune diseases. They therefore conclude that one of the main challenges associated with the use of IL-15 polypeptide-based medicaments for cancer immunotherapy remains maximizing tumour response whilst limiting toxicity. It is thus for a combination of reasons that there remains need for improved IL-15-based medicaments.

### Stress-Sensing Receptors Expressed on NK Cells and Gamma Delta T-Cells.

NK and gamma delta T-cells are immune effector cells of great interest to the field of immuno-oncology. They share a number of upregulated or notably expressed receptors associated with sensing, and responding to stressed or diseased cells. Centrally, the best characterized of these receptors include NKp30, NKp44 and NKp46 (collectively termed Natural Cytotoxicity Receptors or NCRs), NKG2D and DNAM-1. Whilst encoded by different genes, these receptors share the following commonality:
All are transmembrane homodimeric receptors.
All can be highly expressed or upregulated on both NK cells and Gamma Delta T-cells and CD8 T cells
All bind ligands upregulated and expressed on stressed, infected or diseased cells
Upon ligand binding, said receptors trigger NK cells and gamma delta T-cell activation. For general review on expression profile and upregulation of said receptors see (see Wensveen et al (2018) doi.org/10.3389/fimmu.2018.00441; Hudspeth et al doi.org/10.3389/fimmu.2013.00069; Sanchez-Correa et al 2019, doi: 10.3390/cancers11060877 and references therein)
Importantly the ligands to these receptors are membrane-bound and upon binding said receptors, receptor activation is triggered via receptor cross-linking or oligomerization—sometimes termed clustering.
Upon activation, said receptors signal, at least in part, via the same downstream signalling pathways e.g. as mediated via ERK and/or JNK (e.g. see https://doi.org/10.1073/pnas.0611655104; doi.org/10.3389/fimmu.2017.01124))
Equally importantly, these receptors can be blocked by soluble versions of their cognate ligands (e.g. shed extracellular domains)—said soluble version of the ligands do not trigger receptor clustering or cross-linking upon binding.

Consequently, tumours can evade gamma delta T cells and NK cells by shedding these soluble ligands as extracellular domains (ECDs) which then block instead of activate cognate receptors expressed on said cells (for example see doi.org/10.3390/ijms22042189).

Most importantly for the context of this application, many if not all antibodies which bind these receptors also behave as blockers or only weak agonists; particularly if presented in a soluble form. But oftentimes said antibodies become more agonistic, more activating, if surface bound. Such surface bound presentation of said antibodies can be via a tissue culture surfaces, via microbead carriers, or when presented by another membrane or cell type, or as larger order protein aggregates.

This surface-bound versus soluble dichotomy will be explained further via the following case-studies.

### Anti-NKG2D, Anti-NKp30, Anti-NKp44, Anti-NKp46 and Anti-DNAM1 Antibodies; Soluble Versus Surface-Bound Effects

### NKp30

NKp30 is an exemplar of an NCR class of receptors. It is also known as natural cytotoxicity receptor 3 (NCR3) and CD337. Studies have shown that anti-NKp30 antibodies can behave as an antagonist or agonist dependent on whether the antibodies employed are presented in a soluble format or are surface bound. For example, Vitale et 2004 (doi.org/10.1182/blood-2004-10-4035) demonstrate this. Specifically, they highlight that “plastic bound” anti-NKp30 antibody binding triggers NKp30 receptor activation on NK cells. In contrast, they also highlight that in a soluble format, the same mAb mediates blocking of NKp30 resulting NK cell inhibition.

Separately other studies have outlined this soluble versus surface effect also hold true for NKp30 ligand interactions with the NKp30 receptor. For example, Reiners et al (2013) doi.org/10.1182/blood-2013-01-476606 demonstrate that inhibition of NK cell cytotoxicity was attributed to the soluble ligand BAG6/BAT3 which engages NKp30 receptor expressed on NK cells. In contrast, they outline that the same NK cells were activated when BAG6 was presented from a surface (in this instance from an exosome).

### NKp44

Like NKp30, NKp44 is another member of the NCR family expressed on gamma delta T cells and NK cells. It is also known as natural cytotoxicity receptor 2 (NCR2) and CD336. The triggering of this receptor is also associated with cell activation and can also be achieved by cross-linking. However, anti-NKp44 antibodies such as Z231 are typically reported to operate as blocking antagonists (Vitale et al 1998 doi.org/10.1084/jem.187.12.2065). Consequently, it has been conceived that anti-NKp44 antibodies are best employed to suppress activation and thus could be used for treating or preventing GVHD (see EP3575320A1).

### NKp46

In a similar manner, NKp46 is also a member of the NCR class of receptors expressed on gamma delta T-cells and NK cells. It is also known as natural cytotoxicity receptor 1 (NCR1) and CD335. It has been demonstrated that anti-NKp46 antibodies, if presented from a surface, can be employed to activate the NKp46 receptor. For example, see Guo et al 2018 (doi.org/10.1073/pnas.1804931115) who employed plate-bound anti-NKp46 (29A1.4) to stimulate NK cells. However, and as reported for anti-NKp44 above, anti-NKp46 antibodies are also reported as blocking. Indeed, anti-NKp46 antibodies have been shown to work in cooperation with anti-NKp44 antibodies to sharply inhibit NK cell cytotoxicity towards cancer cells (Vitale et al 1998 IBID doi.org/10.1084/jem.187.12.2065)

### DNAM-1

(DNAM-1, also known as CD226) is a 65 kDa transmembrane glycoprotein containing two immunoglobulin-like domains. It is also expressed on NK and Gamma Delta T-cells. Cell surface presented ligands can bind to this receptor and induce cross-linking and downstream activation. However, of the few anti-DNAM-1 antibodies presented to date, the most exhaustive study reported anti-DNAM-1 antibodies to exhibit blocking/depleting activity (antibodies TX42 or TX92; see DOI: 10.1089/mab.2012.0083) with a proposed medical use in GVHD control. Alternatively, there has been at least one report of agonistic anti-DNAM-1 antibodies. However, to date, the associated reported agonism was modest (when explored in combination with CD28/CD3 co-stimulation; see WO2020023312A1). And of possible relevance is that a clinical trial by the applicants with an anti-DNAM-1 antibody named LY-343515 was terminated early with no results reported.

### NKG2D

Antibodies selective for NKG2D also behave as agonists or antagonists dependant on whether they are surface bound or soluble. And given the relative maturity of this particular case-study, a deeper dive into the background of anti-NKG2D antibodies is warranted:

The NKG2D homodimeric receptor (natural killer group 2, member D receptor) is expressed on a variety of cells including NK cells and γδ T cells. It is a member of the NKG2 (CD94) class of receptors. This membrane-bound receptor is activated upon binding a variety of membrane associated ‘stress’ ligands decorating dysregulated cells (e.g. infected or cancerous cells).

To date, many such NKG2D ligands have been identified (MICA, MICB, Rae-1 etc.). These ligands have affinities to the NKG2D receptor in the range of 6 to 9000 nM. For a review of such ligands see Spear et al 2013; Cancer Immunity Vol. 13, p. 8.

The NKG2D receptor comprises an extracellular domain, a transmembrane domain, and an intracellular domain. Like the NCR class of receptors, it is also of interest to the field of immunotherapy and for similar reasons. For example, previously and specifically, the NKG2D extracellular domain (ECD) has been generated as an Fc fusion domain to promote antitumour immunity (Feng et al; Cancer Immunol Immunother 2020 October; 69(10):2147-2155). In this instance the NKG2D ECD targets certain ligands decorating stressed cells found in the tumour microenvironment (TME) whilst the Fc domain recruits Fc receptor positive immune cells to stimulate or ‘heat up’ immune cell activity in the TME.

Yet another, quite separate, immunotherapy approach with the potential to treat cancers has involved the development of anti-NKG2D antibodies. These antibodies are typically raised to selectively target NKG2D. Upon binding NKG2D receptor expressed on a cell surface, such antibodies either function as NKG2D receptor antagonists or agonists. Indeed, and as per the prior anti-NCR examples above, how they function is typically dependant on experimental conditions and antibody format. For example such dual agonist/antagonist characteristics exhibited by some—if not all—anti-NKG2D antibody binding domains is demonstrated by studies undertaken and described by Kwong et al; J Mol Biol. 2008, December 31; 384(5): 1143-1156. During their discovery of an anti-NKG2D antibody termed “KYK-2.0” they highlight that when used in a soluble format, KYK-2.0 exhibits antagonistic activity through interfering with effector-cell to target-cell recognition otherwise mediated by NKG2D receptor/ligand interactions. However, they also demonstrated that when KYK-2.0 is first immobilized to a surface it can operate as an agonist through triggering the NKG2D receptor on NKG2D positive effector cells. A very similar situation was also described concurrently by Steigerwald et al (2009) mAbs 1:2, 115-127) wherein their panel of leads mAb such as “E4” also exhibited antagonist activity (when soluble) or agonist activity (when immobilized or presented). Consequently, they considered that such anti-NKG2D mAbs exhibit an “ambivalent” nature before ultimately concluding that their lead was likely best utilized as an antagonistic anti-NKG2D antibody for the treatment of patients with inflammatory diseases.

These observations have been further explored by others in this space. For example, other studies have involved fusing anti-NKG2D antibody fragments to anti-CD16 binding domains such that the resulting molecules can be immobilized or presented on the surface of CD16-receptor positive cells, (see Hagelstein et al (2021) Front Immunol. 2021; 12: 653081). This thereby encourages the anti-NKG2D antibody fragments to operate agonistically upon subsequent binding to the NKG2D receptor. Additionally, it is also quite commonplace for such research teams involved with the discovery and development of anti-NKG2D antibodies to coat them onto tissue culture well surfaces, or capture the antibodies on protein-A beads, prior to exploring agonistic properties. Further it is quite typical to employ wild-type IgG1 constant domains to ensure Fc receptor engagement and antibody presentation (e.g. via CD16, CD32 and/or CD64 binding).

Alongside this commonplace need to immobilize such antibodies on a surface to ensure agonistic rather than blocking or antagonistic effects predominate, many such research groups often stimulate or pre-stimulate the NKG2D receptor positive immune cells with IL-2 and/or anti-CD3 antibodies ahead of, or in parallel to, the addition of the anti-NKG2D antibodies under investigation.

Therefore, whilst anti-NKG2D antibodies might offer potential as a class of antibodies designed to activate NKG2D receptor-positive cells and thereby immunologically stimulate immune cells and “heat up” the TME, to date such antibodies have not progressed rapidly. Indeed, the most advanced anti-NKG2D antibody in the clinic to date is tesnatilimab. By design this anti-NKG2D operates predominantly as an antagonist blocker of NKG2D receptor/ligand engagement. It is being explored for the treatment of autoimmune diseases whereby dampening down the immune system is preferred (rather than heating it up). To further ensure tesnatilimab operates as an antagonist, it is formatted as a modified IgG4 such as to reduce Fc receptor binding and potential agonistic effects mediated by Fc receptor immobilization and trans-presentation.

### Anti-NKG2D, Anti-NKp30, Anti-NKp44, Anti-NKp46 and Anti-DNAM1 Antibodies; Summary

In sum, antibodies selective for the aforementioned homodimeric receptors typically exhibit ambivalent dual properties and do not reliably operate as agonists. This class of antibodies typically require surface presentation to ensure agonism. Oftentimes additional stimulants such as IL-2 pre-treatment or the addition of anti-CD3 antibodies can be employed to ensure they operate as effective agonists to activate rather than bind and block said receptors on receptor-positive immune cells. However, with such additional broad stimulants comes significant additional toxicity concerns if employed as part of a therapeutic regimen. Hence there is a need for improved anti-NKG2D, anti-NKp30, anti-NKp44, anti-NKp46, and anti-DNAM-1 antibody-based medicaments in this field. Ideal such medicaments would robustly and consistently and selectively activate or agonize immune cells such as NK cells and gamma delta T-cells.

### Valency, Aggregation, and Immunostimulatory Receptor Clustering—More General Considerations

Publications as long ago as 1972 by Hornick and Karush (doi.org/10.1016/0019-2791(72)90096-1) highlight the importance of valency (and multivalency) when considering ligand mediated receptor cross-linking. Therein they also highlight that monovalent binders can exhibit 100-fold decrease in functional affinity.

However, for immunostimulatory antibodies, for example such as those which target members of the tumour necrosis factor superfamily receptors (see Zhang et al 2016, doi: 10.1074/jbc.M116.757773) oftentimes, and even despite comprising a bivalent format, one antibody molecule may not be able to cluster sufficient receptors to activate the cells. Instead, antibody cross-linking via attachment methods (e.g. on beads or surface of assay plate) may still be needed for receptor activation.

Equally worth noting at this juncture is the fact that antibody aggregation can also result in an effective increase in functional agonism. Indeed, those skilled in the art will be aware that antibody aggregates can result in enhanced agonism and negate the need for coating onto beads etc. However, whilst increasing the levels of higher order structures and aggregate in an antibody preparation could be one method by which one might increase the agonistic properties of an antibody preparation, it is highly undesirable as said antibody preparations may also be immunogenic. Furthermore, the degree of aggregation and so agonism is highly variable by such methods. Hence typically those skilled in art do not develop aggregated antibodies but rather ensure antibody preparation are preferably 80% or more preferably 90% monomer as measured by established analytical HPLC methods designed to separate purified antibody and recombinant protein monomers from their higher order structures and aggregate. Paradoxically in some instances this will mean the resulting more controlled, more monomeric antibody preparation is less agonistic or indeed may not function as an agonist whatsoever. Interestingly, and whilst for the reasons discussed here, monomeric antibodies are most preferred, nature does provide alternative solutions. For example, soluble IgGA exists primarily as a dimeric, “dog bone” like structure. Said antibody dimers have four Fab binding domains that are presumed to enhance binding avidity and antigen cross-linking potential when compared to typical IgG antibodies with two Fab binding domains (e.g. see Bharathkar et al (2020) doi.org/10.7554/eLife.56098). However, for technical reasons soluble IgGA dimers are not as typically developable as medicines given the propensity for IgA to former higher order aggregates (e.g. see Xie et al (2021 doi.org/10.1172/jci.insight.150551)). Instead IgG antibodies are more preferred.

For further general issues associated with aggregation in this context see Rombach-Riegraf et al 2014 (doi: 10.1371/journal.pone.0086322) for discussion on the influence of aggregate on the innate immune system. And see discussion by Komatsu et al 2020 (doi.org/10.1021/jacs.0c05331) for more specific discussion on the (positive) impact of aggregating antibodies when activating receptors—albeit in a controlled inducible manner in their example.

### Co-Activating Gamma Delta and NK Cells

Gamma delta (γδ) T cells represent a relatively small fraction of the total lymphocyte population. They are known to play an important role in innate and adaptive immunity. They have been described as polyfunctional and can produce an array of immunomodulatory cytokines. They can infiltrate the tumour microenvironment and their presence has been reported as a positive prognostic factor associated with improved outcomes in cancer.

Natural killer cells are innate lymphocytes so named for their cytolytic activity. They can also control tumour growth and also produce an array of cytokines.

Both these cell types can express NKG2D, NKp30, NKp44, NKp46, DNAM-1, CD122, and CD132 and both cells type are capable of rapid response to diseased and cancerous cells and both can influence the broader immune system including B cells and αβ T cells

For various legacy reasons, NK and gamma delta T cell biology and function are often explored separately. Nevertheless, the design and use of medicaments to selectively distribute to, and selectively activate both these two cell types in concert may be highly desirable in a variety of cancer settings.

### SUMMARY OF THE INVENTION

In general, the invention provides immunoconjugates which result in the targeted co-presentation of immunoconjugates to cells of the immune system in either cis- or in trans-manner to ensure selective dual agonism. The immunoconjugates have enhanced functionality to selectively target and activate immune cells. More specifically, the invention relates to enhancing the agonistic properties of antibodies which selectively target one receptor from a group consisting of NKG2D, NKp30, NKp46, NKp44 and DNAM-1 by the immunoconjugate formats of the invention. Particularly this invention relates to immunoconjugates having at least two antibody binding domains, at least two IL-15 cytokine domains, and at least two CD215 domains (such as sushi domains). The resulting multivalent “dog-bone” immunoconjugate contains two antibody binding domains and two IL-15 cytokine binding domains and as such can be termed a 2+2 multivalent approach. This ensures dual agonism and the immunoconjugates of the invention have augmented ability to target and activate specific immune cell types.

In a first aspect, the invention provides an immunoconjugate comprising:
a) at least two IL-15 polypeptides, each comprising
   i. an IL-15 cytokine; and
   ii. IL-15 receptor alpha (CD215) or a functional fragment thereof; and
b) a multivalent antibody or antigen-binding fragment thereof, comprising at least two antibody binding domains that each specifically bind to an antigen selected from the group consisting of NKG2D, NKp30, NKp46, NKp44 and DNAM-1.

In some embodiments of the invention, the IL-15 polypeptide component of the first aspect may be an IL-15 superagonist

In some embodiments of the invention, the IL-15 polypeptide component may be fused to the C-terminal end of a chain of the multivalent antibody or antigen-binding fragment thereof, on either the heavy or light chain.

In some embodiments of the invention, the IL-15 polypeptide and antibody binding domain may be separated by a linker, such as a flexible linker.

In some embodiments of the invention the IL-15 polypeptide may comprise a human IL-15 cytokine and a human IL-15 receptor alpha (CD215) sushi domain and the multivalent antibody component may bind to human and/or cynomologous NKG2D, NKp30, NKp46, NKp44 or DNAM-1.

In some embodiments, the antibody binding domains may specifically bind to NKG2D and may comprise a heavy chain variable region (VH) comprising a heavy chain complementary determining region (HCDR) 1 of SEQ ID NO: 6, a HCDR 2 of SEQ ID NO: 19, and a HCDR 3 of SEQ ID NO: 32; and a light chain variable region (VL) comprising a light chain complementarity determining region (LCDR) of SEQ ID NO: 45, a LCDR 2 of SEQ ID NO: 58, and a LCDR 3 of SEQ ID NO: 71.

In some embodiments, the antibody binding domains may specifically bind to NKG2D and may comprise a heavy chain variable region (VH) comprising a heavy chain complementary determining region (HCDR) 1 of SEQ ID NO: 1, a HCDR 2 of SEQ ID NO: 14, and a HCDR 3 of SEQ ID NO: 27; and a light chain variable region (VL) comprising a light chain complementarity determining region (LCDR) of SEQ ID NO: 40, a LCDR 2 of SEQ ID NO: 53, and a LCDR 3 of SEQ ID NO: 66.

In some embodiments, the antibody binding domains may specifically bind to NKp30 and may comprise a heavy chain variable region (VH) comprising a heavy chain complementary determining region (HCDR) 1 of SEQ ID NO: 11, a HCDR 2 of SEQ ID NO: 24, and a HCDR 3 of SEQ ID NO: 37; and a light chain variable region (VL) comprising a light chain complementarity determining region (LCDR) of SEQ ID NO: 50, a LCDR 2 of SEQ ID NO: 63, and a LCDR 3 of SEQ ID NO: 76.

In some embodiments, the antibody binding domains may specifically bind to NKp46 and may comprise a heavy chain variable region (VH) comprising a heavy chain complementary determining region (HCDR) 1 of SEQ ID NO: 13, a HCDR 2 of SEQ ID NO: 26, and a HCDR 3 of SEQ ID NO: 39; and a light chain variable region (VL) comprising a light chain complementarity determining region (LCDR) of SEQ ID NO: 52, a LCDR 2 of SEQ ID NO: 65, and a LCDR 3 of SEQ ID NO: 78.

In any embodiment the IL-15 polypeptide component may comprise an IL-15 superagonist. In preferred embodiments the IL-15 superagonist comprises or consists of SEQ ID NO: 154. This IL-15 polypeptide comprises a sushi domain (SEQ ID NO: 149), a linker (SEQ ID NO: 183) and an IL-15 cytokine (SEQ ID NO: 151). In some embodiments the IL-15 superagonist comprises or consists of SEQ ID NO: 206. This IL-15 polypeptide comprises a sushi domain (SEQ ID NO: 149), a linker (SEQ ID NO: 184) and an IL-15 cytokine (SEQ ID NO: 151). In some embodiments the IL-15 superagonist comprises or consists of SEQ ID NO: 207. This IL-15 polypeptide comprises a sushi domain (SEQ ID NO: 149), a linker (SEQ ID NO: 201 and/or 183) and an IL-15 cytokine (SEQ ID NO: 151).

In some embodiments the flexible linker, for example the linker separating the IL-15 component from the antibody component, or the linker separating the IL-15 cytokine from the IL-15 receptor alpha domain, may comprises or consists of glycine and serine residues. In some embodiments the flexible linker separating the IL-15 component from the antibody component, or the flexible linker separating the IL-15 cytokine from the IL-15 receptor alpha domain, comprises or consists of the sequence [GGGGS]ₙX, or [GGGS]ₙX, or [GGS]ₙX, wherein n is from 1 to 6 and wherein X is G or is absent. In some embodiments, the linker comprises or consists of the sequence [[G]ₘS]ₙX, wherein m is from 2 to 4, n is from 1 to 6 and X is G or is absent. In some embodiments the flexible linker separating the IL-15 component from the antibody component, or the flexible linker separating the IL-15 cytokine from the IL-15 receptor alpha domain, comprises or consists of a sequence selected from the group consisting of SEQ ID NOs: 182 to 187 and 204 to 205.

In some embodiments the sushi domain contained within the multivalent immunocytokines comprises additional sequences of the human IL-15 receptor alpha gene exon 3. For example, the addition of 13 amino acids (SEQ ID NO: 201) derived from Exon 3 has previously been included at the C-termini of the sushi component as additional linker or ‘hinge’ in certain variant sushi domain—IL15 fusions.

In some embodiments, the multivalent antibody component comprises an Fc (CH2 and CH3) domain. In some embodiments this is combined with a CH1 domain and form a contiguous CH1-hinge-CH2-CH3 IGHC. In some embodiments, the CH1 domain, the hinge domain, and the Fc domain, are human. In some embodiments, the antibody is an IgG class, particularly an IgG1 subclass immunoglobulin (see SEQ ID NO:105 for one such example and preferred IGHC heavy chain allotype. This is also described by Uniprot as entry PO1857-1 inclusive of C-terminal glycine and lysine (see https://uniprot.org).

In some embodiments, wherein the antibody component comprises an Fc domain, the Fc domain comprises one or more amino acid substitution that reduces binding to an Fc receptor. In some such embodiments, said one or more amino acid substitution is at one or more position selected from the group of L234, L235, G236, G237 and P329 (EU index numbering). In some embodiments, each subunit of the Fc domain comprises the amino acid substitutions L235A, G237A and P329G (EU index numbering). In some embodiments, the antibody is an IgG class, particularly an IgG1 subclass immunoglobulin which is “Fc disabled” or “effector disabled” (SEQ ID NO:106 or SEQ ID NO:203).

Generally, in embodiments of the invention, the at least two IL-15 polypeptides are identical to each other, or may be at least 95%, at least 96%, at least 97%, at least 98% or at least 99% identical to each other.

In some embodiments, the immunoconjugate comprises:
a) a first chain comprising a first IL-15 polypeptide and a first heavy chain of an antibody or antigen-binding fragment thereof,
b) a second chain comprising a first light chain of an antibody or antigen-binding fragment thereof;
c) a third chain comprising a second IL-15 polypeptide and a second heavy chain of an antibody or antigen-binding fragment thereof; and
d) a fourth chain comprising second light chain of an antibody or antigen-binding fragment thereof. The four chains may be covalently or non-covalently associated in an immunoglobulin format, whereby the first heavy chain and first light chain form a first antigen binding domain and the second heavy chain and second light chain form a second antigen binding domain.

In some embodiments, the immunoconjugate comprises:
a) a first chain comprising a first IL-15 polypeptide and a first light chain of an antibody or antigen-binding fragment thereof,
b) a second chain comprising a first heavy chain of an antibody or antigen-binding fragment thereof;
c) a third chain comprising a second IL-15 polypeptide and a second light chain of an antibody or antigen-binding fragment thereof; and
d) a fourth chain comprising second heavy chain of an antibody or antigen-binding fragment thereof. The four chains may be covalently or non-covalently associated in an immunoglobulin format, whereby the first heavy chain and first light chain form a first antigen binding domain and the second heavy chain and second light chain form a second antigen binding domain.

Generally, the multivalent antibody or antigen-binding fragment thereof of the immunoconjugates of the invention is monospecific. In such embodiments, the at least two antibody binding domains have the same binding specificity.

Further information and guidance on the design of a non-limiting immunoconjugate as provided herein is provided in FIG. 2.

In a second aspect of the invention there is provided a method of producing an immunoconjugate of the invention, comprising (a) culturing the host cell of the invention under conditions suitable for the expression of the immunoconjugate, and optionally (b) recovering and purifying the immunoconjugate. Immunoconjugates obtained or obtainable by such methods are also provided.

In a third aspect of the invention there is provided one or more isolated polynucleotides encoding an immunoconjugate of the invention or encoding one or more chains of an immunoconjugate of the invention.

In a fourth aspect of the invention, there is provided vector, particularly expression vector, comprising a polynucleotide of the invention

In a fifth aspect of the invention there is provided a host cell comprising one or more polynucleotides or one or more vectors of the invention.

In a sixth aspect of the invention there is provided a composition comprising an immunoconjugate of the invention. The composition may be a pharmaceutical composition and may comprise one or more pharmaceutically acceptable carriers.

In a seventh aspect of the invention there is provided an immunoconjugate, composition or pharmaceutical composition according to the invention for use as a medicament, or for use in the treatment of a disease. In some embodiments, said disease is cancer. There is also provided the use of an immunoconjugate according to the invention in the manufacture of a medicament for the treatment of a disease. In some embodiments, said disease is cancer. Also provided is an immunoconjugate, composition or pharmaceutical composition according to the invention for use in stimulating the immune system of an individual. Also provided is the use of an immunoconjugate according to the invention in the manufacture of a medicament for stimulating the immune system of an individual.

In an eighth aspect of the invention there is provided an immunoconjugate, comprising at least two IL-15 polypeptides, each comprising an IL-15 cytokine; and IL-15 receptor alpha (CD215) or a functional fragment thereof; and a multivalent antibody or antigen-binding fragment thereof, comprising at least two antibody binding domains, wherein the multivalent antibody or antigen-binding fragment thereof specifically binds to NKG2D. In some embodiments the multivalent antibody specifically binds NKG2D and another antigen.

In an ninth aspect of the invention there is provided a method of treating disease in an individual, comprising administering to said individual a therapeutically effective amount of an immunoconjugate, composition or pharmaceutical composition of the invention to a subject in need thereof. In some embodiments, said disease is cancer.

In a tenth aspect of the invention there is provided an immunoconjugate of the invention which has been selectively purified to generate a tetramer consisting of two sequence-identical heterodimeric sub-units.

Also provided is a method of stimulating the immune system of an individual, comprising administering to said individual an effective amount of an immunoconjugate, composition or pharmaceutical composition of the invention to a subject in need thereof.

### BRIEF DESCRIPTION OF THE DRAWINGS

FIG. 1: Schematic representation of an immunoconjugate, its component parts, and example non-limiting modus operandi. (A) Schematic representation of component parts of the immunoconjugate and its target receptors. (B) Example of the immunoconjugate operating in trans-presentation mode (involving one cell (“cell A”) expressing IL-15 receptor complex (also known as CD122/132 receptor) and a second cell (“cell B”) expressing NKG2D, NKp30, NKp46, NKp44, or DNAM-1 receptor). (C) Example of the immunoconjugate operating in cis-presentation mode (involving one cell expressing both IL-15 receptor complex and NKG2D, NKp30, NKp46, NKp44, or DNAM-1 receptor). (D) Graphical representation of how said immunoconjugates could operate upon provision to, and contact of, NK cells and/or gamma delta T-cells. This schematic includes a representation of tumour adapted from https://arxiv.org/pdf/1602.02077.pdf).

FIG. 2: Non-limiting guidance on the design of immunoconjugate examples of the invention provided herein.

FIG. 3: (A) Human protein tissue expression profile. Annotation methodology as described by Human Protein Atlas: “ . . . annotation parameters include an evaluation of i) staining intensity (negative, weak, moderate or strong), ii) fraction of stained cells (<25%, 25-75% or >75%) and iii) subcellular localization (nuclear and/or cytoplasmic/membranous) . . . ”. This table comprises data adapted from proteinatlas.org and is focused on tissue rather than cell type expression profiles. For example, where spleen is positive for NKG2D, this may be due to NKGD positive immune cells found more abundantly there. In contrast where tissues are positive for CD215 such as heart tissue, this may be due to an abundance CD215 positive endothelial cells found there. However, cell type detail is beyond this Table representation. Immune cells distributed to tissues in smaller numbers may not be represented here. For example, NKG2D positive cells such as CD8 T-cells present in skin may not be represented by this summary. Regardless, this table helps highlight that CD215 positive cells are generally more ubiquitous and thus could act as a sink for standard IL-15 cytokine based therapies. (B) Single cell RNA profile data for CD122, NKG2D, NKp30 and NKp46 from proteinatlas.org.

FIG. 4: Examples of preferred formats of immunoconjugates of the invention. The immunoconjugates are multivalent for both targets to enhance cross-linking and dual, two-way agonism. (A) SushiCD215/IL15 positioned at C-terminus of the heavy chain. (B) SushiCD215/IL15 positioned at C-terminus of the light chain.

FIG. 5: Non-exhaustive schematic representations of example formats. Associated control and comparator formats also shown. All cartoons shown are orientated with N-termini at the top and C-termini at the bottom of the page. The more complex (e.g. beta-sheeted) domains are represented as ovals. Ig hinge and ‘less structured’ linkers are represented as curved lines. For further grey-scaled shading guidance refer to Format A. VH/VL domains are represented by black shading, Ig framework regions are represented by light grey shading and the Sushi+IL15 domain is represented by dark grey shading. (A) Format A; the N-termini comprise multivalent anti-NKG2D, or anti-NKp30, or anti-NKp44, or anti-NKp46, or anti-DNAM1 Vh/Vl pairs (black, indicated). The C-termini comprise multivalent sushi-IL15 domains fused to the C-termini of the heavy chain IgG (darker grey, indicated). For further detail on Format A also see FIG. 4A; (B) Format B; the N-termini comprise sushi-IL15 binding domains fused to the N-termini of the heavy chains of multivalent anti-NKG2D, or anti-NKp30, or anti-NKp44, or anti-NKp46, or anti-DNAM1 Vh/Vl; (C) Format C; the N-termini comprise multivalent anti-NKG2D, or anti-NKp30, or anti-NKp44, or anti-NKp46, or anti-DNAM1 Vh/Vl pairs. The C-termini comprise multivalent sushi-IL15 domains fused to the C-termini of the light chain. For further detail on Format C also see FIG. 4B; (D) Format D; the N-termini comprise multivalent anti-NKG2D, or anti-NKp30, or anti-NKp44, or anti-NKp46, or anti-DNAM1 Vh/Vl pairs. The C-termini comprise multivalent IL15 domains fused to the C-termini of the IgG heavy chain without sushi domains (as indicated); (E) Format E; typical isotype controlled multivalent anti-NKG2D, or anti-NKp30, or anti-NKp44, or anti-NKp46, or anti-DNAM1 antibodies employed as controls in our studies; (F) Format F; a monovalent scFV anti-NKG2D fused via linker to IL-15 without a sushi domain (as indicated). (G) Format G; a format comprising three chains. The first chain (most left) comprises a sushi-IL15 domain fused to the N-terminus a heavy chain Fc domain. The second chain (middle) comprises an antibody heavy chain. The third chain (most right) comprises a light chain. The second and third chain pair to create an anti-NKG2D Vh/Vl binding domain. This second/third paired domain heterodimerizes with the first chain via standard CH3 knob-in-hole mutations at the C-termini. Like format F, this format is also monovalent for each target.

FIG. 6: Expression and purification of multivalent immunoconjugates alongside controls and comparators. This Figure summarizes the production of exemplar multivalent immunoconjugates and associated comparators and controls. After two-step HPLC purification comprising standard resin-based capture and elution followed by preparative SEC single-peak purification, all materials aside the CYTB27 comparator molecule exhibited an acceptable profile for this stage of development.

FIG. 7: KD Determination to Human NKG2D and Human IL-15 Receptor beta. The KD values are reported for molecules as indicated using classical Langmuir 1:1 model.

FIG. 8: Avidity Determination; multivalent versus monovalent formats. (A) A Summary of the KD values generated after molecule captures by sensor chips first coated with NKG2D or IL-15 receptor beta (B-E) Cartoon representations (left) and representative sensograms (right) respectively for CYTB05, CYTB27, CYTB29, and CYTB30 as indicated. Sensogram plots presented use typical X/Y plots with response units/time coordinates. Five-fold diluted stepwise responses from 25 nM to 1.56 nM for each molecule are shown. These sensograms were employed to determine the KD summary values.

FIG. 9: Dual Binding Studies. Summary results table and example sensograms (A) Summary results of dual binding properties for all molecules studied in this assay. For the second target injection column, a dash symbol (-) is employed to indicate that the molecule is already pre-bound to the associated target. A ‘NO’ binding event is concluded where the steady state response value upon injection of the target protein is less than 5RU. (B,C) Example dual binding sensogram X/Y plots representing a response/time axis respectively. The overlayed vertical black line on each plot indicates the divide between the first and second target injection profiling switch (at 200 seconds). The principal target engagement event (NKG2D or IL-15 beta receptor) being measured pre- and post-this event is also indicated. (B) A CYTB05 dual binding example sensogram where dual binding is observed irrespective of the order in which the targets are engaged (C) A CYTB07 dual binding example sensogram where dual binding sequence order dependency is observed.

FIG. 10: Summary of binding discoveries associated with the Dual Target Multivalent Design. This summary table provides a comparative overview of the affinity/avidity binding profiles associated with the various molecular formats as indicated.

FIG. 11: Synergistic effects conferred by multivalent immunocytokines on primary human NK cells. The relative number (%) of CD69+/CD107a+ double-positive NK cells after 24-hour treatment with titrated (0.1 nM-10 nM) test articles as indicated (A) An X/Y plot summarising the synergistic effects of attaching sushi-IL15 to anti-NKG2D IgG: A clear titratable increase in the double-positive NK gate is seen with CYTB05 but not when the component building blocks are added separately either alone or in combination (B) An X/Y plot summarising the need for correct positioning and design of the sushi-IL15 complex. CYTB05 and CYTB12 comprise sushi-IL15 domains attached by differing linkers to the C-termini of the heavy chain or light-chain respectively. Both molecules induce similar titratable increases in double-positive NK cells. In contrast no such increases are seen with CYTB07 (sushi-IL15 attached to N-termini) or CYTB14 (C-terminal attached IL-15 cytokine without a sushi domain)

FIG. 12: A comparison of multivalent and monovalent immunocytokines-surface marker upregulation. (A-D) Relative number (%) of CD69+/CD107a+ double-positive cells after 24-hour treatment with titrated (0.1 nM-10 nM) test articles as indicated. The superior effects conferred by the two multivalent immunocytokines exemplars CYTB05 and CYTB11 are highlighted (A) Effects of test articles on NK-gated cells (B) Effect of test articles on NK-T gated cells (C) Effects of test articles on gamma delta T-cells and (D) Effect of test articles on CD8 positive cells. (E) Example flow plots at the 10 nM concentrations are shown—the upper right quadrant in each plots indicates the relative number of CD69+/CD107a+ double positive cells. Rows, ii, iii and iv represent the NK, NKT, gamma delta and CD8 sub-gates respectively. These plots provide a visual aid to further highlight the enhanced effects associated with multivalent exemplars CYTB05 and CYTB11 relative to anti-NKG2D IgG (CYTB21) or monovalent entities CYTB29 and CYTB30.

FIG. 13: A comparison of multivalent and monovalent immunocytokines—immune cell proliferation. The number of gated cells after a 24-hour treatment with titrated (0.1 nM-10 nM) test articles as indicated are presented. The superior effects conferred by the two multivalent immunocytokines exemplars CYTB05 and CYTB11 are highlighted (A) Effects of test articles on NK-gated cells (B) Effect of test articles on NK-T gated cells (C) Effects of test articles on gamma delta T-cells and (D) Effect of test articles on CD8 T-cells

FIG. 14: Multivalent immunocytokines—alternate stress-sensing receptor targeting optionality: Effects conferred by three multivalent immunocytokines targeting differing stress receptors. CYTB05 targets NKG2D, CYTB15 targets NKp30, and CYTB16 targets NKp46. Plus respective cognate control IgG1 CYTB21, CYTB24 and CYTB25. X/Y plots present relative number (%) CD69+/CD107a+ double-positive NK cells after 24-hour treatment with titrated (0.1 nM-10 nM) test articles as indicated. (A) Effects of test articles on NK-gated cells (B) Effect of test articles on NK-T gated cells (C) Effects of test articles on gamma delta T-cells and (D) Effect of test articles on CD8-gate.

FIG. 15: Cytokine induction—This figure summarises the fold-change in the levels of interferon-gamma in cultured PBMCs after 24-hour incubation with titrated (0.1-10 nM) test articles or vehicle controls as indicated. (A) An X/Y plot presentation highlighting the marked and significant induction of interferon-gamma induced by the exemplar CYTB05 and CYTB11 multivalent immunocytokines relative to that observed by the sushi-IL-15 complex and CYTB21 (anti-NKG2D IgG1) control. (B) An extended overlay of part A in which CYTB16 (the anti-NKp46 exemplar variant) and monovalent immunocytokine comparators CYTB29 and CYTB30 are also included.

FIG. 16: Multivalent immunocytokine augmented immune cell activation and cancer cell killing—The results of a cytotoxicity assay are presented. Assay comprised culture/co-culture of PBMCs and labelled K562s with at an E:T ratio of 10:0 or 10:1 for 144 hours. Test articles as indicated were added at 1 nM and 10 nM concentrations and compared to a vehicle control. (A) A bar-chart summary highlighting the number of live K562 present at the end of the experiment. Results show that in the presence of the multivalent immunocytokine (CYTB05) a marked reduction in the number of live K562 cells is recorded (B) A bar-chart presentation summarising the number and phenotype of live immune cells observed in the absence or presence of K562s as indicated. Results show that the multivalent immunocytokine induces proliferation of desired immune cell compartments. They also reveal that further enhanced proliferation of certain immune-cell compartments is conditional on the presence of K562 cancer cells.

FIG. 17: Multivalent immunocytokine target engagement profiling—The summary results of a binding profile assessment of Zenon-labelled CYTB05 undertaken with ice-chilled PBMCs first pre-blocked with unlabelled excess molecule (as indicated) prior to flow-cytometry based sub-gating. In all cases, the bar charts represent the relative number (%) of cells bound by the labelled CYTB05 molecule at the concentrations (1 nM and 5 nM) indicated. In each study, the key indicates which unlabelled molecule was first pre-bound at excess concentrations (100 nM). See Example 15 for further details on how to interpret the data (A) Relative number (%) of NK cells bound by the labelled CYTB05 (B) Relative number (%) of NKT cells bound by the labelled CYTB05 (C) Relative number (%) of gamma delta T-cells bound by the labelled CYTBD5 (D) Relative number (%) of CD8 T-cells bound by the labelled CYTB05.

FIG. 18: Multivalent immunocytokines—In this alternate design format, the 2+2 multivalency is achieved with a dimeric 2-chain format rather than a 4-chain format as described in FIG. 4. In this alternate design, the IL-15 polypeptide is attached at the N-terminus of a IgG hinge-CH2-CH3 domain whilst the two anti-NKG2D binding domains (or anti-NKp30, or anti-NKp44, or anti-NKp46, or anti-DNAM1 binding domains) are formatted as scFVs and fused to the C-terminus of the IgG CH3 chain. scFV are well understood in the art and typically comprise a Vh/Vl format (or a VI/Vh format) wherein the Vh and VI domains are separated by a linker. In this 2-chain design, optionally both the N-terminal sushi-IL-15 domains and the C-terminal scFVs are joined to the antibody constant sequences via standard linkers as described herein.

### DETAILED DESCRIPTION OF THE INVENTION

### Definitions

Terms are used herein as generally used in the art, unless otherwise defined in the following.

The singular terms “a,” “an,” and “the” include plural referents unless context clearly indicates otherwise.

The term “immunoconjugate” as used herein refers to an antibody format which is joined to a second molecule. The second molecule may be a a cytokine, a radioactive agent, a cytotoxin, an interferon, a target or reporter moiety, an enzyme, a toxin, a peptide or protein, a therapeutic agent or a chemotherapeutic agent. When the second molecule is a cytokine the immunoconjugate can also be known interchangeably as an “immunocytokine”. Other examples of immunoconjugates include antibody drug conjugates and antibody-toxin fusion proteins. The antibody format may be any of those described herein.

The term “fragment” as used herein in the context of a peptide or polypeptide, refers to a peptide or polypeptide that comprises less than the full length amino acid sequence. Such a fragment may arise, for example, from a truncation at the amino terminus, a truncation at the carboxy terminus, and/or an internal deletion of a residue(s) from the amino acid sequence. Fragments may, for example, result from alternative RNA splicing or from in vivo protease activity. The term “functional fragment” refers to a peptide or polypeptide that comprises less than the full length amino acid sequence but retains the functional activity of the full length amino acid sequence from which the fragment is derived (for example the binding profile, the KD, or the agonistic properties).

The term “antigen” as used herein refers to any substance that has antigenic or immunogenic activity in an animal, preferably a mammal, and most preferably in a human, that is capable of evoking an immune response. Antigens may also be referred to as “immunogens”.

The term “epitope” as used herein refers to a localised region of the antigen or target which is specifically bound by the antibody or antigen-binding fragment thereof. Epitopes usually consist of chemically active surface groupings of molecules such as amino acids or sugar side chains and have specific three dimensional structural characteristics as well as specific charge characteristics. Epitopes found on protein targets may be defined as “linear epitopes” or “conformational epitopes”. Linear epitopes are formed by a continuous sequence of amino acids in a protein antigen. Conformational epitopes are formed of amino acids that are discontinuous in the protein sequence, but which are brought together upon folding of the protein into its three-dimensional structure. Epitopes may also be referred to as “antigenic determinants”. An antibody binds “essentially the same epitope” as another antibody when they both recognize identical or sterically overlapping epitopes. Commonly used methods to determine whether two antibodies bind to identical or overlapping epitopes are competition assays, which can be configured in a number of different formats (e.g. well plates using radioactive or enzyme labels, or flow cytometry on antigen-expressing cells) using either labelled antigen or labelled antibody. An antibody binds “the same epitope” as another antibody when they both recognize identical epitopes (i.e. all contact points between the antigen and the antibody are the same).

The term “directly fused” as used herein refers to multiple molecules being physically or chemically joined together without an intermediate joining moiety, such as linker. This can refer to fusions created through the joining of two or more genes that originally coded for separate proteins or peptides or through the post-translation fusion of two or more proteins or peptides.

The term “about” as used herein includes up to and including 10% greater and up to and including 10% lower than the value specified, suitably up to and including 5% greater and up to and including 5% lower than the value specified, especially the value specified. The term “between”, includes the values of the specified boundaries.

The term “agonist” as used herein refers to a substance that binds to a target receptor and change the receptor activity to produce a response, e.g. activate the receptor. Agonists therefore have both affinity and intrinsic efficacy. An “agonistic antibody” or “agonistic immunoconjugate” will therefore bind to a receptor and cause the same action as the substance that normally binds to the receptor. An “inverse agonist” is one which binds to a receptor and causes the opposite action as the substance that normally binds to the receptor e.g. reduces receptor activity.

The term “antagonist” as used herein refers to a substance that binds to a target receptor but does not produce a response, e.g. blocks the receptor but does not activate it. Antagonists therefore have affinity but not intrinsic efficacy. An “antagonistic antibody” or “antagonistic immunoconjugate” will therefore bind to a receptor and by virtue of occupying a fraction of the receptor population, reduces the probability of occupancy by an agonist. Such antibodies or immunoconjugates are therefore referred to as “blocking antibodies” or “blocking immunoconjugates”.

The term “immunostimulant” or “immunostimulators” are substances that promote the activation of immune system components or cells, therefore stimulating the immune system into producing or increasing an immune response. Immunostimulants may be specific, for example an antigen or vaccine, or may be non-specific and operate without antigen specificity, for example an adjuvant. An “immune response” is a measurable change in at least one cell, or one cell-type, or one endocrine pathway, or one exocrine pathway, of the immune system (including but not limited to a cell-mediated response, a humoral response, a cytokine response, a chemokine response), for example upon addition of a modulating immunocytokine.

The term “immune cell” as used herein, refers to a cell of the immune system including, but not limited to, CD34+ cells, B-Cells, CD45+ (lymphocyte common antigen) cells, Alpha-Beta T-cells, Cytotoxic T-cells, Helper T-cells, Plasma Cells, Neutrophils, Monocytes, Macrophages, Red Blood Cells, Platelets, Dendritic Cells, Phagocytes, Granulocytes, Innate lymphoid cells, Natural Killer (NK) cells and Gamma Delta T-cells. Typically, immune cells are classified with the aid of combinatorial cell surface molecule analysis (e.g., via flow cytometry) to identify or group or cluster to differentiate immune cells into sub-populations.

The term “subject” or “patient” refers to any animal, including, but not limited to, mammals.

The term “composition” as used herein, is intended to encompass a product containing the specified ingredients (e.g. an immunoconjugate of the invention) in, optionally, the specified amounts, as well as any product which results, directly or indirectly, from combination of the specified ingredients in, optionally, the specified amounts.

The term “cancer,” as used herein, refers to the abnormal growth or division of cells. Cancers may be benign, pre-malignant or malignant. Generally, the growth and/or life span of a cancer cell exceeds, and is not coordinated with, that of the normal cells and tissues around it. Cancer occurs in a variety of cells and tissues, including the oral cavity (e.g., mouth, tongue, pharynx, etc.), digestive system (e.g., oesophagus, stomach, small intestine, colon, rectum, liver, bile duct, gall bladder, pancreas, etc.), respiratory system (e.g., larynx, lung, bronchus, etc.), bones, joints, skin (e.g., basal cell, squamous cell, meningioma, etc.), breast, genital system, (e.g., uterus, ovary, prostate, testis, etc.), urinary system (e.g., bladder, kidney, ureter, etc.), eye, nervous system (e.g., brain, etc.), endocrine system (e.g., thyroid, etc.), and hematopoietic system (e.g., lymphoma, myeloma, leukaemia, acute lymphocytic leukaemia, chronic lymphocytic leukaemia, acute myeloid leukaemia, chronic myeloid leukaemia, etc.).

The term “host” as used herein refers to an animal, preferably a mammal, and most preferably a human.

The term “host cell” or “recombinant host cell” as used herein refers to the particular subject cell transfected with a (recombinant) nucleic acid molecule and the progeny or potential progeny of such a cell. Progeny of such a cell may not be identical to the parent cell transfected with the nucleic acid molecule due to mutations or environmental influences that may occur in succeeding generations or integration of the nucleic acid molecule into the host cell genome. For example, a Chinese hamster ovary (CHO) cell, which is an epithelial cell line derived from the ovary of the Chinese hamster, may be used as a host cell.

The term “effective amount” refers to an amount of an immunocytokine or composition or pharmaceutical composition containing said immunocytokine that is effective, at dosages and for periods of time necessary, to achieve the desired effect, including a therapeutic or prophylactic result. For example the term could refer to the amount of an immunocytokine of the invention to achieve a specified result (e.g. an agonistic effect).

The term “therapeutically effective amount” refers to the minimum concentration of an immunocytokine or composition or pharmaceutical composition containing said immunocytokine required to effect a measurable improvement or prevention of a particular disease or disorder. A therapeutically effective amount herein may vary according to factors such as the disease state, age, sex, and weight of the patient, and the ability of the immunocytokine to elicit a desired response in the individual. A therapeutically effective amount is also one in which toxic or detrimental effects of the antibody are outweighed by the therapeutically beneficial effects.

The term “affinity” as used herein is a measure of the binding strength between an antigen and an antigen-binding site on the antibody (or antigen-binding fragment thereof). Affinity is represented by the equilibrium constant for the dissociation of an antigen with an antigen-binding polypeptide (KD). The lower the KD value, the stronger the binding strength between an antigen and the antigen-binding site. Alternatively, the affinity can also be expressed as the affinity constant (KA), which is 1/KD. Any KD value less than 10⁻⁶ is considered to indicate binding. Affinity can be determined by known methods, depending on the specific antigen of interest. For example. KD may be determined by surface plasmon resonance. KD may also be determined by Scatchard analysis and/or competitive binding assays, such as radioimmunoassays (RIA), enzyme immunoassays (EIA) and sandwich competition assays, equilibrium dialysis, equilibrium binding, gel filtration, ELISA, or spectroscopy (e.g. using a fluorescence assay) and the different variants thereof known in the art. The binding affinity of the immunoconjugate or fragment thereof may be established by coating the immunoconjugate or fragment thereof directly or indirectly (e.g. by capture with an anti-human IgG Fc) onto a sensor surface (e.g. an amine high capacity chip or equivalent), wherein the target bound by the immunoconjugate or fragment thereof (e.g. NKG2D or IL-15Rp) is flowed over the sensor surface to detect binding. Suitably, a MASS-2 instrument (which may also be referred to as Sierra SPR-32) is used at 25° C. in PBS+0.02% Tween 20 running buffer at 30 μl/min.

The term “avidity” as used herein is the measure of the strength of binding between an antibody, or antigen-binding fragment thereof, and the antigen. Avidity is related to both the affinity between an antigen and its antigen-binding site on the antibody and the number of binding sites for that antigen present on the antibody.

Other terms are defined herein within the description of the various aspects of the invention.

### Immunoconjugates

Provided herein are immunoconjugates, comprising at least two IL-15 polypeptides, each IL-15 polypeptide comprising an IL-15 cytokine sequence; and IL-15 receptor alpha (CD215) sequence or a functional fragment thereof; and a multivalent antibody or antigen-binding fragment thereof, comprising at least two antibody binding domains that each specifically bind to an antigen selected from the group consisting of NKG2D, NKp30, NKp46, NKp44 and DNAM-1.

### Linkers

Linkers are described in four positions when detailing the immunoconjugates of the present invention.
1) the IL-15 polypeptide may be covalently joined or conjugated to the multivalent antibody, constant domain, or antigen-binding fragment thereof by means of a linker
2) the IL-15 cytokine may be covalently joined or conjugated to the IL-15 receptor alpha (CD215) or functional fragment thereof by means of a linker.
3) Variable heavy and variable light chains may be covalently joined or conjugated together by means of a linker to form a single-chain variable domain fragment (scFV).
4) A scFv may be covalently joined or conjugated to the multivalent immunocytokine or the antibody constant domain by means of linker.

Therefore when the term “linker” is used, defined or explained herein, it could refer to a linker in any or all of these positions. In some embodiments, each chain of the immunoconjugates of the present invention comprise no linkers, or comprise one linker, or comprise two linkers.

The IL-15 polypeptide may be joined or conjugated to the multivalent antibody or antigen-binding fragment thereof by means of a linker, or fused directly to the antibody, or by chemical conjugation to either the C-terminus or N-terminus of the heavy or the light chain of the antibody format. The antibody may be joined to the IL-15 polypeptide at any location along the antibody or the IL-15 polypeptide as long as it is still able to bind its target.

The IL-15 cytokine may be joined or conjugated to the IL-15 receptor alpha (CD215) or functional fragment thereof by means of a linker, or they may be fused directly together without an additional linker. The IL-15 cytokine may be joined to the IL-15 receptor alpha (CD215) or functional fragment thereof at any position provided the resulting IL-15 polypeptide is still able to bind its target.

In preferred embodiments the linker is flexible. Flexible linkers allow the joined domains a degree of movement which can play a key role in the function of the fused domains. In some embodiments the linker is a peptide linker, optionally from about 1 to about 60 amino acid residues in length. GlySer linkers are known in the art and may be used. For example, in some embodiments, linkers include those comprising or consisting of glycine and serine, for example multiple n repeats of the formulae [GGGGS]ₙX, or [GGGS]ₙX, or [GGS]ₙX, wherein n is typically between 1-6 and X is G or is absent. In some embodiments, the linker comprises or consists of the sequence [[G]ₘS]ₙX, wherein m is from 2 to 4, n is from 1 to 6 and X is G or is absent. In some embodiments, the linker comprises or consists of glycine and serine residues, and optionally the linker comprises more glycine than serine residues. In some embodiments, the linker comprises or consist of glycine and serine residues in a ratio of from about 5:1 to about 2:1. In some embodiments, the linker comprises or consists of glycine and serine residues in a ratio of about 5:1. In some embodiments the linker comprises or consists of about 80% glycine and about 20% serine residues. In some embodiments the linker comprises or consists of the sequence of any of SEQ ID NOs: 182 to 187, 201 and 204 to 205. In some preferred embodiments the linker comprises the sequence of SEQ ID NO: 182. In some embodiments the linker joining the heavy or light chain constant domain to the IL-15 polypeptide comprises or consists of the sequence of any of SEQ ID NOs: 182 to 187, 201 and 204 to 205. In some preferred embodiments the linker joining the heavy or light chain constant domain to the IL-15 polypeptide comprises the sequence of SEQ ID NO: 182.

In some embodiments the linker is a TVAAPS linker, for example as shown in SEQ ID NO:184. This linker is preferably used to join the light chain constant domain with the IL-15 polypeptide, but is also used to join the heavy chain constant domain with the IL-15 polypeptide. This linker may also be used to join the IL-15 cytokine to the IL-15 receptor alpha (CD215) or functional fragment thereof.

In some embodiments the linker is a ASTKGPS linker, for example as shown in SEQ ID NO:185. This linker is preferably used to join the heavy chain constant domain with the IL-15 polypeptide, but is also used to join the light chain constant domain with the IL-15 polypeptide. This linker may also be used to join the IL-15 cytokine to the IL-15 receptor alpha (CD215) or functional fragment thereof.

In some embodiments the linker is an extended linker comprising of 13 amino acids (SEQ ID NO: 201) derived from Exon 3 of the human IL-15 receptor alpha gene. This linker may be used to join the IL-15 cytokine to the IL-15 receptor alpha (CD215) or functional fragment thereof.

Typically, the length and sequence of such linkers can be optimised empirically. Such linkers can also incorporate alternate or additional amino acids. It also well recognized in the art that other synthetic or naturally ordered amino acid sequence combination are able to operate as flexible linkers.

### Arrangement and Design of Immunoconjugates of the Invention

The two components of the immunoconjugates of the invention are the IL-15 polypeptides (also referred to as the IL-15 component) and the multivalent antibody or antigen-binding fragment thereof (also referred to as the antibody component).

In some embodiments, the multivalent antibody or antigen-binding fragment thereof comprises at least two (preferably exactly two) immunoglobulins heavy chains and at least two (preferably exactly two) immunoglobulin light chains, and the IL-15 polypeptides are conjugated (optionally via a linker) to the heavy chains.

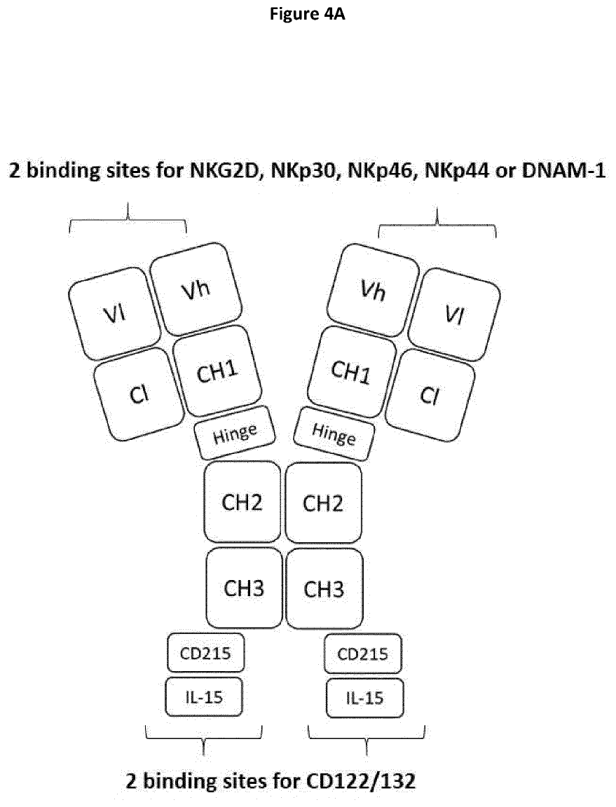

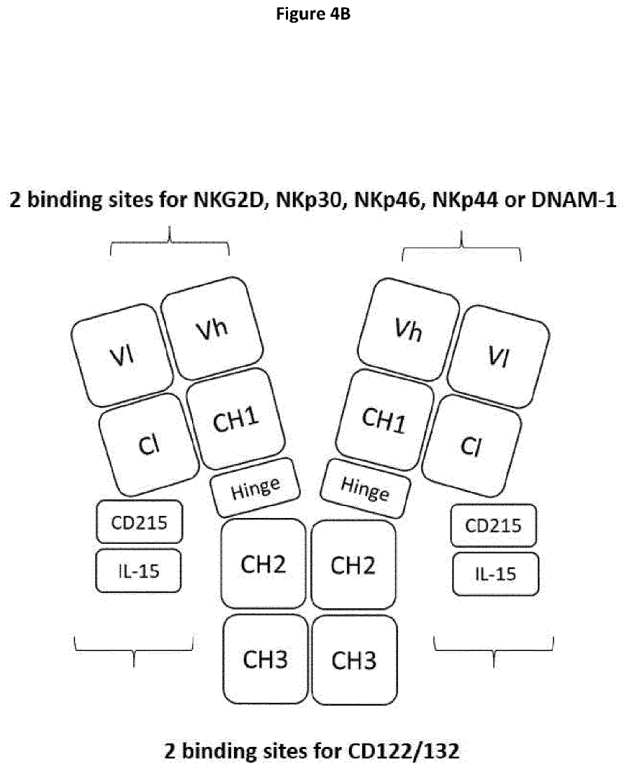

In preferred embodiments of the invention, the IL-15 polypeptides are conjugated (optionally via a linker) to the C-termini of the heavy chains of the antibody component. In some preferred embodiments the IL-15 polypeptides are conjugated (optionally via a linker) to the C-termini of the CH3 domains of the heavy chains. This format is shown in FIG. 4A.

In other embodiments of the invention, the IL-15 polypeptides are conjugated (optionally via a linker) to the N-termini of the heavy chains.

In some embodiments, the multivalent antibody or antigen-binding fragment thereof comprises at least two (preferably exactly two) immunoglobulin heavy chains and at least two (preferably exactly two) immunoglobulin light chains, and the IL-15 polypeptides are conjugated (optionally via a linker) to the light chains.

In preferred embodiments of the invention, the IL-15 polypeptides are conjugated (optionally via a linker) to the C-termini of the light chains of the antibody component. In some preferred embodiments the IL-15 polypeptides are conjugated (optionally via a linker) to the C-termini of the CL domains of the light chains. This format is shown in FIG. 4B.

In other embodiments of the invention, the IL-15 polypeptides are conjugated (optionally via a linker) to the N-termini of the light chains.

The two types of component in the immunoconjugates (the IL-15 component and the antibody component) will now be described in more detail. The arrangement and design of immunoconjugates described herein applies to each of the components now described in more detail.

### Antibodies and Fragments Thereof

The term “antibody” as used herein refers to immunoglobulin molecules and immunologically active portions of immunoglobulin molecules, i.e., molecules that contain an antigen binding site that specifically binds an antigen, whether natural or partly or wholly synthetically produced. This includes molecules that are fusions of different antibody components, for example a IgG constant domain or a fragment thereof joined to an antigen binding domain, for example a ScFv. The term “antibody”, “immunoglobulin” or “Ig” may be used interchangeably herein. The term also covers any polypeptide or protein having a binding domain which is, or is homologous to, an antibody binding domain. Antibodies may be polyclonal or monoclonal. These can be derived from natural sources, or they may be partly or wholly synthetically produced. Antibodies are polypeptides that typically contain two identical heavy chains and two identical light chains, which are smaller than the heavy chains. The immunoglobulin heavy chains are sometimes referred to herein as “a first polypeptide chain”. The immunoglobulin light chains are sometimes referred to herein as “a second polypeptide chain”. In mammals there are two types of light chain, which are called lambda (λ) and kappa (κ). Each of the heavy chains and each of the light chains are composed of a variable region and a constant region. The heavy chain variable region is referred to as the VH region and the light chain variable region is referred to as the VL region. For kappa light chains, the VL region can also be referred to as the VK region. Each of the variable regions of the heavy and light chains comprise three complementarity determining regions (CDRs), CDR1, CDR2 and CDR3. These are named VLCDR1, VLCDR2, VLCDR3, VHCDR1, VHCDR2 and VHCDR3 respectively.

The framework and complementarity determining regions have been precisely defined (Kabat et al. Sequences of Proteins of Immunological Interest, Fifth Edition U.S. Department of Health and Human Services, (1991) NIH Publication Number 91-3242). There are also alternative numbering conventions for CDR sequences, for example those set out in Chothia et al. (1989) Nature 342: 877-883 or as summarized by IMGT.org. In a conventional antibody, each VH and VL is composed of three CDRs and four FRs, arranged from amino-terminus (N-terminus) to carboxy-terminus (C-terminus) in the following order: FR1, CDR1, FR2, CDR2, FR3, CDR3, FR4. The conventional antibody tetramer of two heavy immunoglobulin chains and two light immunoglobulin chains is formed with the heavy and the light immunoglobulin chains inter-connected by e.g. disulphide bonds, and the heavy chains similarly connected. The “constant region” or “constant domain” comprises a heavy chain constant region and a light chain constant region. The heavy chain constant region includes three domains, CH1, CH2 and CH3, with a hinge between the CH1 and CH2 domains. The light chain constant region is comprised of one domain, CL. The variable domain of the heavy chains and the variable domain of the light chains are binding domains that interact with an antigen. The constant regions of the antibodies typically mediate the binding of the antibody to host tissues or factors, including various cells of the immune system (e.g. effector cells) and the first component (C1q) of the classical complement system. The antibodies comprised in the immunoconjugates of the invention may be of any suitable type, including IgA, IgD, IgE, IgG, IgM and IgY, although IgG may be preferred. IgG1 backbones may be most preferred.

The term “antigen binding fragment” (which may also referred to as “antibody fragment”, “immunoglobulin fragment”, or “antigen-binding polypeptide”) as used herein refers to a portion of an antibody (or constructs that contain said portion) that specifically binds to the target (e.g. a molecule in which one or more immunoglobulin chains is not full length, but which still specifically binds to the target). Examples of binding fragments encompassed within the term antibody fragment include Fab, F(ab′)2, Fv, scFv, dAb, Fd; and diabodies. A scFv (single-chain fragment variable) format is a heavy variable region of an antibody (VH) and a light variable region of an antibody (VL) fused together, usually by a flexible peptide linker. The fragment antigen-binding region (“Fab” region) is an antibody binding domain composed of a monovalent fragment consisting of the VL, VH, CL and CH1 domains.

The term “antibody binding domain” (which may also referred to as “antigen binding region,” “antigen binding fragment,” and similar terms) refers to that portion of an antibody which comprises the amino acid residues that interact with an antigen and confer on the binding agent its specificity and affinity for the antigen (e.g. the complementarity determining regions (CDRs)).

The term “Fc domain” or “Fc region” refers to the “fragment crystallizable region” which is a C-terminal region of an immunoglobulin heavy chain, including native- sequence Fc regions and variant Fc regions. The “Fc domain” as used herein refers to a portion of an antibody (or constructs that contain said portion) comprising the CH2 and CH3 domains. An Fc region is dimeric and comprises paired heavy chain constant regions each comprising a CH2 and CH3 domain. The “Fc fragment” refers to the carboxy-terminal portions of both H chains held together by disulfides. The effector functions of antibodies are determined by sequences in the Fc region, the region which is also recognized by Fc receptors (FcR) found on certain types of cells.

The term “Fc enabled” refers to an antibody that comprises a functional Fc region (fragment crystallizable region), i.e. a Fc region that has not been disabled by mutation or otherwise. Fc enabled of “effector enabled” antibodies demonstrate unattenuated Fc function. The term “Fc disabled” refers to an antibody that comprises a non-functional Fc region due to mutation or otherwise. Fc disabled or “effector disabled” antibodies are not able or less able to be recognized by Fc receptors (FcR) found on certain types of cells.

The term “effector function” as used herein is meant to refer to one or more of interactions with Fc receptors, antibody dependant cell mediated cytotoxic activity (ADCC), complement-dependant cytotoxic activity (CDC) mediated responses, Fc-mediated phagocytosis or antibody dependant cellular phagocytosis (ADCP) and antibody recycling via the FcRn receptor.

The term “monoclonal antibody” as used herein refers to an antibody obtained from a population of substantially homogeneous antibodies, i.e. the individual antibodies comprising the population are identical except for possible naturally occurring mutations and/or post-translation modifications (e.g. isomerizations, amidations) that may be present in minor amounts. Monoclonal antibodies are highly specific, and are directed against a single antigenic determinant or epitope. In contrast, polyclonal antibody preparations typically include different antibodies directed against different antigenic determinants (or epitopes). The term “monoclonal antibody” as used herein encompasses both intact and full-length monoclonal antibodies as well as antibody fragments (such as Fab, Fab′, F(ab′)₂, Fv), single chain (scFv) mutants, fusion proteins comprising an antibody portion, and any other modified immunoglobulin molecule comprising an antigen recognition site.

The term “valency” as used herein refers to the number of antigen binding sites on a single antibody molecule. The antibody components of the invention are “multivalent”, meaning they possess more than one antigen binding site on each antibody molecule. For example, the valency of a full human IgG antibody is 2, because it has two antigen binding sites per IgG molecule and the valency of other antibodies may be higher.

The term “multivalent” as used herein is used to describe a molecule with more than one functional binding domain for any given target. For example, a multivalent immunocytokine may have more than one functional binding domain to a cytokine receptor and more than one functional binding domain to an antigen.

The term “human antibody” as used herein refers to antibodies having variable and constant regions derived from human germline immunoglobulin sequences. Human subjects administered with said human antibodies do not generate cross-species antibody responses (for example termed HAMA responses - human-anti-mouse antibody) to the primary amino acids contained within said antibodies. Said human antibodies may include amino acid residues not encoded by human germline immunoglobulin sequences (e.g. mutations introduced by random or site-specific mutagenesis or by somatic mutation), for example in the CDRs. However, the term is not intended to include antibodies in which CDR sequences derived from the germline of another mammalian species, such as a mouse, have been grafted onto human framework sequences. Human antibodies that are prepared, expressed, created or isolated by recombinant means, such as antibodies expressed using a recombinant expression vector transfected into a host cell, antibodies isolated from a recombinant, combinatorial human antibody library, antibodies isolated from an animal (e.g. a mouse) that is transgenic for human immunoglobulin genes or antibodies prepared, expressed, created or isolated by any other means that involves splicing of human immunoglobulin gene sequences to other DNA sequences, may also be referred to as “recombinant human antibodies”.

Substituting at least one amino acid residue in the framework region of a non-human immunoglobulin variable domain with the corresponding residue from a human variable domain is referred to as “humanisation”. Humanisation of a variable domain may reduce immunogenicity in humans.

The term “specificity” as used herein is the ability of an antibody or antibody binding domain to recognise a particular antigen as a unique molecular entity and distinguish it from another. Specificity therefore refers to the number of different types of antigens or antigenic determinants to which a particular antibody or antigen-binding fragment thereof can bind. An antibody that “specifically binds” to an antigen or an epitope is a term well understood in the art. A molecule is said to exhibit “specific binding” if it reacts more frequently, more rapidly, with greater duration and/or with greater affinity with a particular target antigen or epitope, than it does with alternative targets. An antibody “specifically binds” to a target antigen or epitope if it binds with greater affinity, avidity, more readily, and/or with greater duration than it binds to other substances. In one embodiment, the extent of binding of an antibody to an unrelated target is less than about 10% of the binding of the antibody to the target as measured, e.g. by a radioimmunoassay (RIA).

The term “monospecific antibody” as used herein refers to an antibody that only specifically binds to one antigen. Monospecific antibodies may be multivalent (have multiple binding sites for that one antigen) but if the antibody only recognises one antigen, it is still classed as a monospecific antibody. By contrast, the term “multispecific antibody” as used herein refers to an antibody that is capable of binding a plurality of different epitopes simultaneously or sequentially. Generally, the epitopes will not be on the same antigen. Hence a multispecific antibody has the capability to selectively bind to epitopes present on different antigens via a plurality of different binding domains. A “bispecific antibody” is an antibody that is capable of binding two different epitopes simultaneously and/or sequentially. Generally, the epitopes will not be on the same antigen. Hence bispecific antibodies have the capability to selectively bind to two different epitopes present on two different antigens via two different binding domains.

The terms “EU index numbering” and like terms such as “Kabat” or “Kabat numbering” are recognized in the art and refer to systems of numbering amino acid residues which are more variable (i.e. hypervariable) than other amino acid residues in the heavy chain variable regions of an antibody, or an antigen binding portion thereof. Such numbering systems are well known in the art, for example Kabat et al., (1971) Ann. NY Acad. Sci., 190:382-391 and, Kabat et al. (1991) Sequences of Proteins of Immunological Interest, Fifth Edition, U.S. Department of Health and Human Services, NIH Publication No. 91-3242.

### Identity and Homology

“Identity” as known in the art is the relationship between two or more polypeptide sequences or two or more polynucleotide sequences, as determined by comparing the sequences. In the art, identity also means the degree of sequence relatedness between polypeptide or polynucleotide sequences, as the case may be, as determined by the match between strings of such sequences. While there exist a number of methods to measure identity between two polypeptides or two polynucleotide sequences, methods commonly employed to determine identity are codified in computer programs. Preferred computer programs to determine identity between two sequences include, but are not limited to, GCG program package (Devereux, et al., Nucleic Acids Research, 12, 387 (1984), BLASTP, BLASTN, and FASTA (Atschul et al., J. Molec. Biol. 215, 403 (1990)).

One can use a program such as the CLUSTAL program to compare amino acid sequences. This program compares amino acid sequences and finds the optimal alignment by inserting spaces in either sequence as appropriate. It is possible to calculate amino acid identity or similarity (identity plus conservation of amino acid type) for an optimal alignment. A program like BLASTx will align the longest stretch of similar sequences and assign a value to the fit. It is thus possible to obtain a comparison where several regions of similarity are found, each having a different score. Both types of identity analysis are contemplated in the present invention.

The percent identity of two amino acid sequences or of two nucleic acid sequences is determined by aligning the sequences for optimal comparison purposes (e.g., gaps can be introduced in the first sequence for best alignment with the sequence) and comparing the amino acid residues or nucleotides at corresponding positions. The “best alignment” is an alignment of two sequences which results in the highest percent identity. The percent identity is determined by the number of identical amino acid residues or nucleotides in the sequences being compared (i.e., % identity=number of identical positions/total number of positions ×100). Generally, references to % identity herein refer to % identity along the entire length of the molecule, unless the context specifies or implies otherwise.

The determination of percent identity between two sequences can be accomplished using a mathematical algorithm known to those of skill in the art. An example of a mathematical algorithm for comparing two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. The NBLAST and XBLAST programs of Altschul, et al. (1990) J. Mol. Biol. 215:403-410 have incorporated such an algorithm. BLAST nucleotide searches can be performed with the NBLAST program, score=100, wordlength=12 to obtain nucleotide sequences homologous to nucleic acid molecules of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilised as described in Altschul et al. (1997) Nucleic Acids Res. 25:3389-3402. Alternatively, PSI-Blast can be used to perform an iterated search which detects distant relationships between molecules (Id.). When utilising BLAST, Gapped BLAST, and PSI-Blast programs, the default parameters of the respective programs (e.g., XBLAST and NBLAST) can be used. See http://www.ncbi.nlm.nih.gov. Another example of a mathematical algorithm utilised for the comparison of sequences is the algorithm of Myers and Miller, CABIOS (1989). The ALIGN program (version 2.0) which is part of the CGC sequence alignment software package has incorporated such an algorithm. Other algorithms for sequence analysis known in the art include ADVANCE and ADAM as described in Torellis and Robotti (1994) Comput. Appl. Biosci., 10:3-5; and FASTA described in Pearson and Lipman (1988) Proc. Natl. Acad. Sci. 85:2444-8. Within FASTA, ktup is a control option that sets the sensitivity and speed of the search.

Typically, the amino acid sequence of the VH and VL regions of the immunocytokines provided in the invention have at least 90% identity, for example using the default parameters of the BLAST computer program (Atschul et al., J. Mol. Biol. 215, 403-410 (1990)) provided by HGMP (Human Genome Mapping Project), at the amino acid level, to the amino acid sequences of the VH and VL regions described below. More typically, the VH and VL regions have at least 90%, 91%, 92%, 93%, 94%, 95%, 96%, 97%, 98% or at least 99% identity, at the amino acid level, to the sequences shown below. Typically, each of the VH and VL regions of the immunocytokines used in the invention has this level of identity to the amino acid sequences of the VH and VL regions set out below. Alternatively, only one of the VH and VL regions of the immunocytokines used in the invention has this level of identity to the amino acid sequences of the VH and VL regions set out below.

Identity, as used herein, may be used interchangeably with “homology” and “similarity”. References to particular % identities apply equally to % homology and % similarity. Homology and similarity may be determined using appropriate algorithms, such as FASTA, BLAST and Gapped BLAST. Software for performing these analyses are publicly available.

### Variants and Conservative Substitutions

As used herein the term “variant” relates to proteins that have a similar amino acid sequence and/or that retain the same function. For instance, the term “variant” encompasses proteins or polypeptides which include one or more amino acid additions, deletions, substitutions or the like. An example of a variant of the present invention is a protein comprising a peptide as defined below, apart from the substitution of one or more amino acids with one or more other amino acids. Amino acid substitutions may be made to, for example, reduce or eliminate liabilities in the amino acid sequences. Alternatively, amino acid substitutions may be made to improve antigen affinity or to humanise or deimmunise the antibodies, if required. Affinity matured variants, humanised variants and deimmunised variants of the specified antibodies are provided herein, as well as variants comprising amino acid substitutions to reduce or eliminate any liabilities in the sequences of the antibodies.

The skilled person is aware that various amino acids have similar properties. One or more such amino acids of a substance can often be substituted by one or more other such amino acids without eliminating a desired activity of that substance.

Thus, the amino acids glycine, alanine, valine, leucine and isoleucine can often be substituted for one another (amino acids having aliphatic side chains). Of these possible substitutions it is preferred that glycine and alanine are used to substitute for one another (since they have relatively short side chains) and that valine, leucine and isoleucine are used to substitute for one another (since they have larger aliphatic side chains which are hydrophobic). Other amino acids which can often be substituted for one another include: phenylalanine, tyrosine and tryptophan (amino acids having aromatic side chains); lysine, arginine and histidine (amino acids having basic side chains); aspartate and glutamate (amino acids having acidic side chains); asparagine and glutamine (amino acids having amide side chains); and cysteine and methionine (amino acids having sulphur containing side chains).

Substitutions of this nature are often referred to as “conservative” or “semi- conservative” amino acid substitutions.

Using the three letter and one letter codes the naturally occurring amino acids may be referred to as follows: glycine (G or Gly), alanine (A or Ala), valine (V or Val), leucine (L or Leu), isoleucine (I or lie), proline (P or Pro), phenylalanine (F or Phe), tyrosine (Y or Tyr), tryptophan (W or Trp), lysine (K or Lys), arginine (R or Arg), histidine (H or His), aspartic acid (D or Asp), glutamic acid (E or Glu), asparagine (N or Asn), glutamine (Q or Gln), cysteine (C or Cys), methionine (M or Met), serine (S or Ser) and Threonine (T or Thr). Where a residue may be aspartic acid or asparagine, the symbols Asx or B may be used. Where a residue may be glutamic acid or glutamine, the symbols Glx or Z may be used. References to aspartic acid include aspartate, and glutamic acid include glutamate, unless the context specifies otherwise.

Amino acid deletions or insertions can also be made relative to the amino acid sequence for the fusion protein referred to below. Thus, for example, amino acids which do not have a substantial effect on the activity of the polypeptide, or at least which do not eliminate such activity, can be deleted. Such deletions can be advantageous since the overall length and the molecular weight of a polypeptide can be reduced whilst still retaining activity. This can enable the amount of polypeptide required for a particular purpose to be reduced—for example, dosage levels can be reduced.

Thus, the amino acids glycine, alanine, valine, leucine and isoleucine can often be substituted for one another (amino acids having aliphatic side chains). Of these possible substitutions it is preferred that glycine and alanine are used to substitute for one another (since they have relatively short side chains) and that valine, leucine and isoleucine are used to substitute for one another (since they have larger aliphatic side chains which are hydrophobic). Other amino acids which can often be substituted for one another include: phenylalanine, tyrosine and tryptophan (amino acids having aromatic side chains); lysine, arginine and histidine (amino acids having basic side chains); aspartate and glutamate (amino acids having acidic side chains); asparagine and glutamine (amino acids having amide side chains); and cysteine and methionine (amino acids having sulphur containing side chains).

In some embodiments, the following amino acids can be exchange for each other for conservative amino acid substitutions:

| Class | Exchangeable amino acids |
|---|---|
| Aliphatic | Glycine, Alanine, Valine, Leucine, |
| | Isoleucine |
| Hydroxyl or Sulphur/ | Serine, Cysteine, Threonine, Methionine |
| Selenium-containing | |
| Aromatic | Phenylalanine, Tyrosine, Tryptophan |
| Basic | Histidine, Lysine, Arginine |
| Acidic and their Amide | Aspartate, Glutamate, Asparagine, |
| | Glutamine |

Therefore, references to “conservative” amino acid substitutions refer to amino acid substitutions in which one or more of the amino acids in the sequence of the immunoconjugate or component parts (e.g. in the CDRs or in the VH or VL sequences) is substituted with another amino acid in the same class as indicated above. Conservative amino acid substitutions may be preferred in the CDR regions to minimise adverse effects on the function of the antibody. However, conservative amino acid substitutions may also occur in the framework regions.

Amino acid changes relative to the sequence given below can be made using any suitable technique e.g. by using site-directed mutagenesis or solid-state synthesis.

It should be appreciated that amino acid substitutions or insertions within the scope of the present invention can be made using naturally occurring or non-naturally occurring amino acids, although naturally occurring amino acids may be preferred. Whether or not natural or synthetic amino acids are used, it is preferred that only L- amino acids are present.

### Summary of Antibody Components

To outline the applicability of the approach a series of non-limiting example immunoconjugates are provided herein. These immunoconjugates comprise least two IL-15 polypeptides and a multivalent antibody or antigen-binding fragment thereof, comprising at least two antibody binding domains that each specifically bind to an antigen selected from the group consisting of NKG2D, NKp30, NKp46, NKp44 and DNAM-1.

In preferred embodiments the multivalent antibody or antigen-binding fragments thereof are monospecific, meaning both antibody binding domains bind to the same target, and to the same epitope on said target.

A summary of the multivalent antibodies or antigen binding fragments thereof that may form the antibody component of these immunoconjugates is provided below in Table 1.

TABLE 1

| Antibody | | | | | | | | | |
|---|---|---|---|---|---|---|---|---|---|
| component | | SEQ ID NOs | | | | | | | |
| name | Target | HCDR1 | HCDR2 | HCDR3 | LCDR1 | LCDR2 | LCDR3 | VH | VL |
| H1/L1 | NKG2D | 1 | 14 | 27 | 40 | 53 | 66 | 79 | 92 |
| H2/L2 | NKG2D | 2 | 15 | 28 | 41 | 54 | 67 | 80 | 93 |
| H3/L3 | NKG2D | 3 | 16 | 29 | 42 | 55 | 68 | 81 | 94 |
| H4/L4 | NKG2D | 4 | 17 | 30 | 43 | 56 | 69 | 82 | 95 |
| H5/L5 | NKG2D | 5 | 18 | 31 | 44 | 57 | 70 | 83 | 96 |
| H6/L6 | NKG2D | 6 | 19 | 32 | 45 | 58 | 71 | 84 | 97 |
| H7/L7 | NKG2D | 7 | 20 | 33 | 46 | 59 | 72 | 85 | 98 |
| H8/L8 | NKG2D | 8 | 21 | 34 | 47 | 60 | 73 | 86 | 99 |
| H9/L9 | NKG2D | 9 | 22 | 35 | 48 | 61 | 74 | 87 | 100 |
| H10/10 | NKG2D | 10 | 23 | 36 | 49 | 62 | 75 | 88 | 101 |
| H11/11 | NKp30 | 11 | 24 | 37 | 50 | 63 | 76 | 89 | 102 |
| H12/L2 | NKp30 | 12 | 25 | 38 | 51 | 64 | 77 | 90 | 103 |
| H13/L13 | NKp46 | 13 | 26 | 39 | 52 | 65 | 78 | 91 | 104 |

As will be understood, any of the multivalent antibodies or antigen-binding fragments thereof may be suitably combined with the various IL-15 polypeptides presented herein and the components arranged into the different arrangements and formats of immunoconjugate described herein.

The immunoconjugates of the invention comprise a multivalent antibody or antigen-binding fragment thereof, comprising at least two antibody binding domains that each specifically bind to an antigen selected from the group consisting of NKG2D, NKp30, NKp46, NKp44 and DNAM-1. In some preferred embodiments the immunoconjugate comprises at least two antibody binding domains that each specifically bind to an antigen selected from the group consisting of NKG2D, NKp30 and NKp46.

In some embodiments, the immunoconjugate comprises a multivalent antibody or antigen-binding fragment thereof that binds to human and/or cynomologous NKG2D.

In some embodiments, the immunoconjugate comprises a multivalent antibody or antigen-binding fragment thereof that binds to human and/or cynomologous NKp30.

In some embodiments, the immunoconjugate comprises a multivalent antibody or antigen-binding fragment thereof that binds to human and/or cynomologous NKp46.

In some embodiments, the immunoconjugate comprises a multivalent antibody or antigen-binding fragment thereof that binds to human and/or cynomologous NKp44.

In some embodiments, the immunoconjugate comprises a multivalent antibody or antigen-binding fragment thereof that binds to human and/or cynomologous DNAM-1.

The multivalent antibody component of the immunoconjugates of the invention can be derived from antibodies that bind to each of the targets selected from the group consisting of NKG2D, NKp30 and NKp46. For example any of the antibodies disclosed herein (see Table 1) as H1/L1- H13/L13. The skilled person can equally select another suitable antibody that binds to one of the targets and utilise their skill in the art to combine the antibodies with the other components of the immunoconjugates.

In some embodiments, the immunoconjugate comprises a multivalent antibody or antigen-binding fragment thereof that comprises:
a) a heavy chain variable region (VH) comprising:
   i. a heavy chain complementary determining region (HCDR) 1 comprising or consisting of a sequence selected from the group consisting of SEQ ID NOs: 1 to 13, optionally having up to 2 amino acid substitutions therefrom;
   ii. a HCDR2 comprising or consisting of a sequence selected from the group consisting of SEQ ID NOs: 14 to 26, optionally having up to 2 amino acid substitutions therefrom; and
   iii. a HCDR3 comprising or consisting of a sequence selected from the group consisting of SEQ ID NOs: 27 to 39, optionally having up to 2 amino acid substitutions therefrom; and
b) a light chain variable region (VL) comprising:
   i. a light chain complementarity determining region (LCDR) 1 comprising or consisting of a sequence selected from the group consisting of SEQ ID NOs: 40 to 52, optionally having up to 2 amino acid substitutions therefrom;
   ii. a LCDR2 comprising or consisting of a sequence selected from the group consisting of SEQ ID NOs: 53 to 65, optionally having up to 2 amino acid substitutions therefrom; and
   iii. a LCDR3 comprising or consisting of a sequence selected from the group consisting of SEQ ID NOs: 66 to 78, optionally having up to 2 amino acid substitutions therefrom.

In some embodiments the optional amino acid substitutions are conservative substitutions.

In some embodiments, the immunoconjugate comprises a multivalent antibody or antigen-binding fragment thereof that comprises:
a) a heavy chain variable region (VH) comprising or consisting of a sequence having at least 90%, at least 91%, at least 92%, at least 93%, at least 94%, at least 95%, at least 95%, at least 97%, at least 98% or at least 99% identity to a sequence selected from the group consisting of SEQ ID NOs: 79 to 91 and 202; and
b) a light chain variable region (VL) comprising or consisting of a sequence having at least 90%, at least 91%, at least 92%, at least 93%, at least 94%, at least 95%, at least 95%, at least 97%, at least 98% or at least 99% identity to a sequence selected from the group consisting of SEQ ID NOs: 92 to 104.

In some embodiments, the immunoconjugate comprises a multivalent antibody or antigen-binding fragment thereof that comprises:
a) a VH comprising or consisting of a sequence selected from the group consisting of SEQ ID NOs: 79 to 91 and 202; and
b) a VL comprising or consisting of a sequence selected from the group consisting of SEQ ID NOs: 92 to 104.

In some embodiments, the immunoconjugate comprises a multivalent antibody or antigen-binding fragment thereof that comprises:
a) a heavy chain comprising or consisting of a sequence having at least 90%, at least 91%, at least 92%, at least 93%, at least 94%, at least 95%, at least 95%, at least 97%, at least 98% or at least 99% identity to a sequence selected from the group consisting of SEQ ID NOs: 109 to 134; and
b) a light chain comprising or consisting of a sequence having at least 90%, at least 91%, at least 92%, at least 93%, at least 94%, at least 95%, at least 95%, at least 97%, at least 98% or at least 99% identity to a sequence selected from the group consisting of SEQ ID NOs: 135 to 147.

In some embodiments, the immunoconjugate comprises a multivalent antibody or antigen-binding fragment thereof that comprises:
a) a heavy chain comprising or consisting of a sequence selected from the group consisting of SEQ ID NOs: 109 to 134; and
b) a light chain comprising or consisting of a sequence selected from the group consisting of SEQ ID NOs: 135 to 147.

In some embodiments, the immunoconjugate comprises a multivalent antibody or antigen-binding fragment thereof that comprises
a) the six CDR sequences;
b) the VH and or the VL sequences; and/or
c) the heavy chain and/or the light chain sequences
   of an antibody that binds to NKp44.

In some embodiments, the immunoconjugate comprises a multivalent antibody or antigen-binding fragment thereof that comprises
a) the six CDR sequences;
b) the VH and or the VL sequences; and/or
c) the heavy chain and/or the light chain sequences
   of an antibody that binds to DNAM-1.

In some embodiments, the multivalent antibody or antigen binding fragment thereof comprises an Fc domain, optionally a human Fc domain. In some embodiments, the multivalent antibody or antigen binding fragment thereof comprises a human IgG Fc domain. In preferred embodiments the human IgG Fc domain is a human IgG1 Fc domain that may additionally comprise a CH1 domain and hinge domain, e.g. SEQ ID NO:105. 105, 203.

In some embodiments, the Fc domain of the multivalent antibody or fragment thereof is Fc disabled e.g. SEQ ID NO: 106. In some embodiments, the Fc domain of the multivalent antibody or fragment thereof comprises a substitution with respect to a wild-type Fc sequence, wherein the substitution reduces binding of the Fc domain to an Fc receptor. In some embodiments, the substitution is a substitution at a position selected from the group consisting of L234, L235, G236, G237 and P329 (EU index numbering), optionally wherein the substitution is selected from the group consisting of L235A, G237A, P329S and P329G (EU index numbering). These listed substitutions are not intended to be limiting as the skilled person is aware of multiple alternative substitutions which are known in the art and can be utilised to disable to reduce binding of the Fc domain to an Fc receptor. In a preferred embodiment, the substitutions are L234A and L235A (LALA), optionally with P329G or P329S. In another preferred embodiment, the substitutions are L235A and G237A (LAGA), optionally with P329G or P329S.

In some embodiments, the constant domain of the multivalent antibody or fragment thereof comprises:
a) a heavy chain sequence having at least 90%, at least 91%, at least 92%, at least 93%, at least 94%, at least 95%, at least 95%, at least 97%, at least 98%, at least 99% identity or 100% identity to a human IgG1 Fc domain or to a human IgG1 Fc disabled domain; and
b) a light chain sequence having at least 90%, at least 91%, at least 92%, at least 93%, at least 94%, at least 95%, at least 95%, at least 97%, at least 98%, at least 99% identity, or 100% identity to a light chain constant Kappa domain or to a light chain constant Lambda domain.

In some embodiments, the constant domain of the multivalent antibody or fragment thereof comprises:
a) a heavy chain sequence having at least 90%, at least 91%, at least 92%, at least 93%, at least 94%, at least 95%, at least 95%, at least 97%, at least 98%, at least 99% identity or 100% identity to a human IgG1 CH1-hinge-Fc domain or to a human IgG1 CH1-hinge-Fc disabled domain; and
b) a light chain sequence having at least 90%, at least 91%, at least 92%, at least 93%, at least 94%, at least 95%, at least 95%, at least 97%, at least 98%, at least 99% identity, or 100% identity to a light chain constant Kappa domain or to a light chain constant Lambda domain.

In some embodiments, the constant domain of the multivalent antibody or fragment thereof comprises:
a) a heavy chain sequence having at least 90%, at least 91%, at least 92%, at least 93%, at least 94%, at least 95%, at least 95%, at least 97%, at least 98% or at least 99% identity to a sequence selected from the group consisting of SEQ ID NOs: 105, 106 and 203; and
b) a light chain sequence having at least 90%, at least 91%, at least 92%, at least 93%, at least 94%, at least 95%, at least 95%, at least 97%, at least 98% or at least 99% identity to a sequence selected from the group consisting of SEQ ID NOs: 107 to 108.

In some embodiments, the constant domain of the multivalent antibody or fragment thereof comprises:
a) a heavy chain sequence selected from the group consisting of SEQ ID NOs: 105,106 and 203; and
b) a light chain sequence selected from the group consisting of SEQ ID NOs: 107 to 108.

In some embodiments, the constant domain of the multivalent antibody or fragment thereof comprises:
a) a heavy chain sequence comprising or consisting of the sequence of SEQ ID NO: 105 and a light chain sequence comprising or consisting of the sequence of SEQ ID NO: 107;
b) a heavy chain sequence comprising or consisting of the sequence of SEQ ID NO: 105 and a light chain sequence comprising or consisting of the sequence of SEQ ID NO: 108;
c) a heavy chain sequence comprising or consisting of the sequence of SEQ ID NO: 106 and a light chain sequence comprising or consisting of the sequence of SEQ ID NO: 107;
d) a heavy chain sequence comprising or consisting of the sequence of SEQ ID NO: 106 and a light chain sequence comprising or consisting of the sequence of SEQ ID NO: 108;
e) a heavy chain sequence comprising or consisting of the sequence of SEQ ID NO: 203 and a light chain sequence comprising or consisting of the sequence of SEQ ID NO: 107; or
f) a heavy chain sequence comprising or consisting of the sequence of SEQ ID NO: 203 and a light chain sequence comprising or consisting of the sequence of SEQ ID NO: 108.

### NKG2D Antibody Components

To further outline the applicability of the approach a series of non-limiting example immunoconjugates are provided herein. These immunoconjugates comprise least two IL-15 polypeptides and a multivalent antibody or antigen-binding fragment thereof, comprising at least two antibody binding domains that each specifically bind to NKG2D. In preferred embodiments the multivalent antibody or antigen-binding fragments thereof binds to NKG2D in a monospecific manner, meaning both antibody binding domains bind to the NKG2D, and to the same NKG2D epitope.

A summary of the multivalent antibodies or antigen binding fragments thereof that form the antibody component of these NKG2D-IL-15 immunoconjugates is provided below.

### H1/L1 Antibody Component

In some embodiments, the multivalent antibody or antigen binding fragment thereof comprises sequences from the anti-NKG2D antibody described herein as H1/L1.

In some embodiments, the multivalent antibody or antigen binding fragment thereof comprises:
a) a heavy chain variable region (VH) comprising:
   i. a HCDR1 comprising or consisting of the sequence of SEQ ID NO: 1, optionally having up to 2 amino acid substitutions therefrom;
   ii. a HCDR2 comprising or consisting of the sequence of SEQ ID NO: 14, optionally having up to 2 amino acid substitutions therefrom; and
   iii. a HCDR3 comprising or consisting of the sequence of SEQ ID NO: 27, optionally having up to 2 amino acid substitutions therefrom;
b) and a light chain variable region (VL) comprising:
   i. a LCDR1 comprising or consisting of the sequence of SEQ ID NO: 40, optionally having up to 2 amino acid substitutions therefrom;
   ii. a LCDR2 comprising or consisting of the sequence of SEQ ID NO: 53, optionally having up to 2 amino acid substitutions therefrom; and
   iii. a LCDR3 comprising or consisting of the sequence of SEQ ID NO: 66, optionally having up to 2 amino acid substitutions therefrom.

In some embodiments the optional amino acid substitutions are conservative substitutions.

In some embodiments, the multivalent antibody or antigen binding fragment thereof comprises a VH comprising or consisting of the sequence of SEQ ID NO: 79 and a VL comprising or consisting of the sequence of SEQ ID NO: 92.

In some embodiments, the multivalent antibody or antigen binding fragment thereof comprises a VH comprising or consisting of the sequence of SEQ ID NO: 202 and a VL comprising or consisting of the sequence of SEQ ID NO: 92. SEQ ID NO: 202 and SEQ ID NO: 79 comprise the same CDR regions, and only differ from each other in the framework region.

In some embodiments, the multivalent antibody or antigen binding fragment thereof comprises a heavy chain comprising or consisting of the sequence of SEQ ID NO: 109 and a light chain comprising or consisting of the sequence SEQ ID NO: 135.

In some embodiments, the multivalent antibody or antigen binding fragment thereof comprises a heavy chain comprising or consisting of the sequence of SEQ ID NO: 122 and a light chain comprising or consisting of the sequence SEQ ID NO: 135.

As will be understood, any of the multivalent antibodies or antigen-binding fragments thereof comprising sequences from the anti-NKG2D antibody described herein as H1/L1 may be suitably combined with any of the IL-15 polypeptides presented herein and the components arranged into any of the different arrangements and formats of immunoconjugate described herein.

### NKG2D - H2/L2 Antibody Component

In some embodiments, the multivalent antibody or antigen binding fragment thereof comprises sequences from the anti-NKG2D antibody described herein as H2/L2.

In some embodiments, the multivalent antibody or antigen binding fragment thereof comprises:
a) a heavy chain variable region (VH) comprising:
   i. a HCDR1 comprising or consisting of the sequence of SEQ ID NO: 2, optionally having up to 2 amino acid substitutions therefrom;
   ii. a HCDR2 comprising or consisting of the sequence of SEQ ID NO: 15, optionally having up to 2 amino acid substitutions therefrom; and
   iii. a HCDR3 comprising or consisting of the sequence of SEQ ID NO: 28, optionally having up to 2 amino acid substitutions therefrom;
b) and a light chain variable region (VL) comprising:
   i. a LCDR1 comprising or consisting of the sequence of SEQ ID NO: 41, optionally having up to 2 amino acid substitutions therefrom;
   ii. a LCDR2 comprising or consisting of the sequence of SEQ ID NO: 54, optionally having up to 2 amino acid substitutions therefrom; and
   iii. a LCDR3 comprising or consisting of the sequence of SEQ ID NO: 67, optionally having up to 2 amino acid substitutions therefrom;

In some embodiments the optional amino acid substitutions are conservative substitutions.

In some embodiments, the multivalent antibody or antigen binding fragment thereof comprises a VH comprising or consisting of the sequence of SEQ ID NO: 80 and a VL comprising or consisting of the sequence of SEQ ID NO: 93.

In some embodiments, the multivalent antibody or antigen binding fragment thereof comprises a heavy chain comprising or consisting of the sequence of SEQ ID NO: 110 and a light chain comprising or consisting of the sequence SEQ ID NO: 136.

In some embodiments, the multivalent antibody or antigen binding fragment thereof comprises a heavy chain comprising or consisting of the sequence of SEQ ID NO: 123 and a light chain comprising or consisting of the sequence SEQ ID NO: 136.

As will be understood, any of the multivalent antibodies or antigen-binding fragments thereof comprising sequences from the anti-NKG2D antibody described herein as H2/L2 may be suitably combined with any of the IL-15 polypeptides presented herein and the components arranged into any of the different arrangements and formats of immunoconjugate described herein.

### NKG2D - H3/L3 Antibody Component

In some embodiments, the multivalent antibody or antigen binding fragment thereof comprises sequences from the anti-NKG2D antibody described herein as H3/L3.

In some embodiments, the multivalent antibody or antigen binding fragment thereof comprises:
a. a heavy chain variable region (VH) comprising:
   i. a HCDR1 comprising or consisting of the sequence of SEQ ID NO: 3, optionally having up to 2 amino acid substitutions therefrom;
   ii. a HCDR2 comprising or consisting of the sequence of SEQ ID NO: 16, optionally having up to 2 amino acid substitutions therefrom; and
   iii. a HCDR3 comprising or consisting of the sequence of SEQ ID NO: 29, optionally having up to 2 amino acid substitutions therefrom;
b. and a light chain variable region (VL) comprising:
   i. a LCDR1 comprising or consisting of the sequence of SEQ ID NO: 42, optionally having up to 2 amino acid substitutions therefrom;
   ii. a LCDR2 comprising or consisting of the sequence of SEQ ID NO: 55, optionally having up to 2 amino acid substitutions therefrom; and
   iii. a LCDR3 comprising or consisting of the sequence of SEQ ID NO: 68, optionally having up to 2 amino acid substitutions therefrom.

In some embodiments the optional amino acid substitutions are conservative substitutions.

In some embodiments, the multivalent antibody or antigen binding fragment thereof comprises a VH comprising or consisting of the sequence of SEQ ID NO: 81 and a VL comprising or consisting of the sequence of SEQ ID NO: 94.

In some embodiments, the multivalent antibody or antigen binding fragment thereof comprises a heavy chain comprising or consisting of the sequence of SEQ ID NO: 111 and a light chain comprising or consisting of the sequence SEQ ID NO: 137.

In some embodiments, the multivalent antibody or antigen binding fragment thereof comprises a heavy chain comprising or consisting of the sequence of SEQ ID NO: 124 and a light chain comprising or consisting of the sequence SEQ ID NO: 137.

As will be understood, any of the multivalent antibodies or antigen-binding fragments thereof comprising sequences from the anti-NKG2D antibody described herein as H3/L3 may be suitably combined with any of the IL-15 polypeptides presented herein and the components arranged into any of the different arrangements and formats of immunoconjugate described herein.

### NKG2D - H4/L4 Antibody Component

In some embodiments, the multivalent antibody or antigen binding fragment thereof comprises sequences from the anti-NKG2D antibody described herein as H4/L4.

In some embodiments, the multivalent antibody or antigen binding fragment thereof comprises:
i. a heavy chain variable region (VH) comprising:
   a. a HCDR1 comprising or consisting of the sequence of SEQ ID NO: 4, optionally having up to 2 amino acid substitutions therefrom;
   b. a HCDR2 comprising or consisting of the sequence of SEQ ID NO: 17, optionally having up to 2 amino acid substitutions therefrom; and
   c. a HCDR3 comprising or consisting of the sequence of SEQ ID NO: 30, optionally having up to 2 amino acid substitutions therefrom;
ii. and a light chain variable region (VL) comprising:
   a. a LCDR1 comprising or consisting of the sequence of SEQ ID NO: 43, optionally having up to 2 amino acid substitutions therefrom;
   b. a LCDR2 comprising or consisting of the sequence of SEQ ID NO: 56, optionally having up to 2 amino acid substitutions therefrom; and
   c. a LCDR3 comprising or consisting of the sequence of SEQ ID NO: 69, optionally having up to 2 amino acid substitutions therefrom.

In some embodiments the optional amino acid substitutions are conservative substitutions.

In some embodiments, the multivalent antibody or antigen binding fragment thereof comprises a VH comprising or consisting of the sequence of SEQ ID NO: 82 and a VL comprising or consisting of the sequence of SEQ ID NO: 95.

In some embodiments, the multivalent antibody or antigen binding fragment thereof comprises a heavy chain comprising or consisting of the sequence of SEQ ID NO: 112 and a light chain comprising or consisting of the sequence SEQ ID NO: 138.

In some embodiments, the multivalent antibody or antigen binding fragment thereof comprises a heavy chain comprising or consisting of the sequence of SEQ ID NO: 125 and a light chain comprising or consisting of the sequence SEQ ID NO: 138.

As will be understood, any of the multivalent antibodies or antigen-binding fragments thereof comprising sequences from the anti-NKG2D antibody described herein as H4/L4 may be suitably combined with any of the IL-15 polypeptides presented herein and the components arranged into any of the different arrangements and formats of immunoconjugate described herein.

### NKG2D - H5/L5 Antibody Component

In some embodiments, the multivalent antibody or antigen binding fragment thereof comprises sequences from the anti-NKG2D antibody described herein as H5/L5.

In some embodiments, the multivalent antibody or antigen binding fragment thereof comprises:
i. a heavy chain variable region (VH) comprising:
   a. a HCDR1 comprising or consisting of the sequence of SEQ ID NO: 5, optionally having up to 2 amino acid substitutions therefrom;
   b. a HCDR2 comprising or consisting of the sequence of SEQ ID NO: 18, optionally having up to 2 amino acid substitutions therefrom; and
   c. a HCDR3 comprising or consisting of the sequence of SEQ ID NO: 31, optionally having up to 2 amino acid substitutions therefrom;
ii. and a light chain variable region (VL) comprising:
   a. a LCDR1 comprising or consisting of the sequence of SEQ ID NO: 44, optionally having up to 2 amino acid substitutions therefrom;
   b. a LCDR2 comprising or consisting of the sequence of SEQ ID NO: 57, optionally having up to 2 amino acid substitutions therefrom; and
   c. a LCDR3 comprising or consisting of the sequence of SEQ ID NO: 70, optionally having up to 2 amino acid substitutions therefrom.

In some embodiments the optional amino acid substitutions are conservative substitutions.

In some embodiments, the multivalent antibody or antigen binding fragment thereof comprises a VH comprising or consisting of the sequence of SEQ ID NO: 83 and a VL comprising or consisting of the sequence of SEQ ID NO: 96.

In some embodiments, the multivalent antibody or antigen binding fragment thereof comprises a heavy chain comprising or consisting of the sequence of SEQ ID NO: 113 and a light chain comprising or consisting of the sequence SEQ ID NO: 139.

In some embodiments, the multivalent antibody or antigen binding fragment thereof comprises a heavy chain comprising or consisting of the sequence of SEQ ID NO: 126 and a light chain comprising or consisting of the sequence SEQ ID NO: 139.

As will be understood, any of the multivalent antibodies or antigen-binding fragments thereof comprising sequences from the anti-NKG2D antibody described herein as H5/L5 may be suitably combined with any of the IL-15 polypeptides presented herein and the components arranged into any of the different arrangements and formats of immunoconjugate described herein.

### NKG2D - H6/L6 Antibody Component

In some embodiments, the multivalent antibody or antigen binding fragment thereof comprises sequences from the anti-NKG2D antibody described herein as H6/L6.

In some embodiments, the multivalent antibody or antigen binding fragment thereof comprises:
i. a heavy chain variable region (VH) comprising:
   a. a HCDR1 comprising or consisting of the sequence of SEQ ID NO: 6, optionally having up to 2 amino acid substitutions therefrom;
   b. a HCDR2 comprising or consisting of the sequence of SEQ ID NO: 19, optionally having up to 2 amino acid substitutions therefrom; and
   c. a HCDR3 comprising or consisting of the sequence of SEQ ID NO: 32, optionally having up to 2 amino acid substitutions therefrom;
ii. and a light chain variable region (VL) comprising:
   a. a LCDR1 comprising or consisting of the sequence of SEQ ID NO: 45, optionally having up to 2 amino acid substitutions therefrom;
   b. a LCDR2 comprising or consisting of the sequence of SEQ ID NO: 58, optionally having up to 2 amino acid substitutions therefrom; and
   c. a LCDR3 comprising or consisting of the sequence of SEQ ID NO: 71, optionally having up to 2 amino acid substitutions therefrom.

In some embodiments the optional amino acid substitutions are conservative substitutions.

In some embodiments, the multivalent antibody or antigen binding fragment thereof comprises a VH comprising or consisting of the sequence of SEQ ID NO: 84 and a VL comprising or consisting of the sequence of SEQ ID NO: 97.

In some embodiments, the multivalent antibody or antigen binding fragment thereof comprises a heavy chain comprising or consisting of the sequence of SEQ ID NO: 114 and a light chain comprising or consisting of the sequence SEQ ID NO: 140.

In some embodiments, the multivalent antibody or antigen binding fragment thereof comprises a heavy chain comprising or consisting of the sequence of SEQ ID NO: 127 and a light chain comprising or consisting of the sequence SEQ ID NO: 140.

As will be understood, any of the multivalent antibodies or antigen-binding fragments thereof comprising sequences from the anti-NKG2D antibody described herein as H6/L6 may be suitably combined with any of the IL-15 polypeptides presented herein and the components arranged into any of the different arrangements and formats of immunoconjugate described herein.

### NKG2D - H7/L7 Antibody Component

In some embodiments, the multivalent antibody or antigen binding fragment thereof comprises sequences from the anti-NKG2D antibody described herein as H7/L7.

In some embodiments, the multivalent antibody or antigen binding fragment thereof comprises:
i. a heavy chain variable region (VH) comprising:
   a. a HCDR1 comprising or consisting of the sequence of SEQ ID NO: 7, optionally having up to 2 amino acid substitutions therefrom;
   b. a HCDR2 comprising or consisting of the sequence of SEQ ID NO: 20, optionally having up to 2 amino acid substitutions therefrom; and
   c. a HCDR3 comprising or consisting of the sequence of SEQ ID NO: 33, optionally having up to 2 amino acid substitutions therefrom;
ii. and a light chain variable region (VL) comprising:
   a. a LCDR1 comprising or consisting of the sequence of SEQ ID NO: 46, optionally having up to 2 amino acid substitutions therefrom;
   b. a LCDR2 comprising or consisting of the sequence of SEQ ID NO: 59, optionally having up to 2 amino acid substitutions therefrom; and
   c. a LCDR3 comprising or consisting of the sequence of SEQ ID NO: 72, optionally having up to 2 amino acid substitutions therefrom.

In some embodiments the optional amino acid substitutions are conservative substitutions.

In some embodiments, the multivalent antibody or antigen binding fragment thereof comprises a VH comprising or consisting of the sequence of SEQ ID NO: 85 and a VL comprising or consisting of the sequence of SEQ ID NO: 98.

In some embodiments, the multivalent antibody or antigen binding fragment thereof comprises a heavy chain comprising or consisting of the sequence of SEQ ID NO: 115 and a light chain comprising or consisting of the sequence SEQ ID NO: 141.

In some embodiments, the multivalent antibody or antigen binding fragment thereof comprises a heavy chain comprising or consisting of the sequence of SEQ ID NO: 128 and a light chain comprising or consisting of the sequence SEQ ID NO: 141.

As will be understood, any of the multivalent antibodies or antigen-binding fragments thereof comprising sequences from the anti-NKG2D antibody described herein as H7/L7 may be suitably combined with any of the IL-15 polypeptides presented herein and the components arranged into any of the different arrangements and formats of immunoconjugate described herein.

### NKG2D - H8/L8 Antibody Component

In some embodiments, the multivalent antibody or antigen binding fragment thereof comprises sequences from the anti-NKG2D antibody described herein as H8/L8.

In some embodiments, the multivalent antibody or antigen binding fragment thereof comprises:
i. a heavy chain variable region (VH) comprising:
   a. a HCDR1 comprising or consisting of the sequence of SEQ ID NO: 8, optionally having up to 2 amino acid substitutions therefrom;
   b. a HCDR2 comprising or consisting of the sequence of SEQ ID NO: 21, optionally having up to 2 amino acid substitutions therefrom; and
   c. a HCDR3 comprising or consisting of the sequence of SEQ ID NO: 34, optionally having up to 2 amino acid substitutions therefrom;
ii. and a light chain variable region (VL) comprising:
   a. a LCDR1 comprising or consisting of the sequence of SEQ ID NO: 47, optionally having up to 2 amino acid substitutions therefrom;
   b. a LCDR2 comprising or consisting of the sequence of SEQ ID NO: 60, optionally having up to 2 amino acid substitutions therefrom; and
   c. a LCDR3 comprising or consisting of the sequence of SEQ ID NO: 73, optionally having up to 2 amino acid substitutions therefrom.

In some embodiments the optional amino acid substitutions are conservative substitutions.

In some embodiments, the multivalent antibody or antigen binding fragment thereof comprises a VH comprising or consisting of the sequence of SEQ ID NO: 86 and a VL comprising or consisting of the sequence of SEQ ID NO: 99.

In some embodiments, the multivalent antibody or antigen binding fragment thereof comprises a heavy chain comprising or consisting of the sequence of SEQ ID NO: 116 and a light chain comprising or consisting of the sequence SEQ ID NO: 142.

In some embodiments, the multivalent antibody or antigen binding fragment thereof comprises a heavy chain comprising or consisting of the sequence of SEQ ID NO: 129 and a light chain comprising or consisting of the sequence SEQ ID NO: 142.

As will be understood, any of the multivalent antibodies or antigen-binding fragments thereof comprising sequences from the anti-NKG2D antibody described herein as H8/L8 may be suitably combined with any of the IL-15 polypeptides presented herein and the components arranged into any of the different arrangements and formats of immunoconjugate described herein.

### NKG2D - H9/L9 Antibody Component

In some embodiments, the multivalent antibody or antigen binding fragment thereof comprises sequences from the anti-NKG2D antibody described herein as H9/L9.

In some embodiments, the multivalent antibody or antigen binding fragment thereof comprises:
i. a heavy chain variable region (VH) comprising:
   a. a HCDR1 comprising or consisting of the sequence of SEQ ID NO: 9, optionally having up to 2 amino acid substitutions therefrom;
   b. a HCDR2 comprising or consisting of the sequence of SEQ ID NO: 22, optionally having up to 2 amino acid substitutions therefrom; and
   c. a HCDR3 comprising or consisting of the sequence of SEQ ID NO: 35, optionally having up to 2 amino acid substitutions therefrom;
ii. and a light chain variable region (VL) comprising:
   a. a LCDR1 comprising or consisting of the sequence of SEQ ID NO: 48, optionally having up to 2 amino acid substitutions therefrom;
   b. a LCDR2 comprising or consisting of the sequence of SEQ ID NO: 61, optionally having up to 2 amino acid substitutions therefrom; and
   c. a LCDR3 comprising or consisting of the sequence of SEQ ID NO: 74, optionally having up to 2 amino acid substitutions therefrom.

In some embodiments the optional amino acid substitutions are conservative substitutions.

In some embodiments, the multivalent antibody or antigen binding fragment thereof comprises a VH comprising or consisting of the sequence of SEQ ID NO: 87 and a VL comprising or consisting of the sequence of SEQ ID NO: 100.

In some embodiments, the multivalent antibody or antigen binding fragment thereof comprises a heavy chain comprising or consisting of the sequence of SEQ ID NO: 117 and a light chain comprising or consisting of the sequence SEQ ID NO: 143.

In some embodiments, the multivalent antibody or antigen binding fragment thereof comprises a heavy chain comprising or consisting of the sequence of SEQ ID NO: 130 and a light chain comprising or consisting of the sequence SEQ ID NO: 143.

As will be understood, any of the multivalent antibodies or antigen-binding fragments thereof comprising sequences from the anti-NKG2D antibody described herein as H9/L9 may be suitably combined with any of the IL-15 polypeptides presented herein and the components arranged into any of the different arrangements and formats of immunoconjugate described herein.

### NKG2D - H10/L10 Antibody Component

In some embodiments, the multivalent antibody or antigen binding fragment thereof comprises sequences from the anti-NKG2D antibody described herein as H10/L10.

In some embodiments, the multivalent antibody or antigen binding fragment thereof comprises:
i. a heavy chain variable region (VH) comprising:
   a. a HCDR1 comprising or consisting of the sequence of SEQ ID NO: 10, optionally having up to 2 amino acid substitutions therefrom;
   b. a HCDR2 comprising or consisting of the sequence of SEQ ID NO: 23, optionally having up to 2 amino acid substitutions therefrom; and
   c. a HCDR3 comprising or consisting of the sequence of SEQ ID NO: 36, optionally having up to 2 amino acid substitutions therefrom;
ii. and a light chain variable region (VL) comprising:
   a. a LCDR1 comprising or consisting of the sequence of SEQ ID NO: 49, optionally having up to 2 amino acid substitutions therefrom;
   b. a LCDR2 comprising or consisting of the sequence of SEQ ID NO: 62, optionally having up to 2 amino acid substitutions therefrom; and
   c. a LCDR3 comprising or consisting of the sequence of SEQ ID NO: 75, optionally having up to 2 amino acid substitutions therefrom.

In some embodiments the optional amino acid substitutions are conservative substitutions.

In some embodiments, the multivalent antibody or antigen binding fragment thereof comprises a VH comprising or consisting of the sequence of SEQ ID NO: 88 and a VL comprising or consisting of the sequence of SEQ ID NO: 101.

In some embodiments, the multivalent antibody or antigen binding fragment thereof comprises a heavy chain comprising or consisting of the sequence of SEQ ID NO: 118 and a light chain comprising or consisting of the sequence SEQ ID NO: 144.

In some embodiments, the multivalent antibody or antigen binding fragment thereof comprises a heavy chain comprising or consisting of the sequence of SEQ ID NO: 131 and a light chain comprising or consisting of the sequence SEQ ID NO: 144.

As will be understood, any of the multivalent antibodies or antigen-binding fragments thereof comprising sequences from the anti-NKG2D antibody described herein as H10/L10 may be suitably combined with any of the IL-15 polypeptides presented herein and the components arranged into any of the different arrangements and formats of immunoconjugate described herein.

### NKp30 Antibody Components

To further outline the applicability of the approach a series of non-limiting example immunoconjugates are provided herein. These immunoconjugates comprise least two IL-15 polypeptides and a multivalent antibody or antigen-binding fragment thereof, comprising at least two antibody binding domains that each specifically bind to NKp30. In preferred embodiments the multivalent antibody or antigen-binding fragments thereof binds to NKp30 in a monospecific manner, meaning both antibody binding domains bind to the NKp30, and to the same NKp30 epitope.

A summary of the multivalent antibodies or antigen binding fragments thereof that form the antibody component of these NKp30-IL-15 immunoconjugates is provided below.

### H11/L11 Antibody Component

In some embodiments, the multivalent antibody or antigen binding fragment thereof comprises sequences from the anti-NKp30 antibody described herein as H11/L11.

In some embodiments, the multivalent antibody or antigen binding fragment thereof comprises:
a) a heavy chain variable region (VH) comprising:
   i. a HCDR1 comprising or consisting of the sequence of SEQ ID NO: 11, optionally having up to 2 amino acid substitutions therefrom;
   ii. a HCDR2 comprising or consisting of the sequence of SEQ ID NO: 24, optionally having up to 2 amino acid substitutions therefrom; and
   iii. a HCDR3 comprising or consisting of the sequence of SEQ ID NO: 37, optionally having up to 2 amino acid substitutions therefrom;
b) and a light chain variable region (VL) comprising:
   i. a LCDR1 comprising or consisting of the sequence of SEQ ID NO: 50, optionally having up to 2 amino acid substitutions therefrom;
   ii. a LCDR2 comprising or consisting of the sequence of SEQ ID NO: 63, optionally having up to 2 amino acid substitutions therefrom; and
   iii. a LCDR3 comprising or consisting of the sequence of SEQ ID NO: 76, optionally having up to 2 amino acid substitutions therefrom.

In some embodiments the optional amino acid substitutions are conservative substitutions.

In some embodiments the optional amino acid substitutions are conservative substitutions.

In some embodiments, the multivalent antibody or antigen binding fragment thereof comprises a VH comprising or consisting of the sequence of SEQ ID NO: 89 and a VL comprising or consisting of the sequence of SEQ ID NO: 102.

In some embodiments, the multivalent antibody or antigen binding fragment thereof comprises a heavy chain comprising or consisting of the sequence of SEQ ID NO: 119 and a light chain comprising or consisting of the sequence SEQ ID NO: 145.

In some embodiments, the multivalent antibody or antigen binding fragment thereof comprises a heavy chain comprising or consisting of the sequence of SEQ ID NO: 132 and a light chain comprising or consisting of the sequence SEQ ID NO: 145.

As will be understood, any of the multivalent antibodies or antigen-binding fragments thereof comprising sequences from the anti-NKp3D antibody described herein as H11/L11 may be suitably combined with any of the IL-15 polypeptides presented herein and the components arranged into any of the different arrangements and formats of immunoconjugate described herein.

### NKp30 - H12/L12 Antibody Component

In some embodiments, the multivalent antibody or antigen binding fragment thereof comprises sequences from the anti-NKp30 antibody described herein as H12/L12.

In some embodiments, the multivalent antibody or antigen binding fragment thereof comprises:
a) a heavy chain variable region (VH) comprising:
   i. a HCDR1 comprising or consisting of the sequence of SEQ ID NO: 12, optionally having up to 2 amino acid substitutions therefrom;
   ii. a HCDR2 comprising or consisting of the sequence of SEQ ID NO: 25, optionally having up to 2 amino acid substitutions therefrom; and
   iii. a HCDR3 comprising or consisting of the sequence of SEQ ID NO: 38, optionally having up to 2 amino acid substitutions therefrom;
b) and a light chain variable region (VL) comprising:
   i. a LCDR1 comprising or consisting of the sequence of SEQ ID NO: 51, optionally having up to 2 amino acid substitutions therefrom;
   ii. a LCDR2 comprising or consisting of the sequence of SEQ ID NO: 64, optionally having up to 2 amino acid substitutions therefrom; and
   iii. a LCDR3 comprising or consisting of the sequence of SEQ ID NO: 77, optionally having up to 2 amino acid substitutions therefrom.

In some embodiments the optional amino acid substitutions are conservative substitutions.

In some embodiments, the multivalent antibody or antigen binding fragment thereof comprises a VH comprising or consisting of the sequence of SEQ ID NO: 90 and a VL comprising or consisting of the sequence of SEQ ID NO: 103.

In some embodiments, the multivalent antibody or antigen binding fragment thereof comprises a heavy chain comprising or consisting of the sequence of SEQ ID NO: 120 and a light chain comprising or consisting of the sequence SEQ ID NO: 146.

In some embodiments, the multivalent antibody or antigen binding fragment thereof comprises a heavy chain comprising or consisting of the sequence of SEQ ID NO: 133 and a light chain comprising or consisting of the sequence SEQ ID NO: 146.

As will be understood, any of the multivalent antibodies or antigen-binding fragments thereof comprising sequences from the anti-NKp30 antibody described herein as H11/L11 may be suitably combined with any of the IL-15 polypeptides presented herein and the components arranged into any of the different arrangements and formats of immunoconjugate described herein.

### NKp46 Antibody Components

To further outline the applicability of the approach some non-limiting examples of immunoconjugates are provided herein. These immunoconjugates comprise least two IL-15 polypeptides and a multivalent antibody or antigen-binding fragment thereof, comprising at least two antibody binding domains that each specifically bind to NKp46. In preferred embodiments the multivalent antibody or antigen-binding fragments thereof binds to NKp46 in a monospecific manner, meaning both antibody binding domains bind to the NKp46, and to the same NKp46 epitope.

A summary of the multivalent antibodies or antigen binding fragments thereof that form the antibody component of these NKp-IL-15 immunoconjugates is provided below.

### H13/L13 Antibody Component

In some embodiments, the multivalent antibody or antigen binding fragment thereof comprises sequences from the anti-NKp46 antibody described herein as H13/L13.

In some embodiments, the multivalent antibody or antigen binding fragment thereof comprises:
a) a heavy chain variable region (VH) comprising:
   i. a HCDR1 comprising or consisting of the sequence of SEQ ID NO: 13, optionally having up to 2 amino acid substitutions therefrom;
   ii. a HCDR2 comprising or consisting of the sequence of SEQ ID NO: 26, optionally having up to 2 amino acid substitutions therefrom; and
   iii. a HCDR3 comprising or consisting of the sequence of SEQ ID NO: 39, optionally having up to 2 amino acid substitutions therefrom;
b) and a light chain variable region (VL) comprising:
   i. a LCDR1 comprising or consisting of the sequence of SEQ ID NO: 52, optionally having up to 2 amino acid substitutions therefrom;
   ii. a LCDR2 comprising or consisting of the sequence of SEQ ID NO: 65, optionally having up to 2 amino acid substitutions therefrom; and
   iii. a LCDR3 comprising or consisting of the sequence of SEQ ID NO: 78, optionally having up to 2 amino acid substitutions therefrom.

In some embodiments the optional amino acid substitutions are conservative substitutions.

In some embodiments, the multivalent antibody or antigen binding fragment thereof comprises a VH comprising or consisting of the sequence of SEQ ID NO: 91 and a VL comprising or consisting of the sequence of SEQ ID NO: 104.

In some embodiments, the multivalent antibody or antigen binding fragment thereof comprises a heavy chain comprising or consisting of the sequence of SEQ ID NO: 121 and a light chain comprising or consisting of the sequence SEQ ID NO: 147.

In some embodiments, the multivalent antibody or antigen binding fragment thereof comprises a heavy chain comprising or consisting of the sequence of SEQ ID NO: 134 and a light chain comprising or consisting of the sequence SEQ ID NO: 147.

As will be understood, any of the multivalent antibodies or antigen-binding fragments thereof comprising sequences from the anti-NKp46 antibody described herein as H12/L12 may be suitably combined with any of the IL-15 polypeptides presented herein and the components arranged into any of the different arrangements and formats of immunoconjugate described herein.

### IL-15 Polypeptides

The immunoconjugates of the invention comprise at least two IL-15 polypeptides, and the term “IL-15 polypeptide” as used herein, refers to a polypeptide comprising a IL-15 cytokine sequence, and an IL-15 receptor alpha (CD215) sequence or a functional fragment of IL-15 receptor alpha (CD215) sequence. In some embodiments a functional fragment of IL-15 receptor alpha (CD215) sequence comprises a minimum of the IL-15 receptor alpha “sushi domain”.

As will be understood, any of the various IL-15 polypeptides presented herein may be suitably combined with any of the multivalent antibodies or antigen-binding fragments thereof and the components arranged into the different arrangements and formats of immunoconjugates described herein.

The term “interleukin-15” or “IL-15” or “IL-15 cytokine” as used herein, refers to any native IL-15 polypeptide from any vertebrate source, including mammals such as primates (e.g. humans) and rodents (e.g., mice and rats), unless otherwise indicated. The term encompasses unprocessed IL-15 as well as any form of IL-15 that results from processing in the cell. The term also encompasses naturally occurring variants of IL-15, e.g. splice variants or allelic variants. The amino acid sequence of an exemplary mature human IL-15 cytokine is shown in SEQ ID NO: 151. SEQ ID NO: 152 provides the sequence of an IL-15 signal peptide. SEQ ID NO: 153 provides the sequence of an IL-15 pro-peptide. Unprocessed human IL-15 additionally comprises an N-terminal amino acid signal peptide having the sequence typically absent in the mature IL-15 molecule.

In preferred embodiments, the IL-15 cytokine is human IL-15 (hIL-15) cytokine or a variant or fragment thereof. The term “human IL-15” as used herein refers to contiguous sequence that is at least about 90%, at least about 91%, at least about 92%, at least about 93%, at least about 94%, at least about 95% or at least about 96% identical to the equivalent portion of human IL-15 sequence of SEQ ID NO: 151 to which the contiguous sequence relates to. Particularly, the sequence identity is at least about 95%, more particularly at least about 96%. In particular embodiments, the human IL-15 component comprises a full-length, mature IL-15 polypeptide. Such human IL-15 components as defined herein may or may not also contain additional non-IL-15 cytokine sequence located or fused either N-terminal or C-terminal.

The term “IL-15 receptor alpha” as used herein refers to contiguous sequence that is at least about 90%, at least about 91%, at least about 92%, at least about 93%, at least about 94%, at least about 95% or at least about 96% identical to the equivalent portion of human IL-15 receptor alpha sequence of SEQ ID NO: 148 to which the contiguous sequence relates to. Such IL-15 receptor alpha polypeptides as defined herein may or may not also contain additional non-IL-15 receptor alpha sequence located or fused either N-terminal or C-terminal to the region homologous to human IL-15 receptor alpha. In some preferred embodiments, the IL-15 receptor alpha (CD215) is human IL-15 receptor alpha (CD215). In some embodiments, the IL-15 receptor alpha (CD215) comprises or consists of a sequence having at least 90%, at least 91%, at least 92%, at least 93%, at least 94%, at least 95%, at least 95%, at least 97%, at least 98% or at least 99% identity to SEQ ID NO: 148. In some embodiments, the IL-15 receptor alpha (CD215) portion of the IL-15 polypeptide comprises or consists of SEQ ID NO: 148. In some embodiments, the IL-15 polypeptide comprises up to 100 consecutive amino acids of the IL-15 receptor alpha (CD215) sequence and the up to 100 consecutive amino acids comprises the IL-15 receptor alpha (CD215) sushi domain. In particular embodiments, the IL-15 receptor alpha sequence comprises a minimum of the human IL-15 receptor alpha “sushi domain”.

The term “IL-15 receptor alpha (CD215) sushi domain” or “sushi domain” refers to contiguous sequence that is at least about 90%, at least about 91%, at least about 92%, at least about 93%, at least about 94%, at least about 95% or at least about 96% identical to the equivalent portion of IL-15 sushi domain as defined in SEQ ID NO: 149 or SEQ ID NO: 150. SEQ ID NO: 50 refers to an “extended” sushi domain including an additional hinge region. In preferred embodiments, the IL-15 receptor alpha sequence within the IL-15 polypeptide includes the IL-15 receptor alpha “sushi domain”, preferably the human IL-15 receptor alpha sushi domain. Such “sushi domains” as defined herein may or may not also contain additional IL-15 receptor alpha sequence located either N-terminal or C-terminal to said sushi domain.

In some embodiments the IL-15 receptor alpha (CD215) sushi domain comprises or consists of a sequence having at least 90%, at least 91%, at least 92%, at least 93%, at least 94%, at least 95%, at least 95%, at least 97%, at least 98% or at least 99% identity to a sequence selected from the group consisting of SEQ ID NOs: 149 to 150, preferably SEQ ID NO: 149. In some embodiments the IL-15 receptor alpha (CD215) sushi domain comprises or consists of a sequence selected from the group consisting of SEQ ID NOs: 149 to 150. In preferred embodiments the IL-15 receptor alpha (CD215) sushi domain comprises or consists of the sequence SEQ ID NO: 149.

The IL-15 polypeptides of the invention comprise two components: an IL-15 cytokine; and IL-15 receptor alpha (CD215) or a functional fragment thereof. These components can be arranged in either order, and can be joined in different ways. In some embodiments IL-15 cytokine and the IL-15 receptor alpha (CD215) or functional fragment thereof are directly fused to each other. In some embodiments the IL-15 cytokine and the IL-15 receptor alpha (CD215) or functional fragment thereof are joined by a linker, preferably a flexible linker. In some embodiments the linker is a peptide linker, optionally a peptide of from about 1 to about 60 amino acid residues in length. In some embodiments, the linker comprises or consists of glycine and serine. The glycine and serine residues can be arranged in different ways. GlySer linkers are known in the art and may be used. For example, in some embodiments the linker comprises or consists of the sequence [GGGGS]ₙX, or [GGGS]ₙX, or [GGS]ₙX, wherein n is from 1 to 6 and wherein X is G or is absent. In some embodiments, the linker comprises or consists of the sequence [[G]ₘS]ₙX, wherein m is from 2 to 4, n is from 1 to 6 and X is G or is absent. In some embodiments, the linker comprises or consists of glycine and serine residues, and optionally the linker comprises more glycine than serine residues. In some embodiments, the linker comprises or consist of glycine and serine residues in a ratio of from about 5:1 to about 2:1. In some embodiments, the linker comprises or consists of glycine and serine residues in a ratio of about 5:1. In some embodiments the linker comprises or consists of about 80% glycine and about 20% serine. In some embodiments the linker comprises or consists of a sequence selected from the group consisting of SEQ ID NOs: 182 to 187 and 204 to 205. In some embodiments, the linker comprises or consists of the sequence of SEQ ID NO: 184. In some embodiments, the linker comprises or consists of the sequence of SEQ ID NO: 201. In preferred embodiments, the linker comprises or consists of the sequence of SEQ ID NO:183.

In some embodiments the IL-15 polypeptide is conjugated to an intact IgG.

In some embodiments, the IL-15 polypeptide comprises, in an N- to C-terminal direction (wherein the N-terminus of the IL-15 polypeptide is conjugated to the multivalent antibody component), a human IL-15 cytokine, optionally a linker, and IL-15 receptor alpha (CD215) or functional fragment thereof.

In preferred embodiments, the IL-15 polypeptide comprises, in an N- to C-terminal direction (wherein the N-terminus of the IL-15 polypeptide is conjugated to the multivalent antibody component), IL-15 receptor alpha (CD215) or functional fragment thereof, optionally a linker, and a human IL-15 cytokine.

In some embodiments, the IL-15 cytokine is an IL-15 mutein. The term “IL-15 mutein”, or “IL-15 mutant” or “mutant IL-15 polypeptide” as used herein is intended to encompass any mutant forms of the IL-15 cytokine including full-length mutated IL-15, truncated forms of IL-15 and forms where IL-15 is linked to another molecule such as by fusion. “Full-length” when used in reference to IL-15 is intended to mean the mature, natural length IL-15 polypeptide. For example, full-length human IL-15 refers to SEQ ID NO: 151. The various forms of IL-15 mutants are characterized in having at least one amino acid mutation affecting the interaction of IL-15 with at least one receptor of the IL-15 receptor complex, for example the mutation may increase function or reduce function.

In preferred embodiments, the IL-15 cytokine is not mutated to reduce function. In preferred embodiments, the IL-15 cytokine is wildtype human IL-15 cytokine (SEQ ID NO: 151). In some embodiments, the IL-15 polypeptide does not comprise mutations that can impair, reduce or disrupt binding or function of the IL-15 cytokine, such as mutations selected from the group consisting of N1D, N4D, D8N, D30N, D61N, E64Q, N65D, and Q108E. Other mutations to reduce binding are also known in the art. Additionally, dialling up or down the number of cysteine residues is not recommended and should be avoided as these disrupt wild-type function. A non-limiting example of such disruptive cys mutations to be avoided include any one or more from a list of C42S, L45C, Q48C, V49C, L52C, E53C, E87C, or E89C. All numbering of these mutations is based on the mature IL-15 cytokine (SEQ ID NO: 151; N-terminal N=residue number 1).

In some embodiments the IL-15 polypeptide is an IL-15 superagonist. The term “IL-15 superagonist” as referred to herein refers to a sub-set of IL-15 components with enhanced or increased biological activity compared to wildtype IL-15 alone. An “IL-15 superagonist” as used herein has a higher agonistic activity compared to wild-type IL-15. This is also sometimes also referred to as hyperagonism. In preferred embodiments the IL-15 superagonist is a human IL-15 cytokine fused to the sushi domain of the IL-15 receptor alpha (SEQ ID: 149). In some embodiments the IL-15 mutein comprises a substitution at position N72 relative to the sequence of SEQ ID NO: 151. In some embodiments the IL-15 mutein is a human IL-15 component comprising the N72D mutation.

In some embodiments, the IL-15 cytokine comprises or consists of a sequence having at least 90%, at least 91%, at least 92%, at least 93%, at least 94%, at least 95%, at least 95%, at least 97%, at least 98% or at least 99% identity to a sequence selected from the group consisting of SEQ ID NOs: 151 to 153. In some embodiments, the IL-15 cytokine comprises or consists of a sequence selected from the group consisting of SEQ ID NOs: 151 to 153. In preferred embodiments, the IL-15 cytokine is SEQ ID NO: 151.

In some embodiments, the IL-15 polypeptide comprises or consists of a sequence having at least 90%, at least 91%, at least 92%, at least 93%, at least 94%, at least 95%, at least 95%, at least 97%, at least 98% or at least 99% identity to a sequence selected from the group consisting of SEQ ID NOs: 154, 155, 206 and 207, preferably SEQ ID NO: 154. In some embodiments, the IL-1S polypeptide comprises or consists of a sequence selected from the group consisting of SEQ ID NOs: 154, 155, 206 and 207. In preferred embodiments, the IL-15 polypeptide comprises or consists of the sequence of SEQ ID NO: 154, which comprises the sushi domain of SEQ ID NO: 149, the linker of SEQ ID NO: 183 and the IL-15 cytokine of SEQ ID NO: 151. In some embodiments the IL-15 polypeptide comprises or consists of SEQ ID NO: 206. This IL-15 polypeptide comprises a sushi domain (SEQ ID NO: 149), a linker (SEQ ID NO: 184) and an IL-15 cytokine (SEQ ID NO: 151). In some embodiments the IL-15 polypeptide comprises or consists of SEQ ID NO: 207. This IL15 polypeptide comprises a sushi domain (SEQ ID NO: 149), a linker (SEQ ID NO: 201 and/or 183) and an IL-15 cytokine (SEQ ID NO: 151).

### Specific Immunoconjugates

A non-limiting summary of some of the immunoconjugates provided and exemplified herein is provided below in Table 2.

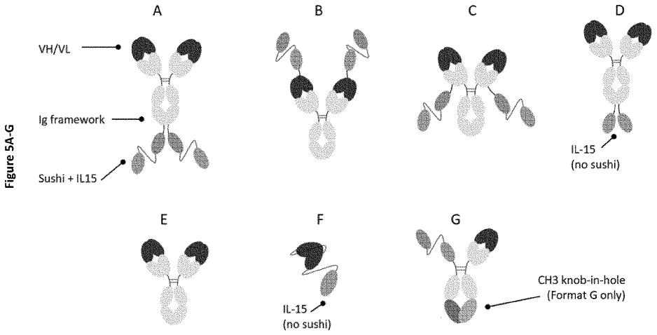

TABLE 2

| | Seq ID | Seq ID | Antibody | | | | |
|---|---|---|---|---|---|---|---|
| Molecule | of heavy | of light | component | | | Format (see | |
| Name | chain | chain | name | VH | VL | FIG. 5) | Target |
| CYTB02 | 156 | 135 | H1/L1 | 79 | 92 | A | NKG2D |
| CYTB03 | 188 | 135 | H1 with | 202 | 92 | A | NKG2D |
| | | | framework | | | | |
| | | | change/L1 | | | | |
| CYTB04 | 160 | 139 | H5/L5 | 83 | 96 | A | NKG2D |
| CYTB05 | 161 | 140 | H6/L6 | 84 | 97 | A | NKG2D |
| CYTB06 | 189 | 140 | H6/L6 | 84 | 97 | A | NKG2D |
| CYTB07 | 190 | 140 | H6/L6 | 84 | 97 | B | NKG2D |
| CYTB08 | 191 | 140 | H6/L6 | 84 | 97 | A | NKG2D |
| CYTB11 | 114 | 174 | H6/L6 | 84 | 97 | C | NKG2D |
| CYTB12 | 192 | 140 | H6/L6 | 84 | 97 | A | NKG2D |
| CYTB13 | 114 | 199 | H6/L6 | 84 | 97 | C | NKG2D |
| CYTB14 | 193 | 140 | H6/L6 | 84 | 97 | D | NKG2D |
| CYTB15 | 167 | 146 | H12/L12 | 90 | 103 | A | Nkp30 |
| CYTB16 | 168 | 147 | H13/L13 | 91 | 104 | A | Nkp46 |
| CYTB21 | 114 | 140 | H6/L6 | 84 | 97 | E | NKG2D |
| CYTB24 | 120 | 146 | H12/L12 | 90 | 103 | E | Nkp30 |
| CYTB25 | 121 | 147 | H13/L13 | 91 | 104 | E | Nkp46 |
| CYTB27 | 194 | NA | H1/— | 79 | — | F | NKG2D |
| CYTB28 | 195 | 140 | H6/L6 | 84 | 97 | A | NKG2D |
| CYTB29 | 196, 197 | 139 | H5/L5 | 83 | 96 | G | NKG2D |
| CYTB30 | 198, 197 | 139 | H5/L5 | 83 | 96 | G | NKG2D |
| CYTB32 | 200 | 140 | H6/L6 | 84 | 97 | A | NKG2D |

### Specific Immunoconjugates - IL-15 Polypeptides are Joined to C-Termini of Heavy Chains

In some embodiments of the invention, the IL-15 polypeptides are conjugated (optionally via a linker) to the C-termini of the heavy chains of the antibody component. In some preferred embodiments the IL-15 polypeptides are conjugated (optionally via a linker) to the C-termini of the CH3 domains of the heavy chains. This format is shown in FIG. 4A and FIG. 5A.

In the immunoconjugate formats described below, the immunoglobulin heavy chain(s) of the multivalent antibody is referred to as the first polypeptide chain. The first and second copy of the first polypeptide chain therefore refer to the two identical heavy chains of the multivalent antibody. In the immunoconjugate formats described below, the immunoglobulin light chain(s) of the multivalent antibody is referred to as the second polypeptide chain. The first and second copy of the second polypeptide chain therefore refer to the two identical light chains of the multivalent antibody. In some embodiments, the first polypeptide (heavy chain) and second polypeptide (light chain) forms one subunit. This then homodimerizes to form the final immunoconjugate comprising two said subunits and thus a total of four polypeptide chains. Hence the immunoconjugates comprise first and second copies of said first and second polypeptide chains. The immunoconjugates can therefore be described as dimers, with one monomer subunit of the dimer comprising a first copy of the first polypeptide chain and a first copy of the second polypeptide chain and the second monomer subunit of the dimer comprising a second copy of the first polypeptide chain and a second copy of the second polypeptide chain. In these embodiments the immunoconjugate is considered homodimeric. In some embodiments, the immunoconjugate may have been selectively purified in order to arrive at such a homodimeric species and remove or reduce any unwanted fragments, higher order aggregates, and alternative heterodimeric or conformer species that may be considered impurities. In some embodiments the homodimeric immunoconjugate is the preferred species in a mixture that includes said impurities

In some embodiments, the anti-NKG2D antibody, anti-NKp30 antibody, anti-NKp46 antibody, anti-NKp44 antibody or anti-DNAM-1 antibody referred to in the immunoconjugate formats described below is any of the antibodies H1/L1 to H13/L13, or could be another antibody against these targets known in the art.

In some embodiments the immunoconjugate comprises:
a) a first polypeptide chain comprising, in an N- to C-terminal direction:
   i. a heavy chain variable region (VH) of an anti-NKG2D antibody, an anti-NKp30 antibody, an anti-NKp46 antibody, an anti-NKp44 antibody or an anti-DNAM-1 antibody;
   ii. a human heavy chain constant region;
   iii. optionally a linker; and
   iv. the IL-15 polypeptide; and
b) a second polypeptide chain comprising, in an N- to C-terminal direction:
   i. a light chain variable region (VL) of an anti-NKG2D antibody, an anti-NKp30 antibody, an anti-NKp46 antibody, an anti-NKp44 antibody or an anti-DNAM-1 antibody; and
   ii. a human light constant region;
      and the immunoconjugate comprises or consists of first and second copies of said first and second polypeptides.

In some embodiments the immunoconjugate comprises:
a) a first polypeptide chain comprising, in an N- to C-terminal direction:
   i. a heavy chain variable region (VH) of an anti-NKG2D antibody, an anti-NKp30 antibody, an anti-NKp46 antibody, an anti-NKp44 antibody or an anti-DNAM-1 antibody;
   ii. a human heavy chain constant region;
   iii. optionally a linker; and
   iv. an IL-15 polypeptide comprising the sequence of SEQ ID NO: 154, SEQ ID NO: 206 or SEQ ID NO: 207; and
b) a second polypeptide chain comprising, in an N- to C-terminal direction:
   i. a light chain variable region (VL) of an anti-NKG2D antibody, an anti-NKp30 antibody, an anti-NKp46 antibody, an anti-NKp44 antibody or an anti-DNAM-1 antibody; and
   ii. a human light constant region;
      and the immunoconjugate comprises or consists of first and second copies of said first and second polypeptides.

In some embodiments the immunoconjugate comprises:
a) a first polypeptide chain comprising, in an N- to C-terminal direction:
   i. a heavy chain variable region (VH) of an anti-NKG2D antibody, an anti-NKp30 antibody, an anti-NKp46 antibody, an anti-NKp44 antibody or an anti-DNAM-1 antibody;
   ii. a human heavy chain IgG sequence;
   iii. optionally a linker; and
   iv. the IL-15 polypeptide; and
b) a second polypeptide chain comprising, in an N- to C-terminal direction:
   i. a light chain variable region (VL) of an anti-NKG2D antibody, an anti-NKp30 antibody, an anti-NKp46 antibody, an anti-NKp44 antibody or an anti-DNAM-1 antibody; and
   ii. a human light chain IgG sequence;
      and the immunoconjugate comprises or consists of first and second copies of said first and second polypeptides.

In some embodiments the immunoconjugate comprises:
a) a first polypeptide chain comprising, in an N- to C-terminal direction:
   i. a heavy chain variable region (VH) of an anti-NKG2D antibody, an anti-NKp30 antibody, an anti-NKp46 antibody, an anti-NKp44 antibody or an anti-DNAM-1 antibody;
   ii. a human heavy chain IgG1 sequence;
   iii. optionally a linker; and
   iv. the IL-15 polypeptide; and
b) a second polypeptide chain comprising, in an N- to C-terminal direction:
   i. a light chain variable region (VL) of an anti-NKG2D antibody, an anti-NKp30 antibody, an anti-NKp46 antibody, an anti-NKp44 antibody or an anti-DNAM-1 antibody; and
   ii. a human light chain IgG1 sequence;
      and the immunoconjugate comprises or consists of first and second copies of said first and second polypeptides.

In some embodiments the immunoconjugate comprises:
a) a first polypeptide chain comprising, in an N- to C-terminal direction:
   i. a heavy chain of an anti-NKG2D antibody, an anti-NKp30 antibody, an anti-NKp46 antibody, an anti-NKp44 antibody or an anti-DNAM-1 antibody;
   ii. optionally a linker; and
   iii. the IL-15 polypeptide; and
b) a second polypeptide chain comprising a light chain of an anti-NKG2D antibody, an anti-NKp30 antibody, an anti-NKp46 antibody, an anti-NKp44 antibody or an anti-DNAM-1 antibody;
   and the immunoconjugate comprises or consists of first and second copies of said first and second polypeptides.

In some embodiments the immunoconjugate comprises:
a) a first polypeptide chain comprising, in an N- to C-terminal direction:
   i. a heavy chain variable region (VH) of an anti-NKG2D antibody, an anti-NKp3D antibody, an anti-NKp46 antibody, an anti-NKp44 antibody or an anti-DNAM-1 antibody, the VH comprising an HCDR1, an HCDR2 and an HCDR3;
   ii. a heavy chain constant region comprising an CH1, a hinge, a CH2 and a CH3 region;
   iii. optionally a linker, and
   iv. the IL-15 polypeptide; and
b) a second polypeptide chain comprising, in an N- to C-terminal direction:
   i. a light chain variable region (VL) of an anti-NKG2D antibody, an anti-NKp30 antibody, an anti-NKp46 antibody, an anti-NKp44 antibody or an anti-DNAM-1 antibody, the VL comprising an LCDR1, an LCDR2 and an LCDR3; and
   ii. a light chain constant (CL) region;
      and the immunoconjugate comprises or consists of first and second copies of said first and second polypeptides.

In any of the above embodiments, the first and second polypeptide chains are covalently or non-covalently associated with each other such that each heavy chain (variable region) and each light chain (variable region) form an antigen binding domain. The first copy of the first and second polypeptide chain and the second copy of the first and second polypeptide chain are covalently or non-covalently associated with each other in an immunoglobulin-type format, thereby forming the immunoconjugate comprising the multivalent antibody or antigen binding fragment thereof.

### NKG2D HC C-Terminus IL-15 Immunoconjugates

To further outline the applicability of the approach a series of non-limiting example immunoconjugates are provided herein. These immunoconjugates comprise at least two IL-15 polypeptides conjugated to the C-termini of the heavy chains of a multivalent antibody or antigen-binding fragment thereof that specifically binds to NKG2D. In preferred embodiments, the two IL-15 polypeptides are conjugated to the CH3 domain as shown in FIG. 4A and FIG. 5A.

In some embodiments the immunoconjugate comprises:
a) a first polypeptide chain comprising, in an N- to C-terminal direction:
   i. a heavy chain variable region (VH) of an anti-NKG2D antibody comprising an HCDR1 comprising or consisting of a sequence selected from the group consisting of SEQ ID NOs: 1 to 10 and optionally having up to 2 amino acid substitutions thereto, an HCDR2 comprising or consisting of a sequence selected from the group consisting of SEQ ID NOs: 14 to 23 and optionally having up to 2 amino acid substitutions thereto, and an HCDR3 comprising or consisting of a sequence selected from the group consisting of SEQ ID NOs: 27 to 36 and optionally having up to 2 amino acid substitutions thereto;
   ii. a heavy chain constant region comprising or consisting of a sequence having at least 90%, at least 91%, at least 92%, at least 93%, at least 94%, at least 95%, at least 95%, at least 97%, at least 98% or at least 99% identity to a sequence selected from the group consisting of SEQ ID NOs: 105, 106 and 203;
   iii. optionally a linker; and
   iv. an IL-15 polypeptide comprising or consisting of a sequence having at least 90%, at least 91%, at least 92%, at least 93%, at least 94%, at least 95%, at least 95%, at least 97%, at least 98% or at least 99% identity to a sequence selected from the group consisting of SEQ ID NOs: 148 to 155, 206 and 207; and
b) a second polypeptide chain comprising, in an N- to C-terminal direction:
   i. a light chain variable region (VL) of an anti-NKG2D antibody comprising an LCDR1 comprising or consisting of a sequence selected from the group consisting of SEQ ID NOs: 40 to 49 and optionally having up to 2 amino acid substitutions thereto, an LCDR2 comprising or consisting of a sequence selected from the group consisting of SEQ ID NOs: 53 to 62 and optionally having up to 2 amino acid substitutions thereto, and an LCDR3 comprising or consisting of a sequence selected from the group consisting of SEQ ID NOs: 66 to 75 and optionally having up to 2 amino acid substitutions thereto; and
   ii. a light chain constant region comprising or consisting of a sequence having at least 90%, at least 91%, at least 92%, at least 93%, at least 94%, at least 95%, at least 95%, at least 97%, at least 98% or at least 99% identity to a sequence selected from the group consisting of SEQ ID NOs: 107 to 108;
      and the immunoconjugate comprises or consists of first and second copies of said first and second polypeptides.

In some embodiments the optional amino acid substitutions are conservative substitutions.

In some embodiments the immunoconjugate comprises:
a) a first polypeptide chain comprising, in an N- to C-terminal direction:
   i. a heavy chain variable region (VH) of an anti-NKG2D antibody comprising an HCDR1 comprising or consisting of a sequence selected from the group consisting of SEQ ID NOs: 1 to 10, an HCDR2 comprising or consisting of a sequence selected from the group consisting of SEQ ID NOs: 14 to 23 and an HCDR3 comprising or consisting of a sequence selected from the group consisting of SEQ ID NOs: 27 to 36;
   ii. a heavy chain constant region comprising or consisting of a sequence selected from the group consisting of SEQ ID NOs: 105, 106 and 203;
   iii. optionally a linker; and
   iv. an IL-15 polypeptide comprising or consisting of a sequence selected from the group consisting of SEQ ID NOs: 148 to 155, 206 and 207; and
b) a second polypeptide chain comprising, in an N- to C-terminal direction:
   i. a light chain variable region (VL) of an anti-NKG2D antibody comprising an LCDR1 comprising or consisting of a sequence selected from the group consisting of SEQ ID NOs: 40 to 49, an LCDR2 comprising or consisting of a sequence selected from the group consisting of SEQ ID NOs: 53 to 62 and an LCDR3 comprising or consisting of a sequence selected from the group consisting of SEQ ID NOs: 66 to 75; and
   ii. a light chain constant region comprising or consisting of a sequence selected from the group consisting of SEQ ID NOs: 107 to 108;
      and the immunoconjugate comprises or consists of first and second copies of said first and second polypeptides.

In some embodiments the immunoconjugate comprises:
a) a first polypeptide chain comprising, in an N- to C-terminal direction:
   i. a heavy chain variable region (VH) of an anti-NKG2D antibody comprising an HCDR1 comprising or consisting of the sequence of SEQ ID NO: 6, an HCDR2 comprising or consisting of the sequence of SEQ ID NO: 19 and an HCDR3 comprising or consisting of the sequence of SEQ ID NO: 32, optionally wherein the VH region comprises or consists of the sequence of SEQ ID NO:84;
   ii. a heavy chain constant region comprising or consisting of a sequence of SEQ ID NO: 105, 106 and 203;
   iii. optionally a linker; and
   iv. an IL-15 polypeptide comprising or consisting of a sequence selected from the group consisting of SEQ ID NOs: 148 to 155, 206 and 207; and
b) a second polypeptide chain comprising, in an N- to C-terminal direction:
   i. a light chain variable region (VL) of an anti-NKG2D antibody comprising an LCDR1 comprising or consisting of the sequence of SEQ ID NO: 45, an LCDR2 comprising or consisting of the sequence of SEQ ID NO: 58 and an LCDR3 comprising or consisting of the sequence of SEQ ID NO: 71, optionally wherein the VL region comprises or consists of the sequence of SEQ ID NO: 97; and
   ii. a light chain constant region comprising or consisting of the sequence of SEQ ID NO: 107;
      and the immunoconjugate comprises or consists of first and second copies of said first and second polypeptides.

In some embodiments the immunoconjugate comprises:
a) a first polypeptide chain comprising, in an N- to C-terminal direction:
i. a heavy chain variable region (VH) of an anti-NKG2D antibody comprising an HCDR1 comprising or consisting of the sequence of SEQ ID NO: 6, an HCDR2 comprising or consisting of the sequence of SEQ ID NO: 19 and an HCDR3 comprising or consisting of the sequence of SEQ ID NO: 32, optionally wherein the VH region comprises or consists of the sequence of SEQ ID NO:84;
   ii. a heavy chain constant region comprising or consisting of a sequence of SEQ ID NO: 105;
   iii. optionally a linker; and
   iv. an IL-15 polypeptide comprising or consisting of a sequence selected from the group consisting of SEQ ID NOs: 148 to 155, 206 and 207; and
b) a second polypeptide chain comprising, in an N- to C-terminal direction:
   i. a light chain variable region (VL) of an anti-NKG2D antibody comprising an LCDR1 comprising or consisting of the sequence of SEQ ID NO: 45, an LCDR2 comprising or consisting of the sequence of SEQ ID NO: 58 and an LCDR3 comprising or consisting of the sequence of SEQ ID NO: 71, optionally wherein the VL region comprises or consists of the sequence of SEQ ID NO: 97; and
   ii. a light chain constant region comprising or consisting of the sequence of SEQ ID NO: 107;
      and the immunoconjugate comprises or consists of first and second copies of said first and second polypeptides.

In some embodiments the immunoconjugate comprises:
a) a first polypeptide chain comprising, in an N- to C-terminal direction:
   i. a heavy chain variable region (VH) of an anti-NKG2D antibody comprising an HCDR1 comprising or consisting of the sequence of SEQ ID NO: 6, an HCDR2 comprising or consisting of the sequence of SEQ ID NO: 19 and an HCDR3 comprising or consisting of the sequence of SEQ ID NO: 32, optionally wherein the VH region comprises or consists of the sequence of SEQ ID NO:84;
   ii. a heavy chain constant region comprising or consisting of a sequence of SEQ ID NO: 105;
   iii. optionally a linker; and
   iv. an IL-15 polypeptide comprising or consisting of a sequence selected from the group consisting of SEQ ID NOs: 154, 206 and 207; and
b) a second polypeptide chain comprising, in an N- to C-terminal direction:
   i. a light chain variable region (VL) of an anti-NKG2D antibody comprising an LCDR1 comprising or consisting of the sequence of SEQ ID NO: 45, an LCDR2 comprising or consisting of the sequence of SEQ ID NO: 58 and an LCDR3 comprising or consisting of the sequence of SEQ ID NO: 71, optionally wherein the VL region comprises or consists of the sequence of SEQ ID NO: 97; and
   ii. a light chain constant region comprising or consisting of the sequence of SEQ ID NO: 107;
      and the immunoconjugate comprises or consists of first and second copies of said first and second polypeptides.

In some embodiments the immunoconjugate comprises:
a) a first polypeptide chain comprising, in an N- to C-terminal direction:
   i. a heavy chain variable region (VH) of an anti-NKG2D antibody comprising an HCDR1 comprising or consisting of the sequence of SEQ ID NO: 6, an HCDR2 comprising or consisting of the sequence of SEQ ID NO: 19 and an HCDR3 comprising or consisting of the sequence of SEQ ID NO: 32, optionally wherein the VH region comprises or consists of the sequence of SEQ ID NO:84;
   ii. a heavy chain constant region comprising or consisting of a sequence of SEQ ID NO: 105;
   iii. optionally a linker; and
   iv. an IL-15 polypeptide comprising or consisting of the sequence of SEQ ID NOs: 154; and
b) a second polypeptide chain comprising, in an N- to C-terminal direction:
   i. a light chain variable region (VL) of an anti-NKG2D antibody comprising an LCDR1 comprising or consisting of the sequence of SEQ ID NO: 45, an LCDR2 comprising or consisting of the sequence of SEQ ID NO: 58 and an LCDR3 comprising or consisting of the sequence of SEQ ID NO: 71, optionally wherein the VL region comprises or consists of the sequence of SEQ ID NO: 97; and
   ii. a light chain constant region comprising or consisting of the sequence of SEQ ID NO: 107;
      and the immunoconjugate comprises or consists of first and second copies of said first and second polypeptides. The linker may comprise or consist of a sequence selected from the group consisting of SEQ ID NO: 182 to 187, 201, 204 and 205.

In some embodiments the immunoconjugate comprises:
a) a first polypeptide chain comprising, in an N- to C-terminal direction:
   i. a heavy chain variable region (VH) of an anti-NKG2D antibody comprising an HCDR1 comprising or consisting of the sequence of SEQ ID NO: 6, an HCDR2 comprising or consisting of the sequence of SEQ ID NO: 19 and an HCDR3 comprising or consisting of the sequence of SEQ ID NO: 32, optionally wherein the VH region comprises or consists of the sequence of SEQ ID NO:84;
   ii. a heavy chain constant region comprising or consisting of a sequence of SEQ ID NO: 105;
   iii. optionally a linker comprising or consisting of glycine and serine residues; and
   iv. an IL-15 polypeptide comprising or consisting of the sequence of SEQ ID NOs: 154; and
b) a second polypeptide chain comprising, in an N- to C-terminal direction:
   i. a light chain variable region (VL) of an anti-NKG2D antibody comprising an LCDR1 comprising or consisting of the sequence of SEQ ID NO: 45, an LCDR2 comprising or consisting of the sequence of SEQ ID NO: 58 and an LCDR3 comprising or consisting of the sequence of SEQ ID NO: 71, optionally wherein the VL region comprises or consists of the sequence of SEQ ID NO: 97; and
   ii. a light chain constant region comprising or consisting of the sequence of SEQ ID NO: 107;
      and the immunoconjugate comprises or consists of first and second copies of said first and second polypeptides.

In some embodiments the immunoconjugate comprises:
a) a first polypeptide chain comprising, in an N- to C-terminal direction:
   i. a heavy chain variable region (VH) of an anti-NKG2D antibody comprising an HCDR1 comprising or consisting of the sequence of SEQ ID NO: 6, an HCDR2 comprising or consisting of the sequence of SEQ ID NO: 19 and an HCDR3 comprising or consisting of the sequence of SEQ ID NO: 32, optionally wherein the VH region comprises or consists of the sequence of SEQ ID NO:84;
   ii. a heavy chain constant region comprising or consisting of a sequence of SEQ ID NO: 105;
   iii. optionally a linker comprising or consisting of the sequence of SEQ ID NO: 182; and
   iv. an IL-15 polypeptide comprising or consisting of the sequence of SEQ ID NOs: 154; and
b) a second polypeptide chain comprising, in an N- to C-terminal direction:
   i. a light chain variable region (VL) of an anti-NKG2D antibody comprising an LCDR1 comprising or consisting of the sequence of SEQ ID NO: 45, an LCDR2 comprising or consisting of the sequence of SEQ ID NO: 58 and an LCDR3 comprising or consisting of the sequence of SEQ ID NO: 71, optionally wherein the VL region comprises or consists of the sequence of SEQ ID NO: 97; and
   ii. a light chain constant region comprising or consisting of the sequence of SEQ ID NO: 107;
      and the immunoconjugate comprises or consists of first and second copies of said first and second polypeptides.

In some embodiments the immunoconjugate comprises:
a) a first polypeptide chain comprising, in an N- to C-terminal direction:
   i. a heavy chain variable region (VH) of an anti-NKG2D antibody comprising an HCDR1 comprising or consisting of the sequence of SEQ ID NO: 6, an HCDR2 comprising or consisting of the sequence of SEQ ID NO: 19 and an HCDR3 comprising or consisting of the sequence of SEQ ID NO: 32, optionally wherein the VH region comprises or consists of the sequence of SEQ ID NO:84;
   ii. a heavy chain constant region comprising or consisting of a sequence of SEQ ID NO: 105;
   iii. optionally a linker comprising or consisting of the sequence of SEQ ID NO: 184; and
   iv. an IL-15 polypeptide comprising or consisting of the sequence of SEQ ID NOs: 154; and
b) a second polypeptide chain comprising, in an N- to C-terminal direction:
   i. a light chain variable region (VL) of an anti-NKG2D antibody comprising an LCDR1 comprising or consisting of the sequence of SEQ ID NO: 45, an LCDR2 comprising or consisting of the sequence of SEQ ID NO: 58 and an LCDR3 comprising or consisting of the sequence of SEQ ID NO: 71, optionally wherein the VL region comprises or consists of the sequence of SEQ ID NO: 97; and
   ii. a light chain constant region comprising or consisting of the sequence of SEQ ID NO: 107;
      and the immunoconjugate comprises or consists of first and second copies of said first and second polypeptides.

In some embodiments the immunoconjugate comprises:
a) a first polypeptide chain comprising, in an N- to C-terminal direction:
   i. a heavy chain variable region (VH) of an anti-NKG2D antibody comprising an HCDR1 comprising or consisting of the sequence of SEQ ID NO: 6, an HCDR2 comprising or consisting of the sequence of SEQ ID NO: 19 and an HCDR3 comprising or consisting of the sequence of SEQ ID NO: 32, optionally wherein the VH region comprises or consists of the sequence of SEQ ID NO:84;
   ii. a heavy chain constant region comprising or consisting of a sequence of SEQ ID NO: 105;
   iii. optionally a linker; and
   iv. an IL-15 polypeptide comprising or consisting of the sequence of SEQ ID NOs: 206; and
b) a second polypeptide chain comprising, in an N- to C-terminal direction:
   i. a light chain variable region (VL) of an anti-NKG2D antibody comprising an LCDR1 comprising or consisting of the sequence of SEQ ID NO: 45, an LCDR2 comprising or consisting of the sequence of SEQ ID NO: 58 and an LCDR3 comprising or consisting of the sequence of SEQ ID NO: 71, optionally wherein the VL region comprises or consists of the sequence of SEQ ID NO: 97; and
   ii. a light chain constant region comprising or consisting of the sequence of SEQ ID NO: 107;
      and the immunoconjugate comprises or consists of first and second copies of said first and second polypeptides. The linker may comprise or consist of a sequence selected from the group consisting of SEQ ID NO: 182 to 187, 201, 204 and 205.

In some embodiments the immunoconjugate comprises:
a) a first polypeptide chain comprising, in an N- to C-terminal direction:
   i. a heavy chain variable region (VH) of an anti-NKG2D antibody comprising an HCDR1 comprising or consisting of the sequence of SEQ ID NO: 6, an HCDR2 comprising or consisting of the sequence of SEQ ID NO: 19 and an HCDR3 comprising or consisting of the sequence of SEQ ID NO: 32, optionally wherein the VH region comprises or consists of the sequence of SEQ ID NO:84;
   ii. a heavy chain constant region comprising or consisting of a sequence of SEQ ID NO: 105;
   iii. optionally a linker; and
   iv. an IL-15 polypeptide comprising or consisting of the sequence of SEQ ID NOs: 207; and
b) a second polypeptide chain comprising, in an N- to C-terminal direction:
   i. a light chain variable region (VL) of an anti-NKG2D antibody comprising an LCDR1 comprising or consisting of the sequence of SEQ ID NO: 45, an LCDR2 comprising or consisting of the sequence of SEQ ID NO: 58 and an LCDR3 comprising or consisting of the sequence of SEQ ID NO: 71, optionally wherein the VL region comprises or consists of the sequence of SEQ ID NO: 97; and
   ii. a light chain constant region comprising or consisting of the sequence of SEQ ID NO: 107;
      and the immunoconjugate comprises or consists of first and second copies of said first and second polypeptides. The linker may comprise or consist of a sequence selected from the group consisting of SEQ ID NO: 182 to 187, 201, 204 and 205.

In some embodiments the immunoconjugate comprises:
a) a first polypeptide chain comprising, in an N- to C-terminal direction:
   i. a heavy chain variable region (VH) of an anti-NKG2D antibody comprising an HCDR1 comprising or consisting of the sequence of SEQ ID NO: 6, an HCDR2 comprising or consisting of the sequence of SEQ ID NO: 19 and an HCDR3 comprising or consisting of the sequence of SEQ ID NO: 32, optionally wherein the VH region comprises or consists of the sequence of SEQ ID NO:84;
   ii. a heavy chain constant region comprising or consisting of a sequence of SEQ ID NO: 105;
   iii. optionally a linker comprising or consisting of the sequence of SEQ ID NO: 182; and
   iv. an IL-15 polypeptide comprising or consisting of the sequence of SEQ ID NOs: 207; and
b) a second polypeptide chain comprising, in an N- to C-terminal direction:
   i. a light chain variable region (VL) of an anti-NKG2D antibody comprising an LCDR1 comprising or consisting of the sequence of SEQ ID NO: 45, an LCDR2 comprising or consisting of the sequence of SEQ ID NO: 58 and an LCDR3 comprising or consisting of the sequence of SEQ ID NO: 71, optionally wherein the VL region comprises or consists of the sequence of SEQ ID NO: 97; and
   ii. a light chain constant region comprising or consisting of the sequence of SEQ ID NO: 107;
      and the immunoconjugate comprises or consists of first and second copies of said first and second polypeptides.

In some embodiments the immunoconjugate comprises:
a) a first polypeptide chain comprising, in an N- to C-terminal direction:
   i. a heavy chain variable region (VH) of an anti-NKG2D antibody comprising an HCDR1 comprising or consisting of the sequence of SEQ ID NO: 1, an HCDR2 comprising or consisting of the sequence of SEQ ID NO: 14 and an HCDR3 comprising or consisting of the sequence of SEQ ID NO: 27, optionally wherein the VH region comprises or consists of the sequence of SEQ ID NO:79 or 202;
   ii. a heavy chain constant region comprising or consisting of a sequence of SEQ ID NO: 105, 106 and 203;
   iii. optionally a linker; and
   iv. an IL-15 polypeptide comprising or consisting of a sequence selected from the group consisting of SEQ ID NOs: 148 to 155, 206 and 207; and
b) a second polypeptide chain comprising, in an N- to C-terminal direction:
   i. a light chain variable region (VL) of an anti-NKG2D antibody comprising an LCDR1 comprising or consisting of the sequence of SEQ ID NO: 40, an LCDR2 comprising or consisting of the sequence of SEQ ID NO: 53 and an LCDR3 comprising or consisting of the sequence of SEQ ID NO: 66, optionally wherein the VL region comprises or consists of the sequence of SEQ ID NO: 92; and
   ii. a light chain constant region comprising or consisting of the sequence of SEQ ID NO: 108;
      and the immunoconjugate comprises or consists of first and second copies of said first and second polypeptides.

In some embodiments the immunoconjugate comprises:
a) a first polypeptide chain comprising, in an N- to C-terminal direction:
   i. a heavy chain variable region (VH) of an anti-NKG2D antibody comprising an HCDR1 comprising or consisting of the sequence of SEQ ID NO: 1, an HCDR2 comprising or consisting of the sequence of SEQ ID NO: 14 and an HCDR3 comprising or consisting of the sequence of SEQ ID NO: 27, optionally wherein the VH region comprises or consists of the sequence of SEQ ID NO: 79 or 202;
   ii. a heavy chain constant region comprising or consisting of a sequence of SEQ ID NO: 105;
   iii. optionally a linker; and
   iv. an IL-15 polypeptide comprising or consisting of a sequence selected from the group consisting of SEQ ID NOs: 148 to 155, 206 and 207; and
b) a second polypeptide chain comprising, in an N- to C-terminal direction:
   i. a light chain variable region (VL) of an anti-NKG2D antibody comprising an LCDR1 comprising or consisting of the sequence of SEQ ID NO: 40, an LCDR2 comprising or consisting of the sequence of SEQ ID NO: 53 and an LCDR3 comprising or consisting of the sequence of SEQ ID NO: 66, optionally wherein the VL region comprises or consists of the sequence of SEQ ID NO: 92; and
   ii. a light chain constant region comprising or consisting of the sequence of SEQ ID NO: 108;
      and the immunoconjugate comprises or consists of first and second copies of said first and second polypeptides.

In some embodiments the immunoconjugate comprises:
a) a first polypeptide chain comprising, in an N- to C-terminal direction:
   i. a heavy chain variable region (VH) of an anti-NKG2D antibody comprising an HCDR1 comprising or consisting of the sequence of SEQ ID NO: 1, an HCDR2 comprising or consisting of the sequence of SEQ ID NO: 14 and an HCDR3 comprising or consisting of the sequence of SEQ ID NO: 27, optionally wherein the VH region comprises or consists of the sequence of SEQ ID NO: 79 or 202;
   ii. a heavy chain constant region comprising or consisting of a sequence of SEQ ID NO: 105;
   iii. optionally a linker; and
   iv. an IL-15 polypeptide comprising or consisting of a sequence selected from the group consisting of SEQ ID NOs: 154, 206 and 207; and
b) a second polypeptide chain comprising, in an N- to C-terminal direction:
   i. a light chain variable region (VL) of an anti-NKG2D antibody comprising an LCDR1 comprising or consisting of the sequence of SEQ ID NO: 40, an LCDR2 comprising or consisting of the sequence of SEQ ID NO: 53 and an LCDR3 comprising or consisting of the sequence of SEQ ID NO: 66, optionally wherein the VL region comprises or consists of the sequence of SEQ ID NO: 92; and
   ii. a light chain constant region comprising or consisting of the sequence of SEQ ID NO: 108;
      and the immunoconjugate comprises or consists of first and second copies of said first and second polypeptides.

In some embodiments the immunoconjugate comprises:
a) a first polypeptide chain comprising, in an N- to C-terminal direction:
   i. a heavy chain variable region (VH) of an anti-NKG2D antibody comprising an HCDR1 comprising or consisting of the sequence of SEQ ID NO: 1, an HCDR2 comprising or consisting of the sequence of SEQ ID NO: 14 and an HCDR3 comprising or consisting of the sequence of SEQ ID NO: 27, optionally wherein the VH region comprises or consists of the sequence of SEQ ID NO: 79 or 202;
   ii. a heavy chain constant region comprising or consisting of a sequence of SEQ ID NO: 105;
   iii. optionally a linker; and
   iv. an IL-15 polypeptide comprising or consisting of the sequence of SEQ ID NOs: 154; and
b) a second polypeptide chain comprising, in an N- to C-terminal direction:
   i. a light chain variable region (VL) of an anti-NKG2D antibody comprising an LCDR1 comprising or consisting of the sequence of SEQ ID NO: 40, an LCDR2 comprising or consisting of the sequence of SEQ ID NO: 53 and an LCDR3 comprising or consisting of the sequence of SEQ ID NO: 66, optionally wherein the VL region comprises or consists of the sequence of SEQ ID NO: 92; and
   ii. a light chain constant region comprising or consisting of the sequence of SEQ ID NO: 108;
      and the immunoconjugate comprises or consists of first and second copies of said first and second polypeptides. The linker may comprise or consist of a sequence selected from the group consisting of SEQ ID NO: 182 to 187, 201, 204 and 205.

In some embodiments the immunoconjugate comprises:
a) a first polypeptide chain comprising, in an N- to C-terminal direction:
   i. a heavy chain variable region (VH) of an anti-NKG2D antibody comprising an HCDR1 comprising or consisting of the sequence of SEQ ID NO: 1, an HCDR2 comprising or consisting of the sequence of SEQ ID NO: 14 and an HCDR3 comprising or consisting of the sequence of SEQ ID NO: 27, optionally wherein the VH region comprises or consists of the sequence of SEQ ID NO: 79 or 202;
   ii. a heavy chain constant region comprising or consisting of a sequence of SEQ ID NO: 105;
   iii. optionally a linker; and
   iv. an IL-15 polypeptide comprising or consisting of the sequence of SEQ ID NOs: 206; and
b) a second polypeptide chain comprising, in an N- to C-terminal direction:
   i. a light chain variable region (VL) of an anti-NKG2D antibody comprising an LCDR1 comprising or consisting of the sequence of SEQ ID NO: 40, an LCDR2 comprising or consisting of the sequence of SEQ ID NO: 53 and an LCDR3 comprising or consisting of the sequence of SEQ ID NO: 66, optionally wherein the VL region comprises or consists of the sequence of SEQ ID NO: 92; and
   ii. a light chain constant region comprising or consisting of the sequence of SEQ ID NO: 108;
      and the immunoconjugate comprises or consists of first and second copies of said first and second polypeptides. The linker may comprise or consist of a sequence selected from the group consisting of SEQ ID NO: 182 to 187, 201, 204 and 205.

In some embodiments the immunoconjugate comprises:
a) a first polypeptide chain comprising, in an N- to C-terminal direction:
   i. a heavy chain variable region (VH) of an anti-NKG2D antibody comprising an HCDR1 comprising or consisting of the sequence of SEQ ID NO: 1, an HCDR2 comprising or consisting of the sequence of SEQ ID NO: 14 and an HCDR3 comprising or consisting of the sequence of SEQ ID NO: 27, optionally wherein the VH region comprises or consists of the sequence of SEQ ID NO: 79 or 202;
   ii. a heavy chain constant region comprising or consisting of a sequence of SEQ ID NO: 105;
   iii. optionally a linker; and
   iv. an IL-15 polypeptide comprising or consisting of the sequence of SEQ ID NOs: 207; and
b) a second polypeptide chain comprising, in an N- to C-terminal direction:
   i. a light chain variable region (VL) of an anti-NKG2D antibody comprising an LCDR1 comprising or consisting of the sequence of SEQ ID NO: 40, an LCDR2 comprising or consisting of the sequence of SEQ ID NO: 53 and an LCDR3 comprising or consisting of the sequence of SEQ ID NO: 66, optionally wherein the VL region comprises or consists of the sequence of SEQ ID NO: 92; and
   ii. a light chain constant region comprising or consisting of the sequence of SEQ ID NO: 108;
      and the immunoconjugate comprises or consists of first and second copies of said first and second polypeptides. The linker may comprise or consist of a sequence selected from the group consisting of SEQ ID NO: 182 to 187, 201, 204 and 205.

In some embodiments the immunoconjugate comprises:
a) a first polypeptide chain comprising or consisting of a sequence having at least 90%, at least 91%, at least 92%, at least 93%, at least 94%, at least 95%, at least 95%, at least 97%, at least 98% or at least 99% identity to a sequence selected from the group consisting of SEQ ID NOs: 156 to 165, SEQ ID NO: 188, 189, 191, 192, 195 and 200; and
b) a second polypeptide chain comprising or consisting of a sequence having at least 90%, at least 91%, at least 92%, at least 93%, at least 94%, at least 95%, at least 95%, at least 97%, at least 98% or at least 99% identity to a sequence selected from the group consisting of SEQ ID NOs: 135 to 144;
   and the immunoconjugate comprises or consists of first and second copies of said first and second polypeptides.

In some embodiments the immunoconjugate comprises:
a) a first polypeptide chain comprising or consisting of a sequence selected from the group consisting of SEQ ID NOs: 156 to 165, SEQ ID NO: 188, 189, 191, 192, 195 and 200; and
b) a second polypeptide chain comprising or consisting of a sequence selected from the group consisting of SEQ ID NOs: 135 to 144;
   and the immunoconjugate comprises or consists of first and second copies of said first and second polypeptides.

In some embodiments the immunoconjugate comprises a first polypeptide chain comprising or consisting of the sequence of SEQ ID NO: 156; and a second polypeptide chain comprising or consisting of the sequence of SEQ ID NO: 135. Such an immunoconjugate is referred to herein as CYTB02.

In some embodiments the immunoconjugate comprises a first polypeptide chain comprising or consisting of the sequence of SEQ ID NO: 157; and a second polypeptide chain comprising or consisting of the sequence of SEQ ID NO: 136.

In some embodiments the immunoconjugate comprises a first polypeptide chain comprising or consisting of the sequence of SEQ ID NO: 158; and a second polypeptide chain comprising or consisting of the sequence of SEQ ID NO: 137.

In some embodiments the immunoconjugate comprises a first polypeptide chain comprising or consisting of the sequence of SEQ ID NO: 159; and a second polypeptide chain comprising or consisting of the sequence of SEQ ID NO: 138.

In some embodiments the immunoconjugate comprises a first polypeptide chain comprising or consisting of the sequence of SEQ ID NO: 160; and a second polypeptide chain comprising or consisting of the sequence of SEQ ID NO: 139. Such an immunoconjugate is referred to herein as CYTB04.

In some embodiments the immunoconjugate comprises a first polypeptide chain comprising or consisting of the sequence of SEQ ID NO: 161; and a second polypeptide chain comprising or consisting of the sequence of SEQ ID NO: 140. Such an immunoconjugate is referred to herein as CYTB05.

In some embodiments the immunoconjugate comprises a first polypeptide chain comprising or consisting of the sequence of SEQ ID NO: 162; and a second polypeptide chain comprising or consisting of the sequence of SEQ ID NO: 141.

In some embodiments the immunoconjugate comprises a first polypeptide chain comprising or consisting of the sequence of SEQ ID NO: 163; and a second polypeptide chain comprising or consisting of the sequence of SEQ ID NO: 142.

In some embodiments the immunoconjugate comprises a first polypeptide chain comprising or consisting of the sequence of SEQ ID NO: 164; and a second polypeptide chain comprising or consisting of the sequence of SEQ ID NO: 143.

In some embodiments the immunoconjugate comprises a first polypeptide chain comprising or consisting of the sequence of SEQ ID NO: 165; and a second polypeptide chain comprising or consisting of the sequence of SEQ ID NO: 144.

In some embodiments the immunoconjugate comprises a first polypeptide chain comprising or consisting of the sequence of SEQ ID NO: 188; and a second polypeptide chain comprising or consisting of the sequence of SEQ ID NO: 135. Such an immunoconjugate is referred to herein as CYTB03.

In some embodiments the immunoconjugate comprises a first polypeptide chain comprising or consisting of the sequence of SEQ ID NO: 189; and a second polypeptide chain comprising or consisting of the sequence of SEQ ID NO: 140. Such an immunoconjugate is referred to herein as CYTB06.

In some embodiments the immunoconjugate comprises a first polypeptide chain comprising or consisting of the sequence of SEQ ID NO: 191; and a second polypeptide chain comprising or consisting of the sequence of SEQ ID NO: 140. Such an immunoconjugate is referred to herein as CYTB08.

In some embodiments the immunoconjugate comprises a first polypeptide chain comprising or consisting of the sequence of SEQ ID NO: 192; and a second polypeptide chain comprising or consisting of the sequence of SEQ ID NO: 140. Such an immunoconjugate is referred to herein as CYTB12.

In some embodiments the immunoconjugate comprises a first polypeptide chain comprising or consisting of the sequence of SEQ ID NO: 195; and a second polypeptide chain comprising or consisting of the sequence of SEQ ID NO: 140. Such an immunoconjugate is referred to herein as CYTB28.

In some embodiments the immunoconjugate comprises a first polypeptide chain comprising or consisting of the sequence of SEQ ID NO: 200; and a second polypeptide chain comprising or consisting of the sequence of SEQ ID NO: 140. Such an immunoconjugate is referred to herein as CYTB32.

### NKp30 HC C-Terminus IL-15 Immunoconjugates

To further outline the applicability of the approach a series of non-limiting example immunoconjugates are provided herein. These immunoconjugates comprise at least two IL-15 polypeptides conjugated to the C-termini of the heavy chains of a multivalent antibody or antigen-binding fragment thereof that specifically binds to NKp30. In preferred embodiments, the two IL-15 polypeptides are conjugated to the CH3 domain as shown in FIG. 4A.

In some embodiments the immunoconjugate comprises:
a) a first polypeptide chain comprising, in an N- to C-terminal direction:
   i. a heavy chain variable region (VH) of an anti-NKp30 antibody comprising an HCDR1 comprising or consisting of a sequence selected from the group consisting of SEQ ID NOs: 11 to 12 and optionally having up to 2 amino acid substitutions thereto, an HCDR2 comprising or consisting of a sequence selected from the group consisting of SEQ ID NOs: 24 to 25 and optionally having up to 2 amino acid substitutions thereto, and an HCDR3 comprising or consisting of a sequence selected from the group consisting of SEQ ID NOs: 37 to 38 and optionally having up to 2 amino acid substitutions thereto;
   ii. a heavy chain constant region comprising or consisting of a sequence having at least 90%, at least 91%, at least 92%, at least 93%, at least 94%, at least 95%, at least 95%, at least 97%, at least 98% or at least 99% identity to a sequence selected from the group consisting of SEQ ID NOs: 105, 106 and 203;
   iii. optionally a linker; and
   iv. an IL-15 polypeptide comprising or consisting of a sequence having at least 90%, at least 91%, at least 92%, at least 93%, at least 94%, at least 95%, at least 95%, at least 97%, at least 98% or at least 99% identity to a sequence selected from the group consisting of SEQ ID NOs: 148 to 155, 206 and 207; and
b) a second polypeptide chain comprising, in an N- to C-terminal direction:
   i. a light chain variable region (VL) of an anti-NKp30 antibody comprising an LCDR1 comprising or consisting of a sequence selected from the group consisting of SEQ ID NOs: 50 to 51 and optionally having up to 2 amino acid substitutions thereto, an LCDR2 comprising or consisting of a sequence selected from the group consisting of SEQ ID NOs: 63 to 64 and optionally having up to 2 amino acid substitutions thereto, and an LCDR3 comprising or consisting of a sequence selected from the group consisting of SEQ ID NOs: 76 to 77 and optionally having up to 2 amino acid substitutions thereto; and
   ii. a light chain constant region comprising or consisting of a sequence having at least 90%, at least 91%, at least 92%, at least 93%, at least 94%, at least 95%, at least 95%, at least 97%, at least 98% or at least 99% identity to a sequence selected from the group consisting of SEQ ID NOs: 107 to 108;
      and the immunoconjugate comprises or consists of first and second copies of said first and second polypeptides.

In some embodiments the optional amino acid substitutions are conservative substitutions.

In some embodiments the immunoconjugate comprises:
a) a first polypeptide chain comprising, in an N- to C-terminal direction:
   i. a heavy chain variable region (VH) of an anti-NKp30 antibody comprising an HCDR1 comprising or consisting of a sequence selected from the group consisting of SEQ ID NOs: 11 to 12, an HCDR2 comprising or consisting of a sequence selected from the group consisting of SEQ ID NOs: 24 to 25 and an HCDR3 comprising or consisting of a sequence selected from the group consisting of SEQ ID NOs: 37 to 38;
   ii. a heavy chain constant region comprising or consisting of a sequence selected from the group consisting of SEQ ID NOs: 105, 106 and 203;
   iii. optionally a linker; and
   iv. an IL-15 polypeptide comprising or consisting of a sequence selected from the group consisting of SEQ ID NOs: 148 to 155, 206 and 207; and
b) a second polypeptide chain comprising, in an N- to C-terminal direction:
   i. a light chain variable region (VL) of an anti-NKp30 antibody comprising an LCDR1 comprising or consisting of a sequence selected from the group consisting of SEQ ID NOs: 50 to 51, an LCDR2 comprising or consisting of a sequence selected from the group consisting of SEQ ID NOs: 63 to 64 and an LCDR3 comprising or consisting of a sequence selected from the group consisting of SEQ ID NOs: 76 to 77; and
   ii. a light chain constant region comprising or consisting of a sequence selected from the group consisting of SEQ ID NOs: 107 to 108;
      and the immunoconjugate comprises or consists of first and second copies of said first and second polypeptides.

In some embodiments the immunoconjugate comprises:
i. a first polypeptide chain comprising or consisting of a sequence having at least 90%, at least 91%, at least 92%, at least 93%, at least 94%, at least 95%, at least 95%, at least 97%, at least 98% or at least 99% identity to a sequence selected from the group consisting of SEQ ID NOs: 166 to 167; and
ii. a second polypeptide chain comprising or consisting of a sequence having at least 90%, at least 91%, at least 92%, at least 93%, at least 94%, at least 95%, at least 95%, at least 97%, at least 98% or at least 99% identity to a sequence selected from the group consisting of SEQ ID NOs: 145 to 146;
   and the immunoconjugate comprises or consists of first and second copies of said first and second polypeptides.

In some embodiments the immunoconjugate comprises:
a) a first polypeptide chain comprising or consisting of a sequence selected from the group consisting of SEQ ID NOs: 166 to 167; and
b) a second polypeptide chain comprising or consisting of a sequence selected from the group consisting of SEQ ID NOs: 145 to 146;
   and the immunoconjugate comprises or consists of first and second copies of said first and second polypeptides.

In some embodiments the immunoconjugate comprises a first polypeptide chain comprising or consisting of the sequence of SEQ ID NO: 166; and a second polypeptide chain comprising or consisting of the sequence of SEQ ID NO: 145.

In some embodiments the immunoconjugate comprises a first polypeptide chain comprising or consisting of the sequence of SEQ ID NO: 167; and a second polypeptide chain comprising or consisting of the sequence of SEQ ID NO: 146.

### NKp46 HC C-Terminus IL-15 Immunoconjugates

To further outline the applicability of the approach a series of non-limiting example immunoconjugates are provided herein. These immunoconjugates comprise at least two IL-15 polypeptides conjugated to the C-termini of the heavy chains of a multivalent antibody or antigen-binding fragment thereof that specifically binds to NKp46. In preferred embodiments, the two IL-15 polypeptides are conjugated to the CH3 domain as shown in FIG. 4A.

In some embodiments the immunoconjugate comprises:
a) a first polypeptide chain comprising, in an N- to C-terminal direction:
   i. a heavy chain variable region (VH) of an anti-NKp46 antibody comprising an HCDR1 comprising or consisting of SEQ ID NO: 13 and optionally having up to 2 amino acid substitutions thereto, an HCDR2 comprising or consisting of SEQ ID NO: 26 and optionally having up to 2 amino acid substitutions thereto, and an HCDR3 comprising or consisting of SEQ ID NO: 39 and optionally having up to 2 amino acid substitutions thereto;
   ii. a heavy chain constant region comprising or consisting of a sequence having at least 90%, at least 91%, at least 92%, at least 93%, at least 94%, at least 95%, at least 95%, at least 97%, at least 98% or at least 99% identity to a sequence selected from the group consisting of SEQ ID NO: 105 to 106;
   iii. optionally a linker; and
   iv. an IL-15 polypeptide comprising or consisting of a sequence having at least 90%, at least 91%, at least 92%, at least 93%, at least 94%, at least 95%, at least 95%, at least 97%, at least 98% or at least 99% identity to a sequence selected from the group consisting of SEQ ID NOs: 148 to 155, 206 and 207; and
b) a second polypeptide chain comprising, in an N- to C-terminal direction:
   i. a light chain variable region (VL) of an anti-NKp46 antibody comprising an LCDR1 comprising or consisting of SEQ ID NO: 52 and optionally having up to 2 amino acid substitutions thereto, an LCDR2 comprising or consisting of SEQ ID NO: 65 and optionally having up to 2 amino acid substitutions thereto, and an LCDR3 comprising or consisting of SEQ ID NO: 78 and optionally having up to 2 amino acid substitutions thereto; and
   ii. a light chain constant region comprising or consisting of a sequence having at least 90%, at least 91%, at least 92%, at least 93%, at least 94%, at least 95%, at least 95%, at least 97%, at least 98% or at least 99% identity to SEQ ID NO: 107;
      and the immunoconjugate comprises or consists of first and second copies of said first and second polypeptides.

In some embodiments the optional amino acid substitutions are conservative substitutions.

In some embodiments the immunoconjugate comprises:
a) a first polypeptide chain comprising, in an N- to C-terminal direction:
   i. a heavy chain variable region (VH) of an anti-NKp46 antibody comprising an HCDR1 comprising or consisting of SEQ ID NO: 13, an HCDR2 comprising or consisting of SEQ ID NO: 26, and an HCDR3 comprising or consisting of SEQ ID NO: 39;
   ii. a heavy chain constant region comprising or consisting of a sequence selected from the group consisting of SEQ ID NOs: 105 to 106;
   iii. optionally a linker; and
   iv. an IL-15 polypeptide comprising or consisting of a sequence selected from the group consisting of SEQ ID NOs: 148 to 155, 206 and 207; and
b) a second polypeptide chain comprising, in an N- to C-terminal direction:
   i. a light chain variable region (VL) of an anti-NKp46 antibody comprising an LCDR1 comprising or consisting of SEQ ID NO: 52, an LCDR2 comprising or consisting of SEQ ID NO: 65 and an LCDR3 comprising or consisting of SEQ ID NO: 78; and
   ii. a light chain constant region comprising or consisting of SEQ ID NO: 107;
      and the immunoconjugate comprises or consists of first and second copies of said first and second polypeptides.

In some embodiments the immunoconjugate comprises:
i. a first polypeptide chain comprising or consisting of a sequence having at least 90%, at least 91%, at least 92%, at least 93%, at least 94%, at least 95%, at least 95%, at least 97%, at least 98% or at least 99% identity to the sequence of SEQ ID NO: 168; and
ii. a second polypeptide chain comprising or consisting of a sequence having at least 90%, at least 91%, at least 92%, at least 93%, at least 94%, at least 95%, at least 95%, at least 97%, at least 98% or at least 99% identity to the sequence of SEQ ID NO: 147;
   and the immunoconjugate comprises or consists of first and second copies of said first and second polypeptides.

In some embodiments the immunoconjugate comprises:
a) a first polypeptide chain comprising or consisting of the sequence of SEQ ID NO: 168; and
b) a second polypeptide chain comprising or consisting of the sequence of SEQ ID NO: 147;
   and the immunoconjugate comprises or consists of first and second copies of said first and second polypeptides.

In some embodiments the immunoconjugate comprises a first polypeptide chain comprising or consisting of the sequence of SEQ ID NO: 168; and a second polypeptide chain comprising or consisting of the sequence of SEQ ID NO: 147.

### Specific Immunoconjugates - IL-15 Polypeptides are Joined to C-Termini of Light Chains

In some embodiments of the invention, the IL-15 polypeptides are conjugated (optionally via a linker) to the C-termini of the light chains of the antibody component. In some preferred embodiments the IL-15 polypeptides are conjugated (optionally via a linker) to the C-termini of the CL domains of the heavy chains. This format is shown in FIG. 4B and FIG. 5C.

In the immunoconjugate formats described below, the immunoglobulin heavy chain(s) of the multivalent antibody is referred to as the first polypeptide chain. The first and second copy of the first polypeptide chain therefore refer to the two identical heavy chains of the multivalent antibody. The heavy chains may be Fc enabled or Fc disabled, as described elsewhere herein. In the immunoconjugate formats described below, the immunoglobulin light chain(s) of the multivalent antibody is referred to as the second polypeptide chain. The first and second copy of the second polypeptide chain therefore refer to the two identical light chains of the multivalent antibody. In some embodiments, the first polypeptide (heavy chain) and second polypeptide (light chain) forms one subunit. This then homodimerizes to form the final immunoconjugate comprising two said subunits and thus a total of four polypeptide chains. Hence in some embodiments, the immunoconjugates comprise first and second copies of said first and second polypeptide chains. The immunoconjugates can therefore be described as dimers, with one monomer of the dimer comprising a first copy of the first polypeptide chain and a first copy of the second polypeptide chain and the second monomer of the dimer comprising a second copy of the first polypeptide chain and a second copy of the second polypeptide chain. In these embodiments the immunoconjugate is considered homodimeric. In some embodiments, the immunoconjugate may have been selectively purified in order to arrive at such a homodimeric species and remove or reduce any heterodimeric species that may be considered impurities. In some embodiments the homodimeric immunoconjugate is the dominant species in a mixture that includes homodimeric and heterodimeric immunoconjugates.

In some embodiments, the anti-NKG2D antibody, anti-NKp30 antibody, anti-NKp46 antibody, anti-NKp44 antibody or anti-DNAM-1 antibody referred to in the immunoconjugate formats described below is any of the antibodies H1/L1 to H13/L13, or could be another antibody against these targets known in the art.

In some embodiments the immunoconjugate comprises:
a) a first polypeptide chain comprising, in an N- to C-terminal direction:
   i. a heavy chain variable region (VH) of an anti-NKG2D antibody, an anti-NKp30 antibody, an anti-NKp46 antibody, an anti-NKp44 antibody or an anti-DNAM-1 antibody;
   ii. a human heavy chain constant region; and
b) a second polypeptide chain comprising, in an N- to C-terminal direction:
   i. a light chain variable region (VL) of an anti-NKG2D antibody, an anti-NKp30 antibody, an anti-NKp46 antibody, an anti-NKp44 antibody or an anti-DNAM-1 antibody; and
   ii. a human light constant region;
   iii. optionally a linker; and
   iv. the IL-15 polypeptide;
      and the immunoconjugate comprises or consists of first and second copies of said first and second polypeptides.

In some embodiments the immunoconjugate comprises:
a) a first polypeptide chain comprising, in an N- to C-terminal direction:
   i. a heavy chain variable region (VH) of an anti-NKG2D antibody, an anti-NKp30 antibody, an anti-NKp46 antibody, an anti-NKp44 antibody or an anti-DNAM-1 antibody;
   ii. a human heavy chain IgG sequence; and
b) a second polypeptide chain comprising, in an N- to C-terminal direction:
   i. a light chain variable region (VL) of an anti-NKG2D antibody, an anti-NKp30 antibody, an anti-NKp46 antibody, an anti-NKp44 antibody or an anti-DNAM-1 antibody; and
   ii. a human light chain IgG sequence;
   iii. optionally a linker; and
   iv. the IL-15 polypeptide;
      and the immunoconjugate comprises or consists of first and second copies of said first and second polypeptides.

In some embodiments the immunoconjugate comprises:
a) a first polypeptide chain comprising, in an N- to C-terminal direction:
   i. a heavy chain variable region (VH) of an anti-NKG2D antibody, an anti-NKp30 antibody, an anti-NKp46 antibody, an anti-NKp44 antibody or an anti-DNAM-1 antibody;
   ii. a human heavy chain IgG1 sequence; and
b) a second polypeptide chain comprising, in an N- to C-terminal direction:
   i. a light chain variable region (VL) of an anti-NKG2D antibody, an anti-NKp30 antibody, an anti-NKp46 antibody, an anti-NKp44 antibody or an anti-DNAM-1 antibody; and
   ii. a human light chain IgG1 sequence;
   iii. optionally a linker; and
   iv. the IL-15 polypeptide;
      and the immunoconjugate comprises or consists of first and second copies of said first and second polypeptides.

In some embodiments the immunoconjugate comprises:
a) a first polypeptide chain comprising a heavy chain of an anti-NKG2D antibody, an anti-NKp30 antibody, an anti-NKp46 antibody, an anti-NKp44 antibody or an anti-DNAM-1 antibody; and
b) a second polypeptide chain comprising in an N- to C-terminal direction:
   i. a light chain of an anti-NKG2D antibody, an anti-NKp30 antibody, an anti-NKp46 antibody, an anti-NKp44 antibody or an anti-DNAM-1 antibody;
   ii. optionally a linker; and
   iii. the IL-15 polypeptide;
      and the immunoconjugate comprises or consists of first and second copies of said first and second polypeptides.

In some embodiments the immunoconjugate comprises:
a) a first polypeptide chain comprising, in an N- to C-terminal direction:
   i. a heavy chain variable region (VH) of an anti-NKG2D antibody, an anti-NKp30 antibody, an anti-NKp46 antibody, an anti-NKp44 antibody or an anti-DNAM-1 antibody, the VH comprising an HCDR1, an HCDR2 and an HCDR3;
   ii. a heavy chain constant region comprising an CH1, a hinge, a CH2 and a CH3 region; and
b) a second polypeptide chain comprising, in an N- to C-terminal direction:
   i. a light chain variable region (VL) of an anti-NKG2D antibody, an anti-NKp30 antibody, an anti-NKp46 antibody, an anti-NKp44 antibody or an anti-DNAM-1 antibody, the VL comprising an LCDR1, an LCDR2 and an LCDR3; and
   ii. a light chain constant (CL) region;
   iii. optionally a linker, and
   iv. the IL-15 polypeptide;
      and the immunoconjugate comprises or consists of first and second copies of said first and second polypeptides.

In any of the above embodiments, the first and second polypeptide chains are covalently or non-covalently associated with each other such that each heavy chain (variable region) and each light chain (variable region) form an antigen binding domain. The first copy of the first and second polypeptide chain and the second copy of the first and second polypeptide chain are covalently or non-covalently associated with each other in an immunoglobulin-type format, thereby forming the immunoconjugate comprising the multivalent antibody or antigen binding fragment thereof.

### NKG2D LC C-Terminus IL-15 Immunoconjugates

To further outline the applicability of the approach a series of non-limiting example immunoconjugates are provided herein. These immunoconjugates comprise at least two IL-15 polypeptides conjugated to the C-termini of the light chains of a multivalent antibody or antigen-binding fragment thereof that specifically binds to NKG2D. In preferred embodiments, the two IL-15 polypeptides are conjugated to the CL domain as shown in FIG. 4B and FIG. 5C.

In some embodiments the immunoconjugate comprises:
a) a first polypeptide chain comprising, in an N- to C-terminal direction:
   i. a heavy chain variable region (VH) of an anti-NKG2D antibody comprising an HCDR1 comprising or consisting of a sequence selected from the group consisting of SEQ ID NOs: 1 to 10 and optionally having up to 2 amino acid substitutions thereto, an HCDR2 comprising or consisting of a sequence selected from the group consisting of SEQ ID NOs: 14 to 23 and optionally having up to 2 amino acid substitutions thereto, and an HCDR3 comprising or consisting of a sequence selected from the group consisting of SEQ ID NOs: 27 to 36 and optionally having up to 2 amino acid substitutions thereto;
   ii. a heavy chain constant region comprising or consisting of a sequence having at least 90%, at least 91%, at least 92%, at least 93%, at least 94%, at least 95%, at least 95%, at least 97%, at least 98% or at least 99% identity to a sequence selected from the group consisting of SEQ ID NOs: 105, 106 and 203; and
b) a second polypeptide chain comprising, in an N- to C-terminal direction:
   i. a light chain variable region (VL) of an anti-NKG2D antibody comprising an LCDR1 comprising or consisting of a sequence selected from the group consisting of SEQ ID NOs: 40 to 49 and optionally having up to 2 amino acid substitutions thereto, an LCDR2 comprising or consisting of a sequence selected from the group consisting of SEQ ID NOs: 53 to 62 and optionally having up to 2 amino acid substitutions thereto, and an LCDR3 comprising or consisting of a sequence selected from the group consisting of SEQ ID NOs: 66 to 75 and optionally having up to 2 amino acid substitutions thereto; and
   ii. a light chain constant region comprising or consisting of a sequence having at least 90%, at least 91%, at least 92%, at least 93%, at least 94%, at least 95%, at least 95%, at least 97%, at least 98% or at least 99% identity to a sequence selected from the group consisting of SEQ ID NOs: 107 to 108;
   iii. optionally a linker; and
   iv. an IL-15 polypeptide comprising or consisting of a sequence having at least 90%, at least 91%, at least 92%, at least 93%, at least 94%, at least 95%, at least 95%, at least 97%, at least 98% or at least 99% identity to a sequence selected from the group consisting of SEQ ID NOs: 148 to 155, 206 and 207; and the immunoconjugate comprises or consists of first and second copies of said first and second polypeptides.

In some embodiments the optional amino acid substitutions are conservative substitutions.

The immunoconjugate of any preceding claim, wherein the immunoconjugate comprises:
a) a first polypeptide chain comprising, in an N- to C-terminal direction:
   i. a heavy chain variable region (VH) of an anti-NKG2D antibody comprising an HCDR1 comprising or consisting of a sequence selected from the group consisting of SEQ ID NOs: 1 to 10, an HCDR2 comprising or consisting of a sequence selected from the group consisting of SEQ ID NOs: 14 to 23 and an HCDR3 comprising or consisting of a sequence selected from the group consisting of SEQ ID NOs: 27 to 36;
   ii. a heavy chain constant region comprising or consisting of a sequence selected from the group consisting of SEQ ID NOs: 105, 106 and 203; and
b) a second polypeptide chain comprising, in an N- to C-terminal direction:
   i. a light chain variable region (VL) of an anti-NKG2D antibody comprising an LCDR1 comprising or consisting of a sequence selected from the group consisting of SEQ ID NOs: 40 to 49, an LCDR2 comprising or consisting of a sequence selected from the group consisting of SEQ ID NOs: 53 to 62 and an LCDR3 comprising or consisting of a sequence selected from the group consisting of SEQ ID NOs: 66 to 75; and
   ii. a light chain constant region comprising or consisting of a sequence selected from the group consisting of SEQ ID NOs: 107 to 108;
   iii. optionally a linker; and
   iv. an IL-15 polypeptide comprising or consisting of a sequence selected from the group consisting of SEQ ID NOs: 148 to 155, 206 and 207;
      and the immunoconjugate comprises or consists of first and second copies of said first and second polypeptides.

In some embodiments, the immunoconjugate comprises:
a) a first polypeptide chain comprising or consisting of a sequence having at least 90%, at least 91%, at least 92%, at least 93%, at least 94%, at least 95%, at least 95%, at least 97%, at least 98% or at least 99% identity to a sequence selected from the group consisting of SEQ ID NOs: 109 to 118 and 122 to 131; and
b) a second polypeptide chain comprising or consisting of a sequence having at least 90%, at least 91%, at least 92%, at least 93%, at least 94%, at least 95%, at least 95%, at least 97%, at least 98% or at least 99% identity to a sequence selected from the group consisting of SEQ ID NOs: 169 to 178 and SEQ ID NO: 199;
   and the immunoconjugate comprises or consists of first and second copies of said first and second polypeptides.

In some embodiments, the immunoconjugate comprises:
a) a first polypeptide chain comprising or consisting of a sequence selected from the group consisting of SEQ ID NOs: 109 to 118 and 122 to 131; and
b) a second polypeptide chain comprising or consisting of a sequence selected from the group consisting of SEQ ID NOs: 169 to 178 and SEQ ID NO: 199;
   and the immunoconjugate comprises or consists of first and second copies of said first and second polypeptides.

In some embodiments the heavy chain is Fc enabled.

In some embodiments, the immunoconjugate comprises a first polypeptide chain comprising or consisting of the sequence of SEQ ID NO: 109; and a second polypeptide chain comprising or consisting of the sequence of SEQ ID NO: 169.

In some embodiments, the immunoconjugate comprises a first polypeptide chain comprising or consisting of the sequence of SEQ ID NO: 110; and a second polypeptide chain comprising or consisting of the sequence of SEQ ID NO: 170.

In some embodiments, the immunoconjugate comprises a first polypeptide chain comprising or consisting of the sequence of SEQ ID NO: 111; and a second polypeptide chain comprising or consisting of the sequence of SEQ ID NO: 171.

In some embodiments, the immunoconjugate comprises a first polypeptide chain comprising or consisting of the sequence of SEQ ID NO: 112; and a second polypeptide chain comprising or consisting of the sequence of SEQ ID NO: 172.

In some embodiments, the immunoconjugate comprises a first polypeptide chain comprising or consisting of the sequence of SEQ ID NO: 113; and a second polypeptide chain comprising or consisting of the sequence of SEQ ID NO: 173.

In some embodiments, the immunoconjugate comprises a first polypeptide chain comprising or consisting of the sequence of SEQ ID NO: 114; and a second polypeptide chain comprising or consisting of the sequence of SEQ ID NO: 174. This immunoconjugate is referred to herein as CYTB11.

In some embodiments, the immunoconjugate comprises a first polypeptide chain comprising or consisting of the sequence of SEQ ID NO: 115; and a second polypeptide chain comprising or consisting of the sequence of SEQ ID NO: 175.

In some embodiments, the immunoconjugate comprises a first polypeptide chain comprising or consisting of the sequence of SEQ ID NO: 116; and a second polypeptide chain comprising or consisting of the sequence of SEQ ID NO: 176.

In some embodiments, the immunoconjugate comprises a first polypeptide chain comprising or consisting of the sequence of SEQ ID NO: 117; and a second polypeptide chain comprising or consisting of the sequence of SEQ ID NO: 177.

In some embodiments, the immunoconjugate comprises a first polypeptide chain comprising or consisting of the sequence of SEQ ID NO: 118; and a second polypeptide chain comprising or consisting of the sequence of SEQ ID NO: 178.

In some embodiments, the immunoconjugate comprises a first polypeptide chain comprising or consisting of the sequence of SEQ ID NO: 114; and a second polypeptide chain comprising or consisting of the sequence of SEQ ID NO: 199. This immunoconjugate is referred to herein as CYTB13.

In some embodiments the heavy chain is Fc disabled (LAGA): In some embodiments, the immunoconjugate comprises a first polypeptide chain comprising or consisting of the sequence of SEQ ID NO: 122; and a second polypeptide chain comprising or consisting of the sequence of SEQ ID NO: 169.

In some embodiments, the immunoconjugate comprises a first polypeptide chain comprising or consisting of the sequence of SEQ ID NO: 123; and a second polypeptide chain comprising or consisting of the sequence of SEQ ID NO: 170.

In some embodiments, the immunoconjugate comprises a first polypeptide chain comprising or consisting of the sequence of SEQ ID NO: 124; and a second polypeptide chain comprising or consisting of the sequence of SEQ ID NO: 171.

In some embodiments, the immunoconjugate comprises a first polypeptide chain comprising or consisting of the sequence of SEQ ID NO: 125; and a second polypeptide chain comprising or consisting of the sequence of SEQ ID NO: 172.

In some embodiments, the immunoconjugate comprises a first polypeptide chain comprising or consisting of the sequence of SEQ ID NO: 126; and a second polypeptide chain comprising or consisting of the sequence of SEQ ID NO: 173.

In some embodiments, the immunoconjugate comprises a first polypeptide chain comprising or consisting of the sequence of SEQ ID NO: 127; and a second polypeptide chain comprising or consisting of the sequence of SEQ ID NO: 174.

In some embodiments, the immunoconjugate comprises a first polypeptide chain comprising or consisting of the sequence of SEQ ID NO: 128; and a second polypeptide chain comprising or consisting of the sequence of SEQ ID NO: 175.

In some embodiments, the immunoconjugate comprises a first polypeptide chain comprising or consisting of the sequence of SEQ ID NO: 129; and a second polypeptide chain comprising or consisting of the sequence of SEQ ID NO: 176.

In some embodiments, the immunoconjugate comprises a first polypeptide chain comprising or consisting of the sequence of SEQ ID NO: 130; and a second polypeptide chain comprising or consisting of the sequence of SEQ ID NO: 177.

In some embodiments, the immunoconjugate comprises a first polypeptide chain comprising or consisting of the sequence of SEQ ID NO: 131; and a second polypeptide chain comprising or consisting of the sequence of SEQ ID NO: 178.

### NKp30 LC C-Terminus IL-15 Immunoconjugates

To further outline the applicability of the approach a series of non-limiting example immunoconjugates are provided herein. These immunoconjugates comprise at least two IL-15 polypeptides conjugated to the C-termini of the light chains of a multivalent antibody or antigen-binding fragment thereof that specifically binds to NKp30. In preferred embodiments, the two IL-15 polypeptides are conjugated to the CL domain as shown in FIG. 4B.

The immunoconjugate of any preceding claim, wherein the immunoconjugate comprises:
a) a first polypeptide chain comprising, in an N- to C-terminal direction:
   i. a heavy chain variable region (VH) of an anti-NKp30 antibody comprising an HCDR1 comprising or consisting of a sequence selected from the group consisting of SEQ ID NOs: 11 to 12 and optionally having up to 2 amino acid substitutions thereto, an HCDR2 comprising or consisting of a sequence selected from the group consisting of SEQ ID NOs: 24 to 25 and optionally having up to 2 amino acid substitutions thereto, and an HCDR3 comprising or consisting of a sequence selected from the group consisting of SEQ ID NOs: 37 to 38 and optionally having up to 2 amino acid substitutions thereto;
   ii. a heavy chain constant region comprising or consisting of a sequence having at least 90%, at least 91%, at least 92%, at least 93%, at least 94%, at least 95%, at least 95%, at least 97%, at least 98% or at least 99% identity to a sequence selected from the group consisting of SEQ ID NOs: 105, 106 and 203; and
b) a second polypeptide chain comprising, in an N- to C-terminal direction:
   i. a light chain variable region (VL) of an anti-NKp30 antibody comprising an LCDR1 comprising or consisting of a sequence selected from the group consisting of SEQ ID NOs: 50 to 51 and optionally having up to 2 amino acid substitutions thereto, an LCDR2 comprising or consisting of a sequence selected from the group consisting of SEQ ID NOs: 63 to 64 and optionally having up to 2 amino acid substitutions thereto, and an LCDR3 comprising or consisting of a sequence selected from the group consisting of SEQ ID NOs: 76 to 77 and optionally having up to 2 amino acid substitutions thereto; and
   ii. a light chain constant region comprising or consisting of a sequence having at least 90%, at least 91%, at least 92%, at least 93%, at least 94%, at least 95%, at least 95%, at least 97%, at least 98% or at least 99% identity to a sequence selected from the group consisting of SEQ ID NOs: 107 to 108;
   iii. optionally a linker; and
   iv. an IL-15 polypeptide comprising or consisting of a sequence having at least 90%, at least 91%, at least 92%, at least 93%, at least 94%, at least 95%, at least 95%, at least 97%, at least 98% or at least 99% identity to a sequence selected from the group consisting of SEQ ID NOs: 148 to 155, 206 and 207;
      and the immunoconjugate comprises or consists of first and second copies of said first and second polypeptides.

In some embodiments the optional amino acid substitutions are conservative substitutions.

The immunoconjugate of any preceding claim, wherein the immunoconjugate comprises:
a) a first polypeptide chain comprising, in an N- to C-terminal direction:
   i. a heavy chain variable region (VH) of an anti-NKp30 antibody comprising an HCDR1 comprising or consisting of a sequence selected from the group consisting of SEQ ID NOs: 11 to 12, an HCDR2 comprising or consisting of a sequence selected from the group consisting of SEQ ID NOs: 24 to 25 and an HCDR3 comprising or consisting of a sequence selected from the group consisting of SEQ ID NOs: 37 to 38;
   ii. a heavy chain constant region comprising or consisting of a sequence selected from the group consisting of SEQ ID NOs: 105, 106 and 203; and
b) a second polypeptide chain comprising, in an N- to C-terminal direction:
   i. a light chain variable region (VL) of an anti-NKp30 antibody comprising an LCDR1 comprising or consisting of a sequence selected from the group consisting of SEQ ID NOs: 50 to 51, an LCDR2 comprising or consisting of a sequence selected from the group consisting of SEQ ID NOs: 63 to 64 and an LCDR3 comprising or consisting of a sequence selected from the group consisting of SEQ ID NOs: 76 to 77; and
   ii. a light chain constant region comprising or consisting of a sequence selected from the group consisting of SEQ ID NOs: 107 to 108;
   iii. optionally a linker; and
   iv. an IL-15 polypeptide comprising or consisting of a sequence selected from the group consisting of SEQ ID NOs: 148 to 155, 206 and 207;
      and the immunoconjugate comprises or consists of first and second copies of said first and second polypeptides.

In some embodiments, the immunoconjugate comprises:
a) a first polypeptide chain comprising or consisting of a sequence selected from the group consisting of SEQ ID NOs: 119 to 120 and 132 to 133; and
b) a second polypeptide chain comprising or consisting of a sequence selected from the group consisting of SEQ ID NOs: 179 to 180;
   and the immunoconjugate comprises or consists of first and second copies of said first and second polypeptides.

In some embodiments, the immunoconjugate comprises:
a) a first polypeptide chain comprising or consisting of a sequence having at least 90%, at least 91%, at least 92%, at least 93%, at least 94%, at least 95%, at least 95%, at least 97%, at least 98% or at least 99% identity to a sequence selected from the group consisting of SEQ ID NOs: 119 to 120 and 132 to 133; and
b) a second polypeptide chain comprising or consisting of a sequence having at least 90%, at least 91%, at least 92%, at least 93%, at least 94%, at least 95%, at least 95%, at least 97%, at least 98% or at least 99% identity to a sequence selected from the group consisting of SEQ ID NOs: 179 to 180;
   and the immunoconjugate comprises or consists of first and second copies of said first and second polypeptides.

In some embodiments the heavy chain is Fc enabled: In some embodiments, the immunoconjugate comprises a first polypeptide chain comprising or consisting of the sequence of SEQ ID NO: 119; and a second polypeptide chain comprising or consisting of the sequence of SEQ ID NO: 179.

In some embodiments, the immunoconjugate comprises a first polypeptide chain comprising or consisting of the sequence of SEQ ID NO: 120; and a second polypeptide chain comprising or consisting of the sequence of SEQ ID NO: 180.

In some embodiments the heavy chain is Fc disabled (LAGA):

In some embodiments, the immunoconjugate comprises a first polypeptide chain comprising or consisting of the sequence of SEQ ID NO: 132; and a second polypeptide chain comprising or consisting of the sequence of SEQ ID NO: 179.

In some embodiments, the immunoconjugate comprises a first polypeptide chain comprising or consisting of the sequence of SEQ ID NO: 133; and a second polypeptide chain comprising or consisting of the sequence of SEQ ID NO: 180.

### NKp46 LC C-Terminus IL-15 Immunoconjugates

To further outline the applicability of the approach a series of non-limiting example immunoconjugates are provided herein. These immunoconjugates comprise at least two IL-15 polypeptides conjugated to the C-termini of the light chains of a multivalent antibody or antigen-binding fragment thereof that specifically binds to NKp46. In preferred embodiments, the two IL-15 polypeptides are conjugated to the CL domain as shown in FIG. 4B.

The immunoconjugate of any preceding claim, wherein the immunoconjugate comprises:
a) a first polypeptide chain comprising, in an N- to C-terminal direction:
   i. a heavy chain variable region (VH) of an anti-NKp46 antibody comprising an HCDR1 comprising or consisting of SEQ ID NO: 13 and optionally having up to 2 amino acid substitutions thereto, an HCDR2 comprising or consisting of SEQ ID NO: 26 and optionally having up to 2 amino acid substitutions thereto, and an HCDR3 comprising or consisting of SEQ ID NO: 39 and optionally having up to 2 amino acid substitutions thereto;
   ii. a heavy chain constant region comprising or consisting of a sequence having at least 90%, at least 91%, at least 92%, at least 93%, at least 94%, at least 95%, at least 95%, at least 97%, at least 98% or at least 99% identity to a sequence selected from the group consisting of SEQ ID NO: 105 to 106; and
b) a second polypeptide chain comprising, in an N- to C-terminal direction:
   i. a light chain variable region (VL) of an anti-NKp46 antibody comprising an LCDR1 comprising or consisting of SEQ ID NO: 52 and optionally having up to 2 amino acid substitutions thereto, an LCDR2 comprising or consisting of SEQ ID NO: 65 and optionally having up to 2 amino acid substitutions thereto, and an LCDR3 comprising or consisting of SEQ ID NO: 78 and optionally having up to 2 amino acid substitutions thereto; and
   ii. a light chain constant region comprising or consisting of a sequence having at least 90%, at least 91%, at least 92%, at least 93%, at least 94%, at least 95%, at least 95%, at least 97%, at least 98% or at least 99% identity to SEQ ID NO: 107;
   iii. optionally a linker; and
   iv. an IL-15 polypeptide comprising or consisting of a sequence having at least 90%, at least 91%, at least 92%, at least 93%, at least 94%, at least 95%, at least 95%, at least 97%, at least 98% or at least 99% identity to a sequence selected from the group consisting of SEQ ID NOs: 148 to 155, 206 and 207;
      and the immunoconjugate comprises or consists of first and second copies of said first and second polypeptides.

In some embodiments the optional amino acid substitutions are conservative substitutions.

The immunoconjugate of any preceding claim, wherein the immunoconjugate comprises:
a) a first polypeptide chain comprising, in an N- to C-terminal direction:
   i. a heavy chain variable region (VH) of an anti-NKp46 antibody comprising an HCDR1 comprising or consisting of SEQ ID NO: 13, an HCDR2 comprising or consisting of SEQ ID NO: 26, and an HCDR3 comprising or consisting of SEQ ID NO: 39;
   ii. a heavy chain constant region comprising or consisting of a sequence selected from the group consisting of SEQ ID NOs: 105 to 106; and
b) a second polypeptide chain comprising, in an N- to C-terminal direction:
   i. a light chain variable region (VL) of an anti-NKp46 antibody comprising an LCDR1 comprising or consisting of SEQ ID NO: 52, an LCDR2 comprising or consisting of SEQ ID NO: 65 and an LCDR3 comprising or consisting of SEQ ID NO: 78; and
   ii. a light chain constant region comprising or consisting of SEQ ID NO: 107;
   iii. optionally a linker; and
   iv. an IL-15 polypeptide comprising or consisting of a sequence selected from the group consisting of SEQ ID NOs: 148 to 155, 206 and 207;
      and the immunoconjugate comprises or consists of first and second copies of said first and second polypeptides.

In some embodiments, the immunoconjugate comprises:
a) a first polypeptide chain comprising or consisting of a sequence selected from the group consisting of SEQ ID NOs: 121 and 134; and
b) a second polypeptide chain comprising or consisting of the sequence of SEQ ID NO: 181;
   and the immunoconjugate comprises or consists of first and second copies of said first and second polypeptides.

In some embodiments, the immunoconjugate comprises:
a) a first polypeptide chain comprising or consisting of a sequence having at least 90%, at least 91%, at least 92%, at least 93%, at least 94%, at least 95%, at least 95%, at least 97%, at least 98% or at least 99% identity to a sequence selected from the group consisting of SEQ ID NOs: 121 and 134; and
b) a second polypeptide chain comprising or consisting of a sequence having at least 90%, at least 91%, at least 92%, at least 93%, at least 94%, at least 95%, at least 95%, at least 97%, at least 98% or at least 99% identity to the sequence of SEQ ID NO: 181;
   and the immunoconjugate comprises or consists of first and second copies of said first and second polypeptides.

In some embodiments the heavy chain is Fc enabled:

In some embodiments, the immunoconjugate comprises a first polypeptide chain comprising or consisting of the sequence of SEQ ID NO: 121; and a second polypeptide chain comprising or consisting of the sequence of SEQ ID NO: 181.

In some embodiments the heavy chain is Fc disabled (LAGA):

In some embodiments, the immunoconjugate comprises a first polypeptide chain comprising or consisting of the sequence of SEQ ID NO: 134; and a second polypeptide chain comprising or consisting of the sequence of SEQ ID NO: 181.

### Specific Immunoconjugates - IL-15 Polypeptides are Joined to N-Termini of Heavy Chains

In some embodiments of the invention, the IL-15 polypeptides are conjugated (optionally via a linker) to the N-termini of the antibody component.

In some embodiments of the invention, the IL-15 polypeptides are conjugated (optionally via a linker) to the N-termini of the heavy chains of the antibody component. In some preferred embodiments the IL-15 polypeptides are conjugated (optionally via a linker) to the N-termini of the VH domains of the heavy chains. This format is shown in FIG. 5B.

In the immunoconjugate formats described below, the immunoglobulin heavy chain(s) of the multivalent antibody is referred to as the first polypeptide chain. The first and second copy of the first polypeptide chain therefore refer to the two identical heavy chains of the multivalent antibody. In the immunoconjugate formats described below, the immunoglobulin light chain(s) of the multivalent antibody is referred to as the second polypeptide chain. The first and second copy of the second polypeptide chain therefore refer to the two identical light chains of the multivalent antibody. In some embodiments, the first polypeptide (heavy chain) and second polypeptide (light chain) forms one subunit. This then homodimerizes to form the final immunoconjugate comprising two said subunits and thus a total of four polypeptide chains. Hence, in some embodiments, the immunoconjugates comprise first and second copies of said first and second polypeptide chains. The immunoconjugates can therefore be described as dimers, with one monomer of the dimer comprising a first copy of the first polypeptide chain and a first copy of the second polypeptide chain and the second monomer of the dimer comprising a second copy of the first polypeptide chain and a second copy of the second polypeptide chain. In these embodiments the immunoconjugate is considered homodimeric. In some embodiments, the immunoconjugate may have been selectively purified in order to arrive at such a homodimeric species and remove or reduce any heterodimeric species that may be considered impurities. In some embodiments the homodimeric immunoconjugate is the dominant species in a mixture that includes homodimeric and heterodimeric immunoconjugates.

In some embodiments, the anti-NKG2D antibody, anti-NKp30 antibody, anti-NKp46 antibody, anti-NKp44 antibody or anti-DNAM-1 antibody referred to in the immunoconjugate formats described below is any of the antibodies H1/L1 to H13/L13, or could be another antibody against these targets known in the art.

In some embodiments the immunoconjugate comprises:
a) a first polypeptide chain comprising, in an N- to C-terminal direction:
   i. the IL-15 polypeptide;
   ii. optionally a linker;
   iii. a heavy chain variable region (VH) of an anti-NKG2D antibody, an anti-NKp30 antibody, an anti-NKp46 antibody, an anti-NKp44 antibody or an anti-DNAM-1 antibody; and
   iv. a human heavy chain constant region;
b) a second polypeptide chain comprising, in an N- to C-terminal direction:
   i. a light chain variable region (VL) of an anti-NKG2D antibody, an anti-NKp30 antibody, an anti-NKp46 antibody, an anti-NKp44 antibody or an anti-DNAM-1 antibody; and
   ii. a human light constant region;
      and the immunoconjugate comprises or consists of first and second copies of said first and second polypeptides.

In some embodiments the immunoconjugate comprises:
a) a first polypeptide chain comprising, in an N- to C-terminal direction:
   i. the IL-15 polypeptide;
   ii. optionally a linker;
   iii. a heavy chain variable region (VH) of an anti-NKG2D antibody, an anti-NKp3D antibody, an anti-NKp46 antibody, an anti-NKp44 antibody or an anti-DNAM-1 antibody; and
   iv. a human heavy chain IgG sequence;
b) a second polypeptide chain comprising, in an N- to C-terminal direction:
   i. a light chain variable region (VL) of an anti-NKG2D antibody, an anti-NKp30 antibody, an anti-NKp46 antibody, an anti-NKp44 antibody or an anti-DNAM-1 antibody; and
   ii. a human light chain IgG sequence;
      and the immunoconjugate comprises or consists of first and second copies of said first and second polypeptides.

In some embodiments the immunoconjugate comprises:
a) a first polypeptide chain comprising, in an N- to C-terminal direction:
   i. the IL-15 polypeptide;
   ii. optionally a linker;
   iii. a heavy chain variable region (VH) of an anti-NKG2D antibody, an anti-NKp3D antibody, an anti-NKp46 antibody, an anti-NKp44 antibody or an anti-DNAM-1 antibody; and
   iv. a human heavy chain IgG1 sequence;
b) a second polypeptide chain comprising, in an N- to C-terminal direction:
   i. a light chain variable region (VL) of an anti-NKG2D antibody, an anti-NKp30 antibody, an anti-NKp46 antibody, an anti-NKp44 antibody or an anti-DNAM-1 antibody; and
   ii. a human light chain IgG1 sequence;
      and the immunoconjugate comprises or consists of first and second copies of said first and second polypeptides.

In some embodiments the immunoconjugate comprises:
a) a first polypeptide chain comprising, in an N- to C-terminal direction:
   i. the IL-15 polypeptide;
   ii. optionally a linker; and
   iii. a heavy chain of an anti-NKG2D antibody, an anti-NKp30 antibody, an anti-NKp46 antibody, an anti-NKp44 antibody or an anti-DNAM-1 antibody;
b) a second polypeptide chain comprising a light chain of an anti-NKG2D antibody, an anti-NKp30 antibody, an anti-NKp46 antibody, an anti-NKp44 antibody or an anti-DNAM-1 antibody;
   and the immunoconjugate comprises or consists of first and second copies of said first and second polypeptides.

In some embodiments the immunoconjugate comprises:
a) a first polypeptide chain comprising, in an N- to C-terminal direction:
   i. the IL-15 polypeptide;
   ii. optionally a linker, and
   iii. a heavy chain variable region (VH) of an anti-NKG2D antibody, an anti-NKp30 antibody, an anti-NKp46 antibody, an anti-NKp44 antibody or an anti-DNAM-1 antibody, the VH comprising an HCDR1, an HCDR2 and an HCDR3;
   iv. a heavy chain constant region comprising an CH1, a hinge, a CH2 and a CH3 region;
b) a second polypeptide chain comprising, in an N- to C-terminal direction:
   i. a light chain variable region (VL) of an anti-NKG2D antibody, an anti-NKp30 antibody, an anti-NKp46 antibody, an anti-NKp44 antibody or an anti-DNAM-1 antibody, the VL comprising an LCDR1, an LCDR2 and an LCDR3; and
   ii. a light chain constant (CL) region;
      and the immunoconjugate comprises or consists of first and second copies of said first and second polypeptides.

In any of the above embodiments, the first and second polypeptide chains are covalently or non-covalently associated with each other such that each heavy chain (variable region) and each light chain (variable region) form an antigen binding domain. The first copy of the first and second polypeptide chain and the second copy of the first and second polypeptide chain are covalently or non-covalently associated with each other in an immunoglobulin-type format, thereby forming the immunoconjugate comprising the multivalent antibody or antigen binding fragment thereof.

In some embodiments the immunoconjugate comprises a first polypeptide chain comprising or consisting of the sequence of SEQ ID NO: 190; and a second polypeptide chain comprising or consisting of the sequence of SEQ ID NO: 140.

### Two Chain Formats

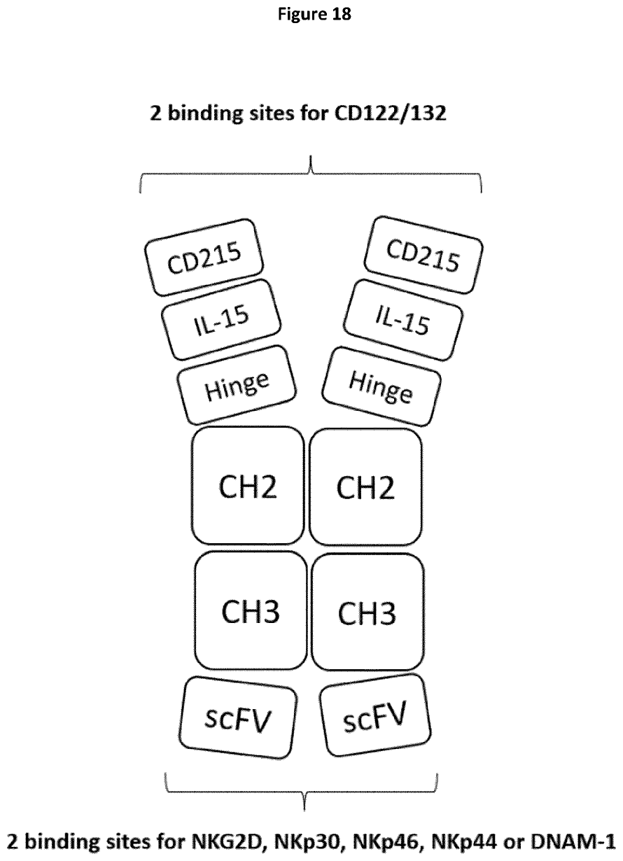

In some embodiments of the invention, the “2+2” multivalency is achieved with a dimeric two chain format, rather than a four chain format. In a preferred embodiment the two chain immunoconjugates are homodimers formed of two identical polypeptide chains. The two chain immunocytokines have two binding sites for CD122/132 and two binding sites for NKG2D, NKp30, NKp46, NKp44 and DNAM-1. Such formats are depicted in FIG. 18.

In some embodiments the immunoconjugate comprises a polypeptide chain comprising (optionally in an N to C terminal direction):
a) an IL-15 polypeptide;
b) optionally a linker;
c) a heavy chain constant region or fragment thereof,
d) optionally a linker;
e) an antibody or antigen-binding fragment thereof comprising at least one antibody binding domain that specifically binds to an antigen selected from the group consisting of NKG2D, NKp30, NKp46, NKp44 and DNAM-1;
   and the immunoconjugate comprises or consists of first and second copies of said polypeptide chain.

The IL-15 polypeptide of the two-chain format immunoconjugates may be any IL-15 polypeptide as described herein. In some embodiments, the IL-15 polypeptide comprises or consists of a sequence selected from the group consisting of SEQ ID NOs: 154 to 155, 206 and 207. In preferred embodiments, the IL-15 polypeptide comprises or consists of a sequence selected from the group consisting of SEQ ID NOs: 154, 206 and 207.

Either or both of the linkers in the two chain format immunoconjugates may be any linker as described herein. In some embodiments, the linker comprises or consists of a sequence selected from the group consisting of SEQ ID NOs: 182 to 187, 204 to 205 and 209.

In some embodiments, the heavy chain constant region or fragment thereof of the two-chain format immunoconjugates is a fragment comprising the hinge domain, the CH2 domain and the CH3 domain. In some embodiments, the heavy chain constant region or fragment thereof of is a fragment comprising the human heavy chain IgG1 hinge domain, the human heavy chain IgG1 CH2 domain and the human heavy chain IgG1 CH3 domain. In some embodiments the heavy chain constant region or fragment thereof comprises or consists of the sequence of SEQ ID NO: 208.

The antibody or antigen-binding fragment of the two-chain format immunoconjugates may be any of the antibody components described herein. In some embodiments, the anti-NKG2D antibody or fragment thereof, anti-NKp30 antibody or fragment thereof, anti-NKp46 antibody or fragment thereof, anti-NKp44 antibody or fragment thereof or anti-DNAM-1 antibody or fragment thereof referred to in the two chain immunoconjugate formats is any of the antibodies H1/L1 to H13/L13 or a fragment thereof, or could be another antibody or fragment thereof against these targets known in the art. In preferred embodiments the antibody or antigen-binding fragment is a scFv domain. In some embodiments the scFv comprises or consists of a VH domain of an anti-NKG2D antibody, an anti-NKp30 antibody, an anti-NKp46 antibody, an anti-NKp44 antibody or an anti-DNAM-1 antibody and a VL domain of an anti-NKG2D antibody, an anti-NKp30 antibody, an anti-NKp46 antibody, an anti-NKp44 antibody or an anti-DNAM-1 antibody joined by a linker. The VH domain may be at the N-termini of the scFv or the VL domain may be at the N-termini of the ScFv and any suitable linker may be used to join the VH domain and VL domain. In some embodiments, the scFv comprises or consists of a sequence selected from the group consisting of SEQ ID NO: 211 to 236.

In some embodiments the scFv comprises or consists of a VH domain of an anti-NKG2D antibody and a VL domain of an anti-NKG2D antibody joined by a linker. The linker may be any suitable linker, such as the linkers described herein. The VH domain may be at the N-termini of the scFv or the VL domain may be at the N-termini of the scFv and any suitable linker may be used to join the VH domain and VL domain. In some embodiments, the scFv comprises or consists of a sequence selected from the group consisting of SEQ ID NO: 211 to 220 and 224 to 233.

In some embodiments the immunoconjugate comprises a polypeptide chain comprising (optionally in an N to C terminal direction):
a) an IL-15 polypeptide comprising or consisting of the sequence of SEQ ID NO: 154;
b) optionally a linker;
c) a heavy chain constant region fragment comprising a hinge, a CH2 and a CH3 region, and comprising or consisting of the sequence of SEQ ID NO: 208;
d) optionally a linker;
e) a scFv domain that specifically binds to NKG2D;
   and the immunoconjugate comprises or consists of first and second copies of said polypeptide chain.

In some embodiments the immunoconjugate comprises a polypeptide chain comprising (optionally in an N to C terminal direction):
a) an IL-15 polypeptide comprising or consisting of the sequence of SEQ ID NO: 154;
b) optionally a linker;
c) a heavy chain constant region fragment comprising a hinge, a CH2 and a CH3 region, and comprising or consisting of the sequence of SEQ ID NO: 208;
d) optionally a linker;
e) a scFv domain that specifically binds to NKG2D, comprising or consisting of a sequence selected from the group consisting of SEQ ID NO: 211 to 220 and 224 to 233;
   and the immunoconjugate comprises or consists of first and second copies of said polypeptide chain.

In some embodiments, the immunoconjugate comprises a polypeptide chain comprising or consisting of a sequence selected from the group consisting of SEQ ID NO: 237 to 246 and 250 to 259, and the immunoconjugate comprises or consists of first and second copies of said polypeptide chain.

In some embodiments, the immunoconjugate comprises a polypeptide chain comprising or consisting of the sequence of SEQ ID NO: 242 or 255, and the immunoconjugate comprises or consists of first and second copies of said polypeptide chain.

In some embodiments, the immunoconjugate comprises a polypeptide chain comprising or consisting of the sequence of SEQ ID NO: 237 or 250, and the immunoconjugate comprises or consists of first and second copies of said polypeptide chain.

In some embodiments the scFv comprises or consists of a VH domain of an anti-NKp30 antibody and a VL domain of an anti-NKp30 antibody joined by a linker. The VH domain may be at the N-termini of the scFv or the VL domain may be at the N-termini of the ScFv and any suitable linker may be used to join the VH domain and VL domain. In some embodiments, the scFv comprises or consists of a sequence selected from the group consisting of SEQ ID NO: 221, 222, 234 and 235.

In some embodiments the immunoconjugate comprises a polypeptide chain comprising:
a) an IL-15 polypeptide comprising or consisting of the sequence of SEQ ID NO: 154;
b) optionally a linker;
c) a heavy chain constant region fragment comprising a hinge, a CH2 and a CH3 region, and comprising or consisting of the sequence of SEQ ID NO: 208;
d) optionally a linker;
e) a scFv domain that specifically binds to NKp30;
   and the immunoconjugate comprises or consists of first and second copies of said polypeptide chain.

In some embodiments the immunoconjugate comprises a polypeptide chain comprising:
a) an IL-15 polypeptide comprising or consisting of the sequence of SEQ ID NO: 154;
b) optionally a linker;
c) a heavy chain constant region fragment comprising a hinge, a CH2 and a CH3 region, and comprising or consisting of the sequence of SEQ ID NO: 208;
d) optionally a linker;
e) a scFv domain that specifically binds to NKp30, comprising or consisting of a sequence selected from the group consisting of SEQ ID NO: 221, 222, 234 and 235;
   and the immunoconjugate comprises or consists of first and second copies of said polypeptide chain.

In some embodiments, the immunoconjugate comprises a polypeptide chain comprising or consisting of a sequence selected from the group consisting of SEQ ID NO: 247 to 248 and 260 to 261, and the immunoconjugate comprises or consists of first and second copies of said polypeptide chain.

In some embodiments the scFv comprises or consists of a VH domain of an anti-NKp46 antibody and a VL domain of an anti-NKp46 antibody joined by a linker. The VH domain may be at the N-termini of the scFv or the VL domain may be at the N-termini of the ScFv and any suitable linker may be used to join the VH domain and VL domain. In some embodiments, the scFv comprises or consists of a sequence selected from the group consisting of SEQ ID NO: 223 and 236.

In some embodiments the immunoconjugate comprises a polypeptide chain comprising:
a) an IL-15 polypeptide comprising or consisting of the sequence of SEQ ID NO: 154;
b) optionally a linker;
c) a heavy chain constant region fragment comprising a hinge, a CH2 and a CH3 region, and comprising or consisting of the sequence of SEQ ID NO: 208;
d) optionally a linker;
e) a scFv domain that specifically binds to NKp46;
   and the immunoconjugate comprises or consists of first and second copies of said polypeptide chain.

In some embodiments the immunoconjugate comprises a polypeptide chain comprising:
a) an IL-15 polypeptide comprising or consisting of the sequence of SEQ ID NO: 154;
b) optionally a linker;
c) a heavy chain constant region fragment comprising a hinge, a CH2 and a CH3 region, and comprising or consisting of the sequence of SEQ ID NO: 208;
d) optionally a linker;
e) a scFv domain that specifically binds to NKp46, comprising or consisting of a sequence selected from the group consisting of SEQ ID NO: 223 and 236;
   and the immunoconjugate comprises or consists of first and second copies of said polypeptide chain.

In some embodiments, the immunoconjugate comprises a polypeptide chain comprising or consisting of a sequence selected from the group consisting of SEQ ID NO: 249 and 262, and the immunoconjugate comprises or consists of first and second copies of said polypeptide chain.

The first copy of the polypeptide chain and the second copy of the polypeptide chain are covalently or non-covalently associated with each other in an immunoglobulin-type format, thereby forming the multivalent immunoconjugate.

### Functional Properties of the Immunoconjugates

There are multiple potential surprising and advantageous properties to the immunoconjugates of the invention, such as those described in the Background, Summary and other sections herein. These properties are exemplified and described in more detail in the Examples section herein. Non-limiting examples may include one or more of the following;

In some embodiments, the multivalent antibody or antigen binding fragment thereof has agonistic activity when administered to a subject or to cell. In some embodiments the agonistic activity is confirmed by an increase in levels of secreted IL-2 or secreted interferon-gamma in a subject or cell, or cell proliferation, or cell cytotoxicity associated with the activated cell or cells. This leads to enhanced, more agonistic antibodies selective for immune cells positive for NKG2D, NKp30, NKp46, NKp44 and/or DNAM-1. This because the same cells which are positive for NKG2D, NKp30, NKp46, NKp44 and/or DNAM-1 can also be positive for the IL-15 CD122/CD132 complex (See CD122 case study in Example 3). Hence an immunoconjugate composition as provided by the methods herein may thus be capable of selectively binding IL-15 receptor complexes on the cell membrane surface via the IL-15 cytokine component. In turn such binding may aid in presentation of the antibody component to its cognate receptor also expressed on the same cell or neighbouring cell. As discussed, presentation of anti-NKG2D, anti-NKp30, anti-NKp46, anti-NKp44 or anti-DNAM-1 antibodies is typically needed for full receptor agonism (e.g. through cross-linking). Hence, through such IL-15 mediated binding and subsequent antibody presentation, enhanced receptor agonism may be exhibited without a need for antibody presentation via alternative Fc receptors, protein-A beads, tissue culture surfaces, protein aggregation, and the like.

Conversely, and equally favourable, more selective IL-15 targeting may be achievable and so more selective IL-15 conferred effects may be observed by the immunoconjugates made by the methods described herein. This is because by fusing a fully biological active IL-15 cytokine to an antibody selective for NKG2D, NKp30, NKp46, NKp44 or DNAM-1 as well as then also including the IL-15 sushi domain, the biological effects of IL-15 may be more focused and more targeted in two key ways;

First, the IL-15 effects may be more targeted towards cells positive for NKG2D, NKp30, NKp46, NKp44 or DNAM-1 as directed by the antibody component binding of said immunoconjugate to said positive cells.

In some embodiments, the immunoconjugates of the present invention bind to NKG2D with a binding affinity (KD) as measured by surface plasmon resonance of less than 5.0 nM. In some embodiments the KD is less than 2.5 nM.

In some embodiments the KD is less than 1.0 nM. In some embodiments, the KD is less than 0.8 nM. In some embodiments, the immunoconjugates of the present invention bind to NKG2D with a binding affinity (KD) as measured by surface plasmon resonance that is less than the KD of the NKG2D antibody or antigen-binding fragment thereof when not part of an immunoconjugate.

In some embodiments, the immunoconjugates of the present invention bind to IL-15Rβ with a binding affinity (KD) as measured by surface plasmon resonance of less than 10.0 nM. In some embodiments the KD is less than 8.0 nM. In some embodiments the KD is less than 5.0 nM. In some embodiments, the KD is less than 4.6 nM. In some embodiments, the immunoconjugates of the present invention bind to IL-15Rβ with a binding affinity (KD) as measured by surface plasmon resonance that is less than the KD of the IL-15 polypeptide or fragment thereof when not part of an immunoconjugate.

In some embodiments, the immunoconjugates of the present invention bind to NKG2D with a binding affinity (KD) as measured by surface plasmon resonance of less than 5.0 nM and bind to IL-15R$ with a binding affinity (KD) as measured by surface plasmon resonance of less than 10.0 nM. In some embodiments, the immunoconjugates of the present invention bind to NKG2D with a binding affinity (KD) as measured by surface plasmon resonance of less than 1.0 nM and bind to IL-15R$ with a binding affinity (KD) as measured by surface plasmon resonance of less than 5.0 nM. In some embodiments, the immunoconjugates of the present invention bind to NKG2D with a binding affinity (KD) as measured by surface plasmon resonance of less than 0.8 nM and bind to IL-15R$ with a binding affinity (KD) as measured by surface plasmon resonance of less than 4.6 nM. In some embodiments, the immunoconjugates of the present invention bind to NKG2D with a binding affinity (KD) as measured by surface plasmon resonance that is less than the KD of the NKG2D antibody or antigen-binding fragment thereof when not part of an immunoconjugate. In some embodiments, the immunoconjugates of the present invention bind to NKG2D with a binding affinity (KD) as measured by surface plasmon resonance that is less than the KD of the NKG2D antibody or antigen-binding fragment thereof when not part of an immunoconjugate, and bind to IL-15Rβ with a binding affinity (KD) as measured by surface plasmon resonance that is less than the KD of the IL-15 polypeptide or fragment thereof when not part of an immunoconjugate.

The immunoconjugates of the present invention may have enhanced binding properties in multivalent format when compared to the equivalent monovalent format. In some embodiments the avidity of the multivalent immunoconjugates for NKG2D is greater than the equivalent monovalent format or soluble component and/or the avidity of the multivalent immunoconjugates for IL-15Rβ is greater than the equivalent monovalent format or soluble component.

Second, the CD215 sushi domain within the immunoconjugate is able to block and so reduce binding of the IL-15 cytokine component to systemically expressed CD215. Hence immunoconjugate distribution towards a ‘sink’ of CD215 positive cells located more broadly in other tissues (again see Example 3) may be reduced.

Thus, the immunoconjugate compositions as created by the methods herein may enhance the potency whilst potentially reducing the sink effects and toxicity associated with less well-considered IL-15 based therapy in multiple ways.

In some embodiments the immunoconjugates of the invention are an immunostimulants. In some embodiments the immunoconjugate is capable of activating NKG2D, NKp30, NKp46, NKp44 and/or DNAM-1 receptor-positive gamma delta T-cells and/or NK cells. In some embodiments the immunoconjugate is capable of stimulating the immune system of a subject when administered to said subject. The immunoconjugates of the present invention are useful in augmenting immune cell functionality. The immunoconjugates of the present invention may stimulate and/or activate human primary immune cells. This effect is most pronounced when multivalent immunocytokines are used. For example, CD69/CD107a double-positive NK cells were used as a model for immune cell activation and cytotoxic potential. The CD69 and CD107a markers were chosen as markers synonymous with NK cell activation and cytotoxic potential. Specifically, CD69 is one of the earliest inducible cell surface glycoprotein acquired during immune cell activation. Whilst CD107a (LAMP1) is marker of immune cell degranulation activity which can also serve as a ligand for selectins and help mediate cell-cell adhesion and migration. It is also associated with more tumour-affecting cytotoxic T-cells and NK cells. As such, the findings that these multivalent immunocytokines can increase the numbers of activated, cytotoxic immune cells is an exciting discovery. It was found that, when added as a soluble agent, an anti-NKG2D IgG1 antibody had a negligible impact on the numbers of immune cells. This aligns with prior conventional thinking whereby soluble entities anti-NKG2D antibodies are not agonists. Similarly, when added as a soluble agent, IL-15 polypeptides (comprising Human IL-15Ralpha Sushi domain and Human IL-15) exhibited only a modest impact on the numbers of immune cells. Even adding the same two soluble agents in combination yields only a modest impact on immune cell number. In contrast, when multivalent immunoconjugates of the invention were used, a remarkable and titratable synergistic increase in the numbers of immune cells was observed. In some embodiments, the multivalent immunoconjugates of the invention may have an activating effect on immune cells, such as NK cells, NKT cells, gamma delta T-cells, and CD8 cells. In some embodiments the IL-15 polypeptide of the immunoconjugates of the present invention is present at the C-terminus of the heavy or light chain (first or second chain) and the immunoconjugates have an activating effect on immune cells, such as NK cells, NKT cells, gamma delta T-cells, and CD8 cells.

The immunoconjugates of the present invention were found to increase cytokine production in immune cells. In some embodiments, the immunocytokines of the present invention increase production of a cytokine, such as interferon gamma, when provided to an immune cell. The multivalent immunoconjugates of the invention increase cytokine production compared to soluble NKG2D antibodies, soluble IL-15 cytokines or fragment thereof, or monovalent immunoconjugates.

In some embodiments, multivalent immunoconjugates of the present invention enhance cell mediated killing of cancer cells. In some embodiments, multivalent immunoconjugates of the present invention lead to increased cell immune cell mobilisation. In some embodiments, multivalent immunoconjugates of the present invention lead to proliferation of immune cells, such as NK, NKT, CD8 and gamma-delta T-cells in a dose-dependent manner. Both cell mediated killing of cancer cells and proliferation of immune cells is further increased in the presence of tumour cells. Multivalent immunoconjugates of the invention may further activate immune cells when they are located proximal to cancer cells. In some embodiments, multivalent immunoconjugates of the present invention activate immune cells in a cancer-cell dependent manner.

In some embodiments, the IL-15 polypeptide is cis- or trans-presented to a cell when administered to said cell or a subject comprising said cell. In some the immunoconjugate is capable of binding to a component of the IL-15 receptor complex and to NKG2D, NKp30, NKp46, NKp44 or DNAM-1 simultaneously when administered to a subject or to a cell. This can lead to more potent IL-15 signalling: As previously outlined, IL-15 typically performs more optimally when trans- or cis-presented rather than operating in a soluble format. In this instance, and given the immunoconjugate provided herein is designed to bind a NKG2D, NKp30, NKp46, NKp44 or DNAM-1 receptor via the antibody component, the IL-15 cytokine component may be trans- or cis-presented from cells positive for such receptors and to which said immunoconjugate is bound. The immunoconjugates of the present invention may result in the targeted trans-presentation of said immunoconjugates to cells of the immune system to ensure selective dual agonism. In particular, immunoconjugates comprising the IL-15 “sushi” domain are effective trans-presenters of the IL-15 cytokine to CD122.

Additive or synergistic effects. As discussed above, whilst IL-15 and antibody-based therapeutics as described herein separately hold promise in cancer immunotherapy, they both also have limitations and notable drawbacks.

In contrast, an immunoconjugate as designed and provided herein potentially overcome a number of these drawbacks. Furthermore, cells positive for said NKG2D, NKp30, NKp46, NKp44 or DNAM-1 receptors and IL-15 receptor complex may be stimulated in a more precise and selective manner via signalling events mediated by the antibody component and the IL-15 cytokine component acting in concert. This in turn may reduce the need for additional stimulants (e.g. IL-2 or anti-CD3) alongside reducing associated more systemic toxicity and also may allow lower doses of the immunoconjugate to be used (i.e. the dose of the immunoconjugate used may be lower than the equivalent dose that would be required of the individual components).

In some embodiments, the immunoconjugates of the present invention bind to both targets (NKG2D and IL15-Rp) simultaneously or substantially simultaneously. In some embodiments, the immunoconjugates of the present invention bind to each target (NKG2D and IL15-Rp) sequentially. The immunoconjugates may bind to NKG2D first, and then subsequently bind to IL15-RP. In other embodiments the immunoconjugates may bind to IL15-RP first, and then subsequently bind to NKG2D. In preferred embodiments, the immunoconjugates of the present invention successfully bind to both targets (NKG2D and IL2-RP) regardless of the order in which the targets are bound. This is termed “sequence order independent dual target co-engagement”. In some embodiments the IL-15 polypeptide of the immunoconjugates of the present invention is present at the C-terminus of the heavy or light chain (first or second chain) and the immunoconjugates display sequence order independent dual target co-engagement.

Alongside such advantages it is also recognized that said immunoconjugate as provided herein may also bind IL-15 and the desired antibody receptors quite separately and independently in any certain scenarios. Such mono receptor occupancy effects may also be favourable under certain situations. For example, it would obviate the need to dose two separate active ingredients (namely separate receptor selective antibody and IL-15 cytokine-based product) in scenarios whereby both are considered desirable. This in turn may reduce the cost of goods associated with dosing regimens, simplify product development and commercialization (of one active ingredient versus two), as well as simplify practitioner/patient experiences.

Reduced sink effects more generally—given the immunoconjugates compositions as described herein are designed to specifically and selectively target the immune cells as described herein, the associated sink effects which can in general result in the need for higher and more repeated dosing regimens and less favourable systemic distribution and receptor occupancy profiles can be reduced—once again, this in turn may reduce the cost of goods associated with dosing regimens, simplify product development and commercialization (of one active ingredient versus two), as well as simplify practitioner/patient experiences.

In some embodiments the immunoconjugate is not a heavy constant domain heterodimer. Heavy chain constant domain heterodimers typically comprise knobs-into-holes amino acid changes. Such changes are rationally designed in antibody engineering and used for heterodimerization of the heavy (H) chains, for example in the production of bispecific IgG antibodies. Such technology focuses on the principle that H chains of human immunoglobulin IgG interact at the level of their CH3 domains directly (whereas, at the level of their CH2 domains, they interact via the carbohydrate attachments). Amino acid changes are engineered in order to create a knob on the CH3 of the H chains of heavy chain 1 and a hole on the CH3 of the heavy chain 2. Non-limiting example of such complimentary amino acid changes are provided by IMGT.org and others. They also include trade-marked approaches such as Xtend™ and Duobody™ platforms. For recent review see Ha et al 2016 (Front. Immunol., 6 Oct. 2016 Sec. Vaccines and Molecular Therapeutics Volume 7-2016|(doi.org/10.3389/fimmu.2016.00394). Whilst such approaches can add undesirable additional complexity to antibody design, production, purification and formulation, they can be essential for certain antibody formats such to ensure desired functionality. For example, through such technology one can ensure antibody monovalency for a certain target or targets is ensured. In one embodiment the immunoconjugate of the invention does not comprise said heavy chain domain heterodimer technology or associated constant domain amino acid changes. In some embodiments, the immunoconjugate of the invention has been selectively purified to remove or reduce heterodimeric species in favour of homodimeric species.

### Specific Comparator Immunoconjugates

Provided herein are a number of immunoconjugates provided for purposes of testing, benchmarking and validating the immunoconjugates described herein.

Some comparators may lack an IL-15 receptor alpha (CD215) or a functional fragment thereof, for example an immunoconjugate as shown in FIG. 5D, comprising:
a) an IL-15 cytokine; and
b) a multivalent antibody or antigen-binding fragment thereof, comprising at least two antibody binding domains that each specifically bind to an antigen selected from the group consisting of NKG2D, NKp30, NKp46, NKp44 and DNAM-1.

For example, the comparator molecule may be an immunoconjugate comprising:
a) a first polypeptide chain comprising, in an N- to C-terminal direction:
   i. a heavy chain variable region (VH) of an anti-NKG2D antibody, an anti-NKp30 antibody, an anti-NKp46 antibody, an anti-NKp44 antibody or an anti-DNAM-1 antibody;
   ii. a human heavy chain constant region;
   iii. optionally a linker; and
   iv. an IL-15 cytokine; and
b) a second polypeptide chain comprising, in an N- to C-terminal direction:
   i. a light chain variable region (VL) of an anti-NKG2D antibody, an anti-NKp30 antibody, an anti-NKp46 antibody, an anti-NKp44 antibody or an anti-DNAM-1 antibody; and
   ii. a human light constant region;
      and the immunoconjugate comprises or consists of first and second copies of said first and second polypeptides.

The immunocytokine termed “CYTB14” herein comprises a first polypeptide chain comprising or consisting of the sequence of SEQ ID NO: 193; and a second polypeptide chain comprising or consisting of the sequence of SEQ ID NO: 140. This may be useful for investigating the role of the IL-15 receptor alpha (CD215) or a functional fragment thereof, for example by comparison with molecules having the format shown in FIG. 5A. Examples 7 and 9 provide further analysis of this molecule.

Some comparators may be monovalent formats for comparison with multivalent immunoconjugates.

FIG. 5F provides a monovalent scFV anti-NKG2D fused via linker to an IL-15 cytokine, without a IL-15 receptor alpha (CD215) domain. The molecule termed “CYTB27” herein comprises a single polypeptide chain comprising or consisting of the sequence of SEQ ID NO: 194. CYTB27 was adapted from a previously described anti-NKG2D-IL2 fusion protein (see WO2017136818) which is monovalent for both the IL-2 receptor and NKG2D target. Specifically, the sequence was modified such that the IL-2 cytokine was replaced with wild-type human IL-15. This novel molecules is used herein as a useful comparator as it is monovalent for both NKG2D and IL-15 receptor beta (CD122). Examples 4 and 5 provide further analysis of this molecule.

FIG. 5G provides a monovalent format comprising three chains. The first chain comprises an IL-15 polypeptide to the N-terminus a heavy chain Fc domain. The second chain comprises an antibody heavy chain. The third chain comprises an antibody light chain. The second and third chain pair to create an anti-NKG2D Vh/Vl binding domain. This second/third paired domain heterodimerizes with the first chain via standard CH3 knob-in-hole mutations at the C-termini. This format is also monovalent for both NKG2D and IL-15 receptor beta (CD122). The molecule termed “CYTB29” herein comprises a first polypeptide chain comprising or consisting of the sequence of SEQ ID NO: 196; and a second polypeptide chain comprising or consisting of the sequence of SEQ ID NO: 197; and a third polypeptide chain comprising or consisting of the sequence of SEQ ID NO: 139. The molecule termed “CYTB30” herein comprises a first polypeptide chain comprising or consisting of the sequence of SEQ ID NO: 198; and a second polypeptide chain comprising or consisting of the sequence of SEQ ID NO: 197; and a third polypeptide chain comprising or consisting of the sequence of SEQ ID NO: 139. Examples 5, 6, 10 and 13 provide further analysis of these molecules.

### Polynucleotide Sequences, Expression Vectors and Host Cells

Provided herein is polynucleotide or pair of polynucleotides encoding any of the immunoconjugates described herein.

In some embodiments the invention provides a polynucleotide encoding the polypeptide of any one of SEQ ID NOs: 156 to 181.

In some embodiments the invention provides a pair of polynucleotides, the first polynucleotide encoding the polypeptide of any one of SEQ ID NOs: 156 to 168, and the second polynucleotide encoding the polypeptide of any one of SEQ ID NOs: 135 to 147.

In some embodiments the polynucleotides are present as a kit. In some embodiments the polynucleotides are disposed in separate containers in the kit. In some embodiments the polynucleotides are present in a single container or composition.

As used herein, the term “polynucleotide,” “nucleotide,” nucleic acid” “nucleic acid molecule” and other similar terms are used interchangeably and include DNA, RNA, mRNA and the like.

Provided herein is a vector or expression vector comprising a polynucleotide of the invention, or a pair of polynucleotides of the invention.

Also provided herein is a host cell comprising a polynucleotide of the invention, a pair of polynucleotides of the invention, or a vector of the invention.

Also provided herein is a host cell comprising a first polynucleotide encoding a or the heavy chain of an immunoconjugate of the invention and a second polynucleotide encoding a or the light chain of an immunoconjugate of the invention. In some embodiments, the host cell is a CHO cell.

The term “vector”, as used herein, is intended to refer to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g. bacterial vectors having a bacterial origin of replication and episomal mammalian and yeast vectors). Other vectors (e.g. non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g. replication defective retroviruses, adenoviruses and adeno-associated viruses), which serve equivalent functions, and also bacteriophage and phagemid systems.

### Methods of Making Immunoconjugates

Also provided by the invention is a method of producing an immunoconjugate comprising at least two IL-15 polypeptides and a multivalent antibody that binds to NKG2D, NKp30, NKp46, NKp44 or DNAM-1, comprising (a) culturing the host cell of the invention under conditions suitable for the expression of the immunoconjugate, and optionally (b) recovering and purifying the immunoconjugate.

Also provided by the invention is an immunoconjugate comprising at least two superagonist IL-15 polypeptides and a multivalent antibody that binds to NKG2D, NKp30, NKp46, NKp44 and DNAM-1, obtained by said method.

Provided herein are methods of making immunoconjugates of the invention, the method comprising culturing a host cell of the invention under conditions in which the immunoconjugate is expressed by the cell. The host cells may comprise both chains (i.e. both the heavy and the light chain) of the immunoconjugate. The method optionally further comprises isolating and optionally purifying the expressed immunoconjugate, optionally wherein purification of the immunoconjugate results in an aqueous solution wherein the soluble immunoconjugate monomer content is greater than 80% or greater than 90%. The purification of the immunoconjugate may comprise multiple purification steps. In one embodiment, the first of the purification steps is undertaken using standard preparative chromatography, optionally followed by standard analytical SEC-HPLC analysis of the eluates. The second purification steps is then undertaken using standard preparative size-exclusion-chromatography (SEC), followed by a final buffer exchange of the purified single peak product eluate into standard antibody formulation buffers. In some embodiments, the SEC-eluted main (or dominant) peak is selected for further purification.

In some embodiments the methods further comprise formulating the isolated and optionally purified immunoconjugate into a pharmaceutical composition comprising one or more pharmaceutically acceptable excipients or diluents.

As the skilled person is aware, other methods for making immunoconjugates are available and could be suitably employed.

The invention also provides an immunoconjugate obtained or obtainable by the method of the invention.

The invention also provides a pharmaceutical composition obtained or obtainable by the methods of the invention.

### Compositions and Pharmaceutical Compositions

The invention provides compositions comprising the immunoconjugates of the invention.

The invention also provides a pharmaceutical composition comprising the immunoconjugates of the invention, and one or more pharmaceutically acceptable excipients or diluents.

Typically, the pharmaceutical composition comprises an immunoconjugate of the invention and a pharmaceutically acceptable carrier or excipient.

As used herein, “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, salts, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable substances such as wetting or minor amounts of auxiliary substances such as wetting or emulsifying agents, preservatives or buffers, which enhance the shelf life or effectiveness of the antibody or antigen-binding fragment thereof. “Pharmaceutically acceptable” as used herein can also mean being approved by a regulatory agency of the Federal or a state government, or listed in the U.S. Pharmacopeia, European Pharmacopeia or other generally recognized Pharmacopeia for use in animals, and more particularly in humans

The term “excipient” as used herein refers to inert substances which are commonly used as a diluent, vehicle, preservatives, binders, or stabilizing agent for drugs and includes, but not limited to, proteins (e.g. serum albumin, etc.), amino acids (e.g. aspartic acid, glutamic acid, lysine, arginine, glycine, histidine, etc.), fatty acids and phospholipids (e.g. alkyl sulfonates, caprylate, etc.), surfactants (e.g. SDS, polysorbate, non-ionic surfactant, etc.), saccharides (e.g. sucrose, maltose, trehalose, etc.) and polyols (e.g. mannitol, sorbitol, etc.).

The compositions of this invention may be in a variety of forms. These include, for example, liquid, semi-solid and solid dosage forms, such as liquid solutions (e.g. injectable and infusible solutions), dispersions or suspensions, tablets, pills, powders, liposomes and suppositories.

The composition in accordance with this invention can be formulated for use by any convenient route. The preferred form depends on the intended mode of administration and therapeutic application. Typical preferred compositions are in the form of injectable or infusible solutions.

The preferred mode of administration is parenteral (e.g. intravenous, subcutaneous, intraperitoneal, intramuscular, intrathecal). In a preferred embodiment, the immunoconjugate is administered by intravenous infusion or injection. In another preferred embodiment, the immunoconjugate is administered by intramuscular or subcutaneous injection.

The pharmaceutical compositions of the invention may be presented in unit dose forms containing a predetermined amount of each active ingredient per dose. Such a unit may be adapted to provide 0.5-50 mg/kg of the compound, preferably either 1-10 mg/kg, 1-5 mg/kg, 5-10 mg/kg or 10-50 mg/kg Such doses can be provided in a single dose or as a number of discrete doses. The ultimate dose will of course depend on the condition being treated, the route of administration and the age, weight and condition of the patient and will be at the doctor's discretion. For example, for potent immunoconjugates lower dose provisions may be warranted; for example in the range of approximately 0.01 to 0.5 mg/Kg.

Therapeutic compositions typically must be sterile and stable under the conditions of manufacture and storage. The composition can be formulated as a solution, microemulsion, dispersion, liposome, or other ordered structure suitable to high drug concentration.

It should be understood that in addition to the ingredients particularly mentioned above, the formulations may also include other agents conventional in the art having regard to the type of formulation in question, for example those suitable for oral administration may include flavouring agents.

The immunoconjugates of the invention may be provided as part of a kit. Such kits may include instructions for use and/or additional pharmaceutically active components. The immunoconjugates and the additional pharmaceutically active components may be disposed separately within the kit, or in some embodiments the immunoconjugates and the additional pharmaceutically active components may be formulated together.

It is within the scope of the invention to use the pharmaceutical composition of the invention in therapeutic methods for the treatment of diseases as described herein as an adjunct to, or in conjunction with, other established therapies normally used in the treatment of such diseases.

In a further aspect of the invention, the immunoconjugate, composition or pharmaceutical composition is administered sequentially, simultaneously or separately with at least one active agent.

### Methods of Treatment

The invention provides immunoconjugates of the invention, compositions of the invention, or pharmaceutical compositions of the invention, for use in medicine.

Particularly, the invention provides immunoconjugates of the invention, compositions of the invention, or pharmaceutical compositions of the invention, for use in treating a disease, such as cancer.

The invention further provides immunoconjugates of the invention, compositions of the invention, or pharmaceutical compositions of the invention, for use in stimulating the immune system of a subject.

The invention further provides use of the immunoconjugates of the invention in the manufacture of a medicament, for example in the manufacture of a medicament for the treatment of cancer or the manufacture of a medicament for stimulating the immune system of a subject.

Provided herein is a method of treating a disease, comprising administering to a subject in need thereof a therapeutically effective amount of an immunoconjugate of the invention, or a composition of the invention, or a pharmaceutical composition of the invention.

Also provided herein is a method of treating cancer, comprising administering to a subject in need thereof a therapeutically effective amount of an immunoconjugate of the invention, or a composition of the invention, or a pharmaceutical composition of the invention.

The method of treatment can be of a human or an animal subject and the invention extends equally to uses in both human and/or veterinary medicine. The immunoconjugate of the invention is preferably administered to an individual in a “therapeutically effective amount”, this being sufficient to show benefit to the individual. As used herein, “treatment” includes any regime that can benefit a human or non-human animal, preferably mammal. The treatment may be in respect of an existing condition or may be prophylactic (preventative treatment). The method may be an in vitro method. The method may be an in vivo method.

As used herein “cancer” relates to a disease caused by an uncontrolled division of abnormal cells. These include, but are not limited to, cardiac, sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: oesophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumours, vipoma), small bowel (adenocarcinoma, lymphoma, carcinoid tumours, Karposi's sarcoma, leiomyoma, hemangioma, lipoma, neurofibroma, fibroma), large bowel (adenocarcinoma, tubular adenoma, villous adenoma, hamartoma, leiomyoma) colorectal; Genitourinary tract: kidney (adenocarcinoma, Wilm's tumour [nephroblastoma], lymphoma, leukaemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumours, lipoma); Liver: hepatoma (hepatocellular carcinoma), cholangiocarcinoma, hepatoblastoma, angiosarcoma, hepatocellular adenoma, hemangioma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumour chordoma, osteochronfroma (osteocartilaginous exostoses), benign chondroma, chondroblastoma, chondromyxofibroma, osteoid osteoma and giant cell tumours; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma [pinealoma], glioblastoma multiform, oligodendroglioma, schwannoma, retinoblastoma, congenital tumours), spinal cord neurofibroma, meningioma, glioma, sarcoma); Gynaecological: uterus (endometrial carcinoma), cervix (cervical carcinoma, pre tumour cervical dysplasia), ovaries (ovarian carcinoma [serous cystadenocarcinoma, mucinous cystadenocarcinoma, unclassified carcinoma], granulosa thecal cell tumours, Sertoli-Leydig cell tumours, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma), breast; Hematologic: blood (myeloid leukaemia [acute and chronic], acute lymphoblastic leukaemia, chronic lymphocytic leukaemia, myeloproliferative diseases, multiple myeloma, myelodysplastic syndrome), Hodgkin's disease, non Hodgkin's lymphoma [malignant lymphoma]; Skin: malignant melanoma, basal cell carcinoma, squamous cell carcinoma, Karposi's sarcoma, moles dysplastic nevi, lipoma, angioma, dermatofibroma, keloids, psoriasis; and Adrenal glands: neuroblastoma. In another embodiment, the cancer is carcinoma, lymphoma, leukaemia, blastoma, and sarcoma. More particular examples of such cancers include squamous cell carcinoma, myeloma, small-cell lung cancer, non-small cell lung cancer, glioma, Hodgkin's lymphoma, non-Hodgkin's lymphoma, acute myeloid leukaemia (AML), multiple myeloma, gastrointestinal (tract) cancer, renal cancer, renal cell carcinoma, ovarian cancer, liver cancer, lymphoblastic leukaemia, lymphocytic leukaemia, colorectal cancer, endometrial cancer, kidney cancer, prostate cancer, thyroid cancer, melanoma, chondrosarcoma, neuroblastoma, pancreatic cancer, glioblastoma multiforme, cervical cancer, brain cancer, stomach cancer, bladder cancer, hepatoma, breast cancer, colon carcinoma, non-Hodgkin's lymphoma, cutaneous T-cell lymphoma, and head and neck cancer.

Also provided herein is a method of stimulating the immune system of a subject, comprising administering to a subject in need thereof a therapeutically effective amount of an immunoconjugate of the invention, or a composition of the invention, or a pharmaceutical composition of the invention.

The immunoconjugates of the invention are useful in treating diseases, in particular cancer. The terms “treat”, “treatment” and “treating” refer to the reduction or amelioration of the progression, severity, and/or duration of least one sign or symptom of a disease or disorder experienced by a subject (compared to an earlier point in time, for example, prior to administration of any immunoconjugate).

In one embodiment, the immunoconjugate of the invention stimulates the immune system of a subject when administered in vitro or in vivo.

In one embodiment, the immunoconjugate of the invention activates an NKG2D, NKp30, NKp46, NKp44 and/or DNAM-1 receptor expressed on gamma delta T-cells and/or NK cells when administered in vitro or in vivo.

Also provided herein is the use of an immunoconjugate of the invention to activate an NKG2D, NKp30, NKp46, NKp44 and/or DNAM-1 receptor expressed on gamma delta T-cells and/or NK cells.

Depending on the condition being treated, the immunoconjugates of the invention may be used in combination with other pharmaceutically active components for simultaneous, separate or sequential use. For example, when treating or preventing cancer, the immunoconjugates of the invention may be used in combination with another therapy or additional therapeutically active agent. Suitable therapies or additional therapeutically active agents include radiation therapy, chemotherapy treatment, targeted therapy, immunotherapy, monoclonal antibody therapy, hormonal therapy, angiogenesis inhibition, cancer vaccines, oncolytic virus, toll like receptor agonists, epigenetic modifications, engineered T cells, T cell co-stimulation agonists, tyrosine kinase inhibitors, other anti-cancer chemical agents, palliative care for cancer therapy, an immune checkpoint inhibitor, an immunosuppressant, an anti-inflammatory, an immune modulators, an immune activator and/or an inhibitor such as an IDO inhibitor, a CSF-1R inhibitor, a TGFB inhibitor, a T cell co-stimulation antagonist, a Treg inhibitor, a macrophage modulator, a natural killer cell modulator and a chemokine receptor inhibitor.

Suitable chemotherapy treatments include gemcitabine, cyclophosphamide, doxorubicin, paclitaxel, cisplatin.

The other therapy or additional therapeutically active agent may be another immunoconjugate or a therapeutic antibody.

### EXAMPLES

### Example 1: Outline of Binding Domains and Configurations

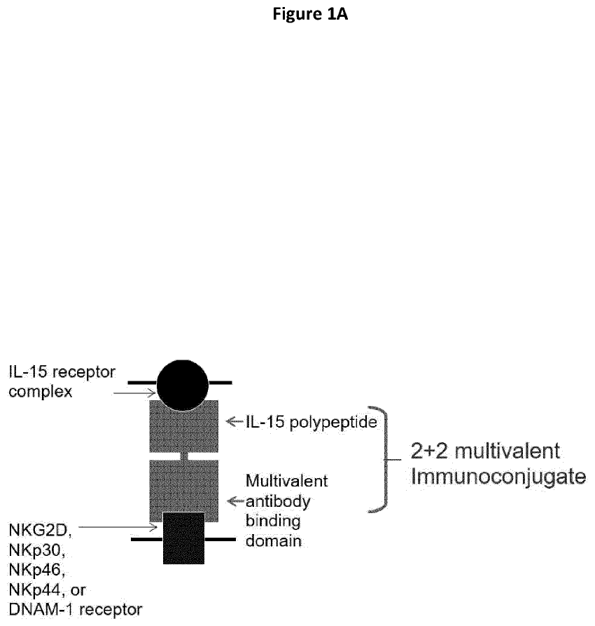

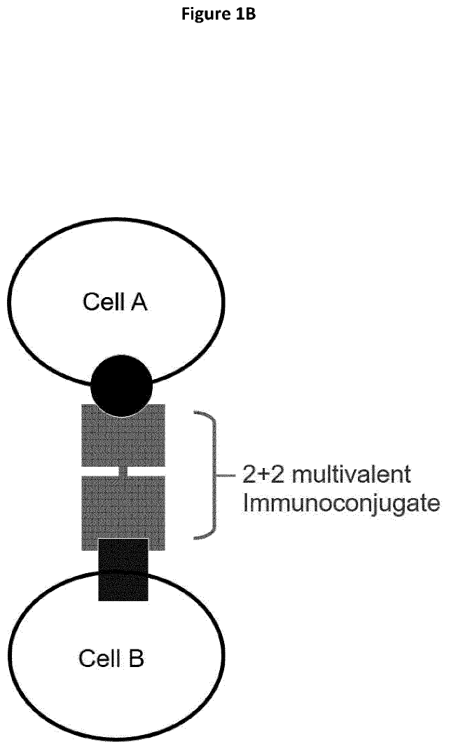

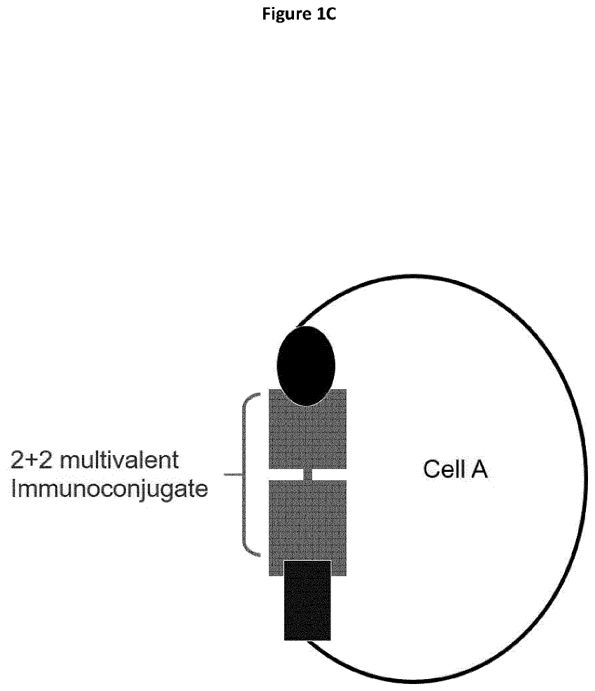

This invention describes immunoconjugates, compositions and associated methods which can result in the targeted co-presentation of immunoconjugates to cells of the immune system in either cis or in trans manner to ensure selective dual agonism. This exciting concept is outlined in FIG. 1. Specifically FIG. 1A describes a simplified schematic to highlight the two principal binding domains of the immunoconjugate, namely the IL-15 polypeptides and the antibody binding domains. In this immunoconjugates of the invention, the multivalent “dog-bone” design contains two antibody binding domains and two IL-15 polypeptides and as such can be termed a 2+2 multivalent approach. FIG. 1B outlines how said immunoconjugate can operate in a cis configuration to activate two cells. FIG. 1C outlines how said immunoconjugate can operate in a trans configuration to activate one cell. FIG. 1D provides a graphical cartoon on how said immunoconjugates could operate upon provision to, and contact of, NK cells and/or gamma delta T-cells.

### Example 2: Construction of Exemplar Multivalent Immunoconjugate

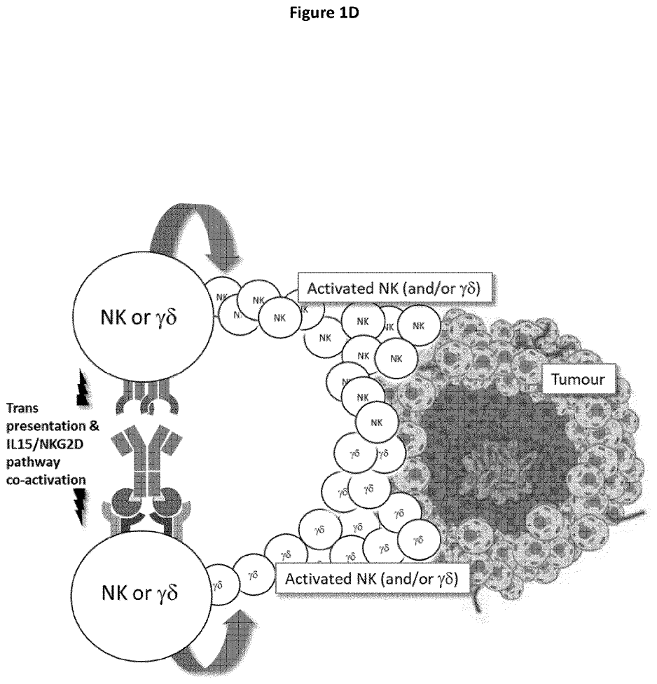

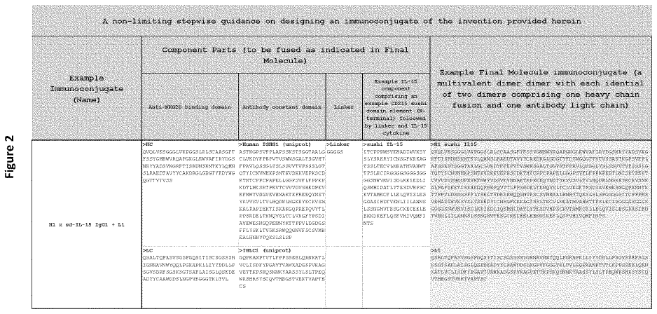

An exemplar immunoconjugate is provided in FIG. 2 and shown as a schematic in FIG. 4. Said example multivalent, dimer recombinant immunoconjugates can be expressed from synthetic open reading frames by standard molecular biology and protein expression techniques known in the art. This example immunoconjugate is created as a 2 bivalent+2 bivalent entity containing two copies of the antibody binding domain, two copies of the sushi domain component, and two copies of the IL-15 cytokine component. All said components in this example immunoconjugates are separated by typical linkers as indicated, for example linkers comprising or consisting of glycine and serine residues. Said linkers can operate as flexible hinges and reduce steric hinderance. Other such linkers are known in the art. Finally, the example presented in FIG. 2 contains the IL-15 sushi domain/IL-15 cytokine fused to the C-terminal of the heavy chain (as shown in FIG. 4A). An alternate approach comprises fusing said IL-15 sushi domain/IL-15 cytokine to the C-terminal of the light chain (as shown in FIG. 4B). The IL-15 sushi domain/IL-15 cytokine can be fused to the antibody binding domain in either orientation (antibody binding domain-IL-15 sushi domain-IL-15 cytokine—or antibody binding domain-IL-15 cytokine-IL-15 sushi domain).

### Example 3: Exploration of Expression Profile Data for the Relevant Receptor Targets

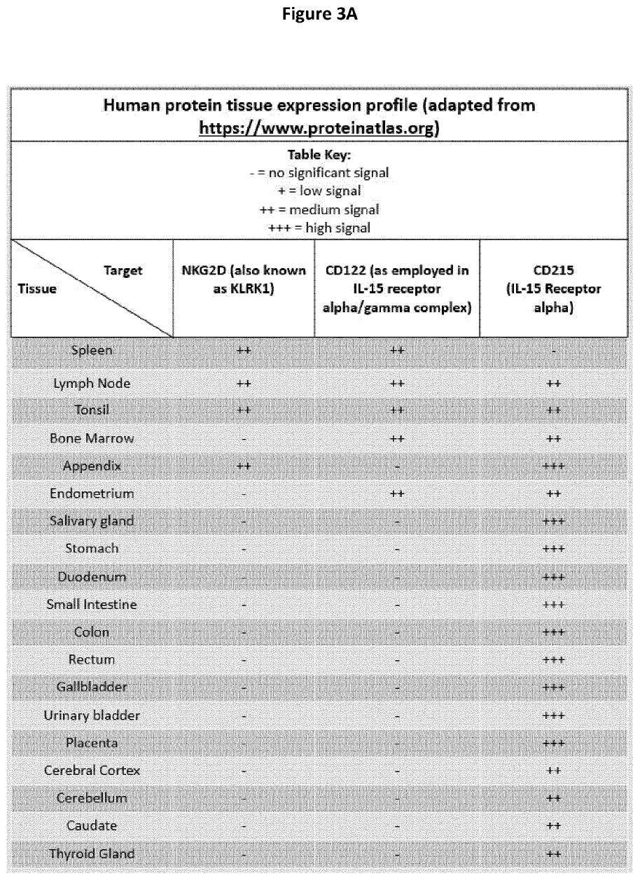

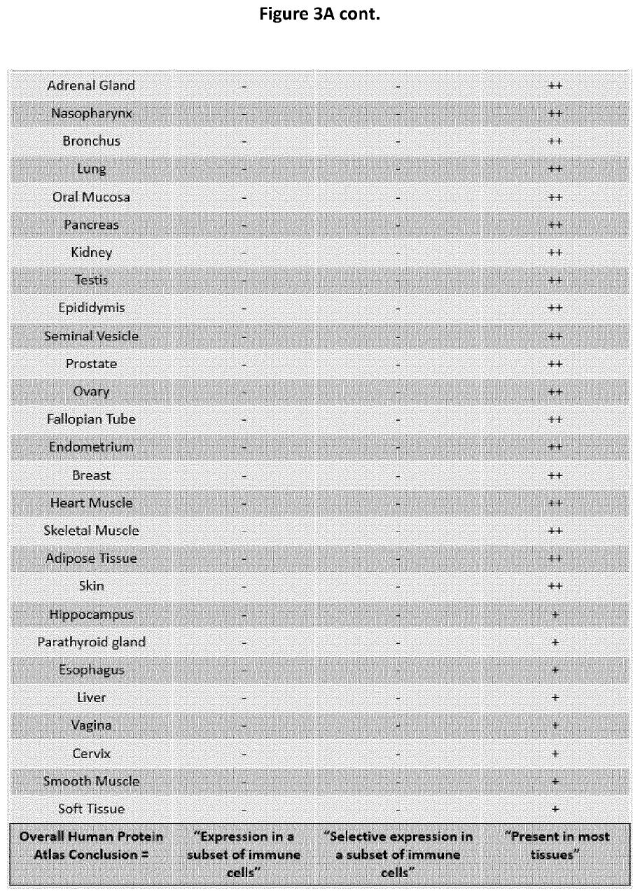

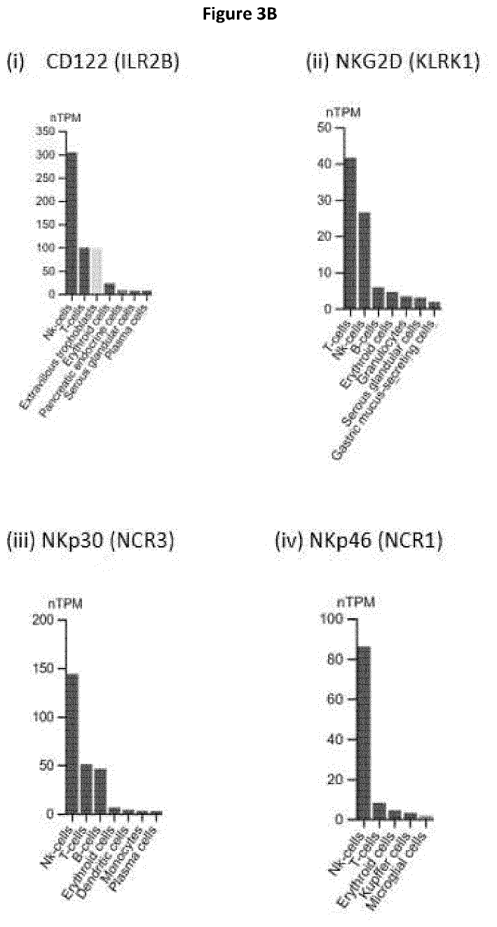

To explore how an immunoconjugate composition as created by the methods described herein may selectively target most desired immune cells, expression profile data for the relevant receptor targets were explored using publicly available data at proteinatlas.org. The results of this study are presented in FIG. 3. During this exploration, additional consideration was made regarding the inclusion of the CD215 sushi domain component which can bind the IL-15 cytokine component of said immunoconjugate. This is because this binding will thereby reduce the ability of the immunoconjugate to bind and distribute to endogenous CD215 (also known as IL-15 receptor alpha). Firstly, for this example, an exploration of human tissue protein expression profiling was performed. For this study, NKG2D was used as an exemplar antibody target. This is because no NKp30 or NKp46 tissue protein profile was available at proteinatlas.org at time of writing. A summary of this exploration of proteinatlas.org data is presented in FIG. 3A. This summary highlights the remarkable tissue protein expression profile overlap between the NKG2D and IL-15 CD122 targets. Indeed both immunoconjugate targets are limited to same immune cell rich tissue compartments. Importantly this exploration also highlights the fact that by inclusion of the CD215 sushi domain (which is capable of binding to the IL-15 cytokine component and thus prevent binding to endogenous CD215), the immunoconjugate is thus more restricted. To further explain—without inclusion of said sushi domain or a IL-15 receptor component comprising the sushi domain, the immunoconjugate composition as generated by the methods described herein would likely be far less selective for immune cells and distribute more widely and to the multiple CD215 positive ‘sink’ tissues across the body as indicated in this Figure.

Secondly, single cell RNA profile data from proteinatlas.org was then explored. A summary of this study is shown in FIG. 3B. Importantly single cell RNA profile is available for CD122, NKG2D, NKp30 and NKp46. And from this analysis, remarkable convergence is highlighted wherein the top two immune cell type for all four targets (CD122, NKG2D, NKp30 and NKp46) are NK cells and T-cells (this profile did not dissect further on which sub-set of T-cell). Such remarkable convergence highlights the fact the immunoconjugate compositions generated by the methods described herein will be highly selective for NK cells and T-cells inclusive of gamma delta T-cells expressing said targets.

### Example 4: Expression and Purification of Multivalent Immunoconjugates

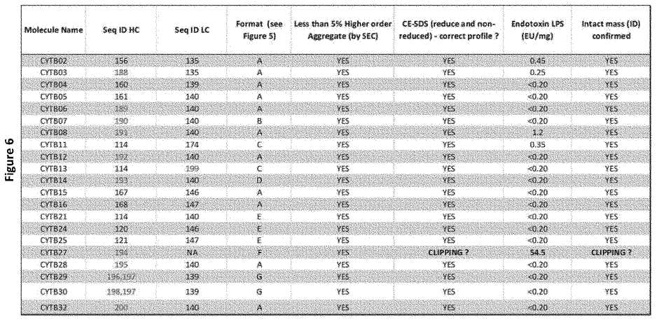

As outlined in Example 2, multivalent immunoconjugates of this invention were designed and produced as follows. First, the DNA open reading frames (ORFs) encoding the multivalent immunoconjugates alongside associated controls and comparator molecules as described herein were synthesized de novo from oligonucleotides. The resulting ORFs generated were then sequenced to confirm identity. These ORFs were then cloned into mammalian expression vectors by standard molecular biology methodology. The resulting expression vectors containing the desired ORFs were then transfected into ExpiCHO™ cells (ThermoFisher Scientific Inc, Waltham, Massachusetts, United States). The transfected cells were then cultured for up to 9 days (100 ml volumes) in standard ExpiCHO™ using standard protocols. After the cultures were harvested, initial first-step purification of the molecules was undertaken using standard preparative chromatography with HiTrap Mabselect Sure™ (Cytiva, Marlborough, Massachusetts, United States) aside for one molecule with a different, non-IgG architecture (CYTB27) where AmpshereA3™ (JSR Sunnyvale, CA, United States) was employed. Interim analysis of all batch eluates was then undertaken using standard analytical SEC-HPLC analysis. A second-step purification was undertaken using standard preparative size-exclusion-chromatography (SEC) before a final buffer exchange of the purified single peak product eluate into standard antibody formulation buffers prior to aliquoting and frozen storage. For all purifications, the SEC-eluted main peak was selected. This was typically the first main or dominant peak. For some but not all sushi-IL15 containing molecules a slower eluting peak was also sometimes observed. This ‘peak 2’ was also purified and characterized separately. This species is likely a conformer or glycan variant given it appeared to be equivalent by all analysis (SDS-PAGE, CE-SDS, cIEF, and intact, deglycosylated LC-MS analysis). It also exhibited similar SPR based binding affinities when studied. During the course of development and once a final molecule is candidate selected, such a species if present is typically further characterized. However, at this junction this species was not the focus of further study. Regardless, all final selected main peaks purified were visually confirmed as the correctly sized, non-aggregated molecules with aid of standard in-process reduced and non-reduced SDS-PAGE. Binding to both targets (NKG2D and CD122) was also confirmed by SPR (see Example 5). Unless otherwise indicated all resulting single peak eluates exhibited a near 100% monomer with less than 5% higher order aggregate. Associated final ‘QC’ analysis of the eluted selected peak was undertaken on representative aliquoted samples first frozen and then thawed prior to analysis. This freeze/thaw of a QC aliquot was undertaken to ensure the material analysed was representative of the long-term −80 C stored frozen material employed in the Example studies described elsewhere below. Standard QC analysis comprised SEC-HPLC (to determine monomer purity, higher order aggregate content), reduced and non-reduced CE-SDS (to confirm product purity/size), intact LC-MS (to confirm identity/size), lipopolysaccharide content (to determine LPS impurity levels). The desired target quality profile is shown below in Table 4. See Table 3 and FIG. 6 for results of this analysis.

TABLE 3

| | | | | | CE-SDS | Endotoxin | Intact |
|---|---|---|---|---|---|---|---|
| Molecule | Seq ID | Seq ID | Format (see | Higher order | (reduce and | LPS | mass (ID) |
| Name | HC | LC | FIG. 5) | aggregate | non-reduced) | (EU/mg) | confirmed |
| CYTB02 | 156 | 135 | A | less than 5% | Correct Profile | 0.45 | YES |
| CYTB03 | 188 | 135 | A | less than 5% | Correct Profile | 0.25 | YES |
| CYTB04 | 160 | 139 | A | less than 5% | Correct Profile | <0.20 | YES |
| CYTB05 | 161 | 140 | A | less than 5% | Correct Profile | <0.20 | YES |
| CYTB06 | 189 | 140 | A | less than 5% | Correct Profile | <0.20 | YES |
| CYTB07 | 190 | 140 | B | less than 5% | Correct Profile | <0.20 | YES |
| CYTB08 | 191 | 140 | A | less than 5% | Correct Profile | 1.2 | YES |
| CYTB11 | 114 | 174 | C | less than 5% | Correct Profile | 0.35 | YES |
| CYTB12 | 192 | 140 | A | less than 5% | Correct Profile | <0.20 | YES |
| CYTB13 | 114 | 199 | C | less than 5% | Correct Profile | <0.20 | YES |
| CYTB14 | 193 | 140 | D | less than 5% | Correct Profile | <0.20 | YES |
| CYTB15 | 167 | 146 | A | less than 5% | Correct Profile | <0.20 | YES |
| CYTB16 | 168 | 147 | A | less than 5% | Correct Profile | <0.20 | YES |
| CYTB21 | 114 | 140 | E | less than 5% | Correct Profile | <0.20 | YES |
| CYTB24 | 120 | 146 | E | less than 5% | Correct Profile | <0.20 | YES |
| CYTB25 | 121 | 147 | E | less than 5% | Correct Profile | <0.20 | YES |
| CYTB27 | 194 | NA | F | less than 5% | Clipping | 54.5 | No, |
| | | | | | observed? | | clipping? |
| CYTB28 | 195 | 140 | A | less than 5% | Correct Profile | <0.20 | YES |
| CYTB29 | 196, 197 | 139 | G | less than 5% | Correct Profile | <0.20 | YES |
| CYTB30 | 198, 197 | 139 | G | less than 5% | Correct Profile | <0.20 | YES |
| CYTB32 | 200 | 140 | A | less than 5% | Correct Profile | <0.20 | YES |

TABLE 4

| Characteristic | Target Results after freeze/thaw |
|---|---|
| In process analysis: Purity | Expected size visually confirmed |
| of selected main peak | by reduced and non-reduced |
| | SDS-PAGE (in process only) |
| Final QC: Purity of SEC eluate | Less than 5% higher-order |
| (by SEC-HPLC) | aggregate |
| Final QC: Size and purity (by | Profiles confirmed as expected |
| reduce and non-reduced CE-SDS) | |
| Final QC: LPS content | Less than 1 EU/mg, |
| Final QC: Intact Mass Analysis after | Expected size |
| deglycosylation (by LC-MS) | |
| Binding to NKG2D and CD122 | Confirmed |
| (by SPR) | |

### Observations of Note:

CYTB27: During production, one comparator molecule monovalent to both targets named CYTB27 exhibited a less desirable analytical profile (see FIG. 6). This molecule was adapted from a previously described anti-NKG2D-IL2 fusion protein (see WO2017136818 Sequence 46) which is monovalent for both the IL-2 receptor and NKG2D target. Specifically, we modified Sequence 46 such that the IL-2 cytokine was replaced with wild-type human IL-15; see FIG. 5 Format F. We do not believe this molecule has previously been designed/generated. However, we considered it a useful comparator given it would be monovalent for both NKG2D and IL-15 receptor beta (CD122). However, we found that CYTB27 did not express well and—possibly related—did not exhibit a desired low lipopolysaccharide profile. Further, additional species observed by CE-SDS and LC-MS also suggested significant clipping. Two repeated attempts to make further batches of CYTB27 using the same standard expression and purification methodologies but at differing scales also failed. Hence whilst CYTB27 has still been employed as a comparator herein, results are caveated where indicated. Further studies would be needed to determine if molecule or process re-design can alleviate the observed lower expression, higher LPS profile associated with CYTB27. However, given this comparator is monovalent to both NKG2D and CD122/IL-15 receptor beta, it was not of primary interest to us and we did not explore this technical observation further.

In conclusion, the main objective of these productions was to generate batches of purified predominantly monomer and intact product. The batches generated needed to be of suitably high purity and suitably low higher-order aggregate such to be able to compare the effects of these molecules in complex primary immune cell assays in order to exemplify this design format. Equally important was a need to avoid high levels of LPS. In both regards, it was therefore encouraging that all multitarget, multivalent immunoconjugates we designed were produced successfully with the aid of standard platform antibody expression, two-step purification, and supporting interim and final QC analytical methodology. See FIG. 6 for a summary of the QC batch profiles generated. These materials were employed in the Examples and studies outlined elsewhere below.

### Example 5: Comparative Binding Affinity Studies of Multivalent Immunoconjugates Targeting IL-15 Receptor Beta (CD122) and NKG2D

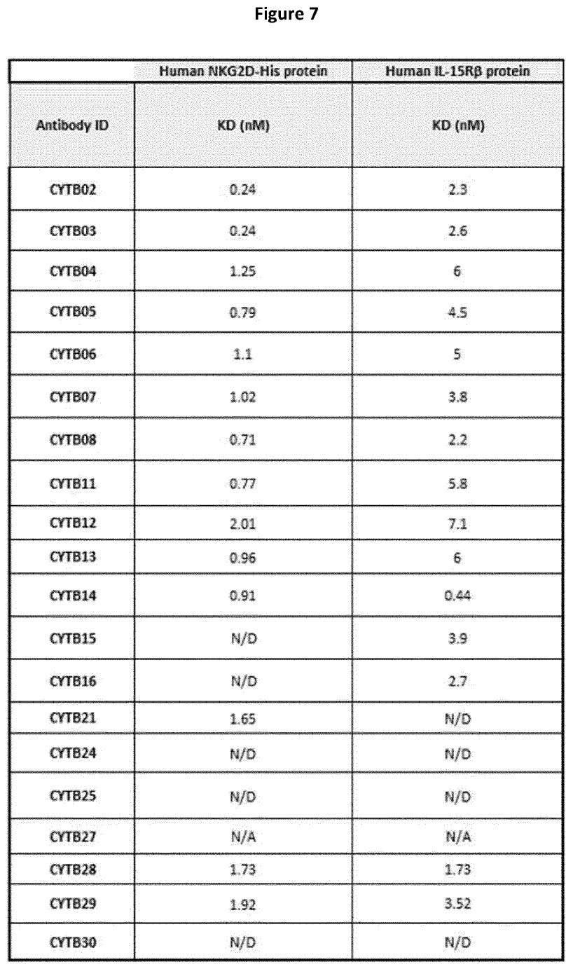

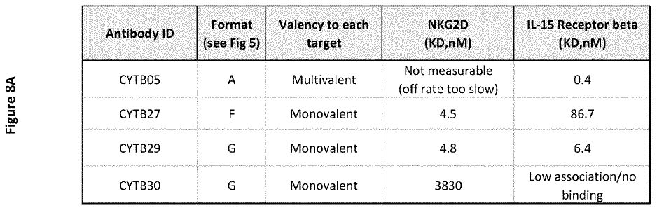

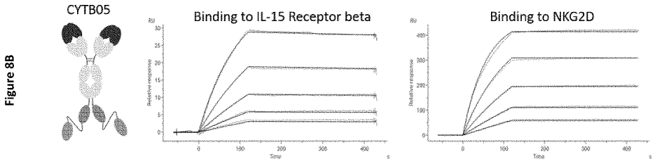

After the successful production of the multivalent immunoconjugates and associated controls and comparators, the kinetic parameters and affinity values (KD) were determined using standard SPR methodology. In brief, the test molecules were first immobilized on a goat anti-human IgG (Fc specific) antibody plated on a Biacore CM5 Series S sensor chip (CFJB1172, Cytiva Cat No. 29149603) by injection at 5 nM at flow rate 30 ul/min. Once captured, binding affinity to target antigens was undertaken using the following recombinant proteins (i) Human his-tagged NKG2D/CD314 (Acro Biosystems, NKD-H5245 and (ii) Human IL-15R beta/CD122 Protein, His Tag SPR-verified (Acro Biosystems CD2-H5221). Binding to each antigen was determined using five-step, two-fold serial dilutions of target antigen starting at 50 nM through to 3.125 nM. Injections flow rates were at 30 ul/min with contact times of 60 seconds and dissociation time of 600 seconds. Standard regeneration solution was injected twice between samples followed by an injection of 1×HBS-EP+pH7.4 biacore buffer (Cytiva, Cat No. BR-100669) at a flow rate of 30 ul/min, contact time 30 seconds. Due to a complex dissociation profile observed for many of the molecules bound to NKG2D target protein, besides the classical Langmuir 1:1 model, the curves were also fitted with a two-state reaction model. The quality of the fits was assessed by visual inspection. Nevertheless, because the resulting KD values calculated by both approaches were very similar, only the 1:1 KD values are reported here in Table 5 (see also FIG. 7):

TABLE 5

| | Human NKG2D-His protein | Human IL-15Rβ protein |
|---|---|---|
| Antibody ID | KD (nM) | KD (nM) |
| CYTB02 | 0.24 | 2.3 |
| CYTB03 | 0.24 | 2.6 |
| CYTB04 | 1.25 | 6 |
| CYTB05 | 0.79 | 4.5 |
| CYTB06 | 1.1 | 5 |
| CYTB07 | 1.02 | 3.8 |
| CYTB08 | 0.71 | 2.2 |
| CYTB11 | 0.77 | 5.8 |
| CYTB12 | 2.01 | 7.1 |
| CYTB13 | 0.96 | 6 |
| CYTB14 | 0.91 | 0.44 |
| CYTB15 | N/D | 3.9 |
| CYTB16 | N/D | 2.7 |
| CYTB21 | 1.65 | N/D |
| CYTB24 | N/D | N/D |
| CYTB25 | N/D | N/D |
| CYTB27 | N/A | N/A |
| CYTB28 | 1.73 | 1.73 |
| CYTB29 | 1.92 | 3.52 |
| CYTB30 | N/D | N/D |

### Observations of Note:

CYTB15 (and CYTB24) affinity measurements: The CYTB15 multivalent immunoconjugate and associated control antibody (CYTB24) contains a previously described anti-NKp30 Vh/Vl (see WO2022046922) in place of anti-NKG2D Vh/Vl pairs. Hence CYTB15 and CYTB24 does not bind NKG2D. Therefore binding to NKG2D was not determined.

CYTB16 (and CYTB25) affinity measurements: The CYTB16 multivalent immunoconjugate and associated control antibody (CYTB25) contains a previously described anti-NKp46 Vh/Vl (see WO2017165464) in place of anti-NKG2D Vh/Vl pairs. Hence CYTB16 and CYTB25 does not bind NKG2D. Therefore binding to NKG2D was not determined.

CYTB27 affinity measurements: Whilst this molecule is monovalent to both NKG2D and IL-15 receptor targets, this format lacks a standard IgG architecture and so does not fit with this SPR assay (use of a goat anti-human IgG to capture the molecule on the CHIP surface). Hence affinity to either target was not determined by this assay. However alternate assay binding studies with CYTB27 have been undertaken later herein (see Example 6 below).

CYTB30 affinity measurements: This 3-chain comparator is monovalent to both IL-15 receptor and NKG2D. FIG. 5 Format G herein also provides a schematic representation. However, in our studies no KD could be determined to the CD122 IL-15 receptor target due to low association/no binding. This is likely due to the intentional mutations (N4D, N65D) of the IL-15 cytokine ORF included in this molecule—so designed to encode a reduced activity IL15 ‘mutein’. Additionally, we could not detect any binding of CYTB30 to NKG2D in our assays either—low association/no binding was observed. This would suggest the lower affinity IL-15 N4D, N65D mutein contained within this molecule also hinders NKG2D engagement. Given NKG2D target binding in this molecule is via a separate Vh/Vl monovalent binding domain, it is unexplained how the IL-15 mutein also impacts NKG2D engagement. It may be due to a gross steric effect of the mutein sequence on overall molecule structure.

CYTB29 affinity measurements: Because CYTB30 contains a mutein IL-15, we explored if a better comparator of this 3-chain monovalent format to both targets would be to create a version of CYTB30 containing a wild-type IL-15. We do not believe this wild-type IL-15 version of the molecule has ever previously been designed/generated. Unlike CYTB30 (see above), an affinity to both NKG2D and CD122 could be recorded for this molecule. Hence this molecule, which appears monovalent to both NKG2D and IL-15 receptor CD122, is a useful tool comparator in our studies focused on the effects conferred by multivalent immunocytokines of this invention.

### Example 6: Superior Avidity of Multivalent Immunoconjugates Targeting IL-15 Receptor Beta (CD122) and NKG2D

Alongside measuring standard binding affinities of all molecules, we also wanted to explore if the multivalent immunoconjugates we had designed exhibited superior avidity to the targets relative to monovalent comparator molecules. In order explore avidity binding effects (rather than affinity), a different assay orientation was employed. Specifically, the recombinant target human antigens (NKG2D-His or IL-15 receptor beta-His antigens, see Example 5) were immobilized on CM5 Series S sensor chips (Cytiva, catalogue number 29149603). Control antibodies were then employed to validate the antigen-bound sensor chips (not shown). Irrelevant mAbs were employed to determine and subtract background. As previously (Example 5), standard regeneration solutions and buffers were employed. To determine and compare avidity effects, test molecules were the injected in stepwise serial dilutions and sensogram responses generated.

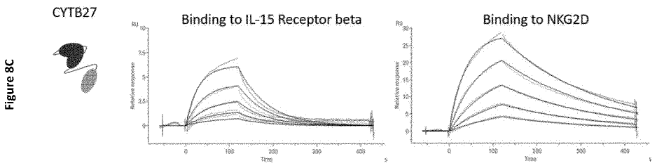

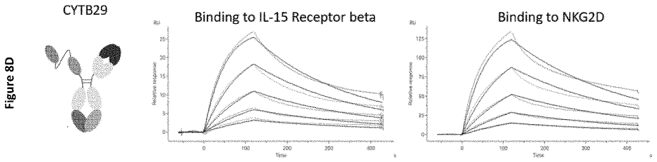

The results of these studies (see Table 6 and FIG. 8) demonstrate the superior binding effects associated with the multivalent format. Specifically, when an example multivalent immunoconjugate molecule of the invention (CYTB05) was compared to monovalent comparators (CYTB27, CYTB29 and CYTB30), the strength of binding to both targets was vastly improved. Indeed, the binding strength to NKG2D was so significantly improved for CYTB05, and the dissociation kinetics (‘off-rates’) so much slower, that a KD value could not be confidently determined for CYTB05 in this assay set-up.

TABLE 6

| | Format | Valency | | IL-15 |
|---|---|---|---|---|
| Antibody | (see | to each | NKG2D | Receptor beta |
| ID | FIG. 5) | target | (KD, nM) | (KD, nM) |
| CYTB05 | A | Multivalent | Not measurable | 0.4 |
| | | | (off rate too slow) | |
| CYTB27 | F | Monovalent | 4.5 | 86.7 |
| CYTB29 | G | Monovalent | 4.8 | 6.4 |
| CYTB30 | G | Monovalent | 3830 | Low association/ |
| | | | | no binding |

Of the monovalent comparators in this study, of note firstly is that both CYTB27 and CYTB30 exhibited very weak binding to IL-15 receptor beta targets. The reasons for these very weak binding characteristics are likely different. Firstly, CYTB27 is a single-chain molecule monovalent to both targets. CYTB27 is first described in Example 4 and 5 above. Whilst there are significant caveats associated with drawing conclusions about the function of this molecule (given the poor QC profile), the lower affinity exhibited to the IL-15 receptor beta target maybe due to the lack of a sushi domain component. Second, CYTB30—first introduced in Example 5—also exhibits extremely weak (if any) binding to IL-15 receptor beta in this assay. This may be due to the use of a significantly inferior IL-15 mutein in this molecule as also discussed in Example 5 previously.

The final comparator CYTB29 (also first described in Example 4 and 5) did at least function in this assay and bind both NKG2D and IL-15 receptor beta targets. Even still, the binding profile of this molecule to both targets is clearly significantly inferior to that exhibited by the multivalent immunocytokine format (CYTB05).

In conclusion, these binding studies clearly highlight the significantly enhanced binding properties of the multivalent immunoconjugate format. These findings encouraged us further to explore the effects conferred by this molecular format in target human immune cells of interest.

### Example 7: Optimal Multivalent Formatting; Sequence Order Independent Dual Target Co-Engagement

Alongside a study on avidity, we also considered it important to further dissect our design format. Specifically, we wanted to confirm that our preferred format was capable of engaging both targets simultaneously and irrespective of which target is engaged first thereby exhibiting sequence order independent dual target co-engagement. Furthermore, whilst our preferred design format describes heavy chain and light chain C-terminal attachment to an intact IgG, it is also possible to attach entities to the N-terminal of an intact IgG. Hence, we wanted to compare and contrast N-terminal versus C-terminal sushi-IL15 attachments to an IgG. For this study on dual target engagement, we therefore compared CYTB05 (heavy chain C-terminally attached; FIG. 5 Format A), CYTB11 (light chain C-terminally attached; FIG. 5 Format C), with CYTB07 (N-terminally attached, FIG. 5 Format B). Also included in this study were the aforementioned monovalent comparators CYTB29 and CYB30 (3-chain, Figure Format G). Finally, we also wished to explore the role of the sushi domain in our C-terminal attached multivalent design. Consequently, we also included CYTB14 (FIG. 5 Format D) in the study wherein the sushi domain is removed.

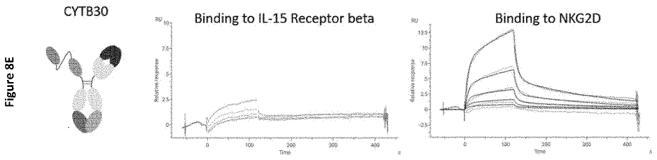

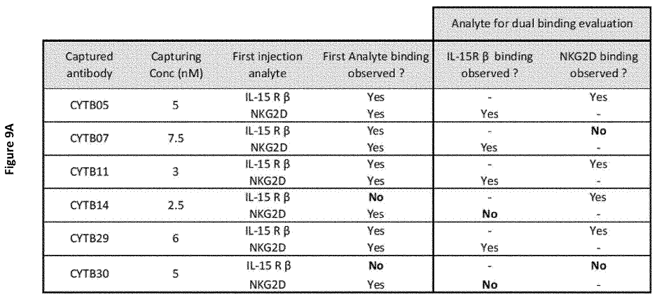

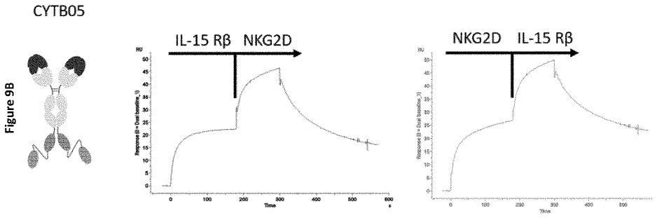

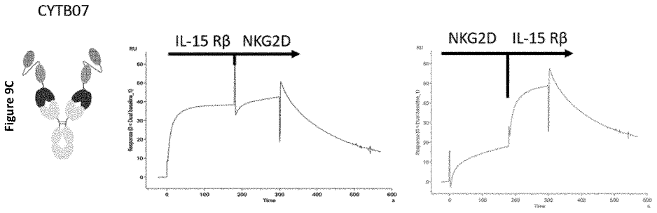

For this more complex binding assay CFJB1172 CM5 Series S sensor chips (Cytiva, cat. nr. 29149603) prepared with goat anti-human IgG for capture were used to first bind the test molecule or isotype reference control (Ultra-LEAF purified human IgG1 isotype control (Biolegend, cat. Nr. 403502) to the sensor Chip. Two-step dual injections were undertaken to explore dual binding characteristics. For the first injection, either the recombinant human IL-15 beta receptor-His protein or the recombinant human NKG2D-His antigen at 200 nM were injected on flow cell 1 (for reference subtraction) and 2 (for dual binding) at a flow rate of 30 μl/min, contact time 180 sec with no dissociation time. For the second injection, human IL-15R beta receptor-His and human NKG2D-His proteins were then injected 200 nM on flow cells 1 and 2 at flow rate of 30 μl/min, contact time 120 sec and dissociation time of 180 sec. Sensograms generated were then analysed. Response units of less 5RU were considered as no binding. The results of this study are presented in Table 7 and FIG. 9.

TABLE 7

| | | | | Analyte for dual | |
|---|---|---|---|---|---|
| | | | First | binding evaluation | |
| | Capturing | First | Analyte | IL-15R β | NKG2D |
| Captured | Conc | injection | binding | binding | binding |
| antibody | (nM) | analyte | observed? | observed? | observed? |
| CYTB05 | 5 | IL-15 R β | Yes | — | Yes |
| | | NKG2D | Yes | Yes | — |
| CYTB07 | 7.5 | IL-15 R β | Yes | — | No |
| | | NKG2D | Yes | Yes | — |
| CYTB11 | 3 | IL-15 R β | Yes | — | Yes |
| | | NKG2D | Yes | Yes | — |
| CYTB14 | 2.5 | IL-15 R β | No | — | Yes |
| | | NKG2D | Yes | No | — |
| CYTB29 | 6 | IL-15 R β | Yes | — | Yes |
| | | NKG2D | Yes | Yes | — |
| CYTB30 | 5 | IL-15 R β | No | — | No |
| | | NKG2D | Yes | No | — |

These results demonstrate that only when the IL-15 sushi domain is fused at the C-terminus of the heavy chain or light chain does one see sequence order independent binding and co-engagement of both CD122 and NKG2D. In contrast, N-terminal attachments where not able to engage NKG2D if sushi-IL15 domain first bound CD122. This study also demonstrates the importance of the sushi domain given no CD122 binding was observed when this domain was removed.

### Example 8: a Comparative Summary of the Differing Binding Profiles

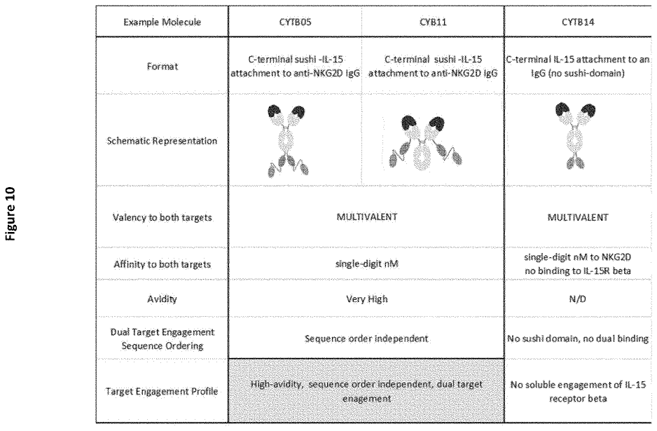

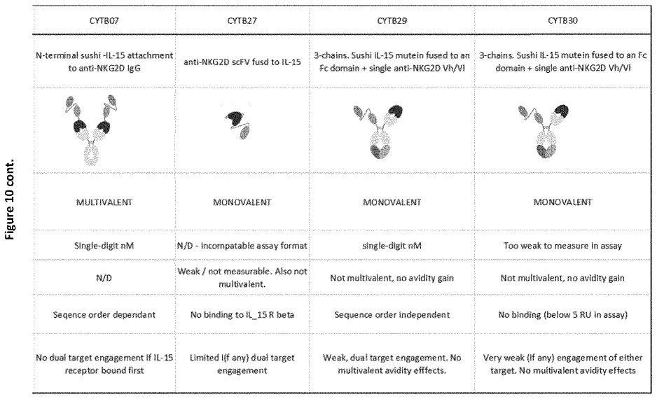

An overall summary of the comparative binding properties discovered in Examples 5, 6 and 7 are presented in Table 8 and FIG. 10. As indicated, of particular interest are the high-avidity, sequence order independent, dual target co-engagement profiles observed by multivalent formats.

TABLE 8

| Example | | | | | | | |
|---|---|---|---|---|---|---|---|
| Molecule | CYTB05 | CYB11 | CYTB14 | CYTB07 | CYTB27 | CYTB29 | CYTB30 |
| Format | C-terminal | C-terminal | C-terminal IL-15 | N-terminal | anti-NKG2D scFV | 3-chains. | 3-chains. |
| | sushi -IL-15 | sushi -IL-15 | attachment to | sushi -IL-15 | fused to IL-15 | Sushi IL-15 | Sushi IL-15 |
| | attachment | attachment | an IgG (no sushi- | attachment | | mutein fused | mutein fused |
| | to anti- | to anti- | domain) | to anti- | | to an Fc | to an Fc |
| | NKG2D IgG | NKG2D IgG | | NKG2D lgG | | domain + | domain + |
| | | | | | | single anti- | single anti- |
| | | | | | | NKG2D Vh/VI | NKG2D Vh/VI |

| | | | | | | |
|---|---|---|---|---|---|---|
| Valency to | MULTIVALENT | MULTIVALENT | MULTIVALENT | MONOVALENT | MONOVALENT | MONOVALENT |
| both targets | | | | | | |
| Affinity to | single-digit nM | single-digit nM | Single-digit nM | N/D - incompatible | single-digit nM | Too weak to |
| both targets | | to NKG2D | | assay format | | measure in assay |
| | | no binding to | | | | |
| | | IL-15R beta | | | | |
| Avidity | Very High | N/D | N/D | Weak/not | Not multivalent, | Not multivalent, |
| | | | | measurable. Also | no avidity gain | no avidity gain |
| | | | | not multivalent. | | |
| Dual Target | Sequence order | No sushi domain, | Sequence order | No binding to | Sequence order | No binding |
| En-gagement | independent | no dual binding | dependant | IL_15 R beta | independent | (below 5 RU |
| Sequence | | | | | | in assay) |
| Ordering | | | | | | |
| Target | High-avidity, sequence | No soluble | No dual target | Limited i(f any) | Weak, dual | Very weak (if any) |
| Engagement | order independent, | engagement of | engagement if | dual target | target | engagement of |
| Profile | dual target engagement | IL-15 receptor | IL-15 receptor | engagement | engagement. | either target. No |
| | | beta | bound first | | No multivalent | multivalent |
| | | | | | avidity | avidity effects |
| | | | | | effects. | |

### Example 9: Multivalent Immunoconjugate Design Options and Impact on Functionality

Given the uniquely different binding profiles of multivalent immunoconjugate designs, one question to consider is how might this design effect human primary immune cells?Specifically, could attaching anti-NKG2D binding domains and sushi-IL-15 binding moieties in the manner we describe augment immune cell functionality in some way? To explore this further, in an arm of one experiment, we compared and contrasted the multivalent immunoconjugate exemplars with the sushi-IL-15 complex and anti-NKG2D building blocks. For this study, healthy human PBMCs were first seeded at 200,000 cells per well in flat-bottomed TC plates with X-VIVO™ cell culture media (Lonza, Basel, Switzerland) containing 1% penicillin/streptomycin and 2 nM Glutamax™ (ThermoFisher, Waltham USA). Next the test articles as indicated (see FIG. 11) were added to the seeded wells. 24 hours later, any stimulatory effects on the gated NK cells were measured with aid of antibody staining and flow cytometry-based analysis. Specifically, NK cells were identified using a CD56+, CD3−, TCR γδ−, CD14− gate and their activation status measured using a CD69+/CD107a+ double positive gate. The results of this experiment are shown in FIG. 11 and highlight the remarkable impact of molecule design on conferred effects. Specifically, these results highlight the following discoveries:

FIG. 11 (A) Results and Discoveries:
When added as a soluble agent, the anti-NKG2D IgG1 antibody test article (termed CYTB21) had a negligible impact on the numbers of CD69+/CD107a+ double positive NK cells. This aligns with prior conventional thinking whereby as soluble entities anti-NKG2D antibodies are not agonists.
When added as a soluble agent, the sushi-IL15 complex test article (comprising Human IL-15Ralpha Sushi domain—Miltenyi catalogue no. 130-104-920 preincubated with Human IL-15—Miltenyi catalogue no. 130-095-760) exhibited only a modest impact on the numbers of CD69/CD107a double-positive NK cells.
Moreover, when these same two test articles (CYB21 and the sushi/IL15 complex) were added in combination, again there was only a modest impact on CD69+/CD107a+ double-positive NK cell number.
In contrast, when the multivalent CYTB05 test article was added, a remarkable and titratable synergistic increase in the numbers of CD69+/CD107a+ double-positive NK cells was observed. For reference CYTB05 comprises the same anti-NKG2D IgG1 moiety as CYTB21 but with sushi-IL15 domain complexes attached to the C-termini. Also note and of direct relevance, in similar but separate experiments we have also observed similarly favourable and titratable effects for other variants of the CYTB05 format A design wherein differing anti-NKG2D Vh/Vl domains were trialled. Namely CYTB02 and CYTB03 (data not shown).

FIG. 11 (B) Results and Discoveries:

To extend this remarkable finding further, FIG. 11 (B) includes an overlay of additional test articles explored in this study arm. These additional molecules highlight further design discoveries as follows:

First, the effects conferred by CYTB12 are also now presented. We observed this molecule to induce a similar titratable increase on the numbers of CD69+/CD107a+ double-positive NKs cells to that of CYTB05 (and CYTB02/CYTB03). For reference, CYTB12 comprises the same format as CYTB05 but with a differing linker separating the heavy chain C-termini with the sushi domain.

Secondly, and in direct contrast to the desirable effects conferred by CYT05 and CYTB12, we noted that CYTB07 induces no such titratable effect on the numbers of double-positive CD69+/CD107a+ immune cells. For reference, CYTB07 comprises the same anti-NKG2D IgG as CYTB05 and CYTB12, however the sushi-IL15 domains are attached to the N-termini of the IgG rather than at the C-termini.

Finally, we also noted that CYTB14 induced negligible effects. For reference, CYTB14 is the same format as CYTB05 but lacks the sushi domains.

To conclude, in this example and for the first time, we present the effects conferred by multivalent immunocytokines as described herein on gated human NK cells. These findings are remarkable. First they demonstrate that when the sushi-IL15 complex is attached to the C-termini of an anti-NKG2D antibody, we see a significant, synergistic and titratable stimulatory effect on immune cells as measured by the numbers of double-positive CD69+/CD107a+ NK cells. Mechanistically these findings suggest that co-attaching the two independent molecular building blocks in a particular way is fundamentally important for synergy to be achieved. By way of example, such molecules may thus be useful when stimulating NK cells to control or alleviate the sign and symptoms of disease in cancer patients.

Additionally of interest is the observation that differing C-terminal linkers can be accommodated in molecule design. Often there is a need to separate differing functional domains with flexible linkers. Typically, the length and indeed the need for these linkers is explored empirically. Nevertheless, we at least wanted to confirm that multivalent immunocytokines of this invention were not dependent on a single type or length of linker. Hence, we designed CYTB05 with a gly/ser based linker separating the sushi-domain and the C-termini of the heavy chain, whilst we designed CYTB12 with a TVAAP linker at this junction. It is thus very encouraging to see that multivalent immunocytokines can accommodate differing linkers. During the normal course of product development, further empirical work is typically undertaken to determine which linker is most preferred from a stability/physical chemistry perspective

Furthermore, we have also observed similar effects with alternate molecules of the same design as CYTB05 but with differing anti-NKG2D Vh/Vl binding domains. This suggests this approach can accommodate a variety of differing anti-NKG2D Vh/Vl based binding domains. The NKG2D Vh/Vl employed in CYB05 is known in an IgG format to suppress NKG2D agonism. Likewise for the anti-NKG2D Vh/Vl domains of CYB02 and CYTB03—they have also previously been described as antagonistic binders when employed as soluble IgG agents. The fact that we see agonism with soluble multivalent immunocytokine formats containing these very same anti-NKG2D Vh/Vl domains is thus very encouraging. This topic is further discussed in Example 14.

In stark contrast, during this study we also discovered that when the sushi-IL-15 complex is N-terminally attached (instead of C-terminally attached), the resulting molecule is less able to induce significant increases in CD69+/CD107a+ double-positive NK cells. This remarkable finding directly correlates with the sensogram binding observation of Example 7 wherein N-termini attachments where unable to co-engage NKG2D target if IL-15 sushi-domain bound first. These combined findings therefore suggest a mechanism wherein sequence order independent co-engagement to both targets is important for functionality of these multivalent immunoconjugates. Whilst not bound by the theory we believe such co-engagement may confer dual agonism effects via both the CD122 and NKG2D signalling axes.

Finally, it is also of interest to note that deletion of the sushi-domain (CYTB14) also reduces molecule function in this assay. This supports the concept that the sushi-domain is an important trans-presenter of IL-15 cytokine to CD122.

### Example 10: Comparison of Multivalent Immunocytokines and Monovalent Immunocytokines—Surface Marker Upregulation

The synergistic effects conferred by the multivalent immunocytokines described in Example 9 were of interest. However, another important consideration is the need for a multivalent format instead of a monovalent format. Hence in one arm of a separate experiment, we specifically explored the effects of multivalency. For this comparative study, we first seeded fresh human PBMCs cultures in the same manner as described in Example 9. Test articles were then added prior to flow-based gating and surface-marker based analysis at 24 hours. For this analysis, four main gated populations are presented as follows: NK cells (gated as CD56+, CD3−, TCRγδ−, CD14−) NKT cells (gated as CD56+, CD3+, TCRγδ−, CD14−), TCRγδ T-cells (gated as CD3+, TCRγδ+, CD56+/−, CD14−) and CD8 T cells (gated as CD8+, CD3+, +, TCRγδ−, CD56−, CD14−). The test articles in this study included CYTB05 and CYTB11—as exemplars of the multivalent immunocytokine format with sushi-IL-15 complexes attached to the C-termini of the heavy chain or light chain respectively. Also included in this assay were the two building block component parts as employed in example 9—namely an anti-NKG2D IgG (CYTB21) and a sushi-IL15 complex. And finally, also compared in this study were CYTB29 and CYTB30. These molecules were first described in Example 5—they are monovalent to both targets and contain either a wild-type or disabled (mutated) versions of the IL-15 cytokine respectively.

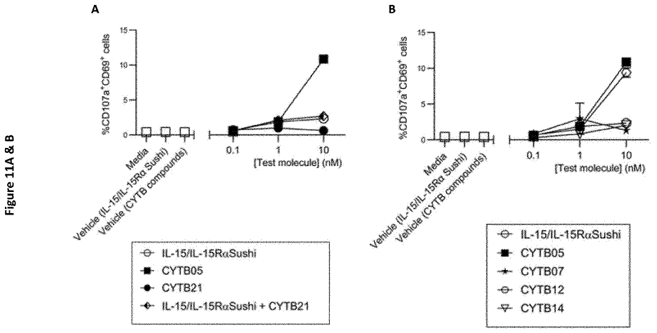

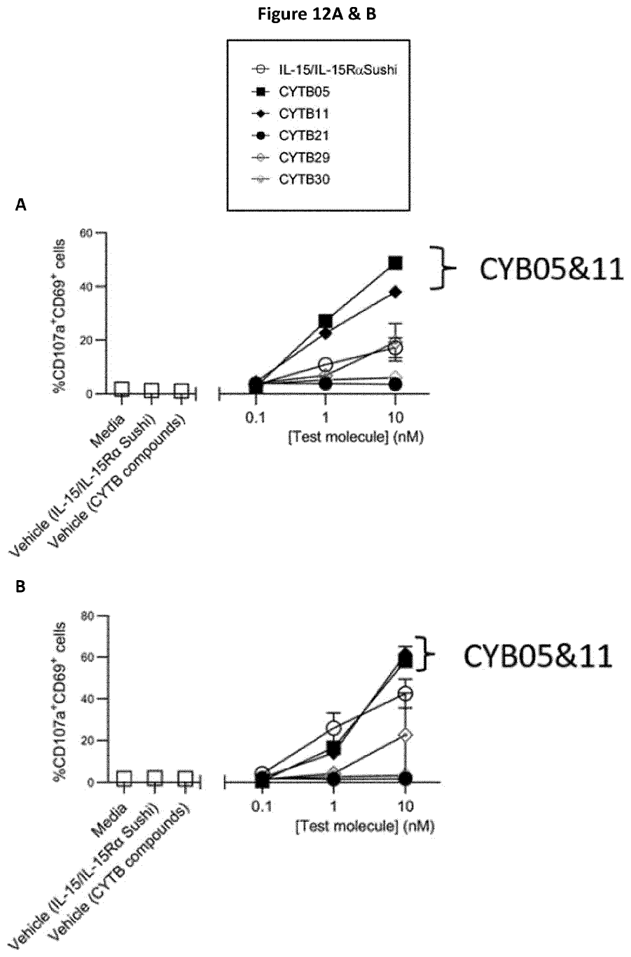

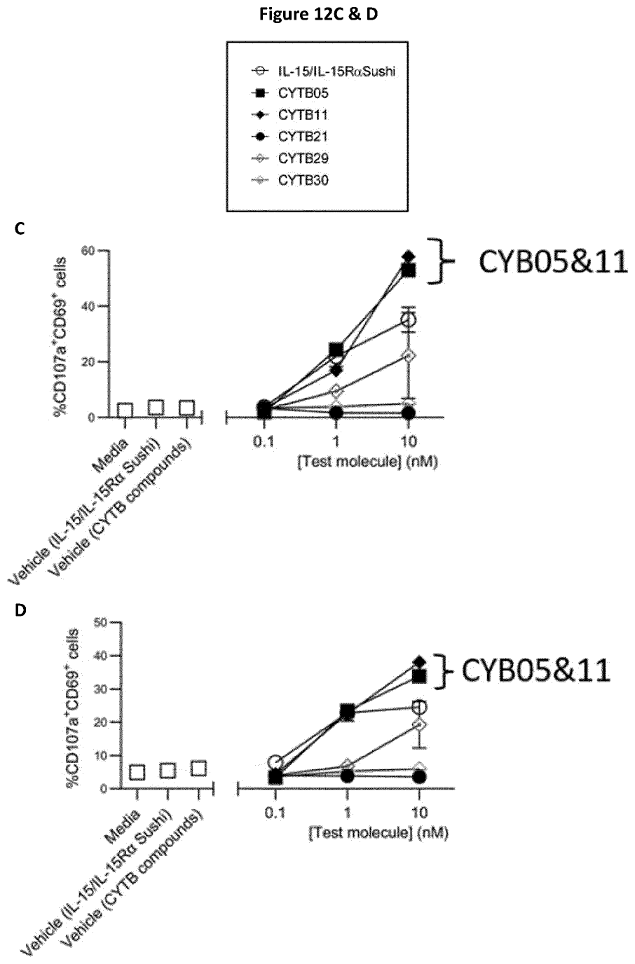

The results of this experiment were also remarkable. They are presented in FIG. 12. First, they reveal that the multivalent immunocytokines CYTB05 and CYTB11 outperformed all other comparators in the multiple effector sub-classes analysed. Specifically, these two molecules induced more CD69+/CD107a+ double positive populations in NK cells, NKT cells, gamma delta T-cells, and CD8 cells. This discovery therefore enforces and extends the findings of Example 10. Specifically, it demonstrates that attaching the sushi-IL15 domain to the C-termini of either the light chain or heavy chain is an effective design format. This again aligns with the sensogram data (Example 7) which demonstrates these two formats also exhibit sequence order independent binding co-engagement of both targets. Next, and equally remarkably, the results of this example highlight that the monovalent format represented by CYTB29 and CYTB30 performed less effectively than either the standard control sushi-IL15 complex alone or control anti-NKG2D IgG1 (in the case of CYTB30). It is interesting to speculate why this is observed. For CYTB30 molecule we had previously shown that the mutations introduced into the IL-15 cytokine resulted in a defective molecule with a severely impacted binding profile to both CD122 and NKG2D (see example 5). However, and partly because of this, we also trialled CYTB29 which contains a wild-type IL-15 cytokine and which we had previously shown monovalent to both NKG2D and CD122. One can therefore only speculate quite why CYTB29 was observed inferior even to a simple sushi-IL15 complex in activating NK, NKT, gamma delta T-cells or CD8 T-cells. Perhaps it is because a monovalent NKG2D binding format acts as a hinderance or blocker in some way thereby also dampening down any sushi-IL15 conferred effects. This would align with conventional thinking wherein anti-NKG2D antibodies are considered antagonistic as soluble agents. Regardless, once again our multivalent format was highly effective at activating many, if not all the main effector immune classes associated with the killing or controlling cancers cells.

### Example 11: Comparison of Multivalent and Monovalent Immunocytokines—Immune Cell Proliferation

To extend the findings of Example 10 further we also explored the effects the same test articles conferred on proliferation of the same desired sub-gates. Consequently, alongside sacrificing cell culture wells for analysis at 24 hours to generate the results of example 10, replicate parallel wells were cultured for an additional 5 days. This resulted in a total incubation time with test articles of 144 hours (6 days). These extended cultures wells were then sacrificed, and cells counts of the same respective sub-gates as per Example 10 recorded.

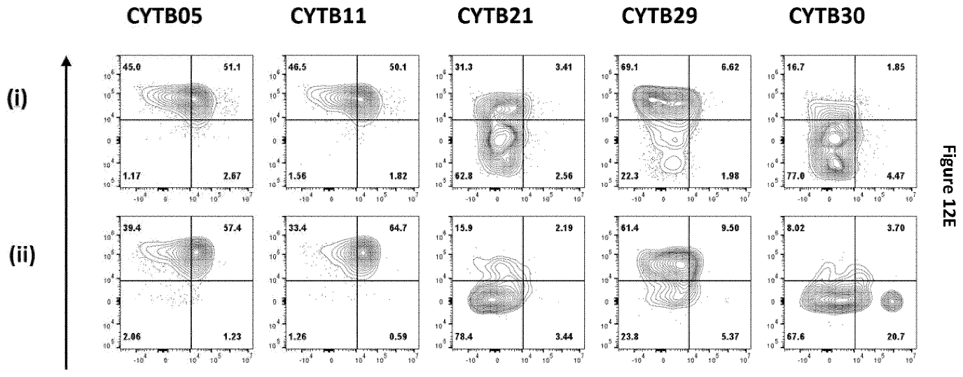

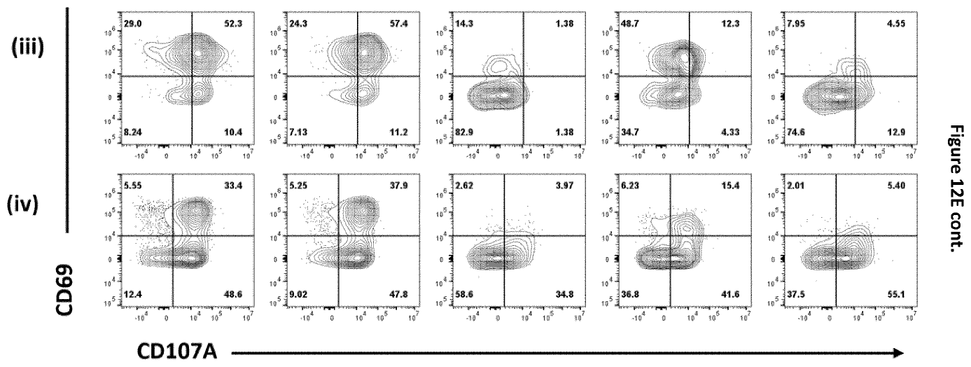

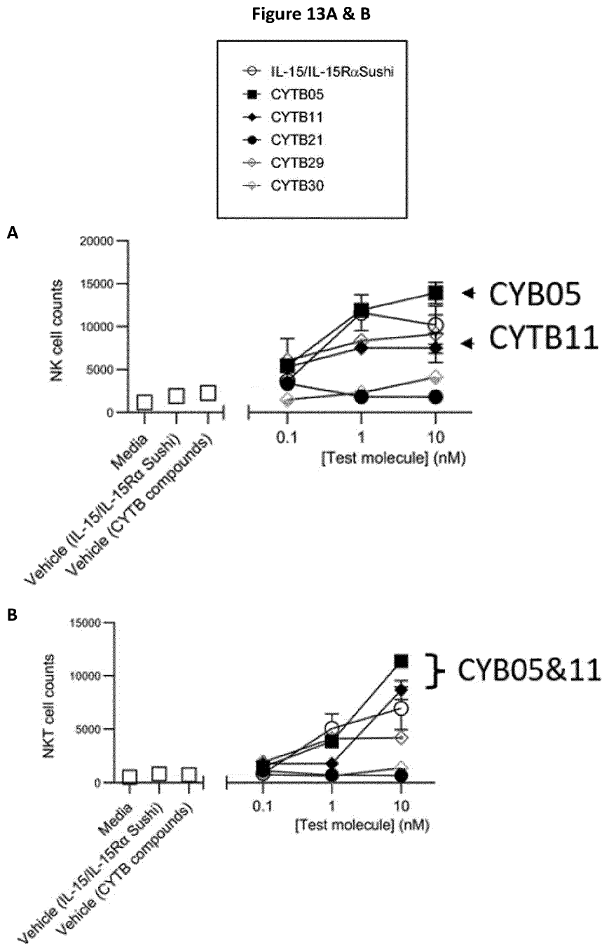

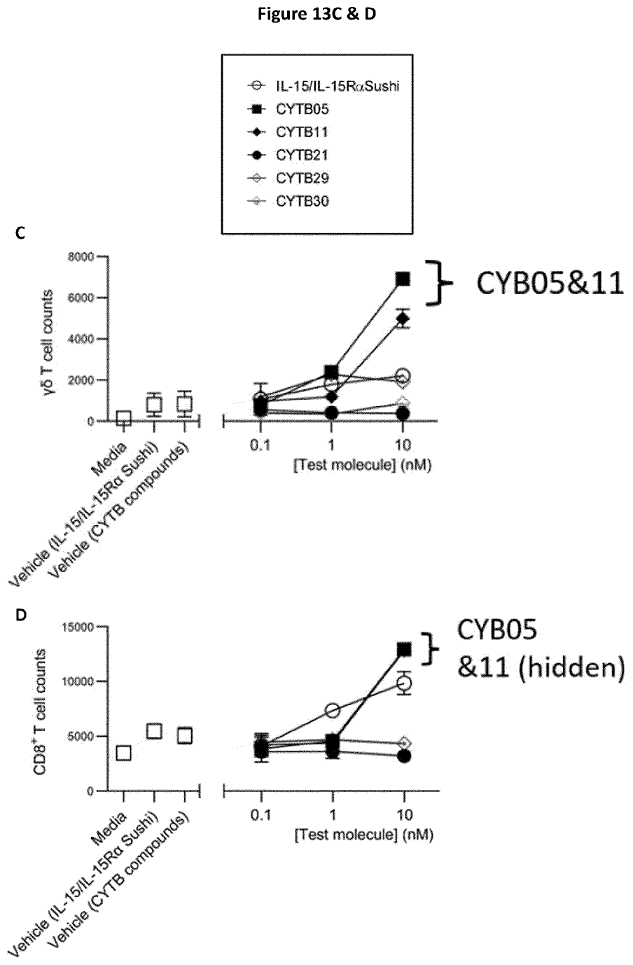

The results of this study are presented in FIG. 13 and align with the Example 10 results. Once again the exemplar multivalent CYTB05 and CYTB11 molecules out performed all other test article in multiple sub-gates with marked increase in all sub-gates noted. Once again, the monovalent comparators were inferior and indeed also inferior to sushi-IL15 complexes in most instances. The combined findings of Example 10 and 11 therefore confirm that multivalent immunocytokines of the invention maybe ideal for use in the treatment of cancers given their capacity to functionally enhance key immune cell effector classes (NK cells, NKT cells, gamma-delta T-cells and CD8) associated with controlling and alleviating the signs and symptoms of the disease.

### Example 12: Multivalent Immunocytokines—Differing Stress-Receptor Targeting Strategies

As discussed earlier, NKG2D is one of a number of stress sensing receptors present on immune cells such as NK and gamma-delta T-cells. Example such receptors include NKp30, NKp44, NKp46 and DNAM-1. Like NKG2D, these receptors are believed to bind cell surface ligands upregulated on infected, stressed, or diseased cells resulting in receptor cross-linking and signalling. Hence, we wanted to explore if our findings associated with multivalent immunocytokines could be extended to variant molecules that target stress-sensing receptors aside NKG2D. Consequently, we designed and generated molecules that mirrored the CYTB05 format but which replaced its anti-NKG2D Vh/Vl binding domains with anti-NKp30 or anti-NKp46 Vh/Vl binding domains. The resulting molecules were termed CYTB15 and CYT16 respectively. As additional controls for this study we also generated cognate standard IgG1 with either NKp30 and NKp46 Vh/Vl binding domains. These controls were named CYTB24 and CYTB25 respectively. We then explored and compared the ability of these molecules to induce the desired double-positive CD69+/CD107+ phenotype in the same way as observed for CYTB05.

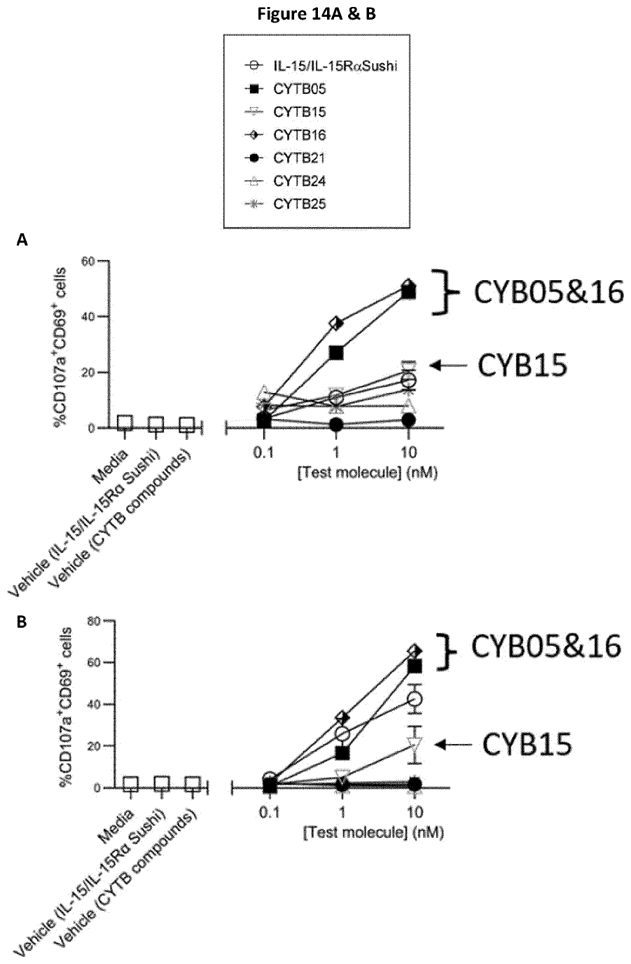

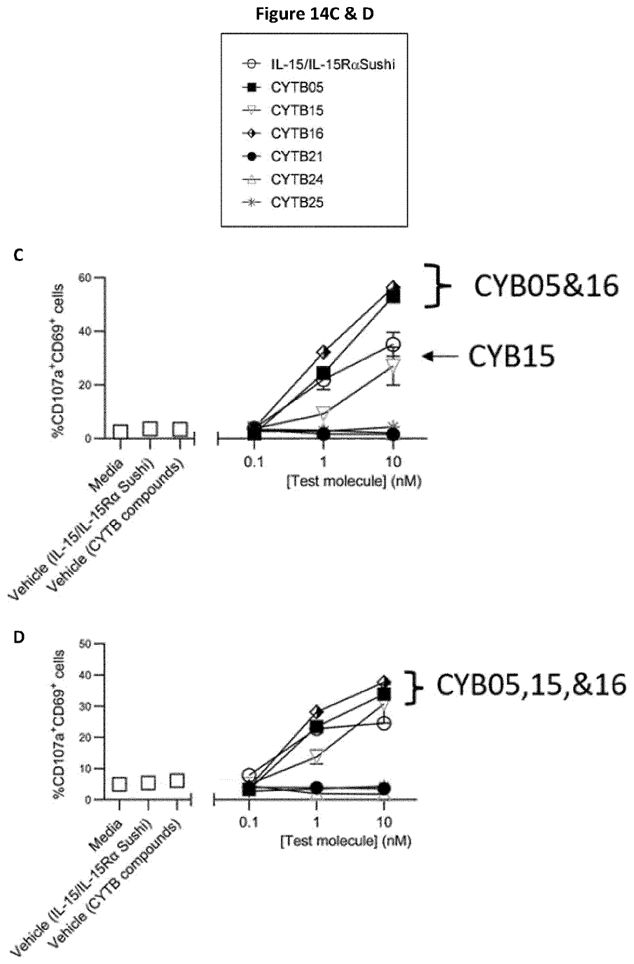

The results of this study are presented in FIG. 14. Firstly, they demonstrate the remarkable findings associated with multivalent immunocytokines targeting NKG2D can be replicated if the anti-NKG2D Vh/Vl binding domains are substituted with anti-NKp46 Vh/Vl binding domains. Indeed, CYTB05 and CYTB16 were characterised as exhibiting equivalent desirable effects with both variant molecules inducing superior levels of the desired CD69+/CD107a+ double-positive phenotype in all four measured immune effector sub-gates. Interestingly the same superior effects were not observed with the anti-NKp30 variant molecule (CYTB15) in this experiment. Further studies would be needed to better understand the reason for this. For example, whilst the anti-NKp30 Vh/Vl domain has been previously described, further studies exploring NKp30 target engagement by this particular binding domain may be revealing. Alternatively, it may be that NKp30 engagement by these multivalent cytokines does not induce enhanced levels of the double-positive phenotype to the same extent as that observed for the anti-NKG2D and anti-NKp46 variants.

### Example 13: Multivalent Versus Monovalent Immunocytokine Induced Cytokine Induction

Alongside stimulating the proliferative and cytotoxic potential of immune cells, we also wanted to explore if our multivalent immunocytokines induced a superior, titratable, cytokine production profile in immune cells. One exemplar cytokine to study in immunotherapy research is interferon-gamma. This is a pivotal cytokine capable to activating antigen-presenting cells and cytotoxic T cells alongside inhibiting the function of certain suppressive immune cells. For these combined reasons interferon-gamma is a key cytokine which is more often than not associated with effective cancer immunotherapy and the control and elimination of tumours. Consequently, we wanted to explore if molecules of the invention could induce interferon-gamma as an exemplar favourable cytokine. For this study, we seeded human PBMCs in flat-bottom wells as previously before adding test articles. At 24 hours the culture supernatants were harvested and interferon-gamma levels determined by standard ELISA methodology. For all supernatants tested, serial dilutions were undertaken to ensure the signal was within the linear range of the ELISA assay with all dilution factors then corrected for in the final results. At least three replicate wells were measured per test article and means determined. Test articles in this study included exemplar multivalent immunocytokines CYTBD5, CYTB11 and CYTB16. As also described elsewhere herein CYTB05 and CYTB11 both target NKG2D and comprise C-terminal sushi-IL15 domains attached to the C-termini of the heavy chain or light chain respectively. In contrast CYTB16 is a variant of CYTB05 in which the anti-NKG2D Vh/Vl domains have been replaced with anti-NKp46 Vh/Vl binding domains. Also included in this study was the control sushi-IL15 complex alongside CYTB29 and CYTB30—the two monovalent immunocytokines also used elsewhere herein as comparator tools. To further aid and contrast the levels induced, all final results have been presented as fold-induction versus the vehicle (blank formulation) controls.

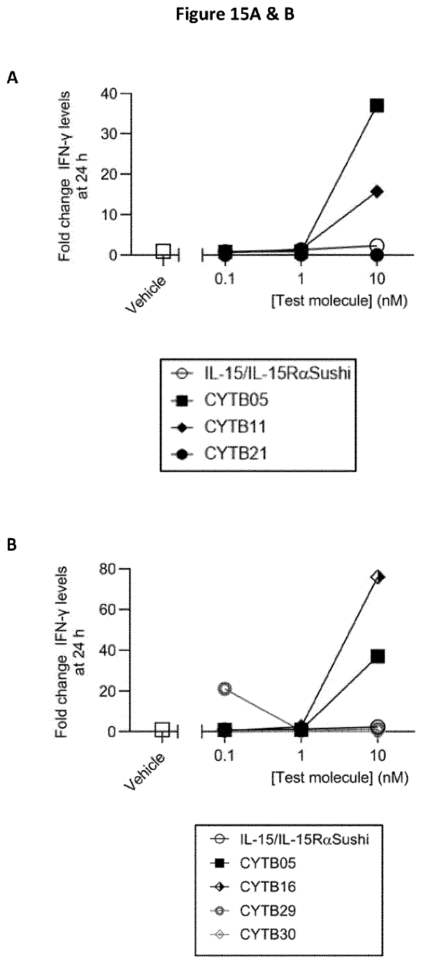

The results of this study are presented in FIG. 15. We considered these results remarkable. First, in FIG. 15 (A) the titratable effects associated with exemplar multivalent immunocytokines CYTB05 and CYTB11 are presented in which we discovered the multivalent immunocytokines induced approximately 10-40 fold more interferon gamma relative to a sushi-IL-15 complex or the control anti-NKG2D IgG1 (CYTB21) at 10 nM concentrations. (For further reference in raw μg/ml quantities, the quantities induced by between CYTB11 and CYTB12 at 10 nM concentrations equate approximately to 500 and 2000 μg/ml of interferon gamma). Equally interesting, and to extend these discoveries further, in FIG. 15 (B) the additional monovalent comparators CYTB29 and CYTB30 data in overlayed. Remarkably we discovered the CYTB29 and CYTB30 comparators were no more effective at inducing a titratable interferon-gamma response than either sushi-IL-15 complex or the anti-NKG2D IgG1 (CYTB21). Whilst additional PBMC donors will be required to characterise these findings further, this study further confirms the concept that multivalent nature of the immunocytokines is key in stimulating desired responses.

### Example 14: Enhancement of Immune-Cell Mediated Killing of Cancer Cells

Having discovered a favourable immunocytokine format, we wanted to explore if such entities were capable of enhancing immune-cell mediated killing of cancer cells. Conventionally, the use of soluble anti-NKG2D antibodies in cytotoxicity assays results in reduced cancer cell killing. Indeed, a non-exhaustive selection of studies describing these inhibitory effects is summarized in Table 9 below (with emphasis added).

TABLE 9

| Immune Cell | Effect conferred by soluble anti-NKG2D | |
|---|---|---|
| Type | antibodies. | Reference |
| NK cells | Soluble anti-NKG2D antibodies significantly | Kwong et al 2008 IBID |
| | block the cytolytic activity of NK cells | |
| | towards target cancer cells | |
| NK cells | Soluble anti-NKG2D abolish NK mediated | Bae et al 2012 |
| | killing of cancer cell targets | doi: 10.1016/j.cellimm.2012.04.011) |
| NKT cells | Soluble anti-NKG2D resulted in consistent | Kuylenstierna et al 2011 |
| | inhibition of cytolytic activity to target | doi:10.1002/eji.200940278. |
| | cancer cells | |
| CD8 cells | Soluble anti-NKG2D almost completely | Verneris et al 2004 |
| | attenuated cytotoxicity towards cancer cell | https://doi.org/10.1182/blood-2003-06-2125 |
| | targets | |
| Gamma delta | Soluble anti-NKG2D antibodies significantly | Dokouhaki et al 2013 |
| T-cells | reduced gamma delta T-cell mediated | https://doi.org/10.1002/eji.201243150 |
| | cytotoxicity. | |

Consequently, whilst we had discovered that the multivalent immunocytokines conferred favourable effects on desired immune cell compartments, we considered it important to explore if these molecules as soluble agents in cytotoxicity assays actually enhanced the ability of immune cells to kill cancer cells.

For these studies we employed a cytotoxicity assay comprising fresh human PBMCs co-incubated with the K562 target cancer cell line. K562s were chosen as they are often used as target cells when demonstrating the blocking effects associated with soluble anti-NKG2D entities. For example, see Kwong et al, Bae et al, and Kuylenstierna et al as referenced in Table 9. Specifically, the experiment involved labelling 20,000 K562 cancer cells with a CSFE dye such to be able to distinguish and measure live/dead cancer cell numbers by flow cytometry. These labelled cells were then co-cultured with human PBMCs at ratios of 1:10 (and in the same X-VIVO™ media as previously described herein). The resulting co-cultures were then incubated with CYTB05, an irrelevant isotype control IgG1 (CYTB26), or vehicle control. Because initial 24-hour time points revealed minimal effects (not shown), the assay was extended to 144 hours prior to final flow cytometry based analysis.

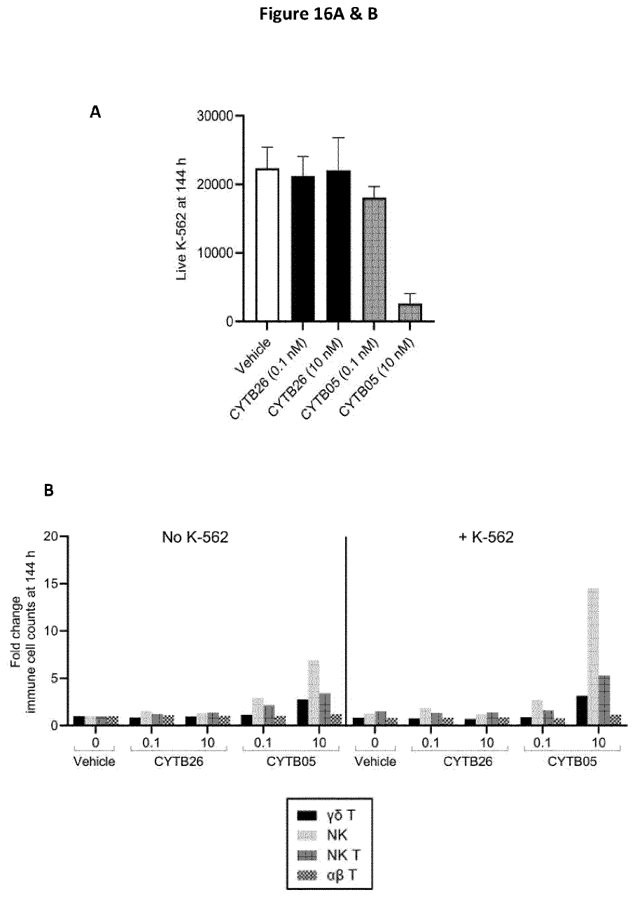

The results of this killing assay are shown in FIG. 16 A. First, they reveal that instead of blocking or reducing cell killing, the multivalent format significantly enhanced PBMC mediated cell killing of K562 cancer cells. We believe this remarkable discovery counters conventional thinking which assumes that use of soluble anti-NKG2D binding domains will block or reduce immune cell mediated killing. Second, these results also demonstrate that no tumour associated antigen (TAA) binding domain is required by our multivalent format to mediate cancer cell killing. Once again, we believe this counters conventional thinking which might assume a TAA-targeting domain is needed to bridge NKG2D-positive immune cells with the TAA-positive target tumour cells and direct tumour cell killing.

Alongside these discoveries we also decided to measure the phenotype/numbers of immune cells in the co-culture and compare this with the phenotype/numbers of immune cells in parallel PBMC cultures where no K562s cells were added. The results of this analysis are shown in FIG. 16 B. First these results confirm prior discoveries described herein showing that these molecule entities increase the numbers of NK, NKT, and gamma-delta T-cells in a dose-dependent manner. Additionally, this study also revealed that these activator effects are further augmented in the presence of tumour cells. This suggests that this multivalent format further activates immune cells when they are located proximal to cancer cells.

From these combined results, one can speculate that full activation of these desired immune cell compartments is dependent on signals provided by both (i) the cancer cells and (ii) the multivalent immunocytokines. Such conditional, cancer-cell dependant full activation of immune cells by an immunotherapeutic is extremely desirable. This discovery, combined with the fact that these molecules can enhance killing independently of TAA mediated bridging, is particularly desirable

### Example 15: Mechanisms of Immune Cell Engagement

Finally, we wanted to explore the mechanism by which our molecules engage immune cells. As outlined above, given the design of these molecules, and given that both the stress responsive receptors (NKG2D, NKp30, NKp44, NKp46) and the CD122 IL-15 receptor beta targets are often present on the same immune cell sub-types (see FIG. 3B), then determining which receptors are engaged or co-engaged by the multivalent immunocytokine format required a more complex assay design.

In brief this assay comprised the steps of (i) cooling fresh PBMCs for 30 minutes on ice to slow down receptor internalization; (ii) pre-incubation with variant unlabelled molecules in excess quantities (100 mM) to block cognate receptors and (iii) the addition of Zenon-AF647 labelled multivalent immunocytokine (CYTB05) to the now receptor-blocked cells. Subsequent analysis was then undertaken to gate the NK, NKT, gamma-delta T-cells and CD8-T cells with aid of flow cytometry as previously alongside measuring the levels of Zenon-AF647 labelled CYTB05 bound to each gated subset.

For this study, it was therefore important to employ the correct variety of unlabelled blocking molecules in the pre-incubation step. This would then allow us to employ a reductionist approach to determine which receptors mediate CYTB05 engagement on the differing immune cell sub-gates. The unlabelled blocking molecules used in the pre-incubation step included the multivariant immunocytokine itself (CYBT05), an anti-NKG2G IgG1 (CYTB21), and finally an isotype-controlled molecule containing the same C-terminal sushi-IL15 complexes but with an irrelevant Vh/Vl binding domain (CYTB17).

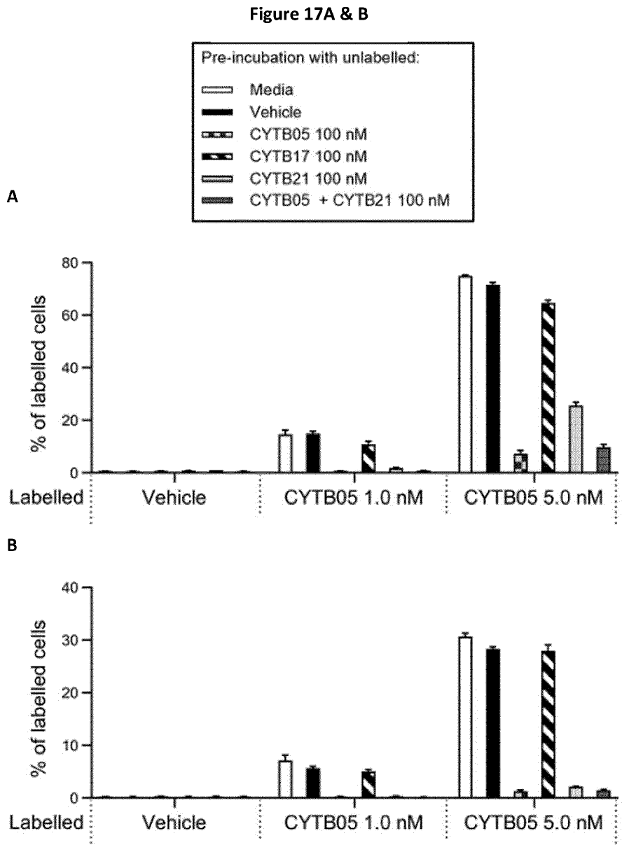

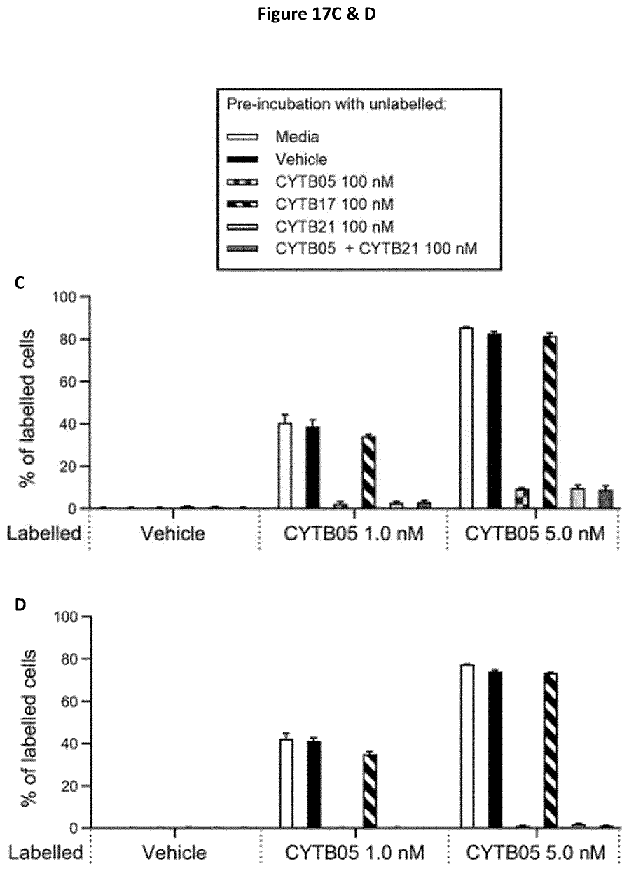

The results of this study are presented in FIG. 17. First no signal (i.e. full blocking) of labelled CYTB05 is observed for all immune sub-gates if excess unlabelled CYTB05 is first added as the pre-block. This is logical and in part validates the approach given that pre-incubated unlabelled CYTB05 at 100 nM excess should indeed pre-block all target receptors available to Zenon-labelled CYTB05 at 5 nM. This pre-blocking was further confirmed as likely 100% effective given no further reduction in signal was achieved by the addition of unlabelled CYTB21 (anti-NKG2D) on top of the unlabelled CYTB05 in a combinatorial pre-block.

Next and interestingly, for the NK sub-gated population, 100 nM (excess) unlabelled anti-NKG2D (CYTB21) alone was unable to fully block the signal generated by labelled CYTB05 at the 5.0 nM concentration. This therefore strongly suggest that the labelled CYTB05 may also binding via its sushi-IL15 domain to the IL-15 receptor expressed in NK cells. This possibility was further confirmed by the finding that pre-blocking NK cells with isotype controlled (irrelevant Vh/Vl) variant CYTB17 reduces the signal of labelled CYTB05 engagement. In this arm, the reduction in signal is likely due to the excess unlabelled CYTB17 blocking CYTB05 engagement via its comparable C-terminal sushi-IL15 domain as shared by both molecules. These combined results suggest that the CYTB05 format is capable of engaging NK cells via both its NKG2D VH/VI binding domains and its sushi-IL5 binding domains. This observed binding profile is likely driven by the sequence order independent co-engagement design of these highly avid, multivalent molecules.

For the other sub-gates (NKT, gamma delta T-cells and CD8) explored in this study, and by the same reductive reasoning, we concluded CYTB05 exhibited a modestly different binding profile. Specifically, these results suggested that for these other sub-gates, binding is driven more by anti-NKG2D domain engagement. This is because we observed that pre-blocking with unlabelled excess anti-NKG2D IgG1 (CYTB21) alone was capable of reducing the Zenon-labelled CYTB05 signal back to baseline levels.

These combined findings align fully with the previously summarised (see Example 3) expression profiles observed for the stress receptors and IL-15 receptor beta (CD122). Specifically, here we have observed that engagement by the multivalent immunocytokines can occur via both NKG2D and IL15 receptors on NK cells where high CD122 levels are observed. In contrast in cell types such as certain T-cells where CD122 levels are expressed at lower levels relative to NKG2D, these multivalent immunocytokines engage primarily via NKG2D binding domain. Nevertheless, and even though engagement is thus primarily via NKG2D receptor in the NKT, gamma-delta T-cell and CD8 T-cell compartments in this study, it would be very interesting to explore any temporal nature of this NKG2D dominant engagement profile given the dynamic nature of CD122 expression in many immune cells (e.g. see Wang et al (2021) https://doi.org/10.1126/science.288.5466.675)

### Sequence Listing Summary

The following is a brief description of the sequences provided in the accompanying sequence listing. See the sequence listing for sequence information, as well as Tables 1 to 3 for further explanation.

| SEQ ID NO(s) | Sequence description |
|---|---|
| 1 to 10 | HCDR1s of NKG2D antibodies (H1 to H10) |
| 11 to 12 | HCDR1s of NKp30 antibodies (H11 to H12) |
| 13 | HCDR1 of NKp46 antibody (H13) |
| 14 to 23 | HCDR2s of NKG2D antibodies (H1 to H10) |
| 24 to 25 | HCDR2s of NKp30 antibodies (H11 to H12) |
| 26 | HCDR2 of NKp46 antibody (H13) |
| 27 to 36 | HCDR3s of NKG2D antibodies (H1 to H10) |
| 37 to 38 | HCDR3s of NKp30 antibodies (H11 to H12) |
| 39 | HCDR3 of NKp46 antibody (H13) |
| 40 to 49 | LCDR1s of NKG2D antibodies (L1 to L10) |
| 50 to 51 | LCDR1s of NKp30 antibodies (L11 to L12) |
| 52 | LCDR1 of NKp46 antibody (L13) |
| 53 to 62 | LCDR2s of NKG2D antibodies (L1 to L10) |
| 63 to 64 | LCDR2s of NKp30 antibodies (L11 to L12) |
| 65 | LCDR2 of NKp46 antibody (L13) |
| 66 to 75 | LCDR3s of NKG2D antibodies (L1 to L10) |
| 76 to 77 | LCDR3s of NKp30 antibodies (L11 to L12) |
| 78 | LCDR3 of NKp46 antibody (L13) |
| 79 to 88 | VHs of NKG2D antibodies (H1 to H10) |
| 89 to 90 | VHs of NKp30 antibodies (H11 to H12) |
| 91 | VH of NKp46 antibody (H13) |
| 92 to 101 | VLs of NKG2D antibodies (L1 to L10) |
| 102 | VLs of NKp30 antibodies (L11 to L12) |
| 104 | VL of NKp46 antibody (L13) |
| 105 | CH1-hinge Fc domain heavy chain sequence (Human IgG1) |
| 106 | CH1-hinge Fc domain heavy chain sequence (LAGA disabled). See also SEQ |
| | ID NO: 203. |
| 107 | Fc domain light chain sequence (constant Kappa). Used in L2, L3, L4, L5, |
| | L6, L8, L9, L10, L12, L13. |
| 108 | Fc domain light chain sequence (constant Lambda). Used in L1, L7, L11. |
| 109 to 118 | Variable Heavy chains H1 to H10 of anti-NKG2D antibodies plus wild-type |
| | human IgG1 constant (without IL-15 components) |
| 119 to 120 | Variable Heavy chains H11 to H12 of anti-NKp30 antibodies plus wild-type |
| | human IgG1 constant (without IL-15 components) |
| 121 | Variable Heavy chain H13 of anti-NKp46 antibody plus wild-type human |
| | IgG1 constant (without IL-15 components) |
| 122 to 131 | Variable Heavy chains H1 to H10 of anti-NKG2D antibodies plus LAGA |
| | disabled human IgG1 constant (without IL-15 components) |
| 132 to 133 | Variable Heavy chains of H11 to H12 anti-NKp30 antibodies plus LAGA |
| | disabled human IgG1 constant (without IL-15 components) |
| 134 | Variable Heavy chain of H13 anti-NKp46 antibody plus LAGA disabled |
| | human IgG1 constant (without IL-15 components) |
| 135 to 144 | Light chains L1 to L10 of anti-NKG2D antibodies (without IL-15 |
| | components) |
| 145 to 146 | Light chains L11 to L12 of anti-NKp30 antibodies (without IL-15 |
| | components) |
| 147 | Light chain of L13 anti-NKp46 antibody (without IL-15 components) |
| 148 | Human mature IL-15 receptor alpha (CD215) sequence (not preferred) |
| 149 | IL-15 receptor alpha (CD125) sushi domain sequences |
| 150 | Extended IL-15 receptor alpha (CD125) sushi domain sequences |
| 151 | Human mature IL-15 cytokine sequence (without receptor sequence) |
| 152 | IL-15 signal peptide |
| 153 | IL-15 propeptide |
| 154 | IL-15 polypeptide sequence (Sushi + linker + IL-15 cytokine). SEQ ID NOs 206 |
| | and 207 are alternatives having different linkers. |
| 155 | IL-15 polypeptide sequence (“Extended Sushi” + linker + IL-15 cytokine) |
| 156 to 165 (and | Example full heavy chain sequences of entire immunoconjugate when |
| 188, 189, 191, 192, | fused to heavy chain at C-terminus (anti-NKG2D). 156 to 165 are H1 to |
| 195 and 200) | H10. 188 is H1 with a framework change (as in CYTB03). 189 is H6 (as |
| | used in CYTB06). 190 is H6 (as used in CYTB07). 191 is H6 (as used in |
| | CYTB08). 192 is H6 (as used in CYTB12). 195 is H6 (as used in CYTB28). |
| | 200 is H6 (as used in CYTB32). |
| 166 to 167 | Example full heavy chain sequences of entire immunoconjugate when |
| | fused to heavy chain at C-terminus (anti-NKp30, H11 and H12) |
| 168 | Example full heavy chain sequence of entire immunoconjugate when |
| | fused to heavy chain at C-terminus (anti-NKp46, H13) |
| 169 to 178 | Example full light chain sequences of entire immunoconjugate when fused |
| | to heavy chain (anti-NKG2D, L1 to L10) |
| 179 to 180 | Example full light chain sequences of entire immunoconjugate when fused |
| | to heavy chain (anti-NKp30, L11 to L12) |
| 181 | Example full light chain sequences of entire immunoconjugate when fused |
| | to heavy chain (anti-NKp46, L13) |
| 182 | Example linker sequence (e.g. used to separate either heavy or light chain |
| | constant domain from IL-15 polypeptide) |
| 183 | Example linker sequence (e.g. used to separate IL-15 receptor (Sushi) and |
| | IL-15 cytokine) |
| 184 | Example linker sequence (e.g. used to separate light chain constant |
| | domain from IL-15 polypeptide) |
| 185 | Example linker sequence (e.g. used to separate heavy chain constant |
| | domain from IL-15 polypeptide) |
| 186 | Example GlySer linker [GGGGS]nX where n is 1 to 5. See also SEQ ID NO |
| | 204, where n is 1 to 6. |
| 187 | Example GlySer linker [GGGS]nX where n is 1 to 5. See also SEQ ID NO |
| | 205, where n is 1 to 6. |
| 188 | CYTB03 full heavy chain sequence of entire immunoconjugate when fused |
| | to C terminus of heavy chain (anti-NKG2D). Employs H1 with framework |
| | change. |
| 189 | CYTB06 full heavy chain sequence of entire immunoconjugate when fused |
| | to C terminus of heavy chain (anti-NKG2D) (same as CYTB05 but with a |
| | longer linker) |
| 190 | CYTB07 full heavy chain sequence of entire immunoconjugate when fused |
| | to N terminus of heavy chain (anti-NKG2D) |
| 191 | CYTB08 full heavy chain sequence of entire immunoconjugate when fused |
| | to C terminus heavy chain (anti-NKG2D). Same as CYTB05 but with N72D |
| | mutation. |
| 192 | CYTB12 full heavy chain sequence of entire immunoconjugate when fused |
| | to C terminus heavy chain (anti-NKG2D). Same as CYTB05 but with TVAAPS |
| | linker between HC and IL-15 polypeptide |
| 193 | CYTB14 full heavy chain sequence of entire immunoconjugate when fused |
| | to C terminus heavy chain (anti-NKG2D). Same as CYTB05 but lacking |
| | Sushi domain. |
| 194 | CYTB27 monovalent scFV anti-NKG2D fused via linker to an IL-15 cytokine, |
| | without a IL-15 receptor alpha (CD215) domain |
| 195 | CYTB28 full heavy chain sequence of entire immunoconjugate when fused |
| | to heavy chain (anti-NKG2D). Same as CYTB05 but with TVAAPS linker |
| | between Sushi and IL-15 cytokine |
| 196 | CYTB29 chain 1, sushi IL15 Fc (wild type IL15) |
| 197 | CYTB29 and CYTB30 chain 2, MS Vh with Knob-in-Hole to be paired with LC |
| | SEQ ID NO: 139 |
| 198 | CYTB30 chain 1, sushi IL15 Fc (mutein IL15) |
| 199 | Light chain with IL-15 polypeptide fused to C-terminus using a TVAAPS |
| | linker, as used in CYTB13 |
| 200 | CYTB32 heavy chain with extended Sushi linker |
| 201 | Example linker sequence, e.g. as included in the CYTB32 linker between |
| | the heavy chain and the IL-15 polypeptide |
| 202 | Heavy chain variable region H1 with framework change (anti-NKG2D). |
| | Used in SEQ ID NO: 188/CYTB03 |
| 203 | CH1-hinge Fc domain heavy chain sequence (LAGA plus P329G disabled). |
| | See also SEQ ID NO: 106 for LAGA disabled. |
| 204 | Example GlySer linker sequence [GGGGS]nX, where n is 1 to 6. See also |
| | SEQ ID NO 186, where n is 1 to 5. |
| 205 | Example GlySer linker sequence [GGGS]nX, where n is 1 to 6. See also SEQ |
| | ID NO 187, where n is 1 to 5. |
| 206 | Alternative IL-15 polypeptide sequence, having a TVAAPS linker (as used in |
| | e.g. CYTB28), otherwise the same as SEQ ID NO: 154 |
| 207 | Alternative IL-15 polypeptide sequence, having alternative linker (as used |
| | in e.g. CYTB32), otherwise the same as SEQ ID NO: 154 |
| 208 | Fc fusion domain (hinge-CH2-CH3) |
| 209 | Example linker at C-terminus of Fc fusion domain |
| 210 | [sushi-linker-IL-15]-linker-[hinge-CH2-CH3]-linker |
| 211 | scFV Vh1 linker VL1 (NKG2D) |
| 212 | scFV Vh2 linker VL2 (NKG2D) |
| 213 | scFV Vh3 linker VL3 (NKG2D) |
| 214 | scFV Vh4 linker VL4 (NKG2D) |
| 215 | scFV Vh5 linker VL5 (NKG2D) |
| 216 | scFV Vh6 linker VL6 (NKG2D) |
| 217 | scFV Vh7 linker VL7 (NKG2D) |
| 218 | scFV Vh8 linker VL8 (NKG2D) |
| 219 | scFV Vh9 linker VL9 (NKG2D) |
| 220 | scFV Vh10 linker VL10 (NKG2D) |
| 221 | scFV Vh11 linker VL11 (NKp30) |
| 222 | scFV Vh12 linker VL12 (NKp30) |
| 223 | scFV Vh13 linker VL13 (NKp46) |
| 224 | scFV VL1 linker Vh1 (NKG2D) |
| 225 | scFV VL2 linker Vh2 (NKG2D) |
| 226 | scFV VL3 linker Vh3 (NKG2D) |
| 227 | scFV VL4 linker Vh4 (NKG2D) |
| 228 | scFV VL5 linker Vh5 (NKG2D) |
| 229 | scFV VL6 linker Vh6 (NKG2D) |
| 230 | scFV VL7 linker Vh7 (NKG2D) |
| 231 | scFV VL8 linker Vh8 (NKG2D) |
| 232 | scFV VL9 linker Vh9 (NKG2D) |
| 233 | scFV VL10 linker Vh10 (NKG2D) |
| 234 | scFV VL11 linker Vh11 (NKp30) |
| 235 | scFV VL12 linker Vh12 (NKp30) |
| 236 | scFV VL13 linker Vh13 (NKp46) |
| 237 | sushi-IL-15-linker-FC linker scFV Vh1 VL1 (NKG2D) |
| 238 | sushi-IL-15-linker-FC linker scFV Vh2 VL2 (NKG2D) |
| 239 | sushi-IL-15-linker-FC linker scFV Vh3 VL3 (NKG2D) |
| 240 | sushi-IL-15-linker-FC linker scFV Vh4 VL4 (NKG2D) |
| 241 | sushi-IL-15-linker-FC linker scFV Vh5 VL5 (NKG2D) |
| 242 | sushi-IL-15-linker-FC linker scFV Vh6 VL6 (NKG2D) |
| 243 | sushi-IL-15-linker-FC linker scFV Vh7 VL7 (NKG2D) |
| 244 | sushi-IL-15-linker-FC linker scFV Vh8 VL8 (NKG2D) |
| 245 | sushi-IL-15-linker-FC linker scFV Vh9 VL9 (NKG2D) |
| 246 | sushi-IL-15-linker-FC linker scFV Vh10 VL10 (NKG2D) |
| 247 | sushi-IL-15-linker-FC linker scFV Vh11 VL11 (NKp30) |
| 248 | sushi-IL-15-linker-FC linker scFV Vh12 VL12 (NKp30) |
| 249 | sushi-IL-15-linker-FC linker scFV Vh13 VL13 (NKp46) |
| 250 | sushi-IL-15-linker-FC linker scFV VL1 Vh1 (NKG2D) |
| 251 | sushi-IL-15-linker-FC linker scFV VL2 Vh2 (NKG2D) |
| 252 | sushi-IL-15-linker-FC linker scFV VL3 Vh3 (NKG2D) |
| 253 | sushi-IL-15-linker-FC linker scFV VL4 Vh4 (NKG2D) |
| 254 | sushi-IL-15-linker-FC linker scFV VL5 Vh5 (NKG2D) |
| 255 | sushi-IL-15-linker-FC linker scFV VL6 Vh6 (NKG2D) |
| 256 | sushi-IL-15-linker-FC linker scFV VL7 Vh7 (NKG2D) |
| 257 | sushi-IL-15-linker-FC linker scFV VL8 Vh8 (NKG2D) |
| 258 | sushi-IL-15-linker-FC linker scFV VL9 Vh9 (NKG2D) |
| 259 | sushi-IL-15-linker-FC linker scFV VL10 Vh10 (NKG2D) |
| 260 | sushi-IL-15-linker-FC linker scFV VL11 Vh11 (NKp30) |
| 261 | sushi-IL-15-linker-FC linker scFV VL12 Vh12 (NKp30) |
| 262 | sushi-IL-15-linker-FC linker scFV VL13 Vh13 (NKp46) |

### Embodiments

The present invention relates to at least the following numbered embodiments:
1. An immunoconjugate, comprising:
   a) at least two IL-15 polypeptides, each comprising
      i. an IL-15 cytokine; and
      ii. IL-15 receptor alpha (CD215) or a functional fragment thereof; and
   b) a multivalent antibody or antigen-binding fragment thereof, comprising at least two antibody binding domains that each specifically bind to an antigen selected from the group consisting of NKG2D, NKp30, NKp46, NKp44 and DNAM-1.
2. The immunoconjugate of embodiment 1, wherein the IL-15 polypeptides and the multivalent antibody or antigen-binding fragment thereof are directly fused to each other.
3. The immunoconjugate of embodiment 1, wherein the IL-15 polypeptides and the multivalent antibody or antigen-binding fragment thereof are joined by a linker.
4. The immunoconjugate of embodiment 3, wherein the linker is a peptide linker.
5. The immunoconjugate of embodiment 3, wherein the linker is a peptide of from about 1 to about 60 amino acid residues in length.
6. The immunoconjugate of any one of embodiments 3 to 5, wherein the linker comprises or consists of glycine and serine.
7. The immunoconjugate of any one of embodiments 3 to 6, wherein the linker comprises or consists of the sequence [GGGGS]ₙX, or [GGGS]ₙX, or [GGS]ₙX, wherein n is from 1 to 6 and wherein X is G or is absent.
8. The immunoconjugate of any one of embodiments 3 to 7, wherein the linker comprises or consists of the sequence [[G]ₘS]ₙX, wherein m is from 2 to 4, n is from 1 to 6 and X is G or is absent.
9. The immunoconjugate of any one of embodiments 3 to 8, wherein the linker comprises or consists of glycine and serine residues, and optionally the linker comprises more glycine than serine residues.
10. The immunoconjugate of any one of embodiments 3 to 9, wherein the linker comprises or consist of glycine and serine residues in a ratio of from about 5:1 to about 2:1, optionally where the linker comprises or consists of glycine and serine residues in a ratio of about 5:1.
11. The immunoconjugate of any one of embodiments 3 to 10, wherein the linker comprises or consists of about 80% glycine and about 20% serine residues.
12. The immunoconjugate of any one of embodiments 3 to 11, wherein the linker comprises or consists of a sequence selected from the group consisting of SEQ ID NOs: 182 to 187 and 204 to 205.
13. The immunoconjugate of any preceding embodiment, wherein the multivalent antibody or antigen-binding fragment thereof comprises at least two heavy chains and at least two light chains, and the IL-15 polypeptides are conjugated to the heavy chains, optionally via a linker.
14. The immunoconjugate of embodiment 13, wherein the IL-15 polypeptides are conjugated to the C-termini of the heavy chains, optionally via a linker.
15. The immunoconjugate of embodiment 13, wherein the IL-15 polypeptides are conjugated to the N-termini of the heavy chains, optionally via a linker.
16. The immunoconjugate of embodiment 13, wherein the IL-15 polypeptides are conjugated to the CH3 domains of the heavy chains, optionally via a linker.
17. The immunoconjugate of any one of embodiments 1 to 12, wherein the multivalent antibody or anti-gen-binding fragment thereof comprises at least two heavy chains and at least two light chains, and the IL-15 polypeptides are conjugated to the light chains, optionally via a linker.
18. The immunoconjugate of embodiment 17, wherein the IL-15 polypeptides are conjugated to an intact IgG.
19. The immunoconjugate of embodiment 17, wherein the IL-15 polypeptides are conjugated to the C-termini of the light chains, optionally via a linker.
20. The immunoconjugate of embodiment 17, wherein the IL-15 polypeptides are conjugated to the N-termini of the light chains, optionally via a linker.
21. The immunoconjugate of embodiment 17, wherein the IL-15 polypeptides are conjugated to the CL domains of the light chains, optionally via a linker.
22. The immunoconjugate of any preceding embodiment, wherein the multivalent antibody or antigen binding fragment thereof comprises:
   a) a heavy chain variable region (VH) comprising:
      i. a heavy chain complementary determining region (HCDR) 1 comprising or consisting of a sequence selected from the group consisting of SEQ ID NOs: 1 to 13, optionally having up to 2 amino acid substitutions therefrom;
      ii. a HCDR2 comprising or consisting of a sequence selected from the group consisting of SEQ ID NOs: 14 to 26, optionally having up to 2 amino acid substitutions therefrom; and
      iii. a HCDR3 comprising or consisting of a sequence selected from the group consisting of SEQ ID NOs: 27 to 39, optionally having up to 2 amino acid substitutions therefrom; and
   b) a light chain variable region (VL) comprising:
      i. a light chain complementarity determining region (LCDR) 1 comprising or consisting of a sequence selected from the group consisting of SEQ ID NOs: 40 to 52, optionally having up to 2 amino acid substitutions therefrom;
      ii. a LCDR2 comprising or consisting of a sequence selected from the group consisting of SEQ ID NOs: 53 to 65, optionally having up to 2 amino acid substitutions therefrom; and
      iii. a LCDR3 comprising or consisting of a sequence selected from the group consisting of SEQ ID NOs: 66 to 78, optionally having up to 2 amino acid substitutions therefrom.
23. The immunoconjugate of any preceding embodiment, wherein the multivalent antibody or antigen binding fragment thereof comprises:
   a) a heavy chain variable region (VH) comprising:
      a HCDR1 comprising or consisting of the sequence of SEQ ID NO: 1, optionally having up to 2 amino acid substitutions therefrom;
      a HCDR2 comprising or consisting of the sequence of SEQ ID NO: 14, optionally having up to 2 amino acid substitutions therefrom; and
      a HCDR3 comprising or consisting of the sequence of SEQ ID NO: 27, optionally having up to 2 amino acid substitutions therefrom;
      and a light chain variable region (VL) comprising:
      a LCDR1 comprising or consisting of the sequence of SEQ ID NO: 40, optionally having up to 2 amino acid substitutions therefrom;
      a LCDR2 comprising or consisting of the sequence of SEQ ID NO: 53, optionally having up to 2 amino acid substitutions therefrom; and
      a LCDR3 comprising or consisting of the sequence of SEQ ID NO: 66, optionally having up to 2 amino acid substitutions therefrom;
   b) a heavy chain variable region (VH) comprising:
      a HCDR1 comprising or consisting of the sequence of SEQ ID NO: 2, optionally having up to 2 amino acid substitutions therefrom;
      a HCDR2 comprising or consisting of the sequence of SEQ ID NO: 15, optionally having up to 2 amino acid substitutions therefrom; and
      a HCDR3 comprising or consisting of the sequence of SEQ ID NO: 28, optionally having up to 2 amino acid substitutions therefrom;
      and a light chain variable region (VL) comprising:
      a LCDR1 comprising or consisting of the sequence of SEQ ID NO: 41, optionally having up to 2 amino acid substitutions therefrom;
      a LCDR2 comprising or consisting of the sequence of SEQ ID NO: 54, optionally having up to 2 amino acid substitutions therefrom; and
      a LCDR3 comprising or consisting of the sequence of SEQ ID NO: 67, optionally having up to 2 amino acid substitutions therefrom;
   c) a heavy chain variable region (VH) comprising:
      a HCDR1 comprising or consisting of the sequence of SEQ ID NO: 3, optionally having up to 2 amino acid substitutions therefrom;
      a HCDR2 comprising or consisting of the sequence of SEQ ID NO: 16, optionally having up to 2 amino acid substitutions therefrom; and
      a HCDR3 comprising or consisting of the sequence of SEQ ID NO: 29, optionally having up to 2 amino acid substitutions therefrom;
      and a light chain variable region (VL) comprising:
      a LCDR1 comprising or consisting of the sequence of SEQ ID NO: 42, optionally having up to 2 amino acid substitutions therefrom;
      a LCDR2 comprising or consisting of the sequence of SEQ ID NO: 55, optionally having up to 2 amino acid substitutions therefrom; and
      a LCDR3 comprising or consisting of the sequence of SEQ ID NO: 68, optionally having up to 2 amino acid substitutions therefrom;
   d) a heavy chain variable region (VH) comprising:
      a HCDR1 comprising or consisting of the sequence of SEQ ID NO: 4, optionally having up to 2 amino acid substitutions therefrom;
      a HCDR2 comprising or consisting of the sequence of SEQ ID NO: 17, optionally having up to 2 amino acid substitutions therefrom; and
      a HCDR3 comprising or consisting of the sequence of SEQ ID NO: 30, optionally having up to 2 amino acid substitutions therefrom;
      and a light chain variable region (VL) comprising:
      a LCDR1 comprising or consisting of the sequence of SEQ ID NO: 43, optionally having up to 2 amino acid substitutions therefrom;
      a LCDR2 comprising or consisting of the sequence of SEQ ID NO: 56, optionally having up to 2 amino acid substitutions therefrom; and
      a LCDR3 comprising or consisting of the sequence of SEQ ID NO: 69, optionally having up to 2 amino acid substitutions therefrom;
   e) a heavy chain variable region (VH) comprising:
      a HCDR1 comprising or consisting of the sequence of SEQ ID NO: 5, optionally having up to 2 amino acid substitutions therefrom;
      a HCDR2 comprising or consisting of the sequence of SEQ ID NO: 18, optionally having up to 2 amino acid substitutions therefrom; and
      a HCDR3 comprising or consisting of the sequence of SEQ ID NO: 31, optionally having up to 2 amino acid substitutions therefrom;
      and a light chain variable region (VL) comprising:
      a LCDR1 comprising or consisting of the sequence of SEQ ID NO: 44, optionally having up to 2 amino acid substitutions therefrom;
      a LCDR2 comprising or consisting of the sequence of SEQ ID NO: 57, optionally having up to 2 amino acid substitutions therefrom; and
      a LCDR3 comprising or consisting of the sequence of SEQ ID NO: 70, optionally having up to 2 amino acid substitutions therefrom;
   f) a heavy chain variable region (VH) comprising:
      a HCDR1 comprising or consisting of the sequence of SEQ ID NO: 6, optionally having up to 2 amino acid substitutions therefrom;
      a HCDR2 comprising or consisting of the sequence of SEQ ID NO: 19, optionally having up to 2 amino acid substitutions therefrom; and
      a HCDR3 comprising or consisting of the sequence of SEQ ID NO: 32, optionally having up to 2 amino acid substitutions therefrom;
      and a light chain variable region (VL) comprising:
      a LCDR1 comprising or consisting of the sequence of SEQ ID NO: 45, optionally having up to 2 amino acid substitutions therefrom;
      a LCDR2 comprising or consisting of the sequence of SEQ ID NO: 58, optionally having up to 2 amino acid substitutions therefrom; and
      a LCDR3 comprising or consisting of the sequence of SEQ ID NO: 71, optionally having up to 2 amino acid substitutions therefrom;
   g) a heavy chain variable region (VH) comprising:
      a HCDR1 comprising or consisting of the sequence of SEQ ID NO: 7, optionally having up to 2 amino acid substitutions therefrom;
      a HCDR2 comprising or consisting of the sequence of SEQ ID NO: 20, optionally having up to 2 amino acid substitutions therefrom; and
      a HCDR3 comprising or consisting of the sequence of SEQ ID NO: 33, optionally having up to 2 amino acid substitutions therefrom;
      and a light chain variable region (VL) comprising:
      a LCDR1 comprising or consisting of the sequence of SEQ ID NO: 46, optionally having up to 2 amino acid substitutions therefrom;
      a LCDR2 comprising or consisting of the sequence of SEQ ID NO: 59, optionally having up to 2 amino acid substitutions therefrom; and
      a LCDR3 comprising or consisting of the sequence of SEQ ID NO: 72, optionally having up to 2 amino acid substitutions therefrom;
   h) a heavy chain variable region (VH) comprising:
      a HCDR1 comprising or consisting of the sequence of SEQ ID NO: 8, optionally having up to 2 amino acid substitutions therefrom;
      a HCDR2 comprising or consisting of the sequence of SEQ ID NO: 21, optionally having up to 2 amino acid substitutions therefrom; and
      a HCDR3 comprising or consisting of the sequence of SEQ ID NO: 34, optionally having up to 2 amino acid substitutions therefrom;
      and a light chain variable region (VL) comprising:
      a LCDR1 comprising or consisting of the sequence of SEQ ID NO: 47, optionally having up to 2 amino acid substitutions therefrom;
      a LCDR2 comprising or consisting of the sequence of SEQ ID NO: 60, optionally having up to 2 amino acid substitutions therefrom; and
      a LCDR3 comprising or consisting of the sequence of SEQ ID NO: 73, optionally having up to 2 amino acid substitutions therefrom;
   i) a heavy chain variable region (VH) comprising:
      a HCDR1 comprising or consisting of the sequence of SEQ ID NO: 9, optionally having up to 2 amino acid substitutions therefrom;
      a HCDR2 comprising or consisting of the sequence of SEQ ID NO: 22, optionally having up to 2 amino acid substitutions therefrom; and
      a HCDR3 comprising or consisting of the sequence of SEQ ID NO: 35, optionally having up to 2 amino acid substitutions therefrom;
      and a light chain variable region (VL) comprising:
      a LCDR1 comprising or consisting of the sequence of SEQ ID NO: 48, optionally having up to 2 amino acid substitutions therefrom;
      a LCDR2 comprising or consisting of the sequence of SEQ ID NO: 61, optionally having up to 2 amino acid substitutions therefrom; and
      a LCDR3 comprising or consisting of the sequence of SEQ ID NO: 74, optionally having up to 2 amino acid substitutions therefrom;
   j) a heavy chain variable region (VH) comprising:
      a HCDR1 comprising or consisting of the sequence of SEQ ID NO: 10, optionally having up to 2 amino acid substitutions therefrom;
      a HCDR2 comprising or consisting of the sequence of SEQ ID NO: 23, optionally having up to 2 amino acid substitutions therefrom; and
      a HCDR3 comprising or consisting of the sequence of SEQ ID NO: 36, optionally having up to 2 amino acid substitutions therefrom;
      and a light chain variable region (VL) comprising:
      a LCDR1 comprising or consisting of the sequence of SEQ ID NO: 49, optionally having up to 2 amino acid substitutions therefrom;
      a LCDR2 comprising or consisting of the sequence of SEQ ID NO: 62, optionally having up to 2 amino acid substitutions therefrom; and
      a LCDR3 comprising or consisting of the sequence of SEQ ID NO: 75, optionally having up to 2 amino acid substitutions therefrom;
   k) a heavy chain variable region (VH) comprising:
      a HCDR1 comprising or consisting of the sequence of SEQ ID NO: 11, optionally having up to 2 amino acid substitutions therefrom;
      a HCDR2 comprising or consisting of the sequence of SEQ ID NO: 24, optionally having up to 2 amino acid substitutions therefrom; and
      a HCDR3 comprising or consisting of the sequence of SEQ ID NO: 37, optionally having up to 2 amino acid substitutions therefrom;
      and a light chain variable region (VL) comprising:
      a LCDR1 comprising or consisting of the sequence of SEQ ID NO: 50, optionally having up to 2 amino acid substitutions therefrom;
      a LCDR2 comprising or consisting of the sequence of SEQ ID NO: 63, optionally having up to 2 amino acid substitutions therefrom; and
      a LCDR3 comprising or consisting of the sequence of SEQ ID NO: 76, optionally having up to 2 amino acid substitutions therefrom;
   l) a heavy chain variable region (VH) comprising:
      a HCDR1 comprising or consisting of the sequence of SEQ ID NO: 12, optionally having up to 2 amino acid substitutions therefrom;
      a HCDR2 comprising or consisting of the sequence of SEQ ID NO: 25, optionally having up to 2 amino acid substitutions therefrom; and
      a HCDR3 comprising or consisting of the sequence of SEQ ID NO: 38, optionally having up to 2 amino acid substitutions therefrom;
      and a light chain variable region (VL) comprising:
      a LCDR1 comprising or consisting of the sequence of SEQ ID NO: 51, optionally having up to 2 amino acid substitutions therefrom;
      a LCDR2 comprising or consisting of the sequence of SEQ ID NO: 64, optionally having up to 2 amino acid substitutions therefrom; and
      a LCDR3 comprising or consisting of the sequence of SEQ ID NO: 77, optionally having up to 2 amino acid substitutions therefrom; or
   m) a heavy chain variable region (VH) comprising:
      a HCDR1 comprising or consisting of the sequence of SEQ ID NO: 13, optionally having up to 2 amino acid substitutions therefrom;
      a HCDR2 comprising or consisting of the sequence of SEQ ID NO: 26, optionally having up to 2 amino acid substitutions therefrom; and
      a HCDR3 comprising or consisting of the sequence of SEQ ID NO: 39, optionally having up to 2 amino acid substitutions therefrom;
      and a light chain variable region (VL) comprising:
      a LCDR1 comprising or consisting of the sequence of SEQ ID NO: 52, optionally having up to 2 amino acid substitutions therefrom;
      a LCDR2 comprising or consisting of the sequence of SEQ ID NO: 65, optionally having up to 2 amino acid substitutions therefrom; and
      a LCDR3 comprising or consisting of the sequence of SEQ ID NO: 78, optionally having up to 2 amino acid substitutions therefrom.
24. The immunoconjugate of any one of embodiments 22 to 23, wherein the optional amino acid substitutions are conservative substitutions.
25. The immunoconjugate of any preceding embodiment, wherein the multivalent antibody or antigen binding fragment thereof comprises:
   a) a heavy chain variable region (VH) comprising or consisting of a sequence having at least 90%, at least 91%, at least 92%, at least 93%, at least 94%, at least 95%, at least 95%, at least 97%, at least 98% or at least 99% identity to a sequence selected from the group consisting of SEQ ID NOs: 79 to 91 and 202; and
   b) a light chain variable region (VL) comprising or consisting of a sequence having at least 90%, at least 91%, at least 92%, at least 93%, at least 94%, at least 95%, at least 95%, at least 97%, at least 98% or at least 99% identity to a sequence selected from the group consisting of SEQ ID NOs: 92 to 104.
26. The immunoconjugate of any preceding embodiment, wherein the multivalent antibody or antigen binding fragment thereof comprises:
   a) a VH comprising or consisting of a sequence selected from the group consisting of SEQ ID NOs: 79 to 91 and 202; and
   b) a VL comprising or consisting of a sequence selected from the group consisting of SEQ ID NOs: 92 to 104.
27. The immunoconjugate of any preceding embodiment, wherein the multivalent antibody or antigen binding fragment thereof comprises:
   a) a VH comprising or consisting of the sequence of SEQ ID NO: 79 and a VL comprising or consisting of the sequence of SEQ ID NO: 92;
   b) a VH comprising or consisting of the sequence of SEQ ID NO: 202 and a VL comprising or consisting of the sequence of SEQ ID NO: 92;
   c) a VH comprising or consisting of the sequence of SEQ ID NO: 80 and a VL comprising or consisting of the sequence of SEQ ID NO: 93;
   d) a VH comprising or consisting of the sequence of SEQ ID NO: 81 and a VL comprising or consisting of the sequence of SEQ ID NO: 94;
   e) a VH comprising or consisting of the sequence of SEQ ID NO: 82 and a VL comprising or consisting of the sequence of SEQ ID NO: 95;
   f) a VH comprising or consisting of the sequence of SEQ ID NO: 83 and a VL comprising or consisting of the sequence of SEQ ID NO: 96;
   g) a VH comprising or consisting of the sequence of SEQ ID NO: 84 and a VL comprising or consisting of the sequence of SEQ ID NO: 97;
   h) a VH comprising or consisting of the sequence of SEQ ID NO: 85 and a VL comprising or consisting of the sequence of SEQ ID NO: 98;
   i) a VH comprising or consisting of the sequence of SEQ ID NO: 86 and a VL comprising or consisting of the sequence of SEQ ID NO: 99;
   j) a VH comprising or consisting of the sequence of SEQ ID NO: 87 and a VL comprising or consisting of the sequence of SEQ ID NO: 100;
   k) a VH comprising or consisting of the sequence of SEQ ID NO: 88 and a VL comprising or consisting of the sequence of SEQ ID NO: 101;
   l) a VH comprising or consisting of the sequence of SEQ ID NO: 89 and a VL comprising or consisting of the sequence of SEQ ID NO: 102;
   m) a VH comprising or consisting of the sequence of SEQ ID NO: 90 and a VL comprising or consisting of the sequence of SEQ ID NO: 103; or
   n) a VH comprising or consisting of the sequence of SEQ ID NO: 91 and a VL comprising or consisting of the sequence of SEQ ID NO: 104.
28. The immunoconjugate of any preceding embodiment, wherein the multivalent antibody or antigen binding fragment thereof comprises:
   a) a heavy chain comprising or consisting of a sequence having at least 90%, at least 91%, at least 92%, at least 93%, at least 94%, at least 95%, at least 95%, at least 97%, at least 98% or at least 99% identity to a sequence selected from the group consisting of SEQ ID NOs: 109 to 134; and
   b) a light chain comprising or consisting of a sequence having at least 90%, at least 91%, at least 92%, at least 93%, at least 94%, at least 95%, at least 95%, at least 97%, at least 98% or at least 99% identity to a sequence selected from the group consisting of SEQ ID NOs: 135 to 147.
29. The immunoconjugate of any preceding embodiment, wherein the multivalent antibody or antigen binding fragment thereof comprises:
   a) a heavy chain comprising or consisting of a sequence selected from the group consisting of SEQ ID NOs: 109 to 134; and
   b) a light chain comprising or consisting of a sequence selected from the group consisting of SEQ ID NOs: 135 to 147.
30. The immunoconjugate of any preceding embodiment, wherein the multivalent antibody or antigen binding fragment thereof comprises:
   a) a heavy chain comprising or consisting of the sequence of SEQ ID NO: 109 and a light chain comprising or consisting of the sequence SEQ ID NO: 135;
   b) a heavy chain comprising or consisting of the sequence of SEQ ID NO: 110 and a light chain comprising or consisting of the sequence SEQ ID NO: 136;
   c) a heavy chain comprising or consisting of the sequence of SEQ ID NO: 111 and a light chain comprising or consisting of the sequence SEQ ID NO: 137;
   d) a heavy chain comprising or consisting of the sequence of SEQ ID NO: 112 and a light chain comprising or consisting of the sequence SEQ ID NO: 138;
   e) a heavy chain comprising or consisting of the sequence of SEQ ID NO: 113 and a light chain comprising or consisting of the sequence SEQ ID NO: 139;
   f) a heavy chain comprising or consisting of the sequence of SEQ ID NO: 114 and a light chain comprising or consisting of the sequence SEQ ID NO: 140;
   g) a heavy chain comprising or consisting of the sequence of SEQ ID NO: 115 and a light chain comprising or consisting of the sequence SEQ ID NO: 141;
   h) a heavy chain comprising or consisting of the sequence of SEQ ID NO: 116 and a light chain comprising or consisting of the sequence SEQ ID NO: 142;
   i) a heavy chain comprising or consisting of the sequence of SEQ ID NO: 117 and a light chain comprising or consisting of the sequence SEQ ID NO: 143;
   j) a heavy chain comprising or consisting of the sequence of SEQ ID NO: 118 and a light chain comprising or consisting of the sequence SEQ ID NO: 144;
   k) a heavy chain comprising or consisting of the sequence of SEQ ID NO: 119 and a light chain comprising or consisting of the sequence SEQ ID NO: 145;
   l) a heavy chain comprising or consisting of the sequence of SEQ ID NO: 120 and a light chain comprising or consisting of the sequence SEQ ID NO: 146;
   m) a heavy chain comprising or consisting of the sequence of SEQ ID NO: 121 and a light chain comprising or consisting of the sequence SEQ ID NO: 147;
   n) a heavy chain comprising or consisting of the sequence of SEQ ID NO: 122 and a light chain comprising or consisting of the sequence SEQ ID NO: 135;
   o) a heavy chain comprising or consisting of the sequence of SEQ ID NO: 123 and a light chain comprising or consisting of the sequence SEQ ID NO: 136;
   p) a heavy chain comprising or consisting of the sequence of SEQ ID NO: 124 and a light chain comprising or consisting of the sequence SEQ ID NO: 137;
   q) a heavy chain comprising or consisting of the sequence of SEQ ID NO: 125 and a light chain comprising or consisting of the sequence SEQ ID NO: 138;
   r) a heavy chain comprising or consisting of the sequence of SEQ ID NO: 126 and a light chain comprising or consisting of the sequence SEQ ID NO: 139;
   s) a heavy chain comprising or consisting of the sequence of SEQ ID NO: 127 and a light chain comprising or consisting of the sequence SEQ ID NO: 140;
   t) a heavy chain comprising or consisting of the sequence of SEQ ID NO: 128 and a light chain comprising or consisting of the sequence SEQ ID NO: 141;
   u) a heavy chain comprising or consisting of the sequence of SEQ ID NO: 129 and a light chain comprising or consisting of the sequence SEQ ID NO: 142;
   v) a heavy chain comprising or consisting of the sequence of SEQ ID NO: 130 and a light chain comprising or consisting of the sequence SEQ ID NO: 143;
   w) a heavy chain comprising or consisting of the sequence of SEQ ID NO: 131 and a light chain comprising or consisting of the sequence SEQ ID NO: 144;
   x) a heavy chain comprising or consisting of the sequence of SEQ ID NO: 132 and a light chain comprising or consisting of the sequence SEQ ID NO: 145;
   y) a heavy chain comprising or consisting of the sequence of SEQ ID NO: 133 and a light chain comprising or consisting of the sequence SEQ ID NO: 146; or
   z) a heavy chain comprising or consisting of the sequence of SEQ ID NO: 134 and a light chain comprising or consisting of the sequence SEQ ID NO: 147.
31. The immunoconjugate of any preceding embodiment, wherein the antibody binding domains each specifically bind to an antigen selected from the group consisting of NKG2D, NKp30 and NKp46.
32. The immunoconjugate of any preceding embodiment, wherein the multivalent antibody or antigen-binding fragment thereof binds to human and/or cynomologous NKG2D.
33. The immunoconjugate of any preceding embodiment, wherein the antibody or antigen-binding frag-ment thereof binds to human and/or cynomologous NKp30.
34. The immunoconjugate of any preceding embodiment, wherein the antibody or antigen-binding frag-ment thereof binds to human and/or cynomologous NKp46.
35. The immunoconjugate of any preceding embodiment, wherein the antibody or antigen-binding frag-ment thereof binds to human and/or cynomologous NKp44.
36. The immunoconjugate of any preceding embodiment, wherein the antibody or antigen-binding frag-ment thereof binds to human and/or cynomologous DNAM-1.
37. The immunoconjugate of any preceding embodiment, wherein the multivalent antibody or antigen binding fragment thereof comprises an Fc domain, optionally linked to a CH1-hinge domain.
38. The immunoconjugate of embodiment 37, wherein the Fc domain is a human Fc domain.
39. The immunoconjugate of embodiment 37, wherein the Fc domain is a human IgG Fc domain.
40. The immunoconjugate of embodiment 39, wherein the human IgG Fc domain is a human IgG1 do-main.
41. The immunoconjugate of any one of embodiments 37 to 40, wherein the Fc domain is Fc disabled.
42. The immunoconjugate of any one of embodiments 37 to 41, wherein the Fc domain comprises a sub-stitution with respect to a wild-type Fc sequence, wherein the substitution reduces binding of the Fc domain to an Fc receptor.
43. The immunoconjugate of embodiment 42, wherein the substitution is a substitution at a position se-lected from the group consisting of L234, L235, G236, G237 and P329 (EU index numbering).
44. The immunoconjugate of embodiment 43, wherein the substitution is selected from the group con-sisting of L235A, G237A and P329G (EU index numbering).
45. The immunoconjugate of any one of embodiments 37 to 44, wherein the constant domain com-prises:
   a) a heavy chain sequence having at least 90%, at least 91%, at least 92%, at least 93%, at least 94%, at least 95%, at least 95%, at least 97%, at least 98% or at least 99% identity to a sequence selected from the group consisting of SEQ ID NOs: 105,106 and 203; and
   b) a light chain sequence having at least 90%, at least 91%, at least 92%, at least 93%, at least 94%, at least 95%, at least 95%, at least 97%, at least 98% or at least 99% identity to a sequence selected from the group consisting of SEQ ID NOs: 107 to 108.
46. The immunoconjugate of any one of embodiments 37 to 45, wherein the constant domain com-prises:
   c) a heavy chain sequence selected from the group consisting of SEQ ID NOs: 105, 106 and 203; and
   d) a light chain sequence selected from the group consisting of SEQ ID NOs: 107 to 108.
47. The immunoconjugate of any one of embodiments 37 to 46, wherein the constant domain com-prises:
   e) a heavy chain sequence comprising or consisting of the sequence of SEQ ID NO: 105 and a light chain sequence comprising or consisting of the sequence of SEQ ID NO: 107;
   f) a heavy chain sequence comprising or consisting of the sequence of SEQ ID NO: 105 and a light chain sequence comprising or consisting of the sequence of SEQ ID NO: 108;
   g) a heavy chain sequence comprising or consisting of the sequence of SEQ ID NO: 106 and a light chain sequence comprising or consisting of the sequence of SEQ ID NO: 107;
   h) a heavy chain sequence comprising or consisting of the sequence of SEQ ID NO: 106 and a light chain sequence comprising or consisting of the sequence of SEQ ID NO: 108;
   i) a heavy chain sequence comprising or consisting of the sequence of SEQ ID NO: 203 and a light chain sequence comprising or consisting of the sequence of SEQ ID NO: 107; or
   j) a heavy chain sequence comprising or consisting of the sequence of SEQ ID NO: 203 and a light chain sequence comprising or consisting of the sequence of SEQ ID NO: 108.
48. The immunoconjugate of any preceding embodiment, wherein the IL-15 cytokine is human IL-15 (hIL-15) cytokine or a variant or fragment thereof.
49. The immunoconjugate of any preceding embodiment, wherein the IL-15 polypeptide is an IL-15 superagonist.
50. The immunoconjugate of any preceding embodiment, wherein the IL-15 receptor alpha (CD215) is human IL-15 receptor alpha (CD215).
51. The immunoconjugate of any preceding embodiment, wherein the IL-15 receptor alpha (CD215) com-prises or consists of a sequence having at least 90%, at least 91%, at least 92%, at least 93%, at least 94%, at least 95%, at least 95%, at least 97%, at least 98% or at least 99% identity to SEQ ID NO: 148.
52. The immunoconjugate of any preceding embodiment, wherein the IL-15 receptor alpha (CD215) com-prises or consists of SEQ ID NO: 148.
53. The immunoconjugate of any preceding embodiment, wherein the IL-15 receptor alpha (CD215) frag-ment comprises up to 100 consecutive amino acids of the IL-15 receptor alpha (CD215) sequence and comprises the IL-15 receptor alpha (CD215) sushi domain.
54. The immunoconjugate of any preceding embodiment, wherein the IL-15 polypeptide comprises the IL-15 receptor alpha (CD215) sushi domain, optionally wherein the IL-15 receptor alpha (CD215) sushi domain is a human IL-15 receptor alpha (CD215) sushi domain.
55. The immunoconjugate of embodiment 54, wherein the IL-15 receptor alpha (CD215) sushi domain comprises or consists of a sequence having at least 90%, at least 91%, at least 92%, at least 93%, at least 94%, at least 95%, at least 95%, at least 97%, at least 98% or at least 99% identity to a sequence selected from the group consisting of SEQ ID NOs: 149 to 150.
56. The immunoconjugate of embodiment 54, wherein the IL-15 receptor alpha (CD215) sushi domain comprises or consists of a sequence selected from the group consisting of SEQ ID NOs: 149 to 150.
57. The immunoconjugate of any preceding embodiment, wherein the or each IL-15 polypeptide com-prises, in an N- to C-terminal direction, a human IL-15 cytokine and IL-15 receptor alpha (CD215) or functional fragment thereof, optionally joined by a linker.
58. The immunoconjugate of any one of embodiments 1 to 56, wherein the or each IL-15 polypeptide comprises, in an N- to C-terminal direction, an IL-15 receptor alpha (CD215) or functional fragment thereof and a human IL-15 cytokine, optionally joined by a linker.
59. The immunoconjugate of any preceding embodiment, wherein the IL-15 cytokine is not mutated to reduce function.
60. The immunoconjugate of any preceding embodiment, wherein the IL-15 cytokine is wildtype human IL-15 cytokine.
61. The immunoconjugate of any one of embodiments 1 to 58, wherein the IL-15 cytokine is an IL-15 mutein.
62. The immunoconjugate of embodiment 61, wherein the IL-15 mutein comprises a substitution at posi-tion N72.
63. The immunoconjugate of embodiment 62, wherein the IL-15 mutein comprises a N72D mutation.
64. The immunoconjugate of any preceding embodiment, wherein the IL-15 cytokine comprises or con-sists of a sequence having at least 90%, at least 91%, at least 92%, at least 93%, at least 94%, at least 95%, at least 95%, at least 97%, at least 98% or at least 99% identity to a sequence selected from the group consisting of SEQ ID NOs: 151 to 153, preferably SEQ ID NO: 151.
65. The immunoconjugate of any preceding embodiment, wherein the IL-15 cytokine comprises or con-sists of a sequence selected from the group consisting of SEQ ID NOs: 151 to 153, preferably SEQ ID NO: 151.
66. The immunoconjugate of any preceding embodiment, wherein the IL-15 polypeptide comprises or consists of a sequence having at least 90%, at least 91%, at least 92%, at least 93%, at least 94%, at least 95%, at least 95%, at least 97%, at least 98% or at least 99% identity to a sequence selected from the group consisting of SEQ ID NOs: 154, 155, 206 and 207.
67. The immunoconjugate of any preceding embodiment, wherein the IL-15 polypeptide comprises or consists of a sequence selected from the group consisting of SEQ ID NOs: 154, 155, 206 and 207.
68. The immunoconjugate of any previous embodiment, wherein the IL-15 cytokine and the IL-15 recep-tor alpha (CD215) or functional fragment thereof are directly fused to each other.
69. The immunoconjugate of any one of embodiments 1 to 67, wherein the IL-15 cytokine and the IL-15 receptor alpha (CD215) or functional fragment thereof are joined by a linker.
70. The immunoconjugate of embodiment 69, wherein the linker is a peptide linker.
71. The immunoconjugate of embodiment 70, wherein the linker is a peptide of from about 1 to about 60 amino acid residues in length.
72. The immunoconjugate of any one of embodiments 69 71, wherein the linker comprises or consists of glycine and serine.
73. The immunoconjugate of any one of embodiments 69 to 72, wherein the linker comprises or consists of the sequence [GGGGS]ₙX, or [GGGS]ₙX, or [GGS]ₙX, wherein n is from 1 to 6 and wherein X is G or is absent.
74. The immunoconjugate of any one of embodiments 69 to 73, wherein the linker comprises or consists of the sequence [[G]ₘS]ₙX, wherein m is from 2 to 4, n is from 1 to 6 and X is G or is absent.
75. The immunoconjugate of any one of embodiments 69 to 74, wherein the linker comprises or consists of glycine and serine residues, and optionally the linker comprises more glycine than serine residues.
76. The immunoconjugate of any one of embodiments 69 to 75, wherein the linker comprises or consist of glycine and serine residues in a ratio of from about 5:1 to about 2:1, optionally where the linker comprises or consists of glycine and serine residues in a ratio of about 5:1
77. The immunoconjugate of any one of embodiments 69 to 76, wherein the linker comprises or consists of about 80% glycine and about 20% serine residues.
78. The immunoconjugate of any one of embodiments 69 to 76, wherein the linker comprises or consists of a sequence selected from the group consisting of SEQ ID NOs: 182 to 187 and 204 to 205.
79. The immunoconjugate of any preceding embodiment, wherein the immunoconjugate comprises:
   a) a first polypeptide chain comprising, in an N- to C-terminal direction:
      i. a heavy chain variable region (VH) of an anti-NKG2D antibody, an anti-NKp30 antibody, an anti-NKp46 antibody, an anti-NKp44 antibody or an anti-DNAM-1 antibody;
      ii. a human heavy chain constant region;
      iii. optionally a linker; and
      iv. the IL-15 polypeptide; and
   b) a second polypeptide chain comprising, in an N- to C-terminal direction:
      i. a light chain variable region (VL) of an anti-NKG2D antibody, an anti-NKp30 antibody, an anti-NKp46 antibody, an anti-NKp44 antibody or an anti-DNAM-1 antibody; and
      ii. a human light constant region;
         and the immunoconjugate comprises or consists of first and second copies of said first and second polypeptides.
80. The immunoconjugate of any preceding embodiment, wherein the immunoconjugate comprises:
   a) a first polypeptide chain comprising, in an N- to C-terminal direction:
      a. a heavy chain variable region (VH) of an anti-NKG2D antibody, an anti-NKp30 antibody, an anti-NKp46 antibody, an anti-NKp44 antibody or an anti-DNAM-1 antibody;
      b. a human heavy chain constant region;
      c. optionally a linker; and
      d. an IL-15 polypeptide comprising the sequence of SEQ ID NO: 154, SEQ ID NO: 206 or SEQ ID NO: 207; and
   b) a second polypeptide chain comprising, in an N- to C-terminal direction:
      a. a light chain variable region (VL) of an anti-NKG2D antibody, an anti-NKp30 antibody, an anti-NKp46 antibody, an anti-NKp44 antibody or an anti-DNAM-1 antibody; and
      b. a human light constant region;
         and the immunoconjugate comprises or consists of first and second copies of said first and second polypeptides.
81. The immunoconjugate of any preceding embodiment, wherein the immunoconjugate comprises:
   a) a first polypeptide chain comprising, in an N- to C-terminal direction:
      i. a heavy chain variable region (VH) of an anti-NKG2D antibody, an anti-NKp30 antibody, an anti-NKp46 antibody, an anti-NKp44 antibody or an anti-DNAM-1 antibody;
      ii. a human heavy chain IgG sequence;
      iii. optionally a linker; and
      iv. the IL-15 polypeptide; and
   b) a second polypeptide chain comprising, in an N- to C-terminal direction:
      i. a light chain variable region (VL) of an anti-NKG2D antibody, an anti-NKp30 antibody, an anti-NKp46 antibody, an anti-NKp44 antibody or an anti-DNAM-1 antibody; and
      ii. a human light chain IgG sequence;
         and the immunoconjugate comprises or consists of first and second copies of said first and second polypeptides.
82. The immunoconjugate of any preceding embodiment, wherein the immunoconjugate comprises:
   a) a first polypeptide chain comprising, in an N- to C-terminal direction:
   i. a heavy chain variable region (VH) of an anti-NKG2D antibody, an anti-NKp30 antibody, an anti-NKp46 antibody, an anti-NKp44 antibody or an anti-DNAM-1 antibody;
   ii. a human heavy chain IgG1 sequence;
   iii. optionally a linker; and
   iv. the IL-15 polypeptide; and
   b) a second polypeptide chain comprising, in an N- to C-terminal direction:
   i. a light chain variable region (VL) of an anti-NKG2D antibody, an anti-NKp30 antibody, an anti-NKp46 antibody, an anti-NKp44 antibody or an anti-DNAM-1 antibody; and
   ii. a human light chain IgG1 sequence;
      and the immunoconjugate comprises or consists of first and second copies of said first and second polypeptides.
83. The immunoconjugate of any preceding embodiment, wherein the immunoconjugate comprises:
   a) a first polypeptide chain comprising, in an N- to C-terminal direction:
   i. a heavy chain of an anti-NKG2D antibody, an anti-NKp30 antibody, an anti-NKp46 antibody, an anti-NKp44 antibody or an anti-DNAM-1 antibody;
   ii. optionally a linker; and
   iii. the IL-15 polypeptide; and
   b) a second polypeptide chain comprising a light chain of an anti-NKG2D antibody, an anti-NKp30 antibody, an anti-NKp46 antibody, an anti-NKp44 antibody or an anti-DNAM-1 antibody;
      and the immunoconjugate comprises or consists of first and second copies of said first and second polypeptides.
84. The immunoconjugate of any preceding embodiment, wherein the immunoconjugate comprises:
   a) a first polypeptide chain comprising, in an N- to C-terminal direction:
      i. a heavy chain variable region (VH) of an anti-NKG2D antibody, an anti-NKp30 antibody, an anti-NKp46 antibody, an anti-NKp44 antibody or an anti-DNAM-1 antibody, the VH comprising an HCDR1, an HCDR2 and an HCDR3;
      ii. a heavy chain constant region comprising an CH1, a hinge, a CH2 and a CH3 re-gion;
      iii. optionally a linker, and
      iv. the IL-15 polypeptide; and
   b) a second polypeptide chain comprising, in an N- to C-terminal direction:
      i. a light chain variable region (VL) of an anti-NKG2D antibody, an anti-NKp30 antibody, an anti-NKp46 antibody, an anti-NKp44 antibody or an anti-DNAM-1 antibody, the VL comprising an LCDR1, an LCDR2 and an LCDR3; and
      ii. a light chain constant (CL) region;
      and the immunoconjugate comprises or consists of first and second copies of said first and second polypeptides.
85. The immunoconjugate of any preceding embodiment, wherein the immunoconjugate comprises:
   a) a first polypeptide chain comprising, in an N- to C-terminal direction:
      i. a heavy chain variable region (VH) of an anti-NKG2D antibody comprising an HCDR1 comprising or consisting of a sequence selected from the group consisting of SEQ ID NOs: 1 to 10 and optionally having up to 2 amino acid substitutions thereto, an HCDR2 comprising or consisting of a sequence selected from the group consisting of SEQ ID NOs: 14 to 23 and optionally having up to 2 amino acid substitutions thereto, and an HCDR3 comprising or consisting of a sequence selected from the group consisting of SEQ ID NOs: 27 to 36 and optionally having up to 2 amino acid substitutions thereto;
      ii. a heavy chain constant region comprising or consisting of a sequence having at least 90%, at least 91%, at least 92%, at least 93%, at least 94%, at least 95%, at least 95%, at least 97%, at least 98% or at least 99% identity to a sequence selected from the group consisting of SEQ ID NOs: 105, 106 and 203;
      iii. optionally a linker; and
      iv. an IL-15 polypeptide comprising or consisting of a sequence having at least 90%, at least 91%, at least 92%, at least 93%, at least 94%, at least 95%, at least 95%, at least 97%, at least 98% or at least 99% identity to a sequence selected from the group consisting of SEQ ID NOs: 148 to 155, 206 and 207; and
   b) a second polypeptide chain comprising, in an N- to C-terminal direction:
      i. a light chain variable region (VL) of an anti-NKG2D antibody comprising an LCDR1 comprising or consisting of a sequence selected from the group consisting of SEQ ID NOs: 40 to 49 and optionally having up to 2 amino acid substitutions thereto, an LCDR2 comprising or consisting of a sequence selected from the group consisting of SEQ ID NOs: 53 to 62 and optionally having up to 2 amino acid substitutions thereto, and an LCDR3 comprising or consisting of a sequence selected from the group consisting of SEQ ID NOs: 66 to 75 and optionally having up to 2 amino acid substitutions thereto; and
      ii. a light chain constant region comprising or consisting of a sequence having at least 90%, at least 91%, at least 92%, at least 93%, at least 94%, at least 95%, at least 95%, at least 97%, at least 98% or at least 99% identity to a sequence selected from the group consisting of SEQ ID NOs: 135 to 144;
         and the immunoconjugate comprises or consists of first and second copies of said first and second polypeptides.
86. The immunoconjugate of any preceding embodiment, wherein the immunoconjugate comprises:
   a) a first polypeptide chain comprising, in an N- to C-terminal direction:
      i. a heavy chain variable region (VH) of an anti-NKG2D antibody comprising an HCDR1 comprising or consisting of a sequence selected from the group con-sisting of SEQ ID NOs: 1 to 10, an HCDR2 comprising or consisting of a sequence selected from the group consisting of SEQ ID NOs: 14 to 23 and an HCDR3 comprising or consisting of a sequence selected from the group con-sisting of SEQ ID NOs: 27 to 36;
      ii. a heavy chain constant region comprising or consisting of a sequence selected from the group consisting of SEQ ID NOs: 105, 106 and 203;
      iii. optionally a linker; and
      iv. an IL-15 polypeptide comprising or consisting of a sequence selected from the group consisting of SEQ ID NOs: 148 to 155, 206 and 207; and
   b) a second polypeptide chain comprising, in an N- to C-terminal direction:
      i. a light chain variable region (VL) of an anti-NKG2D antibody comprising an LCDR1 comprising or consisting of a sequence selected from the group con-sisting of SEQ ID NOs: 40 to 49, an LCDR2 comprising or consisting of a sequence selected from the group consisting of SEQ ID NOs: 53 to 62 and an LCDR3 comprising or consisting of a sequence selected from the group con-sisting of SEQ ID NOs: 66 to 75; and
      ii. a light chain constant region comprising or consisting of a sequence selected from the group consisting of SEQ ID NOs: 107 to 108;
      and the immunoconjugate comprises or consists of first and second copies of said first and second polypeptides.
87. The immunoconjugate of any preceding embodiment, wherein the immunoconjugate comprises:
a) a first polypeptide chain comprising, in an N- to C-terminal direction:
   i. a heavy chain variable region (VH) of an anti-NKG2D antibody comprising an HCDR1 comprising or consisting of the sequence of SEQ ID NO: 6, an HCDR2 comprising or consisting of the sequence of SEQ ID NO: 19 and an HCDR3 comprising or consisting of the sequence of SEQ ID NO: 32, optionally wherein the VH region comprises or consists of the sequence of SEQ ID NO:84;
   ii. a heavy chain constant region comprising or consisting of a sequence of SEQ ID NO: 105, 106 and 203;
   iii. optionally a linker; and
   iv. an IL-15 polypeptide comprising or consisting of a sequence selected from the group consisting of SEQ ID NOs: 148 to 155, 206 and 207; and
b) a second polypeptide chain comprising, in an N- to C-terminal direction:
   i. a light chain variable region (VL) of an anti-NKG2D antibody comprising an LCDR1 comprising or consisting of the sequence of SEQ ID NO: 45, an LCDR2 comprising or consisting of the sequence of SEQ ID NO: 58 and an LCDR3 comprising or consisting of the sequence of SEQ ID NO: 71, optionally wherein the VL region comprises or consists of the sequence of SEQ ID NO: 97; and
   ii. a light chain constant region comprising or consisting of the sequence of SEQ ID NO: 107; and the immunoconjugate comprises or consists of first and second copies of said first and second polypeptides.
88. The immunoconjugate of any preceding embodiment, wherein the immunoconjugate comprises:
a) a first polypeptide chain comprising, in an N- to C-terminal direction:
   i. a heavy chain variable region (VH) of an anti-NKG2D antibody comprising an HCDR1 comprising or consisting of the sequence of SEQ ID NO: 6, an HCDR2 comprising or consisting of the sequence of SEQ ID NO: 19 and an HCDR3 comprising or consisting of the sequence of SEQ ID NO: 32, optionally wherein the VH region comprises or consists of the sequence of SEQ ID NO:84;
   ii. a heavy chain constant region comprising or consisting of a sequence of SEQ ID NO: 105;
   iii. optionally a linker; and
   iv. an IL-15 polypeptide comprising or consisting of a sequence selected from the group consisting of SEQ ID NOs: 148 to 155, 206 and 207; and
b) a second polypeptide chain comprising, in an N- to C-terminal direction:
   i. a light chain variable region (VL) of an anti-NKG2D antibody comprising an LCDR1 comprising or consisting of the sequence of SEQ ID NO: 45, an LCDR2 comprising or consisting of the sequence of SEQ ID NO: 58 and an LCDR3 comprising or consisting of the sequence of SEQ ID NO: 71, optionally wherein the VL region comprises or consists of the sequence of SEQ ID NO: 97; and
   ii. a light chain constant region comprising or consisting of the sequence of SEQ ID NO: 107;
      and the immunoconjugate comprises or consists of first and second copies of said first and second polypeptides.
89. The immunoconjugate of any preceding embodiment, wherein the immunoconjugate comprises:
a) a first polypeptide chain comprising, in an N- to C-terminal direction:
   i. a heavy chain variable region (VH) of an anti-NKG2D antibody comprising an HCDR1 comprising or consisting of the sequence of SEQ ID NO: 6, an HCDR2 comprising or consisting of the sequence of SEQ ID NO: 19 and an HCDR3 comprising or consisting of the sequence of SEQ ID NO: 32, optionally wherein the VH region comprises or consists of the sequence of SEQ ID NO:84;
   ii. a heavy chain constant region comprising or consisting of a sequence of SEQ ID NO: 105;
   iii. optionally a linker; and
   iv. an IL-15 polypeptide comprising or consisting of a sequence selected from the group consisting of SEQ ID NOs: 154, 206 and 207; and
b) a second polypeptide chain comprising, in an N- to C-terminal direction:
   i. a light chain variable region (VL) of an anti-NKG2D antibody comprising an LCDR1 comprising or consisting of the sequence of SEQ ID NO: 45, an LCDR2 comprising or consisting of the sequence of SEQ ID NO: 58 and an LCDR3 comprising or consisting of the sequence of SEQ ID NO: 71, optionally wherein the VL region comprises or consists of the sequence of SEQ ID NO: 97; and
   ii. a light chain constant region comprising or consisting of the sequence of SEQ ID NO: 107;
      and the immunoconjugate comprises or consists of first and second copies of said first and second polypeptides.
90. The immunoconjugate of any one of embodiments 78 to 88, wherein the IL-15 polypeptide comprises or consists of a sequence selected from the group consisting of SEQ ID NOs: 154, 206 and 207.
91. The immunoconjugate of any preceding embodiment, wherein the immunoconjugate comprises:
a) a first polypeptide chain comprising, in an N- to C-terminal direction:
   i. a heavy chain variable region (VH) of an anti-NKG2D antibody comprising an HCDR1 comprising or consisting of the sequence of SEQ ID NO: 6, an HCDR2 comprising or consisting of the sequence of SEQ ID NO: 19 and an HCDR3 comprising or consisting of the sequence of SEQ ID NO: 32, optionally wherein the VH region comprises or consists of the sequence of SEQ ID NO:84;
   ii. a heavy chain constant region comprising or consisting of a sequence of SEQ ID NO: 105;
   iii. optionally a linker; and
   iv. an IL-15 polypeptide comprising or consisting of the sequence of SEQ ID NOs: 154; and
b) a second polypeptide chain comprising, in an N- to C-terminal direction:
   i. a light chain variable region (VL) of an anti-NKG2D antibody comprising an LCDR1 comprising or consisting of the sequence of SEQ ID NO: 45, an LCDR2 comprising or consisting of the sequence of SEQ ID NO: 58 and an LCDR3 comprising or consisting of the sequence of SEQ ID NO: 71, optionally wherein the VL region comprises or consists of the sequence of SEQ ID NO: 97; and
   ii. a light chain constant region comprising or consisting of the sequence of SEQ ID NO: 107;
      and the immunoconjugate comprises or consists of first and second copies of said first and second polypeptides.
92. The immunoconjugate of any one of embodiments 78 to 90, wherein the linker comprises or consists of a sequence selected from the group consisting of SEQ ID NO: 182 to 187, 201, 204 and 205.
93. The immunoconjugate of any preceding embodiment, wherein the immunoconjugate comprises:
a) a first polypeptide chain comprising, in an N- to C-terminal direction:
   i. a heavy chain variable region (VH) of an anti-NKG2D antibody comprising an HCDR1 comprising or consisting of the sequence of SEQ ID NO: 6, an HCDR2 comprising or consisting of the sequence of SEQ ID NO: 19 and an HCDR3 comprising or consisting of the sequence of SEQ ID NO: 32, optionally wherein the VH region comprises or consists of the sequence of SEQ ID NO:84;
   ii. a heavy chain constant region comprising or consisting of a sequence of SEQ ID NO: 105;
   iii. optionally a linker comprising or consisting of glycine and serine residues; and
   iv. an IL-15 polypeptide comprising or consisting of the sequence of SEQ ID NOs: 154; and
b) a second polypeptide chain comprising, in an N- to C-terminal direction:
   i. a light chain variable region (VL) of an anti-NKG2D antibody comprising an LCDR1 comprising or consisting of the sequence of SEQ ID NO: 45, an LCDR2 comprising or consisting of the sequence of SEQ ID NO: 58 and an LCDR3 comprising or consisting of the sequence of SEQ ID NO: 71, optionally wherein the VL region comprises or consists of the sequence of SEQ ID NO: 97; and
   ii. a light chain constant region comprising or consisting of the sequence of SEQ ID NO: 107;
      and the immunoconjugate comprises or consists of first and second copies of said first and second polypeptides.
94. The immunoconjugate of any preceding embodiment, wherein the immunoconjugate comprises:
a) a first polypeptide chain comprising, in an N- to C-terminal direction:
   i. a heavy chain variable region (VH) of an anti-NKG2D antibody comprising an HCDR1 comprising or consisting of the sequence of SEQ ID NO: 6, an HCDR2 comprising or consisting of the sequence of SEQ ID NO: 19 and an HCDR3 comprising or consisting of the sequence of SEQ ID NO: 32, optionally wherein the VH region comprises or consists of the sequence of SEQ ID NO:84;
   ii. a heavy chain constant region comprising or consisting of a sequence of SEQ ID NO: 105;
   iii. optionally a linker comprising or consisting of the sequence of SEQ ID NO: 182; and
   iv. an IL-15 polypeptide comprising or consisting of the sequence of SEQ ID NOs: 154; and
b) a second polypeptide chain comprising, in an N- to C-terminal direction:
   i. a light chain variable region (VL) of an anti-NKG2D antibody comprising an LCDR1 comprising or consisting of the sequence of SEQ ID NO: 45, an LCDR2 comprising or consisting of the sequence of SEQ ID NO: 58 and an LCDR3 comprising or consisting of the sequence of SEQ ID NO: 71, optionally wherein the VL region comprises or consists of the sequence of SEQ ID NO: 97; and
   ii. a light chain constant region comprising or consisting of the sequence of SEQ ID NO: 107;
      and the immunoconjugate comprises or consists of first and second copies of said first and second polypeptides.
95. The immunoconjugate of any preceding embodiment, wherein the immunoconjugate comprises:
a) a first polypeptide chain comprising, in an N- to C-terminal direction:
   i. a heavy chain variable region (VH) of an anti-NKG2D antibody comprising an HCDR1 comprising or consisting of the sequence of SEQ ID NO: 6, an HCDR2 comprising or consisting of the sequence of SEQ ID NO: 19 and an HCDR3 comprising or consisting of the sequence of SEQ ID NO: 32, optionally wherein the VH region comprises or consists of the sequence of SEQ ID NO:84;
   ii. a heavy chain constant region comprising or consisting of a sequence of SEQ ID NO: 105;
   iii. optionally a linker comprising or consisting of the sequence of SEQ ID NO: 184; and
   iv. an IL-15 polypeptide comprising or consisting of the sequence of SEQ ID NOs: 154; and
b) a second polypeptide chain comprising, in an N- to C-terminal direction:
   i. a light chain variable region (VL) of an anti-NKG2D antibody comprising an LCDR1 comprising or consisting of the sequence of SEQ ID NO: 45, an LCDR2 comprising or consisting of the sequence of SEQ ID NO: 58 and an LCDR3 comprising or consisting of the sequence of SEQ ID NO: 71, optionally wherein the VL region comprises or consists of the sequence of SEQ ID NO: 97; and
   ii. a light chain constant region comprising or consisting of the sequence of SEQ ID NO: 107;
      and the immunoconjugate comprises or consists of first and second copies of said first and second polypeptides.
96. The immunoconjugate of any preceding embodiment, wherein the immunoconjugate comprises:
a) a first polypeptide chain comprising, in an N- to C-terminal direction:
   i. a heavy chain variable region (VH) of an anti-NKG2D antibody comprising an HCDR1 comprising or consisting of the sequence of SEQ ID NO: 6, an HCDR2 comprising or consisting of the sequence of SEQ ID NO: 19 and an HCDR3 comprising or consisting of the sequence of SEQ ID NO: 32, optionally wherein the VH region comprises or consists of the sequence of SEQ ID NO:84;
   ii. a heavy chain constant region comprising or consisting of a sequence of SEQ ID NO: 105;
   iii. optionally a linker; and
   iv. an IL-15 polypeptide comprising or consisting of the sequence of SEQ ID NOs: 206; and
b) a second polypeptide chain comprising, in an N- to C-terminal direction:
   i. a light chain variable region (VL) of an anti-NKG2D antibody comprising an LCDR1 comprising or consisting of the sequence of SEQ ID NO: 45, an LCDR2 comprising or consisting of the sequence of SEQ ID NO: 58 and an LCDR3 comprising or consisting of the sequence of SEQ ID NO: 71, optionally wherein the VL region comprises or consists of the sequence of SEQ ID NO: 97; and
   ii. a light chain constant region comprising or consisting of the sequence of SEQ ID NO: 107;
      and the immunoconjugate comprises or consists of first and second copies of said first and second polypeptides.
97. The immunoconjugate of embodiment 96, wherein the linker comprises or consists of a sequence selected from the group consisting of SEQ ID NO: 182 to 187, 201, 204 and 205.
98. The immunoconjugate of any preceding embodiment, wherein the immunoconjugate comprises:
a) a first polypeptide chain comprising, in an N- to C-terminal direction:
   i. a heavy chain variable region (VH) of an anti-NKG2D antibody comprising an HCDR1 comprising or consisting of the sequence of SEQ ID NO: 6, an HCDR2 comprising or consisting of the sequence of SEQ ID NO: 19 and an HCDR3 comprising or consisting of the sequence of SEQ ID NO: 32, optionally wherein the VH region comprises or consists of the sequence of SEQ ID NO:84;
   ii. a heavy chain constant region comprising or consisting of a sequence of SEQ ID NO: 105;
   iii. optionally a linker; and
   iv. an IL-15 polypeptide comprising or consisting of the sequence of SEQ ID NOs: 207; and
a) a second polypeptide chain comprising, in an N- to C-terminal direction:
   i. a light chain variable region (VL) of an anti-NKG2D antibody comprising an LCDR1 comprising or consisting of the sequence of SEQ ID NO: 45, an LCDR2 comprising or consisting of the sequence of SEQ ID NO: 58 and an LCDR3 comprising or consisting of the sequence of SEQ ID NO: 71, optionally wherein the VL region comprises or consists of the sequence of SEQ ID NO: 97; and
   ii. a light chain constant region comprising or consisting of the sequence of SEQ ID NO: 107;
      and the immunoconjugate comprises or consists of first and second copies of said first and second polypeptides.
99. The immunoconjugate of embodiment 98, wherein the linker comprises or consists of a sequence selected from the group consisting of SEQ ID NO: 182 to 187, 201, 204 and 205.
100. The immunoconjugate of any preceding embodiment, wherein the immunoconjugate comprises:
a) a first polypeptide chain comprising, in an N- to C-terminal direction:
   i. a heavy chain variable region (VH) of an anti-NKG2D antibody comprising an HCDR1 comprising or consisting of the sequence of SEQ ID NO: 6, an HCDR2 comprising or consisting of the sequence of SEQ ID NO: 19 and an HCDR3 comprising or consisting of the sequence of SEQ ID NO: 32, optionally wherein the VH region comprises or consists of the sequence of SEQ ID NO:84;
   ii. a heavy chain constant region comprising or consisting of a sequence of SEQ ID NO: 105;
   iii. optionally a linker comprising or consisting of the sequence of SEQ ID NO: 182; and
   iv. an IL-15 polypeptide comprising or consisting of the sequence of SEQ ID NOs: 207; and
b) a second polypeptide chain comprising, in an N- to C-terminal direction:
   i. a light chain variable region (VL) of an anti-NKG2D antibody comprising an LCDR1 comprising or consisting of the sequence of SEQ ID NO: 45, an LCDR2 comprising or consisting of the sequence of SEQ ID NO: 58 and an LCDR3 comprising or consisting of the sequence of SEQ ID NO: 71, optionally wherein the VL region comprises or consists of the sequence of SEQ ID NO: 97; and
   ii. a light chain constant region comprising or consisting of the sequence of SEQ ID NO: 107;
      and the immunoconjugate comprises or consists of first and second copies of said first and second polypeptides.
101. The immunoconjugate of any preceding embodiment, wherein the immunoconjugate comprises:
a) a first polypeptide chain comprising, in an N- to C-terminal direction:
   i. a heavy chain variable region (VH) of an anti-NKG2D antibody comprising an HCDR1 comprising or consisting of the sequence of SEQ ID NO: 1, an HCDR2 comprising or consisting of the sequence of SEQ ID NO: 14 and an HCDR3 comprising or consisting of the sequence of SEQ ID NO: 27, optionally wherein the VH region comprises or consists of the sequence of SEQ ID NO:79 or 202;
   ii. a heavy chain constant region comprising or consisting of a sequence of SEQ ID NO: 105, 106 and 203;
   iii. optionally a linker; and
   iv. an IL-15 polypeptide comprising or consisting of a sequence selected from the group consisting of SEQ ID NOs: 148 to 155, 206 and 207; and
b) a second polypeptide chain comprising, in an N- to C-terminal direction:
   i. a light chain variable region (VL) of an anti-NKG2D antibody comprising an LCDR1 comprising or consisting of the sequence of SEQ ID NO: 40, an LCDR2 comprising or consisting of the sequence of SEQ ID NO: 53 and an LCDR3 comprising or consisting of the sequence of SEQ ID NO: 66, optionally wherein the VL region comprises or consists of the sequence of SEQ ID NO: 92; and
   ii. a light chain constant region comprising or consisting of the sequence of SEQ ID NO: 108;
      and the immunoconjugate comprises or consists of first and second copies of said first and second polypeptides.
102. The immunoconjugate of any preceding embodiment, wherein the immunoconjugate comprises:
a) a first polypeptide chain comprising, in an N- to C-terminal direction:
   i. a heavy chain variable region (VH) of an anti-NKG2D antibody comprising an HCDR1 comprising or consisting of the sequence of SEQ ID NO: 1, an HCDR2 comprising or consisting of the sequence of SEQ ID NO: 14 and an HCDR3 comprising or consisting of the sequence of SEQ ID NO: 27, optionally wherein the VH region comprises or consists of the sequence of SEQ ID NO:79 or 202;
   ii. a heavy chain constant region comprising or consisting of a sequence of SEQ ID NO: 105;
   iii. optionally a linker; and
   iv. an IL-15 polypeptide comprising or consisting of a sequence selected from the group consisting of SEQ ID NOs: 148 to 155, 206 and 207; and
b) a second polypeptide chain comprising, in an N- to C-terminal direction:
   i. a light chain variable region (VL) of an anti-NKG2D antibody comprising an LCDR1 comprising or consisting of the sequence of SEQ ID NO: 40, an LCDR2 comprising or consisting of the sequence of SEQ ID NO: 53 and an LCDR3 comprising or consisting of the sequence of SEQ ID NO: 66, optionally wherein the VL region comprises or consists of the sequence of SEQ ID NO: 92; and
   ii. a light chain constant region comprising or consisting of the sequence of SEQ ID NO: 108;
      and the immunoconjugate comprises or consists of first and second copies of said first and second polypeptides.
103. The immunoconjugate of any preceding embodiment, wherein the immunoconjugate comprises:
a) a first polypeptide chain comprising, in an N- to C-terminal direction:
   i. a heavy chain variable region (VH) of an anti-NKG2D antibody comprising an HCDR1 comprising or consisting of the sequence of SEQ ID NO: 1, an HCDR2 comprising or consisting of the sequence of SEQ ID NO: 14 and an HCDR3 comprising or consisting of the sequence of SEQ ID NO: 27, optionally wherein the VH region comprises or consists of the sequence of SEQ ID NO:79 or 202;
   ii. a heavy chain constant region comprising or consisting of a sequence of SEQ ID NO: 105;
   iii. optionally a linker; and
   iv. an IL-15 polypeptide comprising or consisting of a sequence selected from the group consisting of SEQ ID NOs: 154, 206 and 207; and
b) a second polypeptide chain comprising, in an N- to C-terminal direction:
   i. a light chain variable region (VL) of an anti-NKG2D antibody comprising an LCDR1 comprising or consisting of the sequence of SEQ ID NO: 40, an LCDR2 comprising or consisting of the sequence of SEQ ID NO: 53 and an LCDR3 comprising or consisting of the sequence of SEQ ID NO: 66, optionally wherein the VL region comprises or consists of the sequence of SEQ ID NO: 92; and
   ii. a light chain constant region comprising or consisting of the sequence of SEQ ID NO: 108;
      and the immunoconjugate comprises or consists of first and second copies of said first and second polypeptides.
104. The immunoconjugate of any preceding embodiment, wherein the immunoconjugate comprises:
a) a first polypeptide chain comprising, in an N- to C-terminal direction:
   i. a heavy chain variable region (VH) of an anti-NKG2D antibody comprising an HCDR1 comprising or consisting of the sequence of SEQ ID NO: 1, an HCDR2 comprising or consisting of the sequence of SEQ ID NO: 14 and an HCDR3 comprising or consisting of the sequence of SEQ ID NO: 27, optionally wherein the VH region comprises or consists of the sequence of SEQ ID NO:79 or 202;
   ii. a heavy chain constant region comprising or consisting of a sequence of SEQ ID NO: 105;
   iii. optionally a linker; and
   iv. an IL-15 polypeptide comprising or consisting of the sequence of SEQ ID NOs: 154; and
b) a second polypeptide chain comprising, in an N- to C-terminal direction:
   i. a light chain variable region (VL) of an anti-NKG2D antibody comprising an LCDR1 comprising or consisting of the sequence of SEQ ID NO: 40, an LCDR2 comprising or consisting of the sequence of SEQ ID NO: 53 and an LCDR3 comprising or consisting of the sequence of SEQ ID NO: 66, optionally wherein the VL region comprises or consists of the sequence of SEQ ID NO: 92; and
   ii. a light chain constant region comprising or consisting of the sequence of SEQ ID NO: 108;
      and the immunoconjugate comprises or consists of first and second copies of said first and second polypeptides.
105. The immunoconjugate of embodiment 98, wherein the linker comprises or consists of a sequence selected from the group consisting of SEQ ID NO: 182 to 187, 201, 204 and 205.
106. The immunoconjugate of any preceding embodiment, wherein the immunoconjugate comprises:
a) a first polypeptide chain comprising, in an N- to C-terminal direction:
   i. a heavy chain variable region (VH) of an anti-NKG2D antibody comprising an HCDR1 comprising or consisting of the sequence of SEQ ID NO: 1, an HCDR2 comprising or consisting of the sequence of SEQ ID NO: 14 and an HCDR3 comprising or consisting of the sequence of SEQ ID NO: 27, optionally wherein the VH region comprises or consists of the sequence of SEQ ID NO:79 or 202;
   ii. a heavy chain constant region comprising or consisting of a sequence of SEQ ID NO: 105;
   iii. optionally a linker; and
   iv. an IL-15 polypeptide comprising or consisting of the sequence of SEQ ID Nos: 206; and
b) a second polypeptide chain comprising, in an N- to C-terminal direction:
   i. a light chain variable region (VL) of an anti-NKG2D antibody comprising an LCDR1 comprising or consisting of the sequence of SEQ ID NO: 40, an LCDR2 comprising or consisting of the sequence of SEQ ID NO: 53 and an LCDR3 comprising or consisting of the sequence of SEQ ID NO: 66, optionally wherein the VL region comprises or consists of the sequence of SEQ ID NO: 92; and
   ii. a light chain constant region comprising or consisting of the sequence of SEQ ID NO: 108;
      and the immunoconjugate comprises or consists of first and second copies of said first and second polypeptides.
107. The immunoconjugate of embodiment 106, wherein the linker comprises or consists of a sequence selected from the group consisting of SEQ ID NO: 182 to 187, 201, 204 and 205.
108. The immunoconjugate of any preceding embodiment, wherein the immunoconjugate comprises:
a) a first polypeptide chain comprising, in an N- to C-terminal direction:
   i. a heavy chain variable region (VH) of an anti-NKG2D antibody comprising an HCDR1 comprising or consisting of the sequence of SEQ ID NO: 1, an HCDR2 comprising or consisting of the sequence of SEQ ID NO: 14 and an HCDR3 comprising or consisting of the sequence of SEQ ID NO: 27, optionally wherein the VH region comprises or consists of the sequence of SEQ ID NO:79 or 202;
   ii. a heavy chain constant region comprising or consisting of a sequence of SEQ ID NO: 105;
   iii. optionally a linker; and
   iv. an IL-15 polypeptide comprising or consisting of the sequence of SEQ ID NOs: 207; and
b) a second polypeptide chain comprising, in an N- to C-terminal direction:
   i. a light chain variable region (VL) of an anti-NKG2D antibody comprising an LCDR1 comprising or consisting of the sequence of SEQ ID NO: 40, an LCDR2 comprising or consisting of the sequence of SEQ ID NO: 53 and an LCDR3 comprising or consisting of the sequence of SEQ ID NO: 66, optionally wherein the VL region comprises or consists of the sequence of SEQ ID NO: 92; and
   ii. a light chain constant region comprising or consisting of the sequence of SEQ ID NO: 108;
      and the immunoconjugate comprises or consists of first and second copies of said first and second polypeptides.
109. The immunoconjugate of embodiment 108, wherein the linker comprises or consists of a sequence selected from the group consisting of SEQ ID NO: 182 to 187, 201, 204 and 205.
110. The immunoconjugate of any preceding embodiment, wherein the immunoconjugate comprises:
   a) a first polypeptide chain comprising, in an N- to C-terminal direction:
      i. a heavy chain variable region (VH) of an anti-NKp30 antibody comprising an HCDR1 comprising or consisting of a sequence selected from the group consisting of SEQ ID NOs: 11 to 12 and optionally having up to 2 amino acid substitutions thereto, an HCDR2 comprising or consisting of a sequence selected from the group consisting of SEQ ID NOs: 24 to 25 and optionally having up to 2 amino acid substitutions thereto, and an HCDR3 comprising or consisting of a sequence selected from the group consisting of SEQ ID NOs: 37 to 38 and optionally having up to 2 amino acid substitutions thereto;
      ii. a heavy chain constant region comprising or consisting of a sequence having at least 90%, at least 91%, at least 92%, at least 93%, at least 94%, at least 95%, at least 95%, at least 97%, at least 98% or at least 99% identity to a sequence selected from the group consisting of SEQ ID NOs: 105, 106 and 203;
      iii. optionally a linker; and
      iv. an IL-15 polypeptide comprising or consisting of a sequence having at least 90%, at least 91%, at least 92%, at least 93%, at least 94%, at least 95%, at least 95%, at least 97%, at least 98% or at least 99% identity to a sequence selected from the group consisting of SEQ ID NOs: 148 to 155, 206 and 207; and
   b) a second polypeptide chain comprising, in an N- to C-terminal direction:
      i. a light chain variable region (VL) of an anti-NKp30 antibody comprising an LCDR1 comprising or consisting of a sequence selected from the group consisting of SEQ ID NOs: 50 to 51 and optionally having up to 2 amino acid substitutions thereto, an LCDR2 comprising or consisting of a sequence selected from the group consisting of SEQ ID NOs: 63 to 64 and optionally having up to 2 amino acid substitutions thereto, and an LCDR3 comprising or consisting of a sequence selected from the group consisting of SEQ ID NOs: 76 to 77 and optionally having up to 2 amino acid substitutions thereto; and
      ii. a light chain constant region comprising or consisting of a sequence having at least 90%, at least 91%, at least 92%, at least 93%, at least 94%, at least 95%, at least 95%, at least 97%, at least 98% or at least 99% identity to a sequence selected from the group consisting of SEQ ID NOs: 107 to 108;
         and the immunoconjugate comprises or consists of first and second copies of said first and second polypeptides.
111. The immunoconjugate of any preceding embodiment, wherein the immunoconjugate comprises:
   a) a first polypeptide chain comprising, in an N- to C-terminal direction:
      i. a heavy chain variable region (VH) of an anti-NKp30 antibody comprising an HCDR1 comprising or consisting of a sequence selected from the group con-sisting of SEQ ID NOs: 11 to 12, an HCDR2 comprising or consisting of a sequence selected from the group consisting of SEQ ID NOs: 24 to 25 and an HCDR3 comprising or consisting of a sequence selected from the group con-sisting of SEQ ID NOs: 37 to 38;
      ii. a heavy chain constant region comprising or consisting of a sequence selected from the group consisting of SEQ ID NOs: 105, 106 and 203;
      iii. optionally a linker; and
      iv. an IL-15 polypeptide comprising or consisting of a sequence selected from the group consisting of SEQ ID NOs: 148 to 155, 206 and 207; and
   b) a second polypeptide chain comprising, in an N- to C-terminal direction:
      i. a light chain variable region (VL) of an anti-NKp30 antibody comprising an LCDR1 comprising or consisting of a sequence selected from the group con-sisting of SEQ ID NOs: 50 to 51, an LCDR2 comprising or consisting of a sequence selected from the group consisting of SEQ ID NOs: 63 to 64 and an LCDR3 comprising or consisting of a sequence selected from the group con-sisting of SEQ ID NOs: 76 to 77; and
      ii. a light chain constant region comprising or consisting of a sequence selected from the group consisting of SEQ ID NOs: 107 to 108;
         and the immunoconjugate comprises or consists of first and second copies of said first and second polypeptides.
112. The immunoconjugate of any preceding embodiment, wherein the immunoconjugate comprises:
   a) a first polypeptide chain comprising, in an N- to C-terminal direction:
      i. a heavy chain variable region (VH) of an anti-NKp46 antibody comprising an HCDR1 comprising or consisting of SEQ ID NO: 13 and optionally having up to 2 amino acid substitutions thereto, an HCDR2 comprising or consisting of SEQ ID NO: 26 and optionally having up to 2 amino acid substitutions thereto, and an HCDR3 comprising or consisting of SEQ ID NO: 39 and optionally having up to 2 amino acid substitutions thereto;
      ii. a heavy chain constant region comprising or consisting of a sequence having at least 90%, at least 91%, at least 92%, at least 93%, at least 94%, at least 95%, at least 95%, at least 97%, at least 98% or at least 99% identity to a sequence se-lected from the group consisting of SEQ ID NO: 105 to 106;
      iii. optionally a linker; and
      iv. an IL-15 polypeptide comprising or consisting of a sequence having at least 90%, at least 91%, at least 92%, at least 93%, at least 94%, at least 95%, at least 95%, at least 97%, at least 98% or at least 99% identity to a sequence selected from the group consisting of SEQ ID NOs: 148 to 155, 206 and 207; and
   b) a second polypeptide chain comprising, in an N- to C-terminal direction:
      i. a light chain variable region (VL) of an anti-NKp46 antibody comprising an LCDR1 comprising or consisting of SEQ ID NO: 52 and optionally having up to 2 amino acid substitutions thereto, an LCDR2 comprising or consisting of SEQ ID NO: 65 and optionally having up to 2 amino acid substitutions thereto, and an LCDR3 comprising or consisting of SEQ ID NO: 78 and optionally having up to 2 amino acid substitutions thereto; and
      ii. a light chain constant region comprising or consisting of a sequence having at least 90%, at least 91%, at least 92%, at least 93%, at least 94%, at least 95%, at least 95%, at least 97%, at least 98% or at least 99% identity to SEQ ID NO: 107;
      and the immunoconjugate comprises or consists of first and second copies of said first and second polypeptides.
113. The immunoconjugate of any preceding embodiment, wherein the immunoconjugate comprises:
   a) a first polypeptide chain comprising, in an N- to C-terminal direction:
      i. a heavy chain variable region (VH) of an anti-NKp46 antibody comprising an HCDR1 comprising or consisting of SEQ ID NO: 13, an HCDR2 comprising or consisting of SEQ ID NO: 26, and an HCDR3 comprising or consisting of SEQ ID NO: 39;
      ii. a heavy chain constant region comprising or consisting of a sequence selected from the group consisting of SEQ ID NOs: 105 to 106;
      iii. optionally a linker; and
      iv. an IL-15 polypeptide comprising or consisting of a sequence selected from the group con-sisting of SEQ ID NOs: 148 to 155, 206 and 207; and
   b) a second polypeptide chain comprising, in an N- to C-terminal direction:
      i. a light chain variable region (VL) of an anti-NKp46 antibody comprising an LCDR1 comprising or consisting of SEQ ID NO: 52, an LCDR2 comprising or consisting of SEQ ID NO: 65 and an LCDR3 comprising or consisting of SEQ ID NO: 78; and
      ii. a light chain constant region comprising or consisting of SEQ ID NO: 107;
         and the immunoconjugate comprises or consists of first and second copies of said first and second polypeptides.
114. The immunoconjugate of any one of embodiments 78 to 113, wherein the IL-15 polypeptide is an IL-15 polypeptide as defined in any one of embodiments 48 to 78.
115. The immunoconjugate of any preceding embodiment, wherein the immunoconjugate comprises:
   a) a first polypeptide chain comprising or consisting of a sequence having at least 90%, at least 91%, at least 92%, at least 93%, at least 94%, at least 95%, at least 95%, at least 97%, at least 98% or at least 99% identity to a sequence selected from the group consisting of SEQ ID NOs: 156 to 168; and
   b) a second polypeptide chain comprising or consisting of a sequence having at least 90%, at least 91%, at least 92%, at least 93%, at least 94%, at least 95%, at least 95%, at least 97%, at least 98% or at least 99% identity to a sequence selected from the group consisting of SEQ ID NOs: 135 to 147;
   and the immunoconjugate comprises or consists of first and second copies of said first and second polypeptides.
116. The immunoconjugate of any preceding embodiment, wherein the immunoconjugate comprises:
   a) a first polypeptide chain comprising or consisting of a sequence selected from the group consisting of SEQ ID NOs: 156 to 168; and
   b) a second polypeptide chain comprising or consisting of a sequence selected from the group consisting of SEQ ID NOs: 135 to 147;
   and the immunoconjugate comprises or consists of first and second copies of said first and second polypeptides.
117. The immunoconjugate of any preceding embodiment, wherein the immunoconjugate comprises:
   a) a first polypeptide chain comprising, in an N- to C-terminal direction:
      i. a heavy chain variable region (VH) of an anti-NKG2D antibody, an anti-NKp30 antibody, an anti-NKp46 antibody, an anti-NKp44 antibody or an anti-DNAM-1 antibody;
      ii. a human heavy chain constant region; and
   b) a second polypeptide chain comprising, in an N- to C-terminal direction:
      i. a light chain variable region (VL) of an anti-NKG2D antibody, an anti-NKp30 antibody, an anti-NKp46 antibody, an anti-NKp44 antibody or an anti-DNAM-1 antibody; and
      ii. a human light constant region;
      iii. optionally a linker; and
      iv. the IL-15 polypeptide;
         and the immunoconjugate comprises or consists of first and second copies of said first and second polypeptides.
118. The immunoconjugate of any preceding embodiment, wherein the immunoconjugate comprises:
   a) a first polypeptide chain comprising, in an N- to C-terminal direction:
      i. a heavy chain variable region (VH) of an anti-NKG2D antibody, an anti-NKp30 antibody, an anti-NKp46 antibody, an anti-NKp44 antibody or an anti-DNAM-1 antibody;
      ii. a human heavy chain IgG sequence; and
   b) a second polypeptide chain comprising, in an N- to C-terminal direction:
      i. a light chain variable region (VL) of an anti-NKG2D antibody, an anti-NKp30 antibody, an anti-NKp46 antibody, an anti-NKp44 antibody or an anti-DNAM-1 antibody; and
      ii. a human light chain IgG sequence;
      iii. optionally a linker; and
      iv. the IL-15 polypeptide;
         and the immunoconjugate comprises or consists of first and second copies of said first and second polypeptides.
119. The immunoconjugate of any preceding embodiment, wherein the immunoconjugate comprises:
   a) a first polypeptide chain comprising, in an N- to C-terminal direction:
      i. a heavy chain variable region (VH) of an anti-NKG2D antibody, an anti-NKp30 antibody, an anti-NKp46 antibody, an anti-NKp44 antibody or an anti-DNAM-1 antibody;
      ii. a human heavy chain IgG1 sequence; and
   b) a second polypeptide chain comprising, in an N- to C-terminal direction:
      i. a light chain variable region (VL) of an anti-NKG2D antibody, an anti-NKp30 antibody, an anti-NKp46 antibody, an anti-NKp44 antibody or an anti-DNAM-1 antibody; and
      ii. a human light chain IgG1 sequence;
      iii. optionally a linker; and
      iv. the IL-15 polypeptide;
         and the immunoconjugate comprises or consists of first and second copies of said first and second polypeptides.
120. The immunoconjugate of any preceding embodiment, wherein the immunoconjugate comprises:
   a) a first polypeptide chain comprising a heavy chain of an anti-NKG2D antibody, an anti-NKp30 antibody, an anti-NKp46 antibody, an anti-NKp44 antibody or an anti-DNAM-1 antibody; and
   b) a second polypeptide chain comprising in an N- to C-terminal direction
      i. a light chain of an anti-NKG2D antibody, an anti-NKp30 antibody, an anti-NKp46 antibody, an anti-NKp44 antibody or an anti-DNAM-1 antibody;
      ii. optionally a linker; and
      iii. the IL-15 polypeptide;
         and the immunoconjugate comprises or consists of first and second copies of said first and second polypeptides.
121. The immunoconjugate of any preceding embodiment, wherein the immunoconjugate comprises:
   a) a first polypeptide chain comprising, in an N- to C-terminal direction:
      i. a heavy chain variable region (VH) of an anti-NKG2D antibody, an anti-NKp30 antibody, an anti-NKp46 antibody, an anti-NKp44 antibody or an anti-DNAM-1 antibody, the VH comprising an HCDR1, an HCDR2 and an HCDR3;
      ii. a heavy chain constant region comprising an CH1, a hinge, a CH2 and a CH3 region; and
   b) a second polypeptide chain comprising, in an N- to C-terminal direction:
      i. a light chain variable region (VL) of an anti-NKG2D antibody, an anti-NKp30 antibody, an anti-NKp46 antibody, an anti-NKp44 antibody or an anti-DNAM-1 antibody, the VL comprising an LCDR1, an LCDR2 and an LCDR3; and
      ii. a light chain constant (CL) region;
      iii. optionally a linker, and
      iv. the IL-15 polypeptide;
         and the immunoconjugate comprises or consists of first and second copies of said first and second polypeptides.
122. The immunoconjugate of any preceding embodiment, wherein the immunoconjugate comprises:
   a) a first polypeptide chain comprising, in an N- to C-terminal direction:
      i. a heavy chain variable region (VH) of an anti-NKG2D antibody comprising an HCDR1 comprising or consisting of a sequence selected from the group consisting of SEQ ID NOs: 1 to 10 and optionally having up to 2 amino acid substitutions thereto, an HCDR2 comprising or consisting of a sequence selected from the group consisting of SEQ ID NOs: 14 to 23 and optionally having up to 2 amino acid substitutions thereto, and an HCDR3 comprising or consisting of a sequence selected from the group consisting of SEQ ID NOs: 27 to 36 and optionally having up to 2 amino acid substitutions thereto;
      ii. a heavy chain constant region comprising or consisting of a sequence having at least 90%, at least 91%, at least 92%, at least 93%, at least 94%, at least 95%, at least 95%, at least 97%, at least 98% or at least 99% identity to a sequence selected from the group consisting of SEQ ID NOs: 105, 106 and 203; and
   b) a second polypeptide chain comprising, in an N- to C-terminal direction:
      i. a light chain variable region (VL) of an anti-NKG2D antibody comprising an LCDR1 comprising or consisting of a sequence selected from the group consisting of SEQ ID NOs: 40 to 49 and optionally having up to 2 amino acid substitutions thereto, an LCDR2 comprising or consisting of a sequence selected from the group consisting of SEQ ID NOs: 53 to 62 and optionally having up to 2 amino acid substitutions thereto, and an LCDR3 comprising or consisting of a sequence selected from the group consisting of SEQ ID NOs: 66 to 75 and optionally having up to 2 amino acid substitutions thereto; and
      ii. a light chain constant region comprising or consisting of a sequence having at least 90%, at least 91%, at least 92%, at least 93%, at least 94%, at least 95%, at least 95%, at least 97%, at least 98% or at least 99% identity to a sequence selected from the group consisting of SEQ ID NOs: 107 to 108;
      iii. optionally a linker; and
      iv. an IL-15 polypeptide comprising or consisting of a sequence having at least 90%, at least 91%, at least 92%, at least 93%, at least 94%, at least 95%, at least 95%, at least 97%, at least 98% or at least 99% identity to a sequence selected from the group consisting of SEQ ID NOs: 148 to 155, 206 and 207;
         and the immunoconjugate comprises or consists of first and second copies of said first and second polypeptides.
123. The immunoconjugate of any preceding embodiment, wherein the immunoconjugate comprises:
   a) a first polypeptide chain comprising, in an N- to C-terminal direction:
      i. a heavy chain variable region (VH) of an anti-NKG2D antibody comprising an HCDR1 comprising or consisting of a sequence selected from the group consisting of SEQ ID NOs: 1 to 10, an HCDR2 comprising or consisting of a sequence selected from the group consisting of SEQ ID NOs: 14 to 23 and an HCDR3 comprising or consisting of a sequence selected from the group consisting of SEQ ID NOs: 27 to 36;
      ii. a heavy chain constant region comprising or consisting of a sequence selected from the group consisting of SEQ ID NOs: 105, 106 and 203; and
   b) a second polypeptide chain comprising, in an N- to C-terminal direction:
      i. a light chain variable region (VL) of an anti-NKG2D antibody comprising an LCDR1 comprising or consisting of a sequence selected from the group consisting of SEQ ID NOs: 40 to 49, an LCDR2 comprising or consisting of a sequence selected from the group consisting of SEQ ID NOs: 53 to 62 and an LCDR3 comprising or consisting of a sequence selected from the group consisting of SEQ ID NOs: 66 to 75; and
      ii. a light chain constant region comprising or consisting of a sequence selected from the group consisting of SEQ ID NOs: 107 to 108;
      iii. optionally a linker; and
      iv. an IL-15 polypeptide comprising or consisting of a sequence selected from the group con-sisting of SEQ ID NOs: 148 to 155, 206 and 207;
         and the immunoconjugate comprises or consists of first and second copies of said first and second polypeptides.
124. The immunoconjugate of any preceding embodiment, wherein the immunoconjugate comprises:
   a) a first polypeptide chain comprising, in an N- to C-terminal direction:
      i. a heavy chain variable region (VH) of an anti-NKp30 antibody comprising an HCDR1 comprising or consisting of a sequence selected from the group consisting of SEQ ID NOs: 11 to 12 and optionally having up to 2 amino acid substitutions thereto, an HCDR2 comprising or consisting of a sequence selected from the group consisting of SEQ ID NOs: 24 to 25 and optionally having up to 2 amino acid substitutions thereto, and an HCDR3 comprising or consisting of a sequence selected from the group consisting of SEQ ID NOs: 37 to 38 and optionally having up to 2 amino acid substitutions thereto;
      ii. a heavy chain constant region comprising or consisting of a sequence having at least 90%, at least 91%, at least 92%, at least 93%, at least 94%, at least 95%, at least 95%, at least 97%, at least 98% or at least 99% identity to a sequence selected from the group consisting of SEQ ID NOs: 105, 106 and 203; and
   b) a second polypeptide chain comprising, in an N- to C-terminal direction:
      i. a light chain variable region (VL) of an anti-NKp30 antibody comprising an LCDR1 comprising or consisting of a sequence selected from the group consisting of SEQ ID NOs: 50 to 51 and optionally having up to 2 amino acid substitutions thereto, an LCDR2 comprising or consisting of a sequence selected from the group consisting of SEQ ID NOs: 63 to 64 and optionally having up to 2 amino acid substitutions thereto, and an LCDR3 comprising or consisting of a sequence selected from the group consisting of SEQ ID NOs: 76 to 77 and optionally having up to 2 amino acid substitutions thereto; and
      ii. a light chain constant region comprising or consisting of a sequence having at least 90%, at least 91%, at least 92%, at least 93%, at least 94%, at least 95%, at least 95%, at least 97%, at least 98% or at least 99% identity to a sequence selected from the group consisting of SEQ ID NOs: 107 to 108;
      iii. optionally a linker; and
      iv. an IL-15 polypeptide comprising or consisting of a sequence having at least 90%, at least 91%, at least 92%, at least 93%, at least 94%, at least 95%, at least 95%, at least 97%, at least 98% or at least 99% identity to a sequence selected from the group consisting of SEQ ID NOs: 148 to 155, 206 and 207;
         and the immunoconjugate comprises or consists of first and second copies of said first and second polypeptides.
125. The immunoconjugate of any preceding embodiment, wherein the immunoconjugate comprises:
   a) a first polypeptide chain comprising, in an N- to C-terminal direction:
      i. a heavy chain variable region (VH) of an anti-NKp30 antibody comprising an HCDR1 comprising or consisting of a sequence selected from the group consisting of SEQ ID NOs: 11 to 12, an HCDR2 comprising or consisting of a sequence selected from the group consisting of SEQ ID NOs: 24 to 25 and an HCDR3 comprising or consisting of a sequence selected from the group consisting of SEQ ID NOs: 37 to 38;
      ii. a heavy chain constant region comprising or consisting of a sequence selected from the group consisting of SEQ ID NOs: 105, 106 and 203; and
   b) a second polypeptide chain comprising, in an N- to C-terminal direction:
      i. a light chain variable region (VL) of an anti-NKp30 antibody comprising an LCDR1 comprising or consisting of a sequence selected from the group consisting of SEQ ID NOs: 50 to 51, an LCDR2 comprising or consisting of a sequence selected from the group consisting of SEQ ID NOs: 63 to 64 and an LCDR3 comprising or consisting of a sequence selected from the group consisting of SEQ ID NOs: 76 to 77; and
      ii. a light chain constant region comprising or consisting of a sequence selected from the group consisting of SEQ ID NOs: 107 to 108;
      iii. optionally a linker; and
      iv. an IL-15 polypeptide comprising or consisting of a sequence selected from the group con-sisting of SEQ ID NOs: 148 to 155, 206 and 207;
         and the immunoconjugate comprises or consists of first and second copies of said first and second polypeptides.
126. The immunoconjugate of any preceding embodiment, wherein the immunoconjugate comprises:
   a) a first polypeptide chain comprising, in an N- to C-terminal direction:
      i. a heavy chain variable region (VH) of an anti-NKp46 antibody comprising an HCDR1 comprising or consisting of SEQ ID NO: 13 and optionally having up to 2 amino acid substitutions thereto, an HCDR2 comprising or consisting of SEQ ID NO: 26 and optionally having up to 2 amino acid substitutions thereto, and an HCDR3 comprising or consisting of SEQ ID NO: 39 and optionally having up to 2 amino acid substitutions thereto;
      ii. a heavy chain constant region comprising or consisting of a sequence having at least 90%, at least 91%, at least 92%, at least 93%, at least 94%, at least 95%, at least 95%, at least 97%, at least 98% or at least 99% identity to a sequence selected from the group consisting of SEQ ID NO: 105 to 106; and
   b) a second polypeptide chain comprising, in an N- to C-terminal direction:
      i. a light chain variable region (VL) of an anti-NKp46 antibody comprising an LCDR1 comprising or consisting of SEQ ID NO: 52 and optionally having up to 2 amino acid substitutions thereto, an LCDR2 comprising or consisting of SEQ ID NO: 65 and optionally having up to 2 amino acid substitutions thereto, and an LCDR3 comprising or consisting of SEQ ID NO: 78 and optionally having up to 2 amino acid substitutions thereto; and
      ii. a light chain constant region comprising or consisting of a sequence having at least 90%, at least 91%, at least 92%, at least 93%, at least 94%, at least 95%, at least 95%, at least 97%, at least 98% or at least 99% identity to SEQ ID NO: 107;
      iii. optionally a linker; and
      iv. an IL-15 polypeptide comprising or consisting of a sequence having at least 90%, at least 91%, at least 92%, at least 93%, at least 94%, at least 95%, at least 95%, at least 97%, at least 98% or at least 99% identity to a sequence selected from the group consisting of SEQ ID NOs: 148 to 155, 206 and 207;
         and the immunoconjugate comprises or consists of first and second copies of said first and second polypeptides.
127. The immunoconjugate of any preceding embodiment, wherein the immunoconjugate comprises:
   a) a first polypeptide chain comprising, in an N- to C-terminal direction:
      i. a heavy chain variable region (VH) of an anti-NKp46 antibody comprising an HCDR1 comprising or consisting of SEQ ID NO: 13, an HCDR2 comprising or consisting of SEQ ID NO: 26, and an HCDR3 comprising or consisting of SEQ ID NO: 39;
      ii. a heavy chain constant region comprising or consisting of a sequence selected from the group consisting of SEQ ID NOs: 105 to 106; and
   b) a second polypeptide chain comprising, in an N- to C-terminal direction:
      i. a light chain variable region (VL) of an anti-NKp46 antibody comprising an LCDR1 comprising or consisting of SEQ ID NO: 52, an LCDR2 comprising or consisting of SEQ ID NO: 65 and an LCDR3 comprising or consisting of SEQ ID NO: 78; and
      ii. a light chain constant region comprising or consisting of SEQ ID NO: 107;
      iii. optionally a linker; and
      iv. an IL-15 polypeptide comprising or consisting of a sequence selected from the group con-sisting of SEQ ID NOs: 148 to 155, 206 and 207;
         and the immunoconjugate comprises or consists of first and second copies of said first and second polypeptides.
128. The immunoconjugate of any one of embodiments 117 to 127, wherein the IL-15 polypeptide is an IL-15 polypeptide as defined in any one of embodiments 48 to 78.
129. The immunoconjugate of any preceding embodiment, wherein the immunoconjugate comprises:
   a) a first polypeptide chain comprising or consisting of a sequence having at least 90%, at least 91%, at least 92%, at least 93%, at least 94%, at least 95%, at least 95%, at least 97%, at least 98% or at least 99% identity to a sequence selected from the group consisting of SEQ ID NOs: 109 to 134; and
   b) a second polypeptide chain comprising or consisting of a sequence having at least 90%, at least 91%, at least 92%, at least 93%, at least 94%, at least 95%, at least 95%, at least 97%, at least 98% or at least 99% identity to a sequence selected from the group consisting of SEQ ID NOs: 169 to 181;
      and the immunoconjugate comprises or consists of first and second copies of said first and second polypeptides.
130. The immunoconjugate of any preceding embodiment, wherein the immunoconjugate comprises:
   a) a first polypeptide chain comprising or consisting of a sequence selected from the group con-sisting of SEQ ID NOs: 109 to 134; and
   b) a second polypeptide chain comprising or consisting of a sequence selected from the group consisting of SEQ ID NOs: 169 to 181;
      and the immunoconjugate comprises or consists of first and second copies of said first and second polypeptides.
131. The immunoconjugate of any preceding embodiment, wherein the immunoconjugate comprises:
   a) a first polypeptide chain comprising or consisting of the sequence of SEQ ID NO: 156; and a second polypeptide chain comprising or consisting of the sequence of SEQ ID NO: 135;
   b) a first polypeptide chain comprising or consisting of the sequence of SEQ ID NO: 157; and a second polypeptide chain comprising or consisting of the sequence of SEQ ID NO: 136;
   c) a first polypeptide chain comprising or consisting of the sequence of SEQ ID NO: 158; and a second polypeptide chain comprising or consisting of the sequence of SEQ ID NO: 137;
   d) a first polypeptide chain comprising or consisting of the sequence of SEQ ID NO: 159; and a second polypeptide chain comprising or consisting of the sequence of SEQ ID NO: 138;
   e) a first polypeptide chain comprising or consisting of the sequence of SEQ ID NO: 160; and a second polypeptide chain comprising or consisting of the sequence of SEQ ID NO: 139;
   f) a first polypeptide chain comprising or consisting of the sequence of SEQ ID NO: 161; and a second polypeptide chain comprising or consisting of the sequence of SEQ ID NO: 140;
   g) a first polypeptide chain comprising or consisting of the sequence of SEQ ID NO: 162; and a second polypeptide chain comprising or consisting of the sequence of SEQ ID NO: 141;
   h) a first polypeptide chain comprising or consisting of the sequence of SEQ ID NO: 163; and a second polypeptide chain comprising or consisting of the sequence of SEQ ID NO: 142;
   i) a first polypeptide chain comprising or consisting of the sequence of SEQ ID NO: 164; and a second polypeptide chain comprising or consisting of the sequence of SEQ ID NO: 143;
   j) a first polypeptide chain comprising or consisting of the sequence of SEQ ID NO: 165; and a second polypeptide chain comprising or consisting of the sequence of SEQ ID NO: 144;
   k) a first polypeptide chain comprising or consisting of the sequence of SEQ ID NO: 166; and a second polypeptide chain comprising or consisting of the sequence of SEQ ID NO: 145;
   l) a first polypeptide chain comprising or consisting of the sequence of SEQ ID NO: 167; and a second polypeptide chain comprising or consisting of the sequence of SEQ ID NO: 146
   m) a first polypeptide chain comprising or consisting of the sequence of SEQ ID NO: 168; and a second polypeptide chain comprising or consisting of the sequence of SEQ ID NO: 147;
   n) a first polypeptide chain comprising or consisting of the sequence of SEQ ID NO: 109; and a second polypeptide chain comprising or consisting of the sequence of SEQ ID NO: 169;
   o) a first polypeptide chain comprising or consisting of the sequence of SEQ ID NO: 110; and a second polypeptide chain comprising or consisting of the sequence of SEQ ID NO: 170;
   p) a first polypeptide chain comprising or consisting of the sequence of SEQ ID NO: 111; and a second polypeptide chain comprising or consisting of the sequence of SEQ ID NO: 171
   q) a first polypeptide chain comprising or consisting of the sequence of SEQ ID NO: 112; and a second polypeptide chain comprising or consisting of the sequence of SEQ ID NO: 172;
   r) a first polypeptide chain comprising or consisting of the sequence of SEQ ID NO: 113; and a second polypeptide chain comprising or consisting of the sequence of SEQ ID NO: 173;
   s) a first polypeptide chain comprising or consisting of the sequence of SEQ ID NO: 114; and a second polypeptide chain comprising or consisting of the sequence of SEQ ID NO: 174;
   t) a first polypeptide chain comprising or consisting of the sequence of SEQ ID NO: 115; and a second polypeptide chain comprising or consisting of the sequence of SEQ ID NO: 175;
   u) a first polypeptide chain comprising or consisting of the sequence of SEQ ID NO: 116; and a second polypeptide chain comprising or consisting of the sequence of SEQ ID NO: 176;
   v) a first polypeptide chain comprising or consisting of the sequence of SEQ ID NO: 117; and a second polypeptide chain comprising or consisting of the sequence of SEQ ID NO: 177;
   w) a first polypeptide chain comprising or consisting of the sequence of SEQ ID NO: 118; and a second polypeptide chain comprising or consisting of the sequence of SEQ ID NO: 178;
   x) a first polypeptide chain comprising or consisting of the sequence of SEQ ID NO: 119; and a second polypeptide chain comprising or consisting of the sequence of SEQ ID NO: 179;
   y) a first polypeptide chain comprising or consisting of the sequence of SEQ ID NO: 120; and a second polypeptide chain comprising or consisting of the sequence of SEQ ID NO: 180;
   z) a first polypeptide chain comprising or consisting of the sequence of SEQ ID NO: 121; and a second polypeptide chain comprising or consisting of the sequence of SEQ ID NO: 181;
   aa) a first polypeptide chain comprising or consisting of the sequence of SEQ ID NO: 122; and a second polypeptide chain comprising or consisting of the sequence of SEQ ID NO: 169;
   bb) a first polypeptide chain comprising or consisting of the sequence of SEQ ID NO: 123; and a second polypeptide chain comprising or consisting of the sequence of SEQ ID NO: 170;
   cc) a first polypeptide chain comprising or consisting of the sequence of SEQ ID NO: 124; and a second polypeptide chain comprising or consisting of the sequence of SEQ ID NO: 171;
   dd) a first polypeptide chain comprising or consisting of the sequence of SEQ ID NO: 125; and a second polypeptide chain comprising or consisting of the sequence of SEQ ID NO: 172;
   ee) a first polypeptide chain comprising or consisting of the sequence of SEQ ID NO: 126; and a second polypeptide chain comprising or consisting of the sequence of SEQ ID NO: 173;
   ff) a first polypeptide chain comprising or consisting of the sequence of SEQ ID NO: 127; and a second polypeptide chain comprising or consisting of the sequence of SEQ ID NO: 174;
   gg) a first polypeptide chain comprising or consisting of the sequence of SEQ ID NO: 128; and a second polypeptide chain comprising or consisting of the sequence of SEQ ID NO: 175;
   hh) a first polypeptide chain comprising or consisting of the sequence of SEQ ID NO: 129; and a second polypeptide chain comprising or consisting of the sequence of SEQ ID NO: 176;
   ii) a first polypeptide chain comprising or consisting of the sequence of SEQ ID NO: 130; and a second polypeptide chain comprising or consisting of the sequence of SEQ ID NO: 177;
   jj) a first polypeptide chain comprising or consisting of the sequence of SEQ ID NO: 131; and a second polypeptide chain comprising or consisting of the sequence of SEQ ID NO: 178;
   kk) a first polypeptide chain comprising or consisting of the sequence of SEQ ID NO: 132; and a second polypeptide chain comprising or consisting of the sequence of SEQ ID NO: 179; or
   ll) a first polypeptide chain comprising or consisting of the sequence of SEQ ID NO: 133; and a second polypeptide chain comprising or consisting of the sequence of SEQ ID NO: 180; or
   mm) a first polypeptide chain comprising or consisting of the sequence of SEQ ID NO: 134; and a second polypeptide chain comprising or consisting of the sequence of SEQ ID NO: 181;
   nn) a first polypeptide chain comprising or consisting of the sequence of SEQ ID NO: 188; and a second polypeptide chain comprising or consisting of the sequence of SEQ ID NO: 135;
   oo) a first polypeptide chain comprising or consisting of the sequence of SEQ ID NO: 189; and a second polypeptide chain comprising or consisting of the sequence of SEQ ID NO: 140;
   pp) a first polypeptide chain comprising or consisting of the sequence of SEQ ID NO: 191; and a second polypeptide chain comprising or consisting of the sequence of SEQ ID NO: 140;
   qq) a first polypeptide chain comprising or consisting of the sequence of SEQ ID NO: 192; and a second polypeptide chain comprising or consisting of the sequence of SEQ ID NO: 140;
   rr) a first polypeptide chain comprising or consisting of the sequence of SEQ ID NO: 195; and a second polypeptide chain comprising or consisting of the sequence of SEQ ID NO: 140;
   ss) a first polypeptide chain comprising or consisting of the sequence of SEQ ID NO: 200; and a second polypeptide chain comprising or consisting of the sequence of SEQ ID NO: 140;
   tt) a first polypeptide chain comprising or consisting of the sequence of SEQ ID NO: 190; and a second polypeptide chain comprising or consisting of the sequence of SEQ ID NO: 140; or
   uu) a first polypeptide chain comprising or consisting of the sequence of SEQ ID NO: 114; and a second polypeptide chain comprising or consisting of the sequence of SEQ ID NO: 199;
      and the immunoconjugate comprises or consists of first and second copies of said first and second polypeptides
132. The immunoconjugate of any preceding embodiment, wherein the immunoconjugates are ho-modimeric.
133. The immunoconjugate of embodiment 132, wherein one monomer of the dimer comprises a first copy of the first polypeptide chain and a first copy of the second polypeptide chain and the second monomer of the dimer comprises a second copy of the first polypeptide chain and a second copy of the second polypeptide chain.
134. The immunoconjugate of embodiments 132 or 133 wherein the immunoconjugate has been se-lectively purified to remove or reduce heterodimeric immunoconjugates.
135. The immunoconjugate of any preceding embodiment, wherein the immunoconjugate comprises a polypeptide chain comprising (optionally in an N to C terminal direction):
   a) an IL-15 polypeptide;
   b) optionally a linker;
   c) a heavy chain constant region or fragment thereof,
   d) optionally a linker;
   e) an antibody or antigen-binding fragment thereof comprising at least one antibody binding domain that specifically binds to an antigen selected from the group consisting of NKG2D, NKp30, NKp46, NKp44 and DNAM-1;
   and the immunoconjugate comprises or consists of first and second copies of said polypeptide chain.
136. The immunoconjugate of embodiment 135, wherein the immunoconjugate comprises a polypeptide chain comprising (optionally in an N to C terminal direction):
   a) an IL-15 polypeptide comprising or consisting of the sequence of SEQ ID NO: 154;
   b) optionally a linker;
   c) a heavy chain constant region fragment comprising a hinge, a CH2 and a CH3 region, and comprising or consisting of the sequence of SEQ ID NO: 208;
   d) optionally a linker;
   e) a scFv domain that specifically binds to NKG2D, comprising or consisting of a sequence selected from the group consisting of SEQ ID NO: 211 to 220 and 224 to 233;
   and the immunoconjugate comprises or consists of first and second copies of said polypeptide chain.
137. The immunoconjugate of embodiment 136, wherein the immunoconjugate comprises a polypeptide chain comprising or consisting of the sequence of SEQ ID NO: 242 or 255, and the immunoconjugate comprises or consists of first and second copies of said polypeptide chain.
138. The immunoconjugate of embodiment 136, wherein the immunoconjugate comprises a polypeptide chain comprising or consisting of the sequence of SEQ ID NO: 237 or 250, and the immunoconjugate comprises or consists of first and second copies of said polypeptide chain.
139. The immunoconjugate of embodiment 135, wherein the immunoconjugate comprises a polypeptide chain comprising:
   a) an IL-15 polypeptide comprising or consisting of the sequence of SEQ ID NO: 154;
   b) optionally a linker;
   c) a heavy chain constant region fragment comprising a hinge, a CH2 and a CH3 region, and comprising or consisting of the sequence of SEQ ID NO: 208;
   d) optionally a linker;
   e) a scFv domain that specifically binds to NKp30, comprising or consisting of a sequence selected from the group consisting of SEQ ID NO: 221, 222, 234 and 235;
   and the immunoconjugate comprises or consists of first and second copies of said polypeptide chain.
140. The immunoconjugate of embodiment 135, wherein the immunoconjugate comprises a polypeptide chain comprising:
   a) an IL-15 polypeptide comprising or consisting of the sequence of SEQ ID NO: 154;
   b) optionally a linker;
   c) a heavy chain constant region fragment comprising a hinge, a CH2 and a CH3 region, and comprising or consisting of the sequence of SEQ ID NO: 208;
   d) optionally a linker;
   e) a scFv domain that specifically binds to NKp30, comprising or consisting of a sequence selected from the group consisting of SEQ ID NO: 223 and 236;
   and the immunoconjugate comprises or consists of first and second copies of said polypeptide chain.
141. An immunoconjugate, comprising:
   a) at least two IL-15 polypeptides, each comprising
      i. an IL-15 cytokine; and
      ii. IL-15 receptor alpha (CD215) or a functional fragment thereof; and
   b) a multivalent antibody or antigen-binding fragment thereof, comprising at least two antibody binding domains, wherein the multivalent antibody or antigen-binding fragment thereof specifically binds to NKG2D.
142. The immunoconjugate of embodiment 141, comprising the features of any of embodiments 2 to 140.
143. The immunoconjugate of any preceding embodiment, wherein the multivalent antibody or anti-gen binding fragment thereof has agonistic activity when administered to a subject or to cell.
144. The immunoconjugate of any preceding embodiment, wherein the immunoconjugate is an immunostimulant.
145. The immunoconjugate of any preceding embodiment, wherein the immunoconjugate increase production of a cytokine when provided to an immune cell.
146. The immunoconjugate of embodiment 143, wherein the agonistic activity is confirmed or determined by an increase in levels of secreted IL-2 or secreted interferon-gamma in a subject or cell compared to before administration, or cell proliferation, or cell cytotoxicity associated with the activated cell or cells.
147. The immunoconjugate of any preceding embodiment, wherein the immunoconjugates bind to both targets (NKG2D and IL15-R1D) simultaneously or substantially simultaneously.
148. The immunoconjugate of any preceding embodiment, wherein the immunoconjugates display sequence order independent dual target co-engagement.
149. The immunoconjugate of any preceding embodiment, wherein the immunoconjugate is capable of activating NKG2D, NKp30, NKp46, NKp44 and/or DNAM-1 receptor-positive gamma delta T-cells and/or NK cells.
150. The immunoconjugate of any preceding embodiment, wherein the immunoconjugate binds to NKG2D with a binding affinity (KD) as measured by surface plasmon resonance of less than 5.0 nM, optionally with a KD of less than 2.5 nM, optionally with a KD of less than 1.0 nM, optionally with a KD of less than 0.8 nM.
151. The immunoconjugate of any preceding embodiment, wherein the immunoconjugate binds to IL-15Rβ with a binding affinity (KD) as measured by surface plasmon resonance of less than 10.0 nM, optionally with a KD of less than 8.0 nM, optionally with a KD of less than 5.0 nM, optionally with a KD of less than 4.6 nM.
152. The immunoconjugate of any preceding embodiment, wherein the immunoconjugate is capable of stimulating the immune system of a subject when administered to said subject.
153. The immunoconjugate of any preceding embodiment, wherein the IL-15 polypeptide is cis- or trans-presented to a cell when administered to said cell or a subject comprising said cell.
154. The immunoconjugate of any preceding embodiment, wherein the immunoconjugate is capable of binding to a component of the IL-15 receptor complex and to NKG2D, NKp30, NKp46, NKp44 or DNAM-1 simultaneously when administered to a subject or to a cell.
155. A polynucleotide or pair of polynucleotides encoding the immunoconjugate of any preceding em-bodiment.
156. A polynucleotide encoding the polypeptide of any one of SEQ ID NOs: 156 to 181
157. A polynucleotide encoding the first polypeptide of any one of embodiments 90 to 91.
158. A polynucleotide encoding the second polypeptide of any one of embodiments 104 to 105.
159. A pair of polynucleotides, the first polynucleotide encoding the polypeptide of any one of SEQ ID NOs: 156 to 168, and the second polynucleotide encoding the polypeptide of any one of SEQ ID NOs: 135 to 147.
160. The pair of polynucleotides of embodiment 159, wherein the polynucleotides are present as a kit.
161. The pair of polynucleotides of embodiment 160, wherein the polynucleotides are disposed in separate containers in the kit.
162. The pair of polynucleotides of embodiment 160, wherein the polynucleotides are present in a single container or composition.
163. A vector comprising a polynucleotide of any one of embodiments 155 to 158, or a pair of polynu-cleotides of any one of embodiments 159 to 162.
164. A host cell comprising a polynucleotide of any one of embodiments 155 to 158, a pair of polynu-cleotides of any one of embodiments 159 to 162, or a vector of embodiment 163.
165. The host cell of embodiment 164, wherein the host cell is a CHO cell.
166. A host cell comprising a first polynucleotide encoding a or the heavy chain of an immunoconju-gate of any one of embodiments 1 to 154 and a second polynucleotide encoding a or the light chain of an immunoconjugate of any one of embodiments 1 to 154.
167. A method of making an immunoconjugate comprising culturing the host cell of any one of em-bodiments 164 to 166 under conditions in which the immunoconjugate is expressed by the cell.
168. The method of embodiment 167, wherein the immunoconjugate is an immunoconjugate of any one of embodiments 1 to 154.
169. The method of embodiment 167 or embodiment 168, further comprising isolating and optionally purifying the expressed immunoconjugate.
170. The method of embodiment 169, wherein purification of the immunoconjugate results in an aqueous solution wherein the soluble immunoconjugate monomer content is greater than 80% or greater than 90%.
171. An immunoconjugate obtained or obtainable by the method of any one of embodiments 167 to 170.
172. The method of any one of embodiments 167 to 171, further comprising formulating the isolated and optionally purified immunoconjugate into a pharmaceutical composition comprising one or more pharmaceutically acceptable excipients or diluents.
173. A pharmaceutical composition obtained or obtainable by the method of embodiment 172.
174. A composition comprising the immunoconjugate of any one of embodiments 1 to 154.
175. A pharmaceutical composition comprising the immunoconjugate of any one of embodiments 1 to 154 or 171, and one or more pharmaceutically acceptable excipients or diluents.
176. The immunoconjugate of any one of embodiments 1 to 154 or embodiment 171, the composi-tion of embodiment 174, or the pharmaceutical composition of embodiment 173 or embodiment 175, for use in medicine.
177. The immunoconjugate of any one of embodiments 1 to 154 or embodiment 171, the composi-tion of embodiment 174, or the pharmaceutical composition of embodiment 173 or embodiment 175, for use in treating cancer.
178. The immunoconjugate of any one of embodiments 1 to 154 or embodiment 171, the composi-tion of embodiment 174, or the pharmaceutical composition of embodiment 173 or embodiment 175, for use in stimulating the immune system of a subject.
179. Use of the immunoconjugate of any one of embodiments 1 to 154 or embodiment 171 in the manufacture of a medicament.
180. Use of the immunoconjugate of any one of embodiments 1 to 154 or embodiment 171 in the manufacture of a medicament for the treatment of cancer.
181. Use of the immunoconjugate of any one of embodiments 1 to 154 or embodiment 171 in the manufacture of a medicament for stimulating the immune system of a subject.
182. A method of treating a disease, comprising administering to a subject in need thereof a thera-peutically effective amount of an immunoconjugate of any one of embodiments 1 to 154 or embodi-ment 171, the composition of embodiment 174, or the pharmaceutical composition of embodiment 173 or embodiment 175.
183. A method of treating cancer, comprising administering to a subject in need thereof a therapeuti-cally effective amount of an immunoconjugate of any one of embodiments 1 to 154 or embodiment 171, the composition of embodiment 174, or the pharmaceutical composition of embodiment 173 or embodiment 175.
184. A method of stimulating the immune system of a subject, comprising administering to a subject in need thereof a therapeutically effective amount of an immunoconjugate of any one of embodi-ments 1 to 154 or embodiment 171, the composition of embodiment 174, or the pharmaceutical composition of embodiment 173 or embodiment 175.

1. An immunoconjugate, comprising:
a) at least two IL-15 polypeptides, each comprising
i. an IL-15 cytokine; and
ii. IL-15 receptor alpha (CD215) or a functional fragment thereof; and
b) a multivalent antibody or antigen-binding fragment thereof, comprising at least two antibody binding domains that each specifically bind to an antigen selected from the group consisting of NKG2D, NKp30, NKp46, NKp44 and DNAM-1.

2. The immunoconjugate of claim 1, wherein the IL-15 polypeptides and the multivalent antibody or antigen-binding fragment thereof are directly fused to each other or wherein the IL-15 polypeptides and the multivalent antibody or antigen-binding fragment thereof are joined by a linker.

3. The immunoconjugate of any preceding claim, wherein the multivalent antibody or antigen-binding fragment thereof comprises at least two heavy chains and at least two light chains, and the IL-15 pol-ypeptides conjugated to the C-termini or the N-termini of the heavy chains, or the C-termini or the N-termini of the light chains, optionally via a linker.

4. The immunoconjugate of any preceding claim, wherein the multivalent antibody or antigen binding fragment thereof comprises:
a) a heavy chain variable region (VH) comprising:
a HCDR1 comprising or consisting of the sequence of SEQ ID NO: 1, optionally having up to 2 amino acid substitutions therefrom;
a HCDR2 comprising or consisting of the sequence of SEQ ID NO: 14, optionally having up to 2 amino acid substitutions therefrom; and
a HCDR3 comprising or consisting of the sequence of SEQ ID NO: 27, optionally having up to 2 amino acid substitutions therefrom;
and a light chain variable region (VL) comprising:
a LCDR1 comprising or consisting of the sequence of SEQ ID NO: 40, optionally having up to 2 amino acid substitutions therefrom;
a LCDR2 comprising or consisting of the sequence of SEQ ID NO: 53, optionally having up to 2 amino acid substitutions therefrom; and
a LCDR3 comprising or consisting of the sequence of SEQ ID NO: 66, optionally having up to 2 amino acid substitutions therefrom;
b) a heavy chain variable region (VH) comprising:
a HCDR1 comprising or consisting of the sequence of SEQ ID NO: 2, optionally having up to 2 amino acid substitutions therefrom;
a HCDR2 comprising or consisting of the sequence of SEQ ID NO: 15, optionally having up to 2 amino acid substitutions therefrom; and
a HCDR3 comprising or consisting of the sequence of SEQ ID NO: 28, optionally having up to 2 amino acid substitutions therefrom;
and a light chain variable region (VL) comprising:
a LCDR1 comprising or consisting of the sequence of SEQ ID NO: 41, optionally having up to 2 amino acid substitutions therefrom;
a LCDR2 comprising or consisting of the sequence of SEQ ID NO: 54, optionally having up to 2 amino acid substitutions therefrom; and
a LCDR3 comprising or consisting of the sequence of SEQ ID NO: 67, optionally having up to 2 amino acid substitutions therefrom;
c) a heavy chain variable region (VH) comprising:
a HCDR1 comprising or consisting of the sequence of SEQ ID NO: 3, optionally having up to 2 amino acid substitutions therefrom;
a HCDR2 comprising or consisting of the sequence of SEQ ID NO: 16, optionally having up to 2 amino acid substitutions therefrom; and
a HCDR3 comprising or consisting of the sequence of SEQ ID NO: 29, optionally having up to 2 amino acid substitutions therefrom;
and a light chain variable region (VL) comprising:
a LCDR1 comprising or consisting of the sequence of SEQ ID NO: 42, optionally having up to 2 amino acid substitutions therefrom;
a LCDR2 comprising or consisting of the sequence of SEQ ID NO: 55, optionally having up to 2 amino acid substitutions therefrom; and
a LCDR3 comprising or consisting of the sequence of SEQ ID NO: 68, optionally having up to 2 amino acid substitutions therefrom;
d) a heavy chain variable region (VH) comprising:
a HCDR1 comprising or consisting of the sequence of SEQ ID NO: 4, optionally having up to 2 amino acid substitutions therefrom;
a HCDR2 comprising or consisting of the sequence of SEQ ID NO: 17, optionally having up to 2 amino acid substitutions therefrom; and
a HCDR3 comprising or consisting of the sequence of SEQ ID NO: 30, optionally having up to 2 amino acid substitutions therefrom;
and a light chain variable region (VL) comprising:
a LCDR1 comprising or consisting of the sequence of SEQ ID NO: 43, optionally having up to 2 amino acid substitutions therefrom;
a LCDR2 comprising or consisting of the sequence of SEQ ID NO: 56, optionally having up to 2 amino acid substitutions therefrom; and
a LCDR3 comprising or consisting of the sequence of SEQ ID NO: 69, optionally having up to 2 amino acid substitutions therefrom;
e) a heavy chain variable region (VH) comprising:
a HCDR1 comprising or consisting of the sequence of SEQ ID NO: 5, optionally having up to 2 amino acid substitutions therefrom;
a HCDR2 comprising or consisting of the sequence of SEQ ID NO: 18, optionally having up to 2 amino acid substitutions therefrom; and
a HCDR3 comprising or consisting of the sequence of SEQ ID NO: 31, optionally having up to 2 amino acid substitutions therefrom;
and a light chain variable region (VL) comprising:
a LCDR1 comprising or consisting of the sequence of SEQ ID NO: 44, optionally having up to 2 amino acid substitutions therefrom;
a LCDR2 comprising or consisting of the sequence of SEQ ID NO: 57, optionally having up to 2 amino acid substitutions therefrom; and
a LCDR3 comprising or consisting of the sequence of SEQ ID NO: 70, optionally having up to 2 amino acid substitutions therefrom;
f) a heavy chain variable region (VH) comprising:
a HCDR1 comprising or consisting of the sequence of SEQ ID NO: 6, optionally having up to 2 amino acid substitutions therefrom;
a HCDR2 comprising or consisting of the sequence of SEQ ID NO: 19, optionally having up to 2 amino acid substitutions therefrom; and
a HCDR3 comprising or consisting of the sequence of SEQ ID NO: 32, optionally having up to 2 amino acid substitutions therefrom;
and a light chain variable region (VL) comprising:
a LCDR1 comprising or consisting of the sequence of SEQ ID NO: 45, optionally having up to 2 amino acid substitutions therefrom;
a LCDR2 comprising or consisting of the sequence of SEQ ID NO: 58, optionally having up to 2 amino acid substitutions therefrom; and
a LCDR3 comprising or consisting of the sequence of SEQ ID NO: 71, optionally having up to 2 amino acid substitutions therefrom;
g) a heavy chain variable region (VH) comprising:
a HCDR1 comprising or consisting of the sequence of SEQ ID NO: 7, optionally having up to 2 amino acid substitutions therefrom;
a HCDR2 comprising or consisting of the sequence of SEQ ID NO: 20, optionally having up to 2 amino acid substitutions therefrom; and
a HCDR3 comprising or consisting of the sequence of SEQ ID NO: 33, optionally having up to 2 amino acid substitutions therefrom;
and a light chain variable region (VL) comprising:
a LCDR1 comprising or consisting of the sequence of SEQ ID NO: 46, optionally having up to 2 amino acid substitutions therefrom;
a LCDR2 comprising or consisting of the sequence of SEQ ID NO: 59, optionally having up to 2 amino acid substitutions therefrom; and
a LCDR3 comprising or consisting of the sequence of SEQ ID NO: 72, optionally having up to 2 amino acid substitutions therefrom;
h) a heavy chain variable region (VH) comprising:
a HCDR1 comprising or consisting of the sequence of SEQ ID NO: 8, optionally having up to 2 amino acid substitutions therefrom;
a HCDR2 comprising or consisting of the sequence of SEQ ID NO: 21, optionally having up to 2 amino acid substitutions therefrom; and
a HCDR3 comprising or consisting of the sequence of SEQ ID NO: 34, optionally having up to 2 amino acid substitutions therefrom;
and a light chain variable region (VL) comprising:
a LCDR1 comprising or consisting of the sequence of SEQ ID NO: 47, optionally having up to 2 amino acid substitutions therefrom;
a LCDR2 comprising or consisting of the sequence of SEQ ID NO: 60, optionally having up to 2 amino acid substitutions therefrom; and
a LCDR3 comprising or consisting of the sequence of SEQ ID NO: 73, optionally having up to 2 amino acid substitutions therefrom;
i) a heavy chain variable region (VH) comprising:
a HCDR1 comprising or consisting of the sequence of SEQ ID NO: 9, optionally having up to 2 amino acid substitutions therefrom;
a HCDR2 comprising or consisting of the sequence of SEQ ID NO: 22, optionally having up to 2 amino acid substitutions therefrom; and
a HCDR3 comprising or consisting of the sequence of SEQ ID NO: 35, optionally having up to 2 amino acid substitutions therefrom;
and a light chain variable region (VL) comprising:
a LCDR1 comprising or consisting of the sequence of SEQ ID NO: 48, optionally having up to 2 amino acid substitutions therefrom;
a LCDR2 comprising or consisting of the sequence of SEQ ID NO: 61, optionally having up to 2 amino acid substitutions therefrom; and
a LCDR3 comprising or consisting of the sequence of SEQ ID NO: 74, optionally having up to 2 amino acid substitutions therefrom;
j) a heavy chain variable region (VH) comprising:
a HCDR1 comprising or consisting of the sequence of SEQ ID NO: 10, optionally having up to 2 amino acid substitutions therefrom;
a HCDR2 comprising or consisting of the sequence of SEQ ID NO: 23, optionally having up to 2 amino acid substitutions therefrom; and
a HCDR3 comprising or consisting of the sequence of SEQ ID NO: 36, optionally having up to 2 amino acid substitutions therefrom;
and a light chain variable region (VL) comprising:
a LCDR1 comprising or consisting of the sequence of SEQ ID NO: 49, optionally having up to 2 amino acid substitutions therefrom;
a LCDR2 comprising or consisting of the sequence of SEQ ID NO: 62, optionally having up to 2 amino acid substitutions therefrom; and
a LCDR3 comprising or consisting of the sequence of SEQ ID NO: 75, optionally having up to 2 amino acid substitutions therefrom;
k) a heavy chain variable region (VH) comprising:
a HCDR1 comprising or consisting of the sequence of SEQ ID NO: 11, optionally having up to 2 amino acid substitutions therefrom;
a HCDR2 comprising or consisting of the sequence of SEQ ID NO: 24, optionally having up to 2 amino acid substitutions therefrom; and
a HCDR3 comprising or consisting of the sequence of SEQ ID NO: 37, optionally having up to 2 amino acid substitutions therefrom;
and a light chain variable region (VL) comprising:
a LCDR1 comprising or consisting of the sequence of SEQ ID NO: 50, optionally having up to 2 amino acid substitutions therefrom;
a LCDR2 comprising or consisting of the sequence of SEQ ID NO: 63, optionally having up to 2 amino acid substitutions therefrom; and
a LCDR3 comprising or consisting of the sequence of SEQ ID NO: 76, optionally having up to 2 amino acid substitutions therefrom;
l) a heavy chain variable region (VH) comprising:
a HCDR1 comprising or consisting of the sequence of SEQ ID NO: 12, optionally having up to 2 amino acid substitutions therefrom;
a HCDR2 comprising or consisting of the sequence of SEQ ID NO: 25, optionally having up to 2 amino acid substitutions therefrom; and
a HCDR3 comprising or consisting of the sequence of SEQ ID NO: 38, optionally having up to 2 amino acid substitutions therefrom;
and a light chain variable region (VL) comprising:
a LCDR1 comprising or consisting of the sequence of SEQ ID NO: 51, optionally having up to 2 amino acid substitutions therefrom;
a LCDR2 comprising or consisting of the sequence of SEQ ID NO: 64, optionally having up to 2 amino acid substitutions therefrom; and
a LCDR3 comprising or consisting of the sequence of SEQ ID NO: 77, optionally having up to 2 amino acid substitutions therefrom; or
m) a heavy chain variable region (VH) comprising:
a HCDR1 comprising or consisting of the sequence of SEQ ID NO: 13, optionally having up to 2 amino acid substitutions therefrom;
a HCDR2 comprising or consisting of the sequence of SEQ ID NO: 26, optionally having up to 2 amino acid substitutions therefrom; and
a HCDR3 comprising or consisting of the sequence of SEQ ID NO: 39, optionally having up to 2 amino acid substitutions therefrom;
and a light chain variable region (VL) comprising:
a LCDR1 comprising or consisting of the sequence of SEQ ID NO: 52, optionally having up to 2 amino acid substitutions therefrom;
a LCDR2 comprising or consisting of the sequence of SEQ ID NO: 65, optionally having up to 2 amino acid substitutions therefrom; and
a LCDR3 comprising or consisting of the sequence of SEQ ID NO: 78, optionally having up to 2 amino acid substitutions therefrom, optionally wherein the op-tional amino acid substitutions are conservative substitutions.

5. The immunoconjugate of any preceding claim, wherein the multivalent antibody or antigen binding fragment thereof comprises:
a) a VH comprising or consisting of the sequence of SEQ ID NO: 79 and a VL comprising or consisting of the sequence of SEQ ID NO: 92;
b) a VH comprising or consisting of the sequence of SEQ ID NO: 202 and a VL comprising or consisting of the sequence of SEQ ID NO: 92;
c) a VH comprising or consisting of the sequence of SEQ ID NO: 80 and a VL comprising or consisting of the sequence of SEQ ID NO: 93;
d) a VH comprising or consisting of the sequence of SEQ ID NO: 81 and a VL comprising or consisting of the sequence of SEQ ID NO: 94;
e) a VH comprising or consisting of the sequence of SEQ ID NO: 82 and a VL comprising or consisting of the sequence of SEQ ID NO: 95;
f) a VH comprising or consisting of the sequence of SEQ ID NO: 83 and a VL comprising or consisting of the sequence of SEQ ID NO: 96;
g) a VH comprising or consisting of the sequence of SEQ ID NO: 84 and a VL comprising or consisting of the sequence of SEQ ID NO: 97;
h) a VH comprising or consisting of the sequence of SEQ ID NO: 85 and a VL comprising or consisting of the sequence of SEQ ID NO: 98;
i) a VH comprising or consisting of the sequence of SEQ ID NO: 86 and a VL comprising or consisting of the sequence of SEQ ID NO: 99;
j) a VH comprising or consisting of the sequence of SEQ ID NO: 87 and a VL comprising or consisting of the sequence of SEQ ID NO: 100;
k) a VH comprising or consisting of the sequence of SEQ ID NO: 88 and a VL comprising or consisting of the sequence of SEQ ID NO: 101;
l) a VH comprising or consisting of the sequence of SEQ ID NO: 89 and a VL comprising or consisting of the sequence of SEQ ID NO: 102;
m) a VH comprising or consisting of the sequence of SEQ ID NO: 90 and a VL comprising or consisting of the sequence of SEQ ID NO: 103; or
n) a VH comprising or consisting of the sequence of SEQ ID NO: 91 and a VL comprising or consisting of the sequence of SEQ ID NO: 104.

6. The immunoconjugate of any preceding claim, wherein the multivalent antibody or antigen binding fragment thereof comprises:
a) a heavy chain comprising or consisting of the sequence of SEQ ID NO: 109 and a light chain comprising or consisting of the sequence SEQ ID NO: 135;
b) a heavy chain comprising or consisting of the sequence of SEQ ID NO: 110 and a light chain comprising or consisting of the sequence SEQ ID NO: 136;
c) a heavy chain comprising or consisting of the sequence of SEQ ID NO: 111 and a light chain comprising or consisting of the sequence SEQ ID NO: 137;
d) a heavy chain comprising or consisting of the sequence of SEQ ID NO: 112 and a light chain comprising or consisting of the sequence SEQ ID NO: 138;
e) a heavy chain comprising or consisting of the sequence of SEQ ID NO: 113 and a light chain comprising or consisting of the sequence SEQ ID NO: 139;
f) a heavy chain comprising or consisting of the sequence of SEQ ID NO: 114 and a light chain comprising or consisting of the sequence SEQ ID NO: 140;
g) a heavy chain comprising or consisting of the sequence of SEQ ID NO: 115 and a light chain comprising or consisting of the sequence SEQ ID NO: 141;
h) a heavy chain comprising or consisting of the sequence of SEQ ID NO: 116 and a light chain comprising or consisting of the sequence SEQ ID NO: 142;
i) a heavy chain comprising or consisting of the sequence of SEQ ID NO: 117 and a light chain comprising or consisting of the sequence SEQ ID NO: 143;
j) a heavy chain comprising or consisting of the sequence of SEQ ID NO: 118 and a light chain comprising or consisting of the sequence SEQ ID NO: 144;
k) a heavy chain comprising or consisting of the sequence of SEQ ID NO: 119 and a light chain comprising or consisting of the sequence SEQ ID NO: 145;
l) a heavy chain comprising or consisting of the sequence of SEQ ID NO: 120 and a light chain comprising or consisting of the sequence SEQ ID NO: 146;
m) a heavy chain comprising or consisting of the sequence of SEQ ID NO: 121 and a light chain comprising or consisting of the sequence SEQ ID NO: 147;
n) a heavy chain comprising or consisting of the sequence of SEQ ID NO: 122 and a light chain comprising or consisting of the sequence SEQ ID NO: 135;
o) a heavy chain comprising or consisting of the sequence of SEQ ID NO: 123 and a light chain comprising or consisting of the sequence SEQ ID NO: 136;
p) a heavy chain comprising or consisting of the sequence of SEQ ID NO: 124 and a light chain comprising or consisting of the sequence SEQ ID NO: 137;
q) a heavy chain comprising or consisting of the sequence of SEQ ID NO: 125 and a light chain comprising or consisting of the sequence SEQ ID NO: 138;
r) a heavy chain comprising or consisting of the sequence of SEQ ID NO: 126 and a light chain comprising or consisting of the sequence SEQ ID NO: 139;
s) a heavy chain comprising or consisting of the sequence of SEQ ID NO: 127 and a light chain comprising or consisting of the sequence SEQ ID NO: 140;
t) a heavy chain comprising or consisting of the sequence of SEQ ID NO: 128 and a light chain comprising or consisting of the sequence SEQ ID NO: 141;
u) a heavy chain comprising or consisting of the sequence of SEQ ID NO: 129 and a light chain comprising or consisting of the sequence SEQ ID NO: 142;
v) a heavy chain comprising or consisting of the sequence of SEQ ID NO: 130 and a light chain comprising or consisting of the sequence SEQ ID NO: 143;
w) a heavy chain comprising or consisting of the sequence of SEQ ID NO: 131 and a light chain comprising or consisting of the sequence SEQ ID NO: 144;
x) a heavy chain comprising or consisting of the sequence of SEQ ID NO: 132 and a light chain comprising or consisting of the sequence SEQ ID NO: 145;
y) a heavy chain comprising or consisting of the sequence of SEQ ID NO: 133 and a light chain comprising or consisting of the sequence SEQ ID NO: 146; or
z) a heavy chain comprising or consisting of the sequence of SEQ ID NO: 134 and a light chain comprising or consisting of the sequence SEQ ID NO: 147.

7. The immunoconjugate of any preceding claim, wherein the multivalent antibody or antigen binding fragment thereof comprises an Fc domain, optionally wherein the Fc domain is a human Fc domain, optionally wherein the Fc domain is a human IgG Fc domain, optionally wherein the human IgG Fc domain is a human IgG1 domain, optionally wherein the Fc domain is Fc disabled.

8. The immunoconjugate of any preceding claim, wherein the IL-15 polypeptide is an IL-15 superagonist.

9. The immunoconjugate of any preceding claim, wherein the IL-15 receptor alpha (CD215) fragment comprises up to 100 consecutive amino acids of the IL-15 receptor alpha (CD215) sequence and com-prises the IL-15 receptor alpha (CD215) sushi domain or wherein the IL-15 polypeptide comprises the IL-15 receptor alpha (CD215) sushi domain, optionally wherein the IL-15 receptor alpha (CD215) sushi domain is a human IL-15 receptor alpha (CD215) sushi domain.

10. The immunoconjugate of claim 9, wherein the IL-15 receptor alpha (CD215) sushi domain comprises or consists of a sequence having at least 90%, at least 91%, at least 92%, at least 93%, at least 94%, at least 95%, at least 95%, at least 97%, at least 98%, at least 99% or at least 100% identity to a sequence selected from the group consisting of SEQ ID NOs: 149 to 150.

11. The immunoconjugate of any preceding claim, wherein the IL-15 polypeptide comprises, in an N- to C-terminal direction, a human IL-15 cytokine and IL-15 receptor alpha (CD215) or functional fragment thereof, optionally joined by a linker, or wherein the IL-15 polypeptide comprises, in an N- to C-terminal direction, an IL-15 receptor alpha (CD215) or functional fragment thereof and a human IL-15 cytokine, optionally joined by a linker.

12. The immunoconjugate of any preceding claim, wherein the IL-15 cytokine is an IL-15 mutein, optionally wherein the IL-15 mutein comprises a N72D mutation.

13. The immunoconjugate of any preceding claim, wherein the IL-15 cytokine comprises or consists of a sequence having at least 90%, at least 91%, at least 92%, at least 93%, at least 94%, at least 95%, at least 95%, at least 97%, at least 98%, at least 99% or 100% identity to a sequence selected from the group consisting of SEQ ID NOs: 151 to 153.

14. The immunoconjugate of any preceding claim, wherein the IL-15 polypeptide comprises or consists of a sequence having at least 90%, at least 91%, at least 92%, at least 93%, at least 94%, at least 95%, at least 95%, at least 97%, at least 98%, at least 99% or 100% identity to a sequence selected from the group consisting of SEQ ID NOs: 154 to 155, 206 and 207.

15. The immunoconjugate of any previous claim, wherein the IL-15 cytokine and the IL-15 receptor alpha (CD215) or functional fragment thereof are directly fused to each other or are joined by a linker.

16. The immunoconjugate of any previous claim, wherein one or more of the linkers is a peptide linker, optionally a peptide of from about 1 to about 60 amino acid residues in length, optionally wherein the linker comprises or consists of the sequence [[G]ₘS]ₙX, wherein m is from 2 to 4, n is from 1 to 6 and X is G or is absent.

17. The immunoconjugate of any previous claim, wherein the linker comprises or consists of a sequence selected from the group consisting of SEQ ID NOs: 182 to 187 and 204 to 205.

18. The immunoconjugate of any preceding claim, wherein the immunoconjugate comprises:
b) a first polypeptide chain comprising, in an N- to C-terminal direction:
i. a heavy chain of an anti-NKG2D antibody, an anti-NKp30 antibody, an anti-NKp46 antibody, an anti-NKp44 antibody or an anti-DNAM-1 antibody;
ii. optionally a linker; and
iii. the IL-15 polypeptide; and
b) a second polypeptide chain comprising a light chain of an anti-NKG2D antibody, an anti-NKp30 antibody, an anti-NKp46 antibody, an anti-NKp44 antibody or an anti-DNAM-1 antibody;
and the immunoconjugate comprises or consists of first and second copies of said first and second polypeptides.

19. The immunoconjugate of any preceding claim, wherein the immunoconjugate comprises:
a) a first polypeptide chain comprising, in an N- to C-terminal direction:
v. a heavy chain variable region (VH) of an anti-NKG2D antibody, an anti-NKp30 antibody, an anti-NKp46 antibody, an anti-NKp44 antibody or an anti-DNAM-1 antibody, the VH comprising an HCDR1, an HCDR2 and an HCDR3;
vi. a heavy chain constant region comprising an CH1, a hinge, a CH2 and a CH3 re-gion;
vii. optionally a linker, and
viii. the IL-15 polypeptide; and
b) a second polypeptide chain comprising, in an N- to C-terminal direction:
iii. a light chain variable region (VL) of an anti-NKG2D antibody, an anti-NKp30 antibody, an anti-NKp46 antibody, an anti-NKp44 antibody or an anti-DNAM-1 antibody, the VL comprising an LCDR1, an LCDR2 and an LCDR3; and
iv. a light chain constant (CL) region;
and the immunoconjugate comprises or consists of first and second copies of said first and second polypeptides.

20. The immunoconjugate of any preceding claim, wherein the immunoconjugate comprises:
a) a first polypeptide chain comprising, in an N- to C-terminal direction:
i. a heavy chain variable region (VH) of an anti-NKG2D antibody comprising an HCDR1 comprising or consisting of the sequence of SEQ ID NO: 6, an HCDR2 comprising or consisting of the sequence of SEQ ID NO: 19 and an HCDR3 comprising or consisting of the sequence of SEQ ID NO: 32, optionally wherein the VH region comprises or consists of the sequence of SEQ ID NO:84;
ii. a heavy chain constant region comprising or consisting of a sequence of SEQ ID NO: 105, 106 and 203;
iii. optionally a linker; and
iv. an IL-15 polypeptide comprising or consisting of a sequence selected from the group consisting of SEQ ID NOs: 148 to 155, 206 and 207; and
b) a second polypeptide chain comprising, in an N- to C-terminal direction:
iii. a light chain variable region (VL) of an anti-NKG2D antibody comprising an LCDR1 comprising or consisting of the sequence of SEQ ID NO: 45, an LCDR2 comprising or consisting of the sequence of SEQ ID NO: 58 and an LCDR3 comprising or consisting of the sequence of SEQ ID NO: 71, optionally wherein the VL region comprises or consists of the sequence of SEQ ID NO: 97; and
iv. a light chain constant region comprising or consisting of the sequence of SEQ ID NO: 107;
and the immunoconjugate comprises or consists of first and second copies of said first and second polypeptides.

21. The immunoconjugate of any preceding claim, wherein the immunoconjugate comprises:
a) a first polypeptide chain comprising, in an N- to C-terminal direction:
i. a heavy chain variable region (VH) of an anti-NKG2D antibody comprising an HCDR1 comprising or consisting of the sequence of SEQ ID NO: 6, an HCDR2 comprising or consisting of the sequence of SEQ ID NO: 19 and an HCDR3 comprising or consisting of the sequence of SEQ ID NO: 32, optionally wherein the VH region comprises or consists of the sequence of SEQ ID NO:84;
ii. a heavy chain constant region comprising or consisting of a sequence of SEQ ID NO: 105;
iii. optionally a linker; and
iv. an IL-15 polypeptide comprising or consisting of a sequence selected from the group consisting of SEQ ID NOs: 154, 206 and 207; and
b) a second polypeptide chain comprising, in an N- to C-terminal direction:
v. a light chain variable region (VL) of an anti-NKG2D antibody comprising an LCDR1 comprising or consisting of the sequence of SEQ ID NO: 45, an LCDR2 comprising or consisting of the sequence of SEQ ID NO: 58 and an LCDR3 comprising or consisting of the sequence of SEQ ID NO: 71, optionally wherein the VL region comprises or consists of the sequence of SEQ ID NO: 97; and
vi. a light chain constant region comprising or consisting of the sequence of SEQ ID NO: 107;
and the immunoconjugate comprises or consists of first and second copies of said first and second polypeptides.

22. The immunoconjugate of any preceding claim, wherein the immunoconjugate comprises:
a) a first polypeptide chain comprising, in an N- to C-terminal direction:
i. a heavy chain variable region (VH) of an anti-NKG2D antibody comprising an HCDR1 comprising or consisting of the sequence of SEQ ID NO: 6, an HCDR2 comprising or consisting of the sequence of SEQ ID NO: 19 and an HCDR3 comprising or consisting of the sequence of SEQ ID NO: 32, optionally wherein the VH region comprises or consists of the sequence of SEQ ID NO:84;
ii. a heavy chain constant region comprising or consisting of a sequence of SEQ ID NO: 105;
iii. optionally a linker comprising or consisting of the sequence of SEQ ID NO: 182; and
iv. an IL-15 polypeptide comprising or consisting of the sequence of SEQ ID NOs: 154; and
b) a second polypeptide chain comprising, in an N- to C-terminal direction:
vii. a light chain variable region (VL) of an anti-NKG2D antibody comprising an LCDR1 comprising or consisting of the sequence of SEQ ID NO: 45, an LCDR2 comprising or consisting of the sequence of SEQ ID NO: 58 and an LCDR3 comprising or consisting of the sequence of SEQ ID NO: 71, optionally wherein the VL region comprises or consists of the sequence of SEQ ID NO: 97; and
viii. a light chain constant region comprising or consisting of the sequence of SEQ ID NO: 107;
and the immunoconjugate comprises or consists of first and second copies of said first and second polypeptides.

23. The immunoconjugate of any preceding claim, wherein the immunoconjugate comprises:
a) a first polypeptide chain comprising, in an N- to C-terminal direction:
i. a heavy chain variable region (VH) of an anti-NKG2D antibody comprising an HCDR1 comprising or consisting of the sequence of SEQ ID NO: 6, an HCDR2 comprising or consisting of the sequence of SEQ ID NO: 19 and an HCDR3 comprising or consisting of the sequence of SEQ ID NO: 32, optionally wherein the VH region comprises or consists of the sequence of SEQ ID NO:84;
ii. a heavy chain constant region comprising or consisting of a sequence of SEQ ID NO: 105;
iii. optionally a linker comprising or consisting of the sequence of SEQ ID NO: 184; and
iv. an IL-15 polypeptide comprising or consisting of the sequence of SEQ ID NOs: 154; and
b) a second polypeptide chain comprising, in an N- to C-terminal direction:
ix. a light chain variable region (VL) of an anti-NKG2D antibody comprising an LCDR1 comprising or consisting of the sequence of SEQ ID NO: 45, an LCDR2 comprising or consisting of the sequence of SEQ ID NO: 58 and an LCDR3 comprising or consisting of the sequence of SEQ ID NO: 71, optionally wherein the VL region comprises or consists of the sequence of SEQ ID NO: 97; and
x. a light chain constant region comprising or consisting of the sequence of SEQ ID NO: 107;
and the immunoconjugate comprises or consists of first and second copies of said first and second polypeptides.

24. The immunoconjugate of any preceding claim, wherein the immunoconjugate comprises:
a) a first polypeptide chain comprising, in an N- to C-terminal direction:
i. a heavy chain variable region (VH) of an anti-NKG2D antibody comprising an HCDR1 comprising or consisting of the sequence of SEQ ID NO: 6, an HCDR2 comprising or consisting of the sequence of SEQ ID NO: 19 and an HCDR3 comprising or consisting of the sequence of SEQ ID NO: 32, optionally wherein the VH region comprises or consists of the sequence of SEQ ID NO:84;
ii. a heavy chain constant region comprising or consisting of a sequence of SEQ ID NO: 105;
iii. optionally a linker comprising or consisting of the sequence of SEQ ID NO: 182; and
iv. an IL-15 polypeptide comprising or consisting of the sequence of SEQ ID NOs: 207; and
b) a second polypeptide chain comprising, in an N- to C-terminal direction:
xi. a light chain variable region (VL) of an anti-NKG2D antibody comprising an LCDR1 comprising or consisting of the sequence of SEQ ID NO: 45, an LCDR2 comprising or consisting of the sequence of SEQ ID NO: 58 and an LCDR3 comprising or consisting of the sequence of SEQ ID NO: 71, optionally wherein the VL region comprises or consists of the sequence of SEQ ID NO: 97; and
xii. a light chain constant region comprising or consisting of the sequence of SEQ ID NO: 107;
and the immunoconjugate comprises or consists of first and second copies of said first and second polypeptides.

25. The immunoconjugate of any preceding claim, wherein the immunoconjugate comprises:
a) a first polypeptide chain comprising, in an N- to C-terminal direction:
i. a heavy chain variable region (VH) of an anti-NKG2D antibody comprising an HCDR1 comprising or consisting of the sequence of SEQ ID NO: 1, an HCDR2 comprising or consisting of the sequence of SEQ ID NO: 14 and an HCDR3 comprising or consisting of the sequence of SEQ ID NO: 27, optionally wherein the VH region comprises or consists of the sequence of SEQ ID NO:79 or 202;
ii. a heavy chain constant region comprising or consisting of a sequence of SEQ ID NO: 105, 106 and 203;
iii. optionally a linker; and
iv. an IL-15 polypeptide comprising or consisting of a sequence selected from the group consisting of SEQ ID NOs: 148 to 155, 206 and 207; and
b) a second polypeptide chain comprising, in an N- to C-terminal direction:
xiii. a light chain variable region (VL) of an anti-NKG2D antibody comprising an LCDR1 comprising or consisting of the sequence of SEQ ID NO: 40, an LCDR2 comprising or consisting of the sequence of SEQ ID NO: 53 and an LCDR3 comprising or consisting of the sequence of SEQ ID NO: 66, optionally wherein the VL region comprises or consists of the sequence of SEQ ID NO: 92; and
xiv. a light chain constant region comprising or consisting of the sequence of SEQ ID NO: 108;
and the immunoconjugate comprises or consists of first and second copies of said first and second polypeptides.

26. The immunoconjugate of any preceding claim, wherein the immunoconjugate comprises:
a) a first polypeptide chain comprising, in an N- to C-terminal direction:
i) a heavy chain variable region (VH) of an anti-NKG2D antibody comprising an HCDR1 comprising or consisting of the sequence of SEQ ID NO: 1, an HCDR2 comprising or consisting of the sequence of SEQ ID NO: 14 and an HCDR3 comprising or consisting of the sequence of SEQ ID NO: 27, optionally wherein the VH region comprises or consists of the sequence of SEQ ID NO:79 or 202;
ii) a heavy chain constant region comprising or consisting of a sequence of SEQ ID NO: 105;
iii) optionally a linker; and
iv) an IL-15 polypeptide comprising or consisting of a sequence selected from the group consisting of SEQ ID NOs: 154, 206 and 207; and
b) a second polypeptide chain comprising, in an N- to C-terminal direction:
a. a light chain variable region (VL) of an anti-NKG2D antibody comprising an LCDR1 comprising or consisting of the sequence of SEQ ID NO: 40, an LCDR2 comprising or consisting of the sequence of SEQ ID NO: 53 and an LCDR3 comprising or consisting of the sequence of SEQ ID NO: 66, optionally wherein the VL region comprises or consists of the sequence of SEQ ID NO: 92; and
b. a light chain constant region comprising or consisting of the sequence of SEQ ID NO: 108;
and the immunoconjugate comprises or consists of first and second copies of said first and second polypeptides.

27. The immunoconjugate of any of claims 20, 21, 25 or 26, wherein the linker comprises or consists of a sequence selected from the group consisting of SEQ ID NO: 182 to 187, 201, 204 and 205.

28. The immunoconjugate of any preceding claim, wherein the immunoconjugate comprises:
c) a first polypeptide chain comprising or consisting of a sequence selected from the group consisting of SEQ ID NOs: 156 to 168; and
d) a second polypeptide chain comprising or consisting of a sequence selected from the group consisting of SEQ ID NOs: 135 to 147;
and the immunoconjugate comprises or consists of first and second copies of said first and second polypeptides.

29. The immunoconjugate of any preceding claim, wherein the immunoconjugate comprises:
a) a first polypeptide chain comprising, in an N- to C-terminal direction:
iii. a heavy chain variable region (VH) of an anti-NKG2D antibody, an anti-NKp30 antibody, an anti-NKp46 antibody, an anti-NKp44 antibody or an anti-DNAM-1 antibody, the VH comprising an HCDR1, an HCDR2 and an HCDR3;
iv. a heavy chain constant region comprising an CH1, a hinge, a CH2 and a CH3 region; and
b) a second polypeptide chain comprising, in an N- to C-terminal direction:
v. a light chain variable region (VL) of an anti-NKG2D antibody, an anti-NKp30 antibody, an anti-NKp46 antibody, an anti-NKp44 antibody or an anti-DNAM-1 antibody, the VL comprising an LCDR1, an LCDR2 and an LCDR3; and
vi. a light chain constant (CL) region;
vii. optionally a linker, and
viii. the IL-15 polypeptide;
and the immunoconjugate comprises or consists of first and second copies of said first and second polypeptides.

30. The immunoconjugate of any preceding claim, wherein the immunoconjugate comprises:
a) a first polypeptide chain comprising, in an N- to C-terminal direction:
iii. a heavy chain variable region (VH) of an anti-NKG2D antibody comprising an HCDR1 comprising or consisting of a sequence selected from the group consisting of SEQ ID NOs: 1 to 10 and optionally having up to 2 amino acid substitutions thereto, an HCDR2 comprising or consisting of a sequence selected from the group consisting of SEQ ID NOs: 14 to 23 and optionally having up to 2 amino acid substitutions thereto, and an HCDR3 comprising or consisting of a sequence selected from the group consisting of SEQ ID NOs: 27 to 36 and optionally having up to 2 amino acid substitutions thereto;
iv. a heavy chain constant region comprising or consisting of a sequence having at least 90%, at least 91%, at least 92%, at least 93%, at least 94%, at least 95%, at least 95%, at least 97%, at least 98%, at least 99% or 100% identity to a sequence selected from the group consisting of SEQ ID NOs: 105, 106 and 203; and
b) a second polypeptide chain comprising, in an N- to C-terminal direction:
v. a light chain variable region (VL) of an anti-NKG2D antibody comprising an LCDR1 comprising or consisting of a sequence selected from the group consisting of SEQ ID NOs: 40 to 49 and optionally having up to 2 amino acid substitutions thereto, an LCDR2 comprising or consisting of a sequence selected from the group consisting of SEQ ID NOs: 53 to 62 and optionally having up to 2 amino acid substitutions thereto, and an LCDR3 comprising or consisting of a sequence selected from the group consisting of SEQ ID NOs: 66 to 75 and optionally having up to 2 amino acid substitutions thereto; and
vi. a light chain constant region comprising or consisting of a sequence having at least 90%, at least 91%, at least 92%, at least 93%, at least 94%, at least 95%, at least 95%, at least 97%, at least 98%, at least 99% or 100% identity to a sequence selected from the group consisting of SEQ ID NOs: 107 to 108;
vii. optionally a linker; and
viii. an IL-15 polypeptide comprising or consisting of a sequence having at least 90%, at least 91%, at least 92%, at least 93%, at least 94%, at least 95%, at least 95%, at least 97%, at least 98%, at least 99% or 100% identity to a sequence selected from the group consisting of SEQ ID NOs: 148 to 155, 206 and 207;
and the immunoconjugate comprises or consists of first and second copies of said first and second polypeptides;
or
c) a first polypeptide chain comprising, in an N- to C-terminal direction:
iii. a heavy chain variable region (VH) of an anti-NKp30 antibody comprising an HCDR1 comprising or consisting of a sequence selected from the group consisting of SEQ ID NOs: 11 to 12 and optionally having up to 2 amino acid substitutions thereto, an HCDR2 comprising or consisting of a sequence selected from the group consisting of SEQ ID NOs: 24 to 25 and optionally having up to 2 amino acid substitutions thereto, and an HCDR3 comprising or consisting of a sequence selected from the group consisting of SEQ ID NOs: 37 to 38 and optionally having up to 2 amino acid substitutions thereto;
iv. a heavy chain constant region comprising or consisting of a sequence having at least 90%, at least 91%, at least 92%, at least 93%, at least 94%, at least 95%, at least 95%, at least 97%, at least 98%, at least 99% or 100% identity to a sequence selected from the group consisting of SEQ ID NOs: 105, 106 and 203; and
d) a second polypeptide chain comprising, in an N- to C-terminal direction:
v. a light chain variable region (VL) of an anti-NKp30 antibody comprising an LCDR1 comprising or consisting of a sequence selected from the group consisting of SEQ ID NOs: 50 to 51 and optionally having up to 2 amino acid substitutions thereto, an LCDR2 comprising or consisting of a sequence selected from the group consisting of SEQ ID NOs: 63 to 64 and optionally having up to 2 amino acid substitutions thereto, and an LCDR3 comprising or consisting of a sequence selected from the group consisting of SEQ ID NOs: 76 to 77 and optionally having up to 2 amino acid substitutions thereto; and
vi. a light chain constant region comprising or consisting of a sequence having at least 90%, at least 91%, at least 92%, at least 93%, at least 94%, at least 95%, at least 95%, at least 97%, at least 98%, at least 99% or 100% identity to a sequence selected from the group consisting of SEQ ID NOs: 107 to 108;
vii. optionally a linker; and
viii. an IL-15 polypeptide comprising or consisting of a sequence having at least 90%, at least 91%, at least 92%, at least 93%, at least 94%, at least 95%, at least 95%, at least 97%, at least 98%, at least 99% or 100% identity to a sequence selected from the group consisting of SEQ ID NOs: 148 to 155;
and the immunoconjugate comprises or consists of first and second copies of said first and second polypeptides;
or
e) a first polypeptide chain comprising, in an N- to C-terminal direction:
iii. a heavy chain variable region (VH) of an anti-NKp46 antibody comprising an HCDR1 comprising or consisting of SEQ ID NO: 13 and optionally having up to 2 amino acid substitutions thereto, an HCDR2 comprising or consisting of SEQ ID NO: 26 and optionally having up to 2 amino acid substitutions thereto, and an HCDR3 comprising or consisting of SEQ ID NO: 39 and optionally having up to 2 amino acid substitutions thereto;
iv. a heavy chain constant region comprising or consisting of a sequence having at least 90%, at least 91%, at least 92%, at least 93%, at least 94%, at least 95%, at least 95%, at least 97%, at least 98%, at least 99% or 100% identity to a sequence selected from the group consisting of SEQ ID NO: 105 to 106; and
f) a second polypeptide chain comprising, in an N- to C-terminal direction:
v. a light chain variable region (VL) of an anti-NKp46 antibody comprising an LCDR1 comprising or consisting of SEQ ID NO: 52 and optionally having up to 2 amino acid substitutions thereto, an LCDR2 comprising or consisting of SEQ ID NO: 65 and optionally having up to 2 amino acid substitutions thereto, and an LCDR3 comprising or consisting of SEQ ID NO: 78 and optionally having up to 2 amino acid substitutions thereto; and
vi. a light chain constant region comprising or consisting of a sequence having at least 90%, at least 91%, at least 92%, at least 93%, at least 94%, at least 95%, at least 95%, at least 97%, at least 98%, at least 99% or 100% identity to SEQ ID NO: 107;
vii. optionally a linker; and
viii. an IL-15 polypeptide comprising or consisting of a sequence having at least 90%, at least 91%, at least 92%, at least 93%, at least 94%, at least 95%, at least 95%, at least 97%, at least 98%, at least 99% or 100% identity to a sequence selected from the group consisting of SEQ ID NOs: 148 to 155, 206 and 207;
and the immunoconjugate comprises or consists of first and second copies of said first and second polypeptides.

31. The immunoconjugate of any one of claims 18 to 30, wherein the IL-15 polypeptide is an IL-15 polypeptide as defined in any one of claims 8 to 17.

32. The immunoconjugate of any preceding claim, wherein the immunoconjugate comprises:
a) a first polypeptide chain comprising or consisting of the sequence of SEQ ID NO: 156; and a second polypeptide chain comprising or consisting of the sequence of SEQ ID NO: 135;
b) a first polypeptide chain comprising or consisting of the sequence of SEQ ID NO: 157; and a second polypeptide chain comprising or consisting of the sequence of SEQ ID NO: 136;
c) a first polypeptide chain comprising or consisting of the sequence of SEQ ID NO: 158; and a second polypeptide chain comprising or consisting of the sequence of SEQ ID NO: 137;
d) a first polypeptide chain comprising or consisting of the sequence of SEQ ID NO: 159; and a second polypeptide chain comprising or consisting of the sequence of SEQ ID NO: 138;
e) a first polypeptide chain comprising or consisting of the sequence of SEQ ID NO: 160; and a second polypeptide chain comprising or consisting of the sequence of SEQ ID NO: 139;
f) a first polypeptide chain comprising or consisting of the sequence of SEQ ID NO: 161; and a second polypeptide chain comprising or consisting of the sequence of SEQ ID NO: 140;
g) a first polypeptide chain comprising or consisting of the sequence of SEQ ID NO: 162; and a second polypeptide chain comprising or consisting of the sequence of SEQ ID NO: 141;
h) a first polypeptide chain comprising or consisting of the sequence of SEQ ID NO: 163; and a second polypeptide chain comprising or consisting of the sequence of SEQ ID NO: 142;
i) a first polypeptide chain comprising or consisting of the sequence of SEQ ID NO: 164; and a second polypeptide chain comprising or consisting of the sequence of SEQ ID NO: 143;
j) a first polypeptide chain comprising or consisting of the sequence of SEQ ID NO: 165; and a second polypeptide chain comprising or consisting of the sequence of SEQ ID NO: 144;
k) a first polypeptide chain comprising or consisting of the sequence of SEQ ID NO: 166; and a second polypeptide chain comprising or consisting of the sequence of SEQ ID NO: 145;
l) a first polypeptide chain comprising or consisting of the sequence of SEQ ID NO: 167; and a second polypeptide chain comprising or consisting of the sequence of SEQ ID NO: 146
m) a first polypeptide chain comprising or consisting of the sequence of SEQ ID NO: 168; and a second polypeptide chain comprising or consisting of the sequence of SEQ ID NO: 147;
n) a first polypeptide chain comprising or consisting of the sequence of SEQ ID NO: 109; and a second polypeptide chain comprising or consisting of the sequence of SEQ ID NO: 169;
o) a first polypeptide chain comprising or consisting of the sequence of SEQ ID NO: 110; and a second polypeptide chain comprising or consisting of the sequence of SEQ ID NO: 170;
p) a first polypeptide chain comprising or consisting of the sequence of SEQ ID NO: 111; and a second polypeptide chain comprising or consisting of the sequence of SEQ ID NO: 171
q) a first polypeptide chain comprising or consisting of the sequence of SEQ ID NO: 112; and a second polypeptide chain comprising or consisting of the sequence of SEQ ID NO: 172;
r) a first polypeptide chain comprising or consisting of the sequence of SEQ ID NO: 113; and a second polypeptide chain comprising or consisting of the sequence of SEQ ID NO: 173;
s) a first polypeptide chain comprising or consisting of the sequence of SEQ ID NO: 114; and a second polypeptide chain comprising or consisting of the sequence of SEQ ID NO: 174;
t) a first polypeptide chain comprising or consisting of the sequence of SEQ ID NO: 115; and a second polypeptide chain comprising or consisting of the sequence of SEQ ID NO: 175;
u) a first polypeptide chain comprising or consisting of the sequence of SEQ ID NO: 116; and a second polypeptide chain comprising or consisting of the sequence of SEQ ID NO: 176;
v) a first polypeptide chain comprising or consisting of the sequence of SEQ ID NO: 117; and a second polypeptide chain comprising or consisting of the sequence of SEQ ID NO: 177;
w) a first polypeptide chain comprising or consisting of the sequence of SEQ ID NO: 118; and a second polypeptide chain comprising or consisting of the sequence of SEQ ID NO: 178;
x) a first polypeptide chain comprising or consisting of the sequence of SEQ ID NO: 119; and a second polypeptide chain comprising or consisting of the sequence of SEQ ID NO: 179;
y) a first polypeptide chain comprising or consisting of the sequence of SEQ ID NO: 120; and a second polypeptide chain comprising or consisting of the sequence of SEQ ID NO: 180;
z) a first polypeptide chain comprising or consisting of the sequence of SEQ ID NO: 121; and a second polypeptide chain comprising or consisting of the sequence of SEQ ID NO: 181;
aa) a first polypeptide chain comprising or consisting of the sequence of SEQ ID NO: 122; and a second polypeptide chain comprising or consisting of the sequence of SEQ ID NO: 169;
bb) a first polypeptide chain comprising or consisting of the sequence of SEQ ID NO: 123; and a second polypeptide chain comprising or consisting of the sequence of SEQ ID NO: 170;
cc) a first polypeptide chain comprising or consisting of the sequence of SEQ ID NO: 124; and a second polypeptide chain comprising or consisting of the sequence of SEQ ID NO: 171;
dd) a first polypeptide chain comprising or consisting of the sequence of SEQ ID NO: 125; and a second polypeptide chain comprising or consisting of the sequence of SEQ ID NO: 172;
ee) a first polypeptide chain comprising or consisting of the sequence of SEQ ID NO: 126; and a second polypeptide chain comprising or consisting of the sequence of SEQ ID NO: 173;
ff) a first polypeptide chain comprising or consisting of the sequence of SEQ ID NO: 127; and a second polypeptide chain comprising or consisting of the sequence of SEQ ID NO: 174;
gg) a first polypeptide chain comprising or consisting of the sequence of SEQ ID NO: 128; and a second polypeptide chain comprising or consisting of the sequence of SEQ ID NO: 175;
hh) a first polypeptide chain comprising or consisting of the sequence of SEQ ID NO: 129; and a second polypeptide chain comprising or consisting of the sequence of SEQ ID NO: 176;
ii) a first polypeptide chain comprising or consisting of the sequence of SEQ ID NO: 130; and a second polypeptide chain comprising or consisting of the sequence of SEQ ID NO: 177;
jj) a first polypeptide chain comprising or consisting of the sequence of SEQ ID NO: 131;
and a second polypeptide chain comprising or consisting of the sequence of SEQ ID NO: 178;
kk) a first polypeptide chain comprising or consisting of the sequence of SEQ ID NO: 132; and a second polypeptide chain comprising or consisting of the sequence of SEQ ID NO: 179;
ll) a first polypeptide chain comprising or consisting of the sequence of SEQ ID NO: 133; and a second polypeptide chain comprising or consisting of the sequence of SEQ ID NO: 180;
mm) a first polypeptide chain comprising or consisting of the sequence of SEQ ID NO: 134; and a second polypeptide chain comprising or consisting of the sequence of SEQ ID NO: 181;
nn) a first polypeptide chain comprising or consisting of the sequence of SEQ ID NO: 188; and a second polypeptide chain comprising or consisting of the sequence of SEQ ID NO: 135;
oo) a first polypeptide chain comprising or consisting of the sequence of SEQ ID NO: 189; and a second polypeptide chain comprising or consisting of the sequence of SEQ ID NO: 140;
pp) a first polypeptide chain comprising or consisting of the sequence of SEQ ID NO: 191; and a second polypeptide chain comprising or consisting of the sequence of SEQ ID NO: 140;
qq) a first polypeptide chain comprising or consisting of the sequence of SEQ ID NO: 192; and a second polypeptide chain comprising or consisting of the sequence of SEQ ID NO: 140;
rr) a first polypeptide chain comprising or consisting of the sequence of SEQ ID NO: 195; and a second polypeptide chain comprising or consisting of the sequence of SEQ ID NO: 140;
ss) a first polypeptide chain comprising or consisting of the sequence of SEQ ID NO: 200; and a second polypeptide chain comprising or consisting of the sequence of SEQ ID NO: 140;
tt) a first polypeptide chain comprising or consisting of the sequence of SEQ ID NO: 190; and a second polypeptide chain comprising or consisting of the sequence of SEQ ID NO: 140; or
uu) a first polypeptide chain comprising or consisting of the sequence of SEQ ID NO: 114; and a second polypeptide chain comprising or consisting of the sequence of SEQ ID NO: 199;
and the immunoconjugate comprises or consists of first and second copies of said first and second polypeptides

33. The immunoconjugate of claim 1, wherein the immunoconjugate comprises a polypeptide chain comprising (optionally in an N to C terminal direction):
a) an IL-15 polypeptide;
b) optionally a linker;
c) a heavy chain constant region or fragment thereof,
d) optionally a linker;
e) an antibody or antigen-binding fragment thereof comprising at least one antibody binding domain that specifically binds to an antigen selected from the group consisting of NKG2D, NKp3D, NKp46, NKp44 and DNAM-1;
and the immunoconjugate comprises or consists of first and second copies of said polypeptide chain.

34. The immunoconjugate of claim 33, wherein the immunoconjugate comprises a polypeptide chain comprising (optionally in an N to C terminal direction):
a) an IL-15 polypeptide comprising or consisting of the sequence of SEQ ID NO: 154;
b) optionally a linker;
c) a heavy chain constant region fragment comprising a hinge, a CH2 and a CH3 region, and comprising or consisting of the sequence of SEQ ID NO: 208;
d) optionally a linker;
e) a scFv domain that specifically binds to NKG2D, comprising or consisting of a sequence selected from the group consisting of SEQ ID NO: 211 to 220 and 224 to 233;
and the immunoconjugate comprises or consists of first and second copies of said polypeptide chain.

35. The immunoconjugate of claim 34, wherein the immunoconjugate comprises a polypeptide chain comprising or consisting of
a) the sequence of SEQ ID NO: 242 or 255; or
b) the sequence of SEQ ID NO: 237 or 250
and the immunoconjugate comprises or consists of first and second copies of said polypeptide chain.

36. The immunoconjugate of any preceding claim, wherein the immunoconjugates are homodimeric.

37. An immunoconjugate, comprising:
a) at least two IL-15 polypeptides, each comprising
i. an IL-15 cytokine; and
ii. IL-15 receptor alpha (CD215) or a functional fragment thereof; and
b) a multivalent antibody or antigen-binding fragment thereof, comprising at least two antibody binding domains, wherein the multivalent antibody or antigen-binding fragment thereof specifically binds to NKG2D.

38. The immunoconjugate of any one of claims 1 to 37 for use in treating cancer.
